(19) United States
(12) Patent Application Publication
(10) Pub. No.: US 2008/0319375 A1
(43) Pub. Date: Dec. 25, 2008

(54) Materials, Methods, and Systems for Cavitation-mediated Ultrasonic Drug Delivery in vivo

(75) Inventor: Charles Thomas Hardy, Foster City, CA (US)

Correspondence Address: Biovaluation & Analysis, Inc., 509 Jibstay Lane, Foster City, CA, 94404, US

(73) Assignee: Biovaluation & Analysis, Inc.

(21) Appl. No.: 12/135,130

(22) Filed: Jun. 6, 2008

Related U.S. Application Data
(60) Provisional application No. 60/943,603, filed on Jun. 13, 2007.
(60) Provisional application No. 60/943,589, filed on Jun. 13, 2007.
(60) Provisional application No. 60/943,584, filed on Jun. 13, 2007.
(60) Provisional application No. 60/943,574, filed on Jun. 13, 2007.
(60) Provisional application No. 60/942,453, filed on Jun. 6, 2007.
(60) Provisional application No. 60/942,451, filed on Jun. 6, 2007.
(60) Provisional application No. 60/942,447, filed on Jun. 6, 2007.
(60) Provisional application No. 60/942,443, filed on Jun. 6, 2007.
(60) Provisional application No. 60/942,438, filed on Jun. 6, 2007.

(51) Int. Cl. A61M 37/00; A61B 8/00

(52) U.S. Cl. 604/22; 604/507; 600/431

(57) ABSTRACT

Materials, methods, and systems for targeted and non-targeted therapeutic delivery in vivo utilizing cavitation-mediated ultrasonic drug delivery are described. Noninvasive sonic energy being applied to the patient in a controlled fashion at the treatment area results in controlled acoustic cavitation at said region, and cell and tissue specific drug delivery. Microbubbles, both in the form of contrast agents, and/or other active agents infused into the patient, and/or bubbles formed from previous ultrasound exposure, allow for predictable cavitation thresholds, requiring much lower incident ultrasound intensities for permeating tissue. Further, methods and systems are provided that result in more spatially regular areas of controlled tissue permeability upon treatment, limiting cytotoxicity and sonolysis, and maximizing intracellular drug delivery. Moreover, by using pulsed cavitation-mediated ultrasonic drug delivery as described by the present teachings, a large number of parameters are created, which provided the appropriate monitoring and feedback mechanisms are present, allow the use of a diversity of parameter optimizations and control systems for customizing the methods and systems for a given application. Preferred therapeutics for use with the present invention include nucleic acids, proteins, peptides, and other therapeutic macromolecules.

### CROSS-REFERENCES

The present application claims the benefit of my Provisional Application No. 60/942,438, Biodegradable Triblock Copolymers, and Mixtures of the Same, for Acoustically Mediated Intracellular Drug Delivery in vivo, filed on Jun. 6, 2007; and the benefit of my Provisional Application No. 60/942,443, Dendritic and Branched Chain Polymers, and Mixtures of the Same, for Acoustically Mediated Intracellular Drug Delivery in vivo, filed on Jun. 6, 2007, now abandoned; and the benefit of my Provisional Application No. 60/942,447, Methods and Systems for Pulsed Cavitation-mediated Ultrasonic Drug Delivery, filed on Jun. 6, 2007; and the benefit of my Provisional Application No. 60/942,451, Polymersomes, Peptosomes, and Mixtures of the Same, for Acoustically Mediated Intracellular Drug Delivery in vivo, filed on Jun. 6, 2007; and the benefit of my Provisional Application No. 60/942,447, Supramolecular Assemblies, and Mixtures of the Same, for Acoustically Mediated Intracellular Drug Delivery in vivo, filed on Jun. 6, 2007; and the benefit of my Provisional Application No. 60/943,574, Methods and Systems for Utilizing Biodegradable Triblock Copolymers in Cavitation-mediated Ultrasonic Drug Delivery, filed on Jun. 13, 2007, now abandoned; and the benefit of my Provisional Application No. 60/943,584, Methods and Systems for Utilizing Dendritic and Branched Chain Polymers in Pulsed Cavitation-mediated Ultrasonic Drug Delivery, filed on Jun. 13, 2007; and the benefit of my Provisional Application No. 60/943,589, Methods and Systems for Utilizing Polymersomes and Peptosomes in Pulsed Cavitation-mediated Ultrasonic Drug Delivery, filed on Jun. 13, 2007; and the benefit of my Provisional Application No. 60/943,603, Methods and Systems for Utilizing Supramolecular Assemblies in Pulsed Cavitation-mediated Ultrasonic Drug Delivery, filed on Jun. 13, 2007; and the benefit of my U.S. patent application Ser. No. 12/131,097, Dendritic Polymers for use in acoustically Mediated Intracellular Drug Delivery in vivo, filed on Jun. 1, 2008; and the benefit of my U.S. patent application Ser. No. 12/131,101, Polymersomes for Use in Acoustically Mediated Intracellular Drug Delivery in vivo, filed on Jun. 1, 2008; and the benefit of my U.S. patent application Ser. No. 12/131,105, Peptosomes for Use in Acoustically Mediated Intracellular Drug Delivery in vivo, filed on Jun. 1, 2008; and the benefit of my U.S. patent application Ser. No. 12/131,109, Supramolecular Assemblies for Use in Acoustically Mediated Intracellular Drug Delivery in vivo, filed on Jun. 1, 2008; and the benefit of my U.S. patent application Ser. No. 12/133,631, Biodegradable Triblock Polymers for Use in acoustically Mediated Intracellular Drug Delivery in vivo, filed on Jun. 5, 2008; where I am the sole inventor on all applications. All of the aforementioned specifications (i.e., applications) are incorporated herein by reference in their entirety for all purposes.

### REFERENCES CITED

U.S. PATENT DOCUMENTS

| | | |
|---|---|---|
| Patent | Issue Date | Inventor |

| | | |
|---|---|---|
| 4,084,582 | April 1978 | Nigam |
| 4,162,282 | July 1979 | Fulwyler et al. |
| 4,207,901 | June 1980 | Nigam |
| 4,223,560 | September 1980 | Glenn |
| 4,227,417 | October 1980 | Glenn |
| 4,248,090 | February 1981 | Glenn |
| 4,257,271 | March 1981 | Glenn |
| 4,162,282 | July 1979 | Fulwyler et al. |
| 4,310,505 | January 1982 | Baldeschwieler et al. |
| 4,317,370 | March 1982 | Glenn |
| 4,325,381 | April 1982 | Glenn |
| 4,410,688 | October 1983 | Denkewalter et al. |
| 4,507,466 | March 1985 | Tomalia et al. |
| 4,533,254 | August 1985 | Cook et al. |
| 4,558,120 | December 1985 | Tomalia et al. |
| 4,568,737 | February 1986 | Tomalia et al. |
| 4,586,512 | May 1986 | Do-huu et al. |
| 4,587,329 | May 1986 | Tomalia et al. |
| 4,599,892 | July 1986 | Doshi |
| 4,620,546 | November 1986 | Aida et al. |
| 4,658,828 | April 1986 | Dory |
| 4,631,337 | December 1986 | Tomalia et al. |
| 4,664,121 | May 1987 | Sanghvi et al. |
| 4,694,064 | September 1987 | Tomalia et al. |
| 4,713,975 | December 1987 | Tomalia et al. |
| 4,728,575 | March 1988 | Gamble et al. |
| 4,728,578 | March 1988 | Higgins et al. |
| 4,737,323 | April 1988 | Martin et al. |
| 4,737,550 | April 1988 | Tomalia |
| 4,770,184 | September 1988 | Greene et al. |
| 4,857,599 | August 1989 | Tomalia et al. |
| 4,858,613 | August 1989 | Fry et al. |
| 4,871,779 | October 1989 | Killat et al. |
| 4,921,706 | May 1990 | Roberts et al. |
| 4,951,653 | August 1990 | Fry et al. |
| 4,955,365 | September 1990 | Fry et al. |
| 5,036,855 | August 1991 | Fry et al. |
| 5,041,516 | August 1991 | Frechet et al. |
| 5,054,470 | October 1991 | Fry et al. |
| 5,080,102 | January 1992 | Dory |
| 5,117,832 | June 1992 | Sanghvi et al. |
| 5,120,657 | June 1992 | McCabe et al. |
| 5,128,326 | July 1992 | Balazs et al. |
| 5,149,319 | September 1992 | Unger |
| 5,172,597 | December 1992 | Hedeen |
| 5,196,343 | March 1993 | Zerhouni et al. |
| 5,210,309 | May 1993 | Newkome et al. |
| 5,215,680 | June 1993 | D'Arrigo |
| 5,216,115 | June 1993 | Kohn et al. |
| 5,219,401 | June 1993 | Cathignol et al. |
| 5,247,935 | September 1993 | Cline et al. |
| 5,295,484 | March 1994 | Marcus et al. |
| 5,312,617 | May 1994 | Unger et al. |
| 5,316,000 | May 1994 | Chapelon |
| 5,338,532 | August 1994 | Tomalia et al. |
| 5,376,690 | December 1994 | Newkome et al. |
| 5,391,197 | February 1995 | Burdette et al. |
| 5,409,006 | April 1995 | Buchholtz et al. |
| 5,418,301 | May 1995 | Hult et al. |
| 5,443,069 | August 1995 | Schaetzle |
| 5,470,350 | November 1995 | Buchholtz et al. |
| 5,492,126 | February 1996 | Hennige et al. |
| 5,514,764 | May 1996 | Frechet et al. |
| 5,523,058 | June 1996 | Umemura et al. |
| 5,527,524 | June 1996 | Tomalia et al. |
| 5,573,497 | November 1996 | Chapelon |
| 5,580,960 | December 1996 | Burgeson et al. |
| 5,587,507 | December 1996 | Kohn et al. |
| 5,601,526 | February 1997 | Chapelon et al. |
| 5,610,031 | March 1997 | Burgeson et al. |
| 5,620,479 | April 1997 | Diederich |
| 5,625,040 | April 1997 | Margolis et al. |
| 5,630,837 | May 1997 | Crowley |
| 5,643,179 | July 1997 | Fujimoto |
| 5,648,465 | July 1997 | Margolis et al. |
| 5,670,602 | September 1997 | Kohn et al. |
| 5,676,692 | October 1997 | Sanghvi et al. |
| 5,728,379 | March 1998 | Martuza et al. |
| 5,762,066 | June 1998 | Law et al. |
| 5,766,922 | June 1998 | Peles |
| 5,769,790 | June 1998 | Watkins et al. |
| 5,770,222 | June 1998 | Unger et al. |
| 5,770,565 | June 1998 | Cheng et al. |
| 5,792,743 | August 1998 | Schachner |
| 5,836,905 | November 1998 | Lemelson et al. |
| 5,840,031 | November 1998 | Crowley |
| 5,849,865 | December 1998 | Cheng et al. |
| 5,866,165 | February 1999 | Liu et al. |
| 5,872,231 | February 1999 | Engvall et al. |
| 5,935,553 | August 1999 | Unger et al. |
| 6,020,457 | February 2000 | Klimash et al. |
| 6,063,629 | May 2000 | Knoblauch |
| 6,071,495 | June 2000 | Unger et al. |
| 6,113,946 | September 2000 | Szoka et al. |
| 6,121,231 | September 2000 | Petit et al. |
| 6,139,819 | October 2000 | Unger et al. |
| 6,140,117 | October 2000 | Milbrandt et al. |
| 6,146,657 | November 2000 | Unger et al. |
| 6,159,467 | December 2000 | Chung et al. |
| 6,169,078 | January 2001 | Hughes et al. |
| 6,194,543 | February 2001 | Florence et al. |
| 6,200,547 | March 2001 | Volkonsky et al. |
| 6,204,054 | March 2001 | Sutton et al. |
| 6,231,834 | May 2001 | Unger et al. |
| 6,267,987 | July 2001 | Park et al. |
| 6,319,703 | November 2001 | Speck |
| 6,330,300 | December 2001 | Siochi |
| 6,331,658 | December 2001 | Cooper et al. |
| 6,335,011 | January 2002 | Podsakoff et al. |
| 6,344,446 | February 2002 | Gorman et al. |
| 6,352,972 | March 2002 | Nimni et al. |
| 6,372,720 | April 2002 | Longmuir et al. |
| 6,383,500 | May 2002 | Wooley et al. |
| 6,399,663 | June 2002 | Haces et al. |
| 6,403,056 | June 2002 | Unger |
| 6,416,740 | July 2002 | Unger |
| 6,428,477 | August 2002 | Mason |
| 6,436,709 | August 2002 | Lin et al. |
| 6,437,946 | August 2002 | Todd |
| 6,457,365 | October 2002 | Stephens |
| 6,461,303 | October 2002 | Angelsen |
| 6,461,641 | October 2002 | Fick |
| 6,475,577 | November 2002 | Hopton et al. |
| 6,482,410 | November 2002 | Crossin |
| 6,482,436 | November 2002 | Volkonsky et al. |
| 6,488,615 | December 2002 | Mitchiner et al. |
| 6,514,762 | February 2003 | Wang |
| 6,515,402 | February 2003 | Klee |
| 6,548,740 | April 2003 | Bremel et al. |
| 6,569,528 | May 2003 | Nam et al. |
| 6,585,956 | July 2003 | Malik et al. |
| 6,589,180 | July 2003 | Erikson et al. |
| 6,593,130 | July 2003 | Sen et al. |
| 6,603,998 | August 2003 | King et al. |
| 6,610,290 | August 2003 | Podsakoff et al. |
| 6,616,944 | September 2003 | Kissel et al. |
| 6,626,855 | September 2003 | Weng et al. |
| 6,632,671 | October 2003 | Unger |
| 6,641,534 | November 2003 | Smith |
| 6,649,702 | November 2003 | Rapoport et al. |
| 6,663,555 | December 2003 | Mitchiner et al. |
| 6,685,640 | February 2004 | Fry et al. |
| 6,716,882 | April 2004 | Haces et al. |
| 6,746,860 | June 2004 | Tokusumi et al. |
| 6,773,696 | August 2004 | Unger |
| 6,780,153 | August 2004 | Angelsen et al. |
| 6,780,157 | August 2004 | Stephens et al. |
| 6,780,846 | August 2004 | O'Mahony et al. |
| 6,782,287 | August 2004 | Grzeszczuk et al. |
| 6,790,437 | September 2004 | Malik et al. |
| 6,822,929 | November 2004 | Schubert et al. |
| 6,872,406 | March 2005 | Qi |
| 6,878,541 | April 2005 | Qiao et al. |
| 6,945,937 | September 2005 | Culp et al. |
| 6,972,510 | December 2005 | Klee et al. |
| 6,978,172 | December 2005 | Mori et al. |
| 6,985,430 | January 2006 | Villiard et al. |
| 6,998,107 | February 2006 | Unger |
| 7,001,614 | February 2006 | Smyth-Templeton et al. |
| 7,005,124 | February 2006 | Malik et al. |
| 7,041,058 | May 2006 | Piehler |
| 7,081,495 | July 2006 | Florence et al. |
| 7,083,572 | August 2006 | Unger et al. |
| 7,087,729 | August 2006 | Prive |
| 7,089,053 | August 2006 | Tokudome et al. |
| 7,101,937 | September 2006 | Frechet et al. |
| 7,115,380 | October 2006 | Allinquant |
| 7,123,450 | October 2006 | Villiard et al. |
| 7,125,387 | October 2006 | Kawabata et al. |
| 7,127,284 | October 2006 | Seward |
| 7,138,562 | November 2006 | Bremel et al. |
| 7,153,905 | December 2006 | Banerjee et al. |
| 7,154,991 | December 2006 | Earnst et al. |
| 7,195,780 | March 2007 | Dennis et al. |
| 7,212,608 | May 2007 | Nagamine et al. |
| 7,212,609 | May 2007 | Nagamine et al. |
| 7,226,417 | June 2007 | Eberle et al. |
| 7,241,617 | July 2007 | Fukumura et al. |
| 7,260,426 | August 2007 | Schweikard et al. |
| 7,268,214 | September 2007 | O'Mahony et al. |
| 7,288,266 | October 2007 | Smyth-Templeton et al. |
| 7,297,838 | November 2007 | Chen et al. |
| 7,309,757 | December 2007 | Liu et al. |
| 7,314,614 | January 2008 | Yonemitsu et al. |
| 7,327,865 | February 2008 | Fu et al. |
| 7,345,138 | March 2008 | Wang et al. |
| 7,364,007 | April 2008 | Garcia-Osuna et al. |

### REFERENCES CITED

U.S. PATENT APPLICATIONS

| Application | Submission Date | Inventor |
|---|---|---|
| 10/881,315 | Jun. 30, 2004 | Saracen et al. |
| 11/070,371 | Mar. 2, 2005 | Seip et al. |
| 11/523,201 | Sep. 19, 2006 | Cain et al. |
| 11/890,881 | Aug. 7, 2007 | Thomson et al. |

### REFERENCES CITED

### Other References

Abragam, A., Principles of Nuclear Magnetism (International Series of Monographs on Physics), University Press, 1983, Oxford.
AIUM, “Mechanical bioeffects from diagnostic ultrasound: AIUM consensus statements,” J. Ultrasound Med., 2000, 19, 68-168.
Akhtar et al., “Nonviral Delivery of Synthetic siRNAs in vivo,” J. Clin. Invest., 2007, 117 (12), 3623-3632.
Al-Jamal et al., “Supramolecular Structures from Dendrons and Dendrimers,” Adv. Drug Deliv. Rev., 2005, 57 (15), 2238.
Apfel, R., Acoustic Cavitation, Methods of Experimental Physics Series, Vol. 356, Edmonds, P., Academic Press, 1981a, New York, N.Y.
Apfel, R., “Acoustic Cavitation Prediction,” J. Acoust. Soc. Am., 1981b, 69, 1624-1633.
Apfel, R., “Acoustic Cavitation: A Possible Consequence of Biomedical Uses of Ultrasound,” Br. J. Cancer, 1982, 45 (Suppl.), 140-146.
Apfel, R., “Possibility of Microcavitation from Diagnostic Ultrasound,” Ultrasonics Ferroelectrics Frequency Control, 1986, 33, 139-142.
Apfel et al., “Gauging the Likelihood of Cavitation from Short-pulse, Low-duty Cycle Diagnostic Ultrasound,” Ultrasound Med. Biol., 1991, 17 (2), 179-85.
Arnold, W., Acoustical Imaging, Kluwer Academic Publishers, 2004, Dordrecht, Boston.
Atchley et al., “Thresholds for Cavitation Produced in Water by Pulsed Ultrasound,” Ultrasonics, 1988, 26 (5), 280-285.
Baban et al., “Control of Tumour Vascular Permeability,” Adv. Drug Deliv. Rev., 1998, 34 (1), 109-119.
Bamber et al., “Ultrasonic Attenuation and Propagation Speed in Mammalian Tissues as a Function of Temperature,” Ultrasound Med. Biol., 1979, 5 (2), 149.
Bernard et al., “Water-Soluble Dendrigrafts Bearing Saccharidic Moieties: Elaboration and Application to Enzyme Linked Oligosorbent Assay (Elosa) Diagnostic Tests,” Bioconjug Chem., 2006, 17 (1), 6.
Bishop, P., “The Biochemical Structure of Mammalian Vitreous,” Eye, 1996, 10, 664-670.
Blackstock, D., Fundamentals of Physical Acoustics, Wiley, 2000, New York, N.Y.
Bland et al., Ultrathin Magnetic Structures I: An Introduction to the Electronic Magnetic and Structural Properties, Springer, 1994, Berlin, Germany; New York, N.Y.
Brayman et al., “Transient Poration and Cell Surface Receptor Removal from Human Lymphocytes in vitro by 1-MHz Ultrasound,” Ultrasound Med. Biol., 1999, 25, 999-1008.
Brennen, C., Cavitation and Bubble Dynamics, Oxford University Press, 1995, New York.
Burov, A., “High-Intensity Ultrasonic Vibrations for Action on Animal and Human Malignant Tumours,” Dokl. Akad. Nauk. SSSR, 1956, 106, 239-241.
Capecci, M., “High Efficiency Transformation by Direct Microinjection of DNA into Cultured Mammalian Cells,” Cell 1980, 22 (2 Pt 2), 479-88.
Carson et al., “Ultrasonic Power and Intensities Produced by Diagnostic Ultrasound Equipment,” Ultrasound Med. Biol., 1978, 3 (4), 341.
Chavrier et al., “Modeling of High-intensity Focused Ultrasound-induced Lesions in the Presence of Cavitation Bubbles,” J. Acoust. Soc. Am., 2000, 108, 432-440.
Chen et al., “Magnetically Responsive Polymerized Liposomes as Potential Oral Delivery Vehicles,” Pharm. Res., 1997, 14 (4), 537.
Chen et al., “The Disappearance of Ultrasound Contrast Bubbles: Observations of Bubble Dissolution and Cavitation Nucleation,” Ultrasound Med. Biol., 2002, 28 (6), 793-803.
Coakley et al., Cavitation; Dynamics of Gas Bubbles; Applications, In: Ultrasound: Its Applications in Medicine and Biology, Fry, F., Elsevier, 1978, New York, N.Y., pp. 77-160.
Cochran, et al., “Sonoluminescence as an Indicator of Cell Membrane Disruption by Acoustic Cavitation,” Ultrasound. Med. Biol., 2001, 27 (6), 841-850.
Cohen et al., Microparticulate Systems for the Delivery of Proteins and Vaccines, Marcel Dekker, 1996, New York.
Colowick et al., Drug and Enzyme Targeting, Parts: Volume 112: Drug and Enzyme Targeting (Methods in Enzymology), Academic Press, 2006, New York.
Craik, D., Magnetism: Principles and Applications, Wiley, 1995, Chichester, N.Y.
Dalecki et al., “Hemolysis in vivo from Exposure to Pulsed Ultrasound,” Ultrasound Med. Biol., 1997, 23, 307-313.
Duck et al., Ultrasound in Medicine, Bristol, Institute of Physics Publications, 1998, Philadelphia, Pa.
Duvvuri et al., “Drug Delivery to the Fetina: Challenges and Opportunities,” Expert. Opin. Biol. Ther., 2003, 3, 45-56.
Fellinger et al., “Klinik und Therapies des Chemischen Gelenkreumatismus,” Maudrich Vienna, Austria, 1954, 549-552.
Flynn, H., “Cavitation Dynamics, I: A Mathematical Formulation,” J. Acoust. Soc. Am., 1975a, 57, 1379-1396.
Flynn, H., “Cavitation Dynamics, II: Free Pulsations and Models for Cavitation Bubbles,” J. Acoust. Soc. Am., 1975b, 58, 1160-1170.
Fouassier, J., Photoinitiation, Photopolymerization, and Photocuring: Fundamentals and Applications, distributed by Hanser/Gardner Publications, Hanser, 1995, Munich, Germany; New York, N.Y.; Cincinnati, Ohio.
Golemis et al., Protein-Protein Interactions: A Molecular Cloning Manual, Cold Spring Harbor Laboratory Press, 2005, Cold Spring Harbor, N.Y.
Greenleaf et al., “Artificial Cavitation Nuclei Significantly Enhance Acoustically induced Cell Transfection,” Ultrasound Med. Biol., 1998, 24 (4), 587-95.
Guzman et al., “Ultrasound-mediated Disruption of Cell Membranes. I. Quantification of Molecular Uptake and Cell Viability,” J. Acoust. Soc. Am., 2001a, 110 (1), 588-596.
Guzman et al., “Ultrasound-mediated Disruption of Cell Membranes. II. Heterogeneous Effects on Cells,” J. Acoust. Soc. Am., 2001b, 110 (1), 597-606.
Guzman et al., “Equilibrium Loading of Cells with Macromolecules by Ultrasound: Effects of Molecular Size and Acoustic Energy,” J, Pharm. Sci., 2002, 91 (7), 1693-701.
Guzman et al., “Bioeffects Caused by Changes in Acoustic Cavitation Bubble Density and Cell Concentration: A Unified Explanation Based on Cell-to-Bubble Ratio and Blast Radius,” Ultrasound Med. Biol., 2003, 29 (8), 1211-1222.
Harlow et al., Using Antibodies: A Laboratory Manual, Cold Spring Harbor Laboratory Press, 1999, Cold Spring Harbor, N. Y.
He et al., “Studies on Pharmacologic Disposition of Adriamycin in Dog after Hepatic Arterial Embolization with Adriamycin Carboxymethyl-Dextran-Microspheres,” Yao Xue Xue Bao, 1993, 28 (11), 859.
Hermanson, G., Bioconjugate Techniques, Academic Press, 1996, San Diego, Calif.
Hilgenfeldt et al., “Response of Bubbles to Diagnostic Ultrasound: A Unifying Theoretical Approach,” Eur. Phys. J. Acoust. Soc. Am., 1998, B4, 247-255.
Hilgenfeldt et al., “Sound Scattering and Localized Heat Deposition of Pulse-driven Microbubbles,” J. Acoust. Soc. Am., 2000, 107 (6), 3530-3539.
Hille, B., Ion Channels of Excitable Membranes, Sinauer Associates, Inc., 2001, Sunderland, Mass.
Hoff, L., Acoustic Characterization of Contrast Agents for Medical Ultrasound Imaging, Kluwer Academic Publishers, 2001, Dordrecht, Netherlands.
Holt et al., “Measurements of Bubble-enhanced Heating from Focused, MHz-frequency Ultrasound in a Tissue-mimicking Material,” Ultrasound Med. Biol., 2002, 27 (10), 1399-1412.
Hsieh et al., “Magnetic Modulation of Release of Macromolecules from Polymers,” Proc. Natl. Acad. Sci. U.S.A., 1981, 78 (3), 1863.
Hwang et al., “Vascular effects induced by combined 1-MHz ultrasound and microbubble contrast agent treatments in vivo,” Ultrasound Med. Biol., 2005, 31, 553-564.
Hynynen et al., “Non-invasive Opening of BBB by Focused Ultrasound,” ACTA Neurochir. Suppl., 2003, 86, 555-558.
Jones et al., “Experimental Examination of a Targeted Hyperthermia System Using Inductively Heated Ferromagnetic Microspheres in Rabbit Kidney,” Phys. Med. Biol., 2001, 46 (2), 385.
Kinsler, L., Fundamentals of Acoustics, Wiley, 2000, New York, N.Y.
Kong et al., “Hyperthermia Enables Tumor-specific Nanoparticle Delivery: Effect of Particle Size,” Cancer Res., 2000, 60, 4440-4445.
Kost et al., “Magnetically Enhanced Insulin Release in Diabetic Rats,” J. Biomed. Mater. Res., 1987, 21 (12), 1367.
Lamba et al., Polyurethanes in Biomedical Applications, CRC Press, 1998, Boca Raton, Fla.
Laskey, R., “CIBA Medal Lecture, Regulatory Roles of the Nuclear Membrane,” Biochem. Soc. Trans., 1998, 26 (4), 561-567.
Lechardeur et al., “Determinants of the Nuclear Localization of the Heterodimeric DNA Fragmentation Factor (ICAD/CAD),” J. Cell Biol., 2000, 150 (2), 321-334.
Linke et al., “Localized Tissue Destruction by High-Intensity Focused Ultrasound,” Arch. Surg., 1973, 107 (6), 887.
Leighton, T., The Acoustic Bubble, Academic Press, 1994.
Luby-Phelps, K., “Cytoarchitecture and Physical Properties of Cytoplasm: Volume, Viscosity, Diffusion, Intracellular Surface Area,” Int. Rev. Cytol., 2000, 192, 189-221.
Lu et al., Cellular Drug Delivery: Principles and Practice, Humana Press, 2004, Totowa, N.J.
Lubbe et al., “Clinical Applications of Magnetic Drug Targeting,” J. Surg. Res., 2001, 95 (2), 200.
Mahato, R., Biomaterials for Delivery and Targeting of Proteins and Nucleic Acids, CRC Press, 2005, Boca Raton, Fla.
Mason et al., Applied Sonochemistry: The Uses of Power Ultrasound in Chemistry and Processing, Wiley-VCH, 2001, Weinheim.
Margulis, M., Sonochemistry and Cavitation, Gordon and Breach Publishers, 1995, Australia, [Langhorne, Pa., U.S.]
McNeil et al., “Plasma Membrane Disruption: Repair, Prevention, Adaptation,” Annu. Rev. Cell. Dev. Biol., 2003, 19, 697-731.
Medwin et al., Fundamentals of Acoustical Oceanography, Academic Press, 1998, Boston.
Miller et al., “A Review of in vitro Bioeffects of Inertial Ultrasonic Cavitation from a Mechanistic Perspective,” Ultrasound Med. Biol., 1996, 22 (9), 1131.
Miller et al., “Ultrasonically induced hemolysis at high cell and gas body concentrations in a thin-disc exposure chamber,” Ultrasound Med. Biol., 1997, 23, 625-633.
Miller et al., “Enhancement of ultrasonically-induced hemolysis by perfluorocarbon-based compared to air-based echo-contrast agents,” Ultrasound Med. Biol., 1998a, 24 (2), 285-92.
Miller et al., “Gas-body-based contrast agent enhances vascular bioeffects of 1.09 MHz ultrasound on mouse intestine,” Ultrasound Med. Biol., 1998b, 24 (8), 1201-8.
Miller et al., “Sonoporation of cultured cells in the rotating tube exposure system,” Ultrasound Med. Biol., 1999, 25 (1), 143-149.
Miller et al., “Sonoporation of monolayer cells by diagnostic ultrasound activation of contrast-agent gas bodies,” Ultrasound Med. Biol., 2000, 26 (4), 661-667.
Miller et al., Magnetism: Molecules to Materials, Wiley-VCH, 2001, Weinheim, N.Y.
Molema et al., Drug Targeting: Organ-Specific Strategies, Wiley-VCH, 2001, Weinheim, N.Y.
Moroz et al., “Targeting Liver Tumors with Hyperthermia: Ferromagnetic Embolization in a Rabbit Liver Tumor Model.” J. Surg. Oncol., 2001, 78 (1), 22.
Morton et al., “Subharmonic Emission as an Indicator of Ultrasonically-induced Biological Damage,” Ultrasound Med. Biol., 1983, 9 (6), 629-633.
Moser, W., Advanced Catalysts and Nanostructured Materials: Modern Synthetic Methods, Academic Press, 1996, San Diego, Calif.
Muzykantov et al., Biomedical Aspects of Drug Targeting, Kluwer Academic Pub., 2002, Boston, Mass.
Naugol'Nykh, K., Nonlinear Acoustics, American Institute of Physics, 1994, Woodbury, N.Y.
NCRP, Exposure Criteria for Medical Diagnostic Ultrasound, II: Criteria Based on all Known Mechanisms, National Council on Radiation Protection and Measurements, 2002, Bethesda, Md.
Neppiras, E., “Acoustic Cavitation,” Phys. Rep., 1980, 61, 160.
Neumann et al., “Gene Transfer into Mouse Lyoma Cells by Electroporation in High Electric Fields,” Embo. 1982, 1 (7), 841-845.
Nijenhuis et al., Chemical and Physical Networks: Formation and Control of Properties, Wiley, 1998, Chichester, N.Y.
Nyborg et al., “Biological Effects of Ultrasound: Development of Safety Guidelines. Part II: General Review,” Ultrasound Med. Biol., 2001, 27 (3), 301-333.
O'Brien, W., “Ultrasound-biophysics Mechanisms,” Prog. Biophys. Mol. Biol., 2007, 93, 212-255.
Ottenbrite et al., Polymeric Drugs & Drug Delivery Systems, Technomic Pub., 2001, Lancaster, Pa.
Peeters et al., “Vitreous: a Barrier to Nonviral Ocular Gene Therapy,” Invest. Opthalmol. Vis. Sci., 2005, 46, 3553-3561.
Pierce, A., Acoustics: An Introduction to its Physical Principles and Applications, Acoustical Society of America, 1989, Woodbury, N.Y.
Pitkanen et al., “Vitreous is a Barrier in Nonviral Gene Transfer by Cationic Lipids and Polymers,” Pharm. Res., 2003, 20, 576-583.
Pitt, W., “Defining the Role of Ultrasound in Drug Delivery,” Am. J. Drug Deliv., 2003, 1 (1), 27.
Pitt et al., “Ultrasonic Drug Delivery—A General Review,” Expert. Opin. Drug Deliv., 2004, 1 (1), 37.
Poliachik et al., “Effect of high-intensity focused ultrasound on whole blood with and without microbubble contrast agent,” Ultrasound Med. Biol. Eng., 1999, 25, 991-998.
Raichel, D., The Science and Applications of Acoustics, Springer, 2006, New York.
Rapoport et al., “Micellar Delivery of Doxorubicin and its Paramagnetic Analog, ruboxyl, to HL-60 Cells: Effect of Micelle Structure and Ultrasound on the Intracellular Drug Uptake,” J. Control. Release, 1999, 58 (2), 153-62.
Rapoport et al., “Drug Delivery in Polymeric Micelles: From in vitro to in vivo,” J. Control. Release, 2003, 91 (1-2), 85.
Rapoport et al., “Ultrasound-Triggered Drug Targeting of Tumors in vitro and in vivo,” Ultrasonics, 2004, 42 (1-9), 943.
Reuter et al., “Inhibition of Viral Adhesion and Infection by Sialic-Acid-Conjugated Dendritic Polymers” Bioconjug Chem., 1999, 10 (2), 271.
Rudge et al., “Preparation, Characterization, and Performance of Magnetic Iron-Carbon Composite Microparticles for Chemotherapy,” Biomaterials, 2000, 21 (14), 1411.
Ruponen et al., “Extracellular Glycosaminoglycans Modify Cellular Trafficking of Lipoplexes and Polyplexes,” J. Biol. Chem., 2001, 276, 33875-33880.
Sakthivel et al., “Synthesis and Physicochemical Properties of Lipophilic Polyamide Dendrimers,” Pharm. Res., 1998, 15 (5), 776-782.
Sambrook et al., Molecular Cloning: A Laboratory Manual, Cold Spring Harbor Laboratory Press, 2001, Cold Spring Harbor, N. Y.
Schappacher et al., “Molecular Containers Based on Amphiphilic Ps-B-Pmve Dendrigraft Copolymers: Topology, Organization, and Aqueous Solution Properties,” J. Am. Chem. Soc., 2005, 127 (9), 2990.
Schlachetzki et al., “Observation on the Integrity of the Blood-brain Barrier after Microbubble Destruction by Diagnostic Transcranial Color-coded Sonography,” J. Ultrasound Med., 2002, 21, 419-429.
Schreier, H., Drug Targeting Technology: Physical, Chemical Biological Methods, Marcel Dekker, 2001, New York.
Shah et al., Cavitation Reaction Engineering, Kluwer Academic/Plenum Publishers, 1999, New York.
Simionescu, N., “Cellular Aspects of Trans-capillary Exchange,” Physiol. Rev., 1983, 63, 1536-1579.
Singer et al., “The Fluid Mosaic Model of the Structure of Cell Membranes,” Science, 1972, 175 (23), 720.
Skyba et al., “Direct in vivo visualization of intravascular destruction of microbubbles by ultrasound and its local effects on tissue,” Circulation, 1998, 98 (4), 290-293.
Sokka et al., “MRI-guided gas bubble enhanced ultrasound heating in in vivo rabbit thigh,” Phys. Med. Biol., 48 (2), 223-241.
Suslick, K. Sonochemistry and Sonoluminescence in Encyclopedia of Physical Science and Technology, Academic Press, 2001, San Diego, Calif.
Takakura et al., “Extravasation of Macromolecules,” Adv. Drug Deliver Rev., 1998, 34, 93-108.
Talcott et al., “Getting Across the Nuclear Pore Complex,” 1999, Trends Cell. Biol., 1999, 9 (8), 312-318.
Taylor et al., “Differing Hepatic Lesions Caused by the Same Dose of Ultrasound,” J. Pathol., 1969, 98, 291-293.
Tenkin, S., Suspension Acoustics: An Introduction to the Physics of Suspensions, Cambridge University Press, 2005, Cambridge, N.Y.
Thomas et al., “Dynamics and Control of Cavitation During High-Intensity Focused Ultrasound Application,” Acoustics Research Letters Online, 2005, 6 (3), 182-187.
Tønnesen, H., Photostability of Drugs and Drug Formulations, CRC Press, 2004, Boca Raton, Fla.
Torchilin, V., “Structure and design of polymeric surfactant-based drug delivery systems,” J. Control. Release,
Torchilin et al., Liposomes: A Practical Approach, Oxford University Press, 2003, Oxford, N.Y.
Uchegbu et al., Polymers in Drug Delivery, CRC/Taylor & Francis, 2006, Boca Raton, Fla.
Umemura et al., “In vivo Acceleration of Ultrasonic Tissue Heating by Microbubble Agent,” IEEE Trans. Ultrasonics Ferroelectrics Frequency Control, 2005, 52, 1690-1698.
Villanueva et al., “Microbubbles Targeted to Intercellular Adhesion Molecule-1 Bind to Activated Coronary Artery Endothelial Cells,” Circulation, 1998, 98 (1), 1.
Verral et al., Sonoluminescence, Ultrasound: Its Chemical Physical and Biological Effects, Chapter 6, K. S. Suslick, 1988, New York, N.Y.
Ward et al., “Ultrasound-induced cell lysis and sonoporation enhanced by contrast agents,” J. Acoust. Soc. Am., 1999, 105 (5), 2951-2957.
Wang et al., Drug Delivery: Principles and Applications, Wiley-Interscience, 2005, Hoboken, N.J.
Weller et al., “Modulating Targeted Adhesion of an Ultrasound Contrast Agent to Dysfunctional Endothelium,” Ann. Biomed. Eng, 2002, 30 (8), 1012.
Wible et al., “Microbubbles induce renal hemorrhage when exposed to diagnostic ultrasound in anesthetized rats,” Ultrasound Med. Biol., 2002, 28, 1535-1546.
Widder et al., “Magnetic Microspheres: Synthesis of a Novel Parenteral Drug Carrier.” J. Pharm. Sci., 1979, 68 (1), 79.
Williams et al., “The effects of the microbubble suspension SH U 454 (Echovist) on ultrasound-induced cell lysis in a rotating tube exposure system,” Echocardiography, 1991, 8, 423-433.
Wong et al., “Electric Field Mediated Gene Transfer,” Biochem. Biophys. Res. Commun., 107 (2), 584-587.
Wu et al., “Studies on Distribution of Magnetic Gelatin Microspheres in Rabbits,” Yao Xue Xue Bao, 1993, 28 (6), 464.
Wu et al., “Studies on Magnetic Responsiveness and Renal Embolization with Magnetic Microspheres in Dogs,” Yao Xue Xue Bao, 1994, 29 (4), 311.
Yuan et al., “Vascular-permeability in a Human Tumor Xenograft-Molecular-size Dependence and Cutoff Size,” Cancer Res., 1995, 55, 3752-3756.
Ziskin et al., Ultrasonic Exposimetry, CRC Press, 1993, Boca Raton, Fla.

### BACKGROUND

Ultrasound is, by definition, sound having a frequency greater than 20,000 cycles per second (i.e., sound above the audible range). Acoustic (i.e., sound) waves are merely organized vibrations of the molecules or atoms of a medium capable of supporting the propagation of the wave. Usually, the vibrations are organized in a sinusoidal fashion, which readily reflects areas of compression and rarefaction. These areas of compression and refraction are due to periodic pressure being applied to the surface of the medium which, in the most preferred embodiments of the present invention, is human tissue. Further, as will be detailed throughout the present teachings, acoustic energy and its many unique characteristics can be effectively utilized in mediating region-specific intracellular drug delivery in vivo, one of the most highly prized and sought after goals in the drug delivery industry.

Simplistically, ultrasound is generated by a transducer which converts electrical energy to acoustical energy, or vice versa. Many transducers use piezoelectric materials, with those being either a natural crystalline solid (e.g., quartz) or a manufactured ceramic (e.g., barium titanate or zirconate titanate). To produce ultrasound, a suitable voltage is applied to the transducer. When the frequency of the input voltage reaches the resonance frequency of the piezoelectric material, the piezoelectric material responds by undergoing vibrations. Thus, a piezoelectric crystal can produce a pulse of mechanical energy (i.e., pressure pulse) by electrically exciting the crystal, functioning as a transmitter, and as a transducer, can produce a pulse of electrical energy by mechanically exciting the crystal and, thus, functioning as a receiver. Either single or multiple (e.g., phased) transducers may be utilized in ultrasonic instrumentation.

In 1956, Burov suggested that high-intensity focused ultrasound (HIFU) could be used for the treatment of cancer; in the years following, several studies looked at the effect of ultrasound on tissues (Taylor et al., 1969, Linke et al., 1973; and Bamber et al., 1979). Embodiments of the present invention highlight a new clinical and laboratory use of HIFU, mediating intracellular drug delivery in vivo. Because the goal of this HIFU application is usually not cell death at the treatment focus, for the purposes of the present disclosure, increases in heat at the treatment area must be carefully monitored and controlled.

### Ultrasound's Action on Tissue

While not wishing to be bound by any particular theory, HIFU's mode of action on living tissue is probably through two predominant mechanisms. The first is by a thermal mechanism (i.e., hyperthermia), the conversion of mechanical energy into heat. Whenever ultrasonic energy is propagated into material (e.g., tissue), the amplitude of the wave decreases with distance. This attenuation is due either to energy absorption or scattering. Absorption is a mechanism where a portion of the wave energy is converted into heat, and scattering is where a portion of the wave changes direction. Because tissue can absorb energy to produce heat, a temperature increase may occur as long as the rate heat is produced is greater than the rate heat is removed. This thermal mechanism is relatively well understood because an increase in temperature caused by ultrasound can be calculated using mathematical modeling techniques.

Briefly, healthy cellular activity depends on chemical reactions occurring at the proper location, at the proper rate. The rates of these chemical reactions and, thus, of enzymatic activity, are temperature dependent. The overall effect of temperature on enzymatic activity is described by the relationship known as the 10° temperature coefficient, or Q₁₀ Rule (Hille, 2001). Many enzymatic reactions have a Q₁₀ near 3 which means that for each 10° C. increase in temperature, enzymatic activity increases by a factor of 3. An immediate consequence of a temperature increase is an escalation in biochemical reaction rates. However, when the temperature becomes sufficiently high (i.e., approximately ≧45° C.), enzymes denature. Subsequently, enzymatic activity decreases and ultimately ceases, which can have a significant impact on cell structure and function. The extent of damage induced by hyperthermia will be dependent on the duration of the exposure as well as on the temperature increase achieved. Detrimental effects in vitro are generally noted at temperatures of 39° C.-43° C., if maintained for a sufficient time period; at higher temperatures (>44° C.), coagulation of proteins occurs rapidly (O'Brien, 2007).

HIFU's second major mode of action on living tissue, and whose effect is most important for the purposes of embodiments of this specification, is believed to be acoustic cavitation. Cavitation is a complex phenomena. However, for the purposes of the present teachings, if it is not controlled, the end result, as with hyperthermia, is also cell necrosis, induced through a combination of mechanical stresses and thermal injury (O'Brien, 2007). Ultrasound causes tissues to vibrate, where cellular molecular structure is subjected to alternating periods of compression and rarefaction. During rarefaction, gas can be drawn out of solution to form bubbles, which can oscillate in size or collapse (i.e., implode) rapidly, causing mechanical stresses and generating temperatures of 2,000° K.-5,000° K. in the microenvironment surrounding the bubble. As will be detailed throughout this specification, this energy, if properly controlled, can be utilized to safely disrupt and increase the permeability of cells and tissues, rupture drug-carrying vesicles at specific regions of the patient, and, thus, mediate efficient intracellular drug delivery in vivo.

However, cavitation is a stochastic process that involves a host of variables, especially inertial cavitation, which is dependent, among other things, on acoustic energy pulse length, frequency, intensity, and gas bubble concentration. However, cavitation is unlikely to occur when using diagnostic ultrasound equipment because of the lower energy levels produced by these devices, and for in vivo drug delivery applications mediated by cavitation (e.g., embodiments of the present invention), higher energy levels must be used (i.e., HIFU). At this time, the impact on tissue induced by the hyperthermia caused by nearly all currently used HIFU applications, is both more repeatable and predictable than those induced by cavitation. This is mainly due to instrumentation and method limitations for effectively employing acoustic cavitation, a potentially very useful but violent process, for intracellular drug delivery in vivo.

The prior art contains a variety of different medical uses for cavitation. For example, in U.S. Pat. No. 5,523,058, Umemura et al. (2005) proposed an ultrasound system used to create cavitation and for imaging. Cavitation is created through the interaction of transmitted ultrasound energy at a fundamental frequency and a second harmonic of the fundamental frequency. A special transducer is described for this purpose. In U.S. Pat. No. 7,125,387, Kawabata et al. proposed an ultrasound system to create cavitation for therapeutic purposes, especially tumor treatment, where the apparatus is in a belt wearable position close to a diseased region (on the abdomen of a patient). In U.S. patent application Ser. No. 11/523,201, filed on Sep. 19, 2006, Cain et al. proposed methods and systems for the subdivision or erosion of tissue as well as the liquification of tissue by acoustic cavitation, and other applications associated with noninvasive ultrasonic surgery.

However, concerns about the potential bioeffects of inertial cavitation associated with the interaction of ultrasound with purposely introduced gas bubbles in human beings (i.e., contrast agents) have been addressed (AIUM, 2000; NCRP, 2002). These concerns have been raised by studies documenting hemolysis of erythrocytes in vitro in cell suspensions containing contrast agent, and in mice injected with intravenous contrast agent, and later exposed to pulsed ultrasound (Williams et al., 1991; Dalecki et al., 1997; Miller, 1997; Miller et al., 1998a; Miller et al., 1998b; and Poliachik et al., 1999). Other in vitro studies have reported damage to monolayers of cultured cells whose culture media contained contrast agent and have been exposed to pulsed ultrasound (Brayman et al., 1999; Miller et al., 1999; Ward et al., 1999; and Miller et al., 2000). Hemorrhage in the vascular beds of the intestine and skin (Miller et al., 2000) and damage to cells in the heart (Skyba et al., 1998) have also been demonstrated in mice and dogs, respectively, following intravenous injection of contrast agent and exposure to pulsed ultrasound.

Further, in vivo studies with ultrasound exposed tissues in the presence of contrast agent have reported induction of petechiae (i.e., localized hemorrhages under the skin) and hemolysis (Skyba et al., 1998; and Miller et al., 2000; Wible et al., 2002; and Hwang et al., 2005), damage to the intestinal wall (Miller et al., 1998a; and Miller et al. 1998b), and alteration to the blood-brain barrier (Schlachetzki et al., 2002; and Hynynen et al., 2003).

Even though acoustic cavitation is considered a non-thermal mechanism, ultrasound contrast agents can have an effect on bulk tissue heating (Hilgenfeldt et al., 1998; Chavrier et al., 2000; Hilgenfeldt et al., 2000; Holt et al., 2002; Sokka et al., 2003; and Umemura et al., 2005). With many current insonation methods known in the art, typically, there is at least a 2- to 4-times enhancement of tissue heating by cavitation, or, if the bioeffect is a lesion, the lesion volume is likewise increased. Also when using insonation methods known in the art, single bubbles undergoing inertial collapse can cause plasma formation and temperature elevation (>4,300-5,000° K.) sufficient to induce thermal injury (Suslick, 2001). In addition, such high temperatures in an aqueous medium may result in the formation of chemically reactive free radicals (Verral et al., 1988) that can also cause trauma.

Therefore, in order to use this potentially valuable process safely and effectively in drug delivery in vivo, adequate materials, methods, and systems must be developed to both quantitate the levels of acoustic cavitation at a particular treatment site, correlate the level of said cavitation with bioeffects (e.g., the disruption of biological barriers and other tissue alterations), and the impact of many other variables and parameters. Further, methods need to be devised that allow acoustic cavitation to occur with lower levels of administered ultrasonic energy, keeping increases in temperature at the treatment site under control and within acceptable limits, as well as minimizing undesirable tissue alteration, destruction, and other effects.

### Ultrasonic Drug Delivery In Vivo

Information relevant to attempts to use ultrasound in delivering drugs to specific regions of a patient in vivo, using an ultrasonically active gas or gaseous precursor-filled lipid microspheres (i.e., termed “lipospheres”) with unspecified modes of action, can be found in, for example, U.S. Pat. Nos. 5,770,222; 5,935,553; 6,071,495; 6,139,819; 6,146,657; 6,403,056; 6,416,740; 6,773,696; 6,998,107; and 7,083,572. These preceding applications are seriously limited, because, for example, multicomponent, non-covalently associated systems are challenging to formulate and stabilize; difficulties with storage/stability and short shelf-life; unmodified lipospheres activate complement, a basic component of the immune system, and they cause pseudo-allergic reactions that can damage heart and liver cells; large-scale manufacturing of lipidic-carrying vesicles is still very challenging, even with recent technological advances in sterile techniques and process controls; optimization of the long-term physical stability of liposomal formulations remains a critical task in new product development; and most lipospheres are limited to carrying predominantly hydrophobic drugs, and they have a reduced capacity to deliver higher levels of these therapeutics to the treatment site.

The development of linear copolymer micelles and cross-linked networks for use in ultrasonic drug delivery applications has been studied extensively by Rapoport et al. (1999, 2003, and 2004). Additional information relevant to attempts to use ultrasound in delivering drugs to specific regions of a patient in vivo employing stabilized micelles can be found in, for example, U.S. Pat. No. 6,649,702. However, these applications are seriously limited, because while spherical micelles are monodispersed in size, they are highly dynamic in nature with a monomer exchange rate in the millisecond to microsecond time range. Micelles also possess significant limitations in mediating delivery of only hydrophobic and sparingly soluble drugs, with therefore minimal applications for delivering therapeutic macromolecules, especially nucleic acids. Further, depending on their critical micellar concentration (CMC), mixed micelles, composed of low molecular weight surfactants are thermodynamically unstable in aqueous media and are subject to dissociation upon dilution, resulting in micelle collapse, again depending on the CMC value, immediately upon administration to the patient (Torchilin, 2001). Therefore, stabilization is often necessary, resulting in potentially undesirable responses of the micellar vesicle to disruption by ultrasonic energy, significantly limiting its use in acoustically mediated drug delivery.

### Therapeutic Macromolecules

The rapid developments in biotechnology and molecular biology have made it possible to produce a large number of exciting and novel therapeutics in quantities sufficient enough for large-scale clinical use. Pharmaceutically active peptides and proteins can now be used in the treatment of life-threatening diseases, such as, for example, cancer and diabetes, and of several types of viral, bacterial, and parasitic diseases, as well as, for example, in vaccines for prophylactic purposes. Nucleic acid-based therapeutics, including plasmids containing transgenes for gene therapy, oligonucleotides for antisense and antigene applications, DNAzymes, aptamers, and small interfering RNAs (siRNA), represent an especially promising class of drugs for the treatment of a wide range of diseases. These include, for example, cancer, AIDS, neurological disorders (e.g., Parkinson's and Alzheimer's disease), as well as cardiovascular disorders. The specialized biological activities of these types of novel therapeutics, which hereafter may be referred to as therapeutic macromolecules, provide tremendous advantages over other types of pharmaceuticals. However, most of these macromolecules require delivery to a well-defined compartment of the body for therapeutic effectiveness, and conventional drug delivery technologies are still largely ineffective at meeting these and other challenges.

Elucidation of the human genome has generated a major impetus in identifying human genes implicated in diseases, which should ultimately lead to the development of therapeutic macromolecules for applications such as, for example, gene replacement, potential targets for gene ablation, and the like. In addition, using genomic data, potent nucleic acid drugs may be developed for individualized medicine. Data from the Human Genome Project will continue to assist in determining genetic markers responsible for patient responses to drug therapy, drug interactions, and potential side effects. Developments in human genomics, transcriptomics, and proteomics will provide an additional impetus for the advancement of nucleic acid-based therapeutic macromolecules by supplying novel targets for drug design, screening, and selection.

Unfortunately, developing adequate delivery systems for most of these new drugs remains one of the major challenges in recognizing the full therapeutic potential of many, and probably the most valuable of these therapeutic macromolecules. Indeed, the innate ability of nucleic acid-based drugs to be internalized by target cells is minimal under normal circumstances. Presently, nucleic acid delivery systems are categorized into four broad categories: (1) mechanical transfection, (2) electrical techniques, (3) chemical methods, and (4) vector-assisted delivery systems. Nearly all of these methods have only limited practical applications in nucleic acid delivery; some have proven extremely hazardous to human health.

### Protection of Therapeutics

An area of significant importance in the delivery of therapeutic macromolecules is the necessity of their protection from proteolytic, nucleolytic, and immune degradation, while traversing extracellular spaces. For some applications, a possible solution to these and other problems is targeting drugs using carriers such as liposomes, niosomes, nanosuspensions, microspheres, nanoparticles, micelles, and other carriers. Indeed, preferred embodiments of the present invention include encasing Information relevant to attempts to deliver drugs using these conventional carriers is extensive and includes, for example, U.S. Pat. Nos. 6,372,720; 6,383,500; 6,461,641; 6,569,528; 6,616,944; 7,001,614; 7,195,780; 7,288,266; and 7,345,138. However, when drug carrying vessels reach a diseased target site using one or more of these conventional carriers—a feat that with present drug delivery technology is infrequent, depending on the therapeutic macromolecule and delivery system)—in order to have any biologic or therapeutic effect, the drugs must typically gain entry into the cytoplasm of target cells. The present invention provides many novel methods and strategies to accomplish precisely this critical objective.

### Crossing Biological Barriers

Even though a close proximity of a therapeutic to many target cells can be achieved in some circumstances by employing various transport strategies—including the aforementioned vesicles—the plasma membrane of target cells, composed primarily of a bimolecular lipid matrix (i.e., mostly cholesterol and phospholipids), provides a formidable obstacle for both large and charged molecules. Thus, getting a drug across the plasma membrane into the cytosol, especially if enclosed in one of the aforementioned conventional carriers, is considered one of the greatest rate-limiting steps to intracellular drug delivery, as the majority of cells are not phagocytic and fusion of carriers with target cells is a very rare phenomenon. Unfortunately, the traditional route of internalization of many carriers and therapeutic macromolecules is by endocytosis, with subsequent degradation of the delivered therapeutic nucleic acids by lysosomal nucleases, strongly limiting the efficacy of most approaches known in the art. From this perspective alone, the development of a new, broadly applicable methodology, which can deliver genetic constructs and many other therapeutic macromolecules and other compounds directly into the cytoplasm of target cells in vivo, is highly desirable, both for use in clinical and laboratory settings.

Many organisms have developed processes for introducing macromolecules into living cells, and researchers are exploiting these methods for intracellular drug delivery. Aside from the cell-specific, usually receptor-mediated or active-uptake mechanisms, the major mechanism relies on peptides that have evolved to interact with and insert into lipid bilayer membranes. Drug delivery strategies utilize these peptides to cross both the plasma membrane bounding the cell, as well as intracellular membranes, such as, for example, membranes that enclose endocytic and other vesicles. These peptides include bacterial colicins, human porins, protein transduction domains (PTDs), and the like, from diverse species. Most of these compounds share the motif of a positively charged alpha-helix, frequently with an amphipathic structure, which is capable of inserting into lipid membranes and delivering larger cargoes intracellularly. Information relevant to attempts in intracellular drug delivery using these strategies may be found, for example, in U.S. Pat. Nos. 6,632,671; 6,780,846; 6,872,406; 7,087,729; 7,115,380; and 7,268,214. However, there are significant problems with using this type of approach for the intracellular delivery of pharmaceuticals, such as, for example, the specificity of this type of targeting to particular sites and structures, which greatly limits the technique's clinical application, as well as limiting many derivative methodologies.

### Reduction of Side Effects

Lastly, a common reason for side effects associated with many therapeutics is the high dosages required for most effective treatments. Typically, even modern pharmaceuticals do not accumulate selectively in disease areas and target tissues. Rather, following administration, therapeutics are more or less evenly distributed throughout the body. In order to generate clinically significant concentrations of drug at their desired site of action, a high concentration of the drug is typically administered. Doing this has the potential to cause undesirable complications and can be prohibitively expensive; especially considering the cost of most modern therapeutics. Therefore, development of effective drug delivery systems, (e.g., embodiments of the present invention) that can transport and deliver a drug precisely and safely to its intended site of action, remains greatly sought after by modern medicine and scientific researchers.

### SUMMARY

Embodiments of the present invention comprise materials, methods, and systems for cavitation-mediated ultrasonic drug delivery. Ultrasonic energy is utilized for the safe and effective permeation of patient tissues for mediating intracellular drug delivery in both in vitro and in vivo applications. In the present disclosure, microbubbles, both in the form of contrast agents, and/or other active agents infused into the patient, and/or bubbles formed from previous ultrasound exposure; allow for predictable cavitation thresholds, requiring much lower incident ultrasound intensities for treating tissue. In addition, by following the present teachings, much more spatially regular areas of controlled tissue permeability are produced, limiting cytotoxicity and sonolysis, and maximizing intracellular drug delivery. Moreover, by using pulsed ultrasound in the preferred embodiments of the invention, a large number of system parameters are created, which provided the appropriate monitoring and feedback mechanisms are present, allow for the use of a diversity of parameter optimizations and control systems for “fine tuning” the system for a given drug delivery application. Importantly, this is a delivery method that avoids the endocytic pathway(s) and many other biological barriers to efficient intracellular drug delivery, theoretically maximizing therapeutic efficacy. Another important embodiment of the invention is the delivery of therapeutics to organelles inside target cells, such as, for example, mitochondria, as well as to specific organs or organ regions, such as the anterior and posterior portion of the eye. Optimal embodiments include one or more therapeutics being enclosed in an acoustically responsive delivery vesicle (e.g., a nanocarrier) previous to being administered to the patient.

By assembling a known, and/or optimally sized distribution of microbubbles in the tissue volume under treatment, a lower ultrasound intensity may be utilized, thereby avoiding tissue heating, and in some cases, allowing sound to propagate through intervening bone (e.g., such as the rib cage or skull). Moreover, in a preferred embodiment of the invention, by the proper pre-sizing of, for example, magnetically targeted microbubble ensembles, the therapeutic sound field need not be focused or localized if the therapy volume is the only tissue with said microbubbles that are properly “tuned” to the incident ultrasound frequency. Cavitation also has interesting chemical effects on drugs, which can enhance their intended effect, (e.g., effective activation of anticancer drugs). Finally, in the methods and systems outlined herein, feedback of the permeation process can be accomplished during tissue alteration and drug delivery treatment, either continuously or at intervals.

Methods for administering the therapeutic ultrasonic energy are also set forth herein, as are the design of systems for generating and delivering said energy.

### BRIEF DESCRIPTION OF THE DRAWINGS

The teachings of the present specification may be better understood, as well as its numerous features, benefits, and advantages made apparent to those skilled in the art, by referencing the accompanying drawings:

FIG. 1. Illustrates a system for mediating pulsed ultrasonic energy for cavitation mediated-intracellular drug delivery in vivo.

FIG. 2A. Illustrates a cross-section of a capillary wall of the patent, including a portion of the lumen, following administration of therapeutics by 3 different example embodiments: (1) parenteral administration of free therapeutic; (2) parenteral administration of free therapeutic combined with administration of an encapsulated therapeutic within a vesicle (e.g., in an acoustically responsive nanocarrier); and (3) parenteral administration of one or more encapsulated therapeutics within a vesicle.

FIG. 2B. Illustrates a magnified view of a small section of FIG. 2A (231), showing a free therapeutic and contrast agents close in proximity to the cell wall bordering a capillary of the patient following parenteral administration.

FIG. 2C. Illustrates a magnified view of a small section of FIG. 2A (232), showing drug-containing nanocarriers (i.e., polymersomes) in close proximity to the cell wall bordering a capillary of the patient following parenteral administration.

FIG. 2D. Illustrates a magnified view of a small section of FIG. 2A (233), showing drug-containing nanocarriers (i.e., dendrisomes) in close proximity to the cell wall bordering a capillary of the patient following parenteral administration.

FIG. 3. Illustrates possible pulse sequences for use in the cavitation-mediated drug delivery process.

FIG. 4A. Illustrates a cross-section of a capillary wall of a patient and the portion of the lumen (FIG. 2A) following exposure of the region to pulsed ultrasonic energy.

FIG. 4B. Illustrates a magnified view of a small section of FIG. 4A (431), following exposure of said area to pulsed acoustic energy. Said exposure causes acoustic cavitation and cell and membrane permeation, allowing therapeutics to diffuse into the cytoplasm of target cells.

FIG. 4C. Illustrates a magnified view of a small section of FIG. 4A (432), following exposure of said area to pulsed acoustic energy. Said exposure causes acoustic cavitation, nanocarrier rupture, therapeutic release, and cell and membrane permeation, allowing therapeutics to diffuse into the cytoplasm of target cells.

FIG. 4D. Illustrates a magnified view of a small section of FIG. 4A (433), following exposure of said area to pulsed acoustic energy. Said exposure causes acoustic cavitation, nanocarrier rupture, therapeutic release, and cell and membrane permeation, allowing therapeutics to diffuse into the cytoplasm of target cells.

FIG. 5. Illustrates the Fluid Mosaic Model of membrane structure (Singer et al., 1972), an appreciation of which is important in understanding the importance of the present specification.

FIG. 6. Illustrates some of the basic processes of the endocytic pathway, an appreciation of which is important in understanding the importance of this specification.

FIG. 7. Illustrates that the cavitation-mediated ultrasonic drug delivery process typically includes the subprocesses of initiation, permeation maintenance, enhanced drug delivery, and feedback and monitoring.

FIG. 8. Illustrates that the cavitation-mediated ultrasonic drug delivery process typically includes the subprocesses of initiation, permeation maintenance, enhanced drug delivery, and feedback and monitoring; major subprocesses of initiation, permeation maintenance, enhanced drug delivery, and feedback and monitoring; as well as additional subprocesses such as de-initiation and de-maintenance.

FIG. 9. Illustrates that in one preferred embodiment, the block copolymers used in the supramolecular assemblies of the present specification consist of species with the illustrated formulas.

FIGS. 10A-B. Illustrates flowcharts showing the best mode operation for a single embodiment of the present invention.

FIG. 11A. Illustrates the chemical structure of a lipid-lysine dendron utilized as a preferred embodiment of the present invention (C₁₄)₃Lys₇(C₁₄)₈(NH₂)₈, in the formation of dendrisomes. In an optimal embodiment, said dendrisomes are modified (i.e., circles) for specific levels of acoustic sensitivity. Adapted and modified from (Al-Jamal et al. (2005).

FIG. 11B. Illustrates the proposed structure of a partial cross-section of the bilayer dendrisome membrane formed from eight of the lipid-lysine dendrons diagramed in FIG. 6A. An assumption behind this structure is that the polylysine head is directed toward the aqueous phase, and the hydrophobic alkyl chains interact with the hydrophobic groups of another dendron.

FIG. 12. Illustrates prospective data describing the encapsulation efficiency of dendrisomes.

FIG. 13. Illustrates a prospective experimental system that is used for evaluating parameters that may impact ultrasonic drug delivery.

FIG. 14A-14B. Illustrates prospective acoustic spectra measured during ultrasound exposures at FIG. 14A-low pressure (no cavitation) or FIG. 14B-high pressure (extensive cavitation). Ultrasound is applied at f=24 kHz. Due to apparatus resonance and cavitation, higher harmonics of f (e.g., 2f=48 kHz) are seen. Cavitation also generates other signals: the subharmonic (f/2=12 kHz) and its ultraharmonics (e.g., 3f/2=36 kHz) and an elevated broadband “noise” level (e.g., b1, b2).

FIG. 15A-15B. Illustrates prospective frequency spectra from acoustic emissions of cell samples exposed to FIG. 15A −1.1 MHz and FIG. 15B −3.1 MHz ultrasound. Frequency spectra exhibit characteristic markets of cavitation, including subharmonics, ultraharmonics and high levels of broadband noise.

FIG. 15C. Illustrates prospective data displaying characteristic measurements of cavitation activity, including peak broadband noise magnitude, exposure time at peak broadband noise, and half-life of broadband noise.

FIGS. 16A-16C. Illustrates prospective data describing the impact of contrast agent concentration, ultrasonic pressure and exposure time on cell viability (i.e., white bars), and the percentage with calcein uptake (i.e., black bars) employing the experimental system of FIG. 13.

FIG. 17A. Illustrates prospective data describing the impact of ultrasonic pressure and exposure time, in the presence of contrast agent, on HeLa viability (i.e., white bars) and the percentage with calcein uptake (i.e., black bars) employing the experimental system of FIG. 13.

FIG. 17B. Illustrates prospective data describing the impact of ultrasonic pressure and exposure time, in the presence of contrast agent, on AoSMC cell viability (i.e., white bars) and the percentage with calcein uptake (i.e., black bars) employing the experimental system of FIG. 13.

### DESCRIPTION OF THE PREFERRED EMBODIMENTS

Unless defined otherwise, all technical and scientific terms used herein generally have the same meaning as commonly understood by one of ordinary skill in the art to which embodiments of the present invention belongs. Generally, the nomenclature used herein, unless specifically defined below, and the clinical and laboratory procedures in cell culture, molecular genetics, organic chemistry, polymer chemistry, nucleic acid chemistry, and hybridization, therapeutic and diagnostic ultrasound, and the like, are those well known and commonly employed in the art. In addition, the techniques and procedures are generally performed according to conventional methods in the art. Throughout this specification, various general references describing said techniques and procedures are provided primarily for enablement purposes.

### DEFINITIONS

“Acoustic” generally refers to processes or procedures having to do with the generation, transmission, focusing, sensitivity, and disposition of sound wave energy.

“Acoustic energy” refers to any form of pressure wave, whether audible or inaudible. The frequency of the acoustic energy can be a single frequency or a combination of frequencies. The range of useful frequencies preferably is between approximately 1 Hz and 100 MHz, and more preferably is between approximately 15 kHz and 2 MHz. The waveform of the acoustic energy can be of any shape including a sinewave or a combination of sinewaves. The pressure of the acoustic energy can be up to a few hundred atmospheres, and preferably is applied at a peak positive pressure of up to 100 atmospheres. The optimal pressure is a function of acoustic frequency and other parameters detailed herein. The acoustic energy can be applied continuously or intermittently.

“Acoustic sensitivity,” “acoustic responsiveness,” “ultrasonically sensitive,” or “ultrasonic sensitivity” of a compound, polymer, copolymer, structure, or other material, etc., is generally used herein to refer to materials described in detail under the definition of “ultrasonically sensitive materials.”

As used herein, “administering” means oral administration, administration as a suppository, topical contact, intravenous, intraperitoneal, intramuscular, intralesional, intranasal, or subcutaneous administration, or the implantation of a slow-release device (e.g., a mini-osmotic pump) to the patient. Administration is by any route including parenteral and transmucosal (e.g., oral, nasal, vaginal, rectal, or transdermal). Parenteral administration includes, for example, intravenous, intramuscular, intra-arteriole, intradermal, subcutaneous, intraperitoneal, intraventricular, and intracranial. Moreover, where injection is to treat a tumor (e.g., induce apoptosis), administration may be directly to the tumor and/or into tissues surrounding the tumor.

An “amphiphile” or “amphipathic” chemical species refers to a chemical compound possessing both a hydrophilic and hydrophobic nature. Molecules of amphiphilic compounds have hydrophobic (i.e., usually of a hydrocarbon nature) and hydrophilic structural regions (i.e., represented by either ionic or uncharged polar functional groups). Phospholipids, a double-chain class of amphiphilic molecules, are the main components of biological membranes. The amphiphilic nature of these molecules defines the way in which they form membranes. They arrange themselves into bilayers, by positioning their polar groups toward the surrounding aqueous medium, and their hydrophobic chains toward the inside of the bilayer, defining a non-polar region between two polar ones. Although phospholipids are the principal constituents of biological membranes, there are other amphiphilic molecules (e.g., cholesterol and glycolipids) which are also included in animal cell membranes, giving them different physical and biological properties. Many other amphiphilic compounds strongly interact with biological membranes by insertion of a hydrophobic part into the lipid membrane, while exposing the hydrophilic portion to an aqueous medium, altering the membrane's physical behavior and sometimes disrupting the membrane. For example, surfactants are an example group of amphiphilic compounds, where their polar region can be either ionic or non-ionic. Some typical members of this group include sodium dodecyl sulphate (i.e., anionic), benzalkonium chloride (i.e., cationic), cocamidopropyl betaine (i.e., zwitterionic), and octanol (i.e., long-chain alcohol, non-ionic). In addition, many biological compounds are amphiphilic by nature: phospholipids, cholesterol, glycolipids, fatty acids, bile acids, saponins, etc. Many components of the preferred embodiments of the present invention are either themselves amphiphilic or contain amphiphilic components.

“Amplitude” generally refers to the magnitude of a quantity (e.g., frequency) or of a wave variable (e.g. velocity, displacement, or acceleration).

“Amplitude map” refers to a color Doppler display in which the colors correspond to the amplitude of the Doppler signal, rather than to the Doppler shift frequency.

“Angle of phase” generally refers to a way of describing a location within a periodic wave, measured in degrees or radians, where the periodic cycle contains 360° or 2π radians.

“Aptamer” generally refers to a single-stranded, partially single-stranded, partially double-stranded, or double-stranded nucleotide sequence, advantageously a replicatable nucleotide sequence capable of specifically recognizing a selected nonoligonucleotide molecule, or group of molecules, by a mechanism other than Watson-Crick base pairing or triplex formation. Aptamers referred to herein include, without limitation, defined sequence segments and sequences comprising nucleotides, ribonucleotides, deoxyribonucleotides, nucleotide analogs, modified nucleotides, and nucleotides comprising backbone modifications, branchpoints and nonnucleotide residues, groups, or bridges. Aptamers for use with embodiments of the present invention also include partially and fully single-stranded and double-stranded nucleotide molecules and sequences, synthetic RNA, DNA, chimeric nucleotides, hybrids, duplexes, heteroduplexes, and any ribonucleotide, deoxyribonucleotide, or chimeric counterpart thereof, and/or the corresponding complementary sequence, promoter or primer-annealing sequence needed to amplify, transcribe, or replicate all or part of the aptamer molecule or sequence. Unlike many prior art aptamers that specifically bind to soluble, insoluble, or immobilized selected molecules (e.g., ligands, receptors, effector molecules, etc.), in this specification, the term “aptamer” includes nucleotides capable of shape-specific recognition of surfaces by a mechanism distinctly different from specific binding. An aptamer may be a molecule unto itself or a sequence segment comprising a nucleotide molecule or group of molecules (i.e., a defined sequence segment or aptameric sequence comprising a synthetic heteropolymer or a multivalent heteropolymeric hybrid structure).

“Array” generally refers herein to a spatial arrangement of two or more transducers or transducer elements. An array may be linear (i.e., elements arranged along a line), curvilinear (i.e., elements arranged along a convex curve), rectangular (elements arranged in a rectangular pattern), or annular (i.e., elements arranged in concentric circles).

“Attenuation” refers to a decrease in the intensity of sound as it travels through a material. Three factors contribute to acoustic attenuation: (1) absorption, (2) scattering, and (3) beam divergence.

“Background noise” generally refers to the extraneous signals caused by random signal sources within or exterior to the ultrasonic testing system, including the patient and any testing material, such as, for example, cells in culture.

“Backscatter” generally refers to the energy reradiated by a scatterer in a direction opposite to that of the incident wave.

“Backscatter energy” refers to the portion of the incident acoustic energy scattered back toward the source.

“Bandwidth” generally refers to the range of frequencies present in a signal. The bandwidth is defined as that portion of the signal's frequency spectrum between upper and lower frequency bounds.

The “beam-vessel angle” refers to the angle between the axis of the ultrasound beam and the axis of a vessel lumen. This will only be equal to the Doppler angle when flow is parallel to the vessel axis. Also known as angle of attack.

A “bilayer membrane” [or simply “bilayer(s)”] refers to a self-assembled membrane of amphiphiles in an aqueous solution.

“Bioactive” refers to the ability of a therapeutic or other agent to interact with the patient, living tissue, cell, or other system. “Bioactive agent” refers to a substance which may be used in connection with an application that is therapeutic or diagnostic, such as, for example, in methods for diagnosing the presence or absence of a disease in a patient and/or methods for the treatment of a disease in a patient. “Bioactive agent” also refers to substances which are capable of exerting a biological effect in vitro and/or in vivo. The bioactive agents may be neutral or positively or negatively charged. Exemplary bioactive agents include, for example, prodrugs, targeting ligands, diagnostic agents, pharmaceutical agents, drugs, synthetic organic molecules, proteins, peptides, vitamins, steroids, steroid analogs, and genetic material (e.g., nucleosides, nucleotides, and polynucleotides).

“Biocompatible” generally refers to materials which are not injurious to biological functions and which will not result in any degree of unacceptable toxicity including allergenic responses and disease states in the patient. A biocompatible substance, when implanted in or juxtaposed against a living body or placed in contact with fluid or material actively leaving and reentering said body, does not cause an adverse pathophysiological event that would raise significant concerns about the health of said patient.

“Biodegradable” or a “biodegradable substance” refers generally to a substance that, when in a living body and/or in contact with the patient will, over a period of time, disintegrate and/or decompose in a manner, for example, that alleviates the necessity for a procedure to remove said substance from said patient. Biodegradation may result from active processes such as enzymatic means or from spontaneous (i.e., non-enzymatic) processes, such as the chemical hydrolysis of, for example, ester bonds of polylactides that occur at bodily temperature in an aqueous solution.

“Biodendrimer” or “biodendritic macromolecules” generally refers to a class of dendritic macromolecules, including many of the dendritic polymers of embodiments of the present invention, composed entirely, or almost entirely of building blocks known to be biocompatible or biodegradable to natural metabolites in vivo. These biocompatible or natural metabolite monomers include, but are not limited to, glycerol, lactic acid, glycolic acid, succinic acid, ribose, adipic acid, malic acid, glucose, and citic acid.

“Biodistribution” refers to the pattern and process of a chemical substance's distribution throughout the tissues, cells, and other bodily structures or fluids of the patient.

“Block copolymer” refers to a polymer with at least two tandem, interconnected regions of differing chemistry (i.e., “blocks”). Each region comprises a repeating sequence of monomers. Thus, a diblock copolymer comprises two such connected regions (i.e., A-B); a triblock copolymer comprises three such connected regions (i.e., A-B-C). For example, PS-β-PMMA is short for polystyrene-β-poly(methyl methacrylate); it is made by first polymerizing styrene and then subsequently polymerizing MMA. This polymer is a diblock copolymer because it contains two different chemical blocks. Triblocks, tetrablocks, pentablocks, etc., can also be synthesized. Diblock copolymers may be synthesized, for example, using living polymerization techniques, such as atom transfer free radical polymerization (ATRP), reversible addition fragmentation chain transfer (RAFT), living cationic or living anionic polymerizations, etc. Block copolymers are especially important in many embodiments of the present invention because they can microphase separate to form periodic nanostructures. If there is a hydrophobic first block and a hydrophilic second block, the block copolymers undergo microphase separation, where the hydrophobic and hydrophilic blocks form nanometer-sized structures. The interaction parameter, also called “chi” (χ), gives an indication of how different, chemically, the two blocks are and whether or not they will microphase separately. If the product of χ and the molecular weight are large (i.e., >10.5), the blocks will likely microphase separately. If the product of χ and the molecular weight are too small (i.e., <10.5), the different blocks are likely to mix. “Branched polymer” generally refers to polymers with side chains or branches of significant length which are bonded to the main chain at branch points, also known as junctional points. Branch polymers are characterized in terms of the number and size of the branches. For the purposes of this specification, dendrimers, dendrons, and dendrigrafts are separate and distinct branched chain polymers that possess a full or partial dendritic or cascade architecture.

“Broadband” or “wideband” generally refers to a wide range of frequencies in a spectrum.

“Broadband noise,” “wideband noise,” “background noise,” or “white noise” generally refers to the total of all sources of interference in a system used for the production, detection, measurement, or recording of a signal, independent of the presence of the signal, wherein “interference” refers to the process in which two or more sound or electromagnetic waves of the same frequency combine to reinforce or cancel each other, the amplitude of the resulting wave being equal to the sum of the amplitudes of the combining waves. In the context of the present invention, acoustic energy is measured at one or more frequencies which do not correspond to peaks in the acoustic spectrum and are taken from the broadband signal of the spectrum (i.e., “broadband noise”).

“Broadband signal” or “wideband noise” refers to an acoustic or electromagnetic signal which has a significant amount of its energy distributed over a wide range of frequencies.

“Bubble destruction” or “microbubble destruction” refers to the disruption of the shell of a contrast agent microbubble by a single pulse or series of pulses of ultrasound. The tendency of ultrasound to disrupt a bubble increases with increasing peak negative pressure and decreasing frequency, both of which are reflected in the Mechanical Index (MI). In general, the higher the MI, the more likely a bubble is to be disrupted. Correlation imaging methods (e.g., color Doppler) rely on bubble disruption to detect bubbles in small vessels.

“Bubble population,” “bubble ensemble,” “microbubble ensemble,” “microbubble population,” or “bubble cloud” refers to the ensemble (i.e., group) of bubbles which comprises a contrast agent dose. Note that the distribution of such parameters as bubble radius and shell thickness changes once the agent has experienced transpulmonary passage.

“Bubble specific imaging” or “microbubble specific imaging” refers to an imaging method, generally nonlinear, designed to suppress the echo from tissue in relation to that from a microbubble contrast agent.

A “capsule” refers to the encapsulating membrane plus the space enclosed within the membrane. A “carrier” refers to a pharmaceutically acceptable vehicle which is a nonpolar, hydrophobic solvent, and which may serve as a reconstituting medium. The carrier may be aqueous-based or organic-based. Carriers include lipids, proteins, polysaccharides, sugars, polymers, copolymers, acrylates, and the like.

“Cavitation” or “acoustic cavitation” refers to the oscillation of bubbles in an acoustic field, as well as the sequential formation and collapse of vapor bubbles, voids, and in many embodiments of the present invention, microbubbles, in a liquid, including liquids within or composing the patient, subjected to acoustic energy. Cavitation is usually divided into two classes of behavior (1) inertial (i.e., transient or collapse) cavitation and (2) gas body activation (i.e., non-inertial) cavitation. “Inertial cavitation,” “transient cavitation,” or “collapse cavitation” refers to the process where a void or a bubble in a liquid rapidly collapses, producing a shock wave. Such cavitation often occurs in pumps, propellers, impellers, and in the vascular tissues of plants. Non-inertial or “gas body activation”—formerly “stable cavitation”—refers to the process where a bubble in a fluid is forced to oscillate in size or shape due to some form of energy input such as, for example, an acoustic field. This phenomenon is analogous to thermal boiling but without the associated rise in temperature of a liquid mass, although localized temperatures on the molecular level can be extremely high. A “cavitation field” refers to that volume, within a processing container, flow system, or biological system—including the patient—in which active cavitation is generated. Other forces they may be acting up drug delivery vesicles exposed to HIFU include radiation forces. For the purposes of this specification, said radiation forces are considered distinctly different than the influences on drug delivery vesicles caused by acoustic cavitation.

“Cavitation” can produce strong stresses on cells, leading to various “bioeffects” which may increase drug interaction by upregulating pathways of various types of stress response, or by physically shearing the cell membrane to allow direct passage of therapeutics into the cell cytoplasm. Ultrasound has the ability to excite a wide range of bobble sizes, but the bubbles that can achieve the highest level of oscillation are those whose natural resonant frequencies are near the applied ultrasonic frequency. While not wishing to be bound by any particular theory, at relatively low acoustic amplitude, bubbles likely oscillate at the same frequency as the applied sound waves, and with relatively small expansion and contraction in size. During this “gas body activation” or “stable cavitation,” the bubbles likely accumulate dissolved gas from the surrounding liquid and slowly grow in size. As the acoustic pressure increases or as the size of the bubble approaches the resonance size, the oscillations likely increase in amplitude, become nonlinear, and eventually result in the total collapse of the bubble. This collapse event, referred to herein as “inertial cavitation” or “transient cavitation,” creates a shock wave, generates extremely high pressure and temperature, and is capable of causing substantial damage to cells. However, even with “gas body activation” or “stable cavitation” alone, the rapidly oscillating surfaces of the bubble create high fluid shear forces that can stress insonated cells and tissues, as well as producing porosity in the membranes of some cells.

A “cell” refers to any one of the minute protoplasmic masses which makes up organized mammalian or other tissues, including those of the patient, comprising a mass of protoplasm surrounded by a membrane, including nucleated and unnucleated cells and organelles. The “cell membrane” or “plasma membrane” refers to a complex, contiguous, self-assembled, complex fluid structure comprised of amphiphilic lipids in a bilayer, with associated proteins, and which defines the boundary of every cell. The structure is also referred to as a “biomembrane.” Phospholipids comprise lipid substances which occur in cellular membranes and contain esters of phosphoric acid (e.g., sphingomyelins) and include phosphatides, phospholipins, and phospholipoids.

“Center frequency” refers to the median frequency of the transmitted pulse in a pulsed ultrasound system, (i.e., the systems of the present specification). The pulse contains a range of frequencies.

“Cineloop” refers to a period of image, color, or spectral Doppler data stored digitally as a sequence of individual frames in system memory. A cineloop can be played at any speed, and transferred to videotape or an archiving medium. Cineloops, recorded at high frame rates, will contain more frames than were displayed to the operator on the video display during the examination.

“Clinical” generally refers to a clinic, or conducted in or as if in a clinic, where “clinic” refers to a medical establishment run by a group of medical specialists, where medical or healthcare problems and concerns are diagnosed and solutions possibly devised, as well as where remedial work may be performed.

“Clutter” generally refers to unwanted structural components of the received signal (e.g., Doppler shifts from moving solid tissues). In contrast imaging, clutter often refers to the portion of the received echo that is not from microbubble contrast.

“Coherence” generally refers to the degree of phase agreement among the signals making up a composite wave; if all of the signals are in phase, the wave is said to be coherent. There are various degrees of coherence that describe waves with less than full coherence. If no phase agreement exists, the wave is called incoherent.

“Coherent contrast imaging” generally refers to the name given to a nonlinear imaging method in which the phase of each transmitted pulse is alternated during the scanning process. Received echoes are then calculated, which correspond to the scanline between the two transmitted pulses, and summed. Linear echoes then cancel, but nonlinear echoes do not. The method is closely related to pulse inversion imaging, but because it requires half of the number of transmit pulses, it is capable of double the framerate. A sequence of pulses is transmitted into tissue whose phase or amplitude is changed from pulse to pulse in an incremental way. The received echoes are detected using fundamental or harmonic Doppler processing. The result is a Doppler spectrum whose Doppler shift frequencies reflect not only target velocity, but whether the target echoes are linear or nonlinear. This method forms the basis of the separation of microbubble echoes at low mechanical index, allowing real-time perfusion imaging.

“Color Doppler imaging” refers to a form of pulsed Doppler in which a large number of estimates of Doppler shift frequency are color-coded and overlaid in the location of their detection on the greyscale image. Color Doppler imaging systems operate within the range of real-time frame rates. Also referred to as “color flow imaging.” Color Doppler images generally exploit autocorrelation methods in order to detect change in the phase of returning echoes from a sequence of pulses. For this reason, they are well suited to the detection of microbubble contrast agents when they are undergoing disruption.

“Complex fluids” are fluids that are made from molecules that interact and self-associate, conferring novel technological, optical, or mechanical properties on the fluid itself. Complex fluids are found throughout biological and chemical systems, and include materials such as biological membranes or biomembranes, polymer melts and blend, and liquid crystals. The self-association and ordering of the molecules within the fluid depend on the interaction between component parts of the molecules, relative to their interaction with the solvent, if present.

As used herein, the transitional term “comprising,” which is synonymous with “including,” “containing,” or “characterized by,” is inclusive or open-ended and does not exclude additional, unrecited elements, method steps, or the like, in, for example, a patent claim. The transitional phrase “consisting of” excludes any element, step, or ingredient not specified in, for example, a patent claim. Further, a patent claim, for example, which depends on a patent claim which “consists of” the recited elements or steps cannot add an element or step. When the phrase “consists of” appears in a clause, for example, of the body of a patent claim, rather than immediately following the preamble, it limits only the element set forth in that clause; other elements are not excluded from the claim as a whole. The transitional phrase “consisting essentially of” limits, for example, the scope of a patent claim to the specified materials or steps and those that do not materially affect the basic and novel characteristic(s) of an invention. A “consisting essentially of” patent claim occupies a middle ground between closed claims that are written in a “consisting of” format and fully open, for example, patent claims that are drafted in a “comprising” format.

“Contrast agent” generally refers to a vesicle or compound that is injected into the body of the patient to make certain tissues are more visible during diagnostic imaging (e.g., ultrasound, angiography, computer topography [CT], myelogram, magnetic resonance imaging [MRI], and the like). The term “microbubble” may be used interchangeably with “contrast agent.” As exemplified in many of the preferred embodiments of the present invention, gaseous ultrasound and/or other contrast agents may be used as, or in conjunction with, therapeutic procedures or processes.

“Controlled delivery” or “controlled release” refers to delivery of a substance by a device in a manner that affords control by said device over the rate and duration of the exit of said substance from said device. For example, delivery from controlled release devices can be modulated by diffusion out of a device, dissociation of chemical bonds, and the like.

The term “copolypeptide” or “block copolypeptide” refers to polypeptides containing at least two covalently linked domains (“blocks”), one block having amino acid residues that differ in composition from the composition of amino acid residues of another block. The number, length, order, and composition of these blocks can vary to include all possible amino acids in any number of repeats. Preferably the total number of overall monomer units (i.e., residues) in the block copolypeptide is greater than 100, and the distribution of chain-lengths in the block copolymer is approximately 1.01<M_{w}/Mₙ<1.25, where M_{w}/Mₙ=weight average molecular weight, divided by the number average molecular weight.

“Cooperative non-covalent bonding” refers to interactions between two or more molecules or substances that result from two or more non-covalent chemical bonds; among them are hydrogen bonds, ionic bonds, hydrophobic interactions, and the like.

“Covalent bond” or “covalent association” refers to an intermolecular association or bond which involves the sharing of electrons in the bonding orbitals of two atoms.

“Cross-link,” “cross-linked,” or “cross-linking” generally refers to the linking of two or more stabilizing materials, including lipid, protein, polymer, carbohydrate, surfactant stabilizing materials, and/or bioactive agents, by one or more bridges. The bridges may be composed of one or more elements, groups, or compounds, and generally serve to join an atom from first a stabilizing material/molecule to an atom of a second stabilizing material/molecule. The cross-link bridges may involve covalent and/or non-covalent associations. Any of a variety of elements, groups, and/or compounds may form said bridges in the cross-links, and the stabilizing materials may be cross-linked naturally or through synthetic means. For example, cross-linking may occur in nature in material formulated from peptide chains which are joined by disulfide bonds of cysteine residues, as in keratins, insulins, and other proteins. Alternatively, cross-linking may be effected by suitable chemical modification, such as, for example, by combining a compound, (i.e., a stabilizing material) and a chemical substance that may serve as a cross-linking agent, which may cause to react by, for example, exposure to heat, high-energy radiation, ultrasonic radiation, and the like. Examples include cross-linking by sulfur to form disulfide linkages, cross-linking using organic peroxides, cross-linking of unsaturated materials by means of high-energy radiation, cross-linking with dimethylol carbamate, and the like. Photopolymerization represents a preferred method of cross-linking the polymers and/or other structures comprising the nanocarriers of embodiments of the present invention. If desired, the stabilizing compounds and/or bioactive agents may be substantially cross-linked. The term “substantially” means that greater than approximately 50% of the stabilizing compounds contain cross-linking bridges. If desired, greater than approximately 60%, 70%, 80%, 90%, 95%, or even 100% of the stabilizing compounds contain such cross-linking bridges. Alternatively, the stabilizing materials may be non-cross-linked (i.e., such that greater than approximately 50% of the stabilizing compounds are devoid of cross-linking bridges), and if desired, greater than approximately 60%, 70%, 80%, 90%, 95%, or even 100% of the stabilizing compounds are devoid of cross-linking bridges.

“Cytotoxicity” refers to the quality of being toxic to living cells or tissues of for example, those composing or belonging to the patient. Examples of toxic agents are chemical substances, as well as physical processes or procedures such as, for example, thermal treatment, or from exposure to natural agents, such as, for example, immune cells.

A “dendrigraft” generally refers to “hyper comb-branched,” “hyperbranched,” and “non-symmetrical hyperbranched” polymers. These may comprise non-cross-linked, poly branched polymers prepared by, for example (1) forming a first set of linear polymer branches by initiating the polymerization of a first set of monomers, which are either protected against or non-reactive to branching and grafting, during polymerization, each of the branches having a reactive end unit upon completion of polymerization, the reactive end units being incapable of reacting with each other; (2) grafting the branches to a core molecule or core polymer having a plurality of reactive sites capable of reacting with the reactive end groups on the branches; (3) either deprotecting or activating a plurality of monomeric units on each of the branches to create reactive sites; (4) separately forming a second set of linear polymer branches by repeating step (1) with a second set of monomers; and (5) attaching the second set of branches to the first set of branches by reacting the reactive end groups of the second set of branches with the reactive sites on the first set of branches, and then repeating steps (3), (4), and (5) above to add one or more subsequent sets of branches. In several preferred embodiments of the present invention, dendrigrafts are designed for a specified level of acoustic sensitivity; including dendrigrafts comprising dendritic supramolecular complexes.

A “dendrimer” refers to a dendritic polymer in which the atoms are arranged in many branches and subbranches along a central backbone of carbon atoms, with perfect dendrimers having an f_{br}=1.0, where dendrimers follow a dendritic or cascade architecture. Dendrimers are also called cascade molecules with a form like the branches of a tree. The name comes from the Greek ‘δ∈νδρον’/dendron, meaning “tree.” The structures were first synthesized in 1981 (U.S. Pat. Nos. 4,410,688 and 4,507,466). In their synthesis, monomers lead to a monodispersed tree like polymer, or generational structure. There are two defined methods of dendrimer synthesis: (1) divergent synthesis and (2) convergent synthesis. The former assembles the molecule from the core to the periphery, and the latter from the outside, terminating at the core. The properties of dendrimers are dominated by the functional groups on their molecular surface. For example, a dendrimer can be water-soluble when its end group is hydrophilic, like a carboxyl group. The inside of a dendrimer has a unique chemical microenvironment because of its high density.

A “dendrisome” generally refers to a vesicle formed from dendritic polymers, a structure that is capable of transporting hydrophilic as well as hydrophobic therapeutics. Dendrisomes are reminiscent of cationic liposomes, except that no cationic lipid is added to impart a positive charge. Dendrisomes may be, for example, supramolecular complexes, or may be stabilized or otherwise cross-linked. Thus, this type of vesicular dendritic structure, structurally optimized for specific levels of acoustic sensitivity, is a preferred embodiment of the present invention. A “dendron” generally refers to polymeric structures that can be broadly classified as “partial” dendrimers and represent a diverse number of compounds with widely varying characteristics. This variety of structures has led to systems which have the ability to self-associate or to form with agents such as surfactants and lipids, more complex secondary structures which are preferred embodiments of the present invention. The self-assembly of dendrons can involve hydrogen bonding or hydrophobic or electrostatic interactions. Self-assembly can also be directed by a template which interacts with functional group(s) on the dendron. Such interactions can be mediated by ligand-metal interactions, hydrogen bonding, or electrostatic interactions. Dendrons that self-associate into structures for drug delivery, either alone or in combination with other polymers and/or compounds, designed for a specified level of acoustic sensitivity, represent an optimal embodiment of the present invention.

“Dendritic polymer” and “dendritic” generally refer to polymers characterized by a relatively high degree of branching, which is defined as the number average fraction of branching groups per molecule (i.e., the ratio of terminal groups plus branch groups to the total number of terminal groups, branched groups, and linear groups). For ideal dendrons and dendrimers, the degree of branching is 1; for linear polymers, the degree of branching is 0. “Hyperbranched polymers” have a degree of branching that is intermediate to that of linear polymers and ideal dendrimers—preferably of at least 0.5 or higher. The degree of branching is expressed in the following equation:

$${f}_{br} = \frac{{N}_{t} + {N}_{b}}{{N}_{t} + {N}_{b} + {N}_{1}} \qquad Equation 1$$

wherein Nₓ is the number of type x units in the structure. Both terminal (i.e., type t) and branched (i.e., type b) units contribute to the fully branched structure, while linear (i.e., type 1) units reduce the branching factor. Therefore, 0≦f_{br}≦1, where f_{br}=O represents the case of a linear polymer and f_{br}=1 represents the case of a fully branched macromolecule. Thus, the dendritic polymers of embodiments of the present invention are composed substantially of polymers with a high degree of branching (e.g., f_{br}>0.5), and are composed substantially of dendritic or other branched chain polymeric species. The term “substantially” means that greater than approximately 50 mole percent (%) of the nanocarrier components are composed of dendritic polymers or other branched chain polymeric species. If desired, greater than approximately 60%, 70%, 80%, 90%, 95%, or even 100 mole % of the nanocarrier components are composed of dendritic polymers and/or other branched chain polymers.

“Destruction-reperfusion” refers to an indicator dilution method to measure flow which exploits the ability of ultrasound to disrupt a population of bubbles in a region of interest. Following their disruption, the rate at which the region is reoccupied by bubbles is used to deduce flowrate. See “Negative bolus.”

A “diagnostic agent” refers to any agent which may be used in connection with methods for imaging an internal region of the patient and/or diagnosing the presence or absence of a disease in the patient. Exemplary diagnostic agents include, for example, contrast agents for use in connection with ultrasound imaging, magnetic resonance imaging (MRI), or computed tomography (CT) imaging of the patient. Diagnostic agents may also include any other agents useful in facilitating diagnosis of a disease or other condition in a patient, whether or not an imaging methodology is employed.

“Directional Doppler detection” refers to the detection of Doppler signals in such away that Doppler shifts due to targets approaching the transducer are distinguished from Doppler shifts due to targets moving away from the transducer. Directional detection is usually achieved by means of quadrature demodulation.

“Doppler, continuous wave” refers to an ultrasonic system that detects Doppler-shifted signals by continuous and simultaneous transmission of sound and reception of echoes. The CW Doppler provides no range resolution.

“Doppler, pulsed” refers to a range-measuring ultrasonic system that detects Doppler-shifted signals by collecting samples, each sample taken from a separate ultrasonic pulse collected from the same location in space.

“Doppler effect” refers to the apparent change in observed sound frequency caused by relative motion between the sound source or scatterer and the observer. In diagnostic ultrasound, three classes of Doppler detectors are used: (1) continuous wave Doppler, (2) pulsed wave Doppler, and (3) color Doppler imaging systems.

“Doppler angle” refers to the angle between the direction of movement of reflectors (e.g., red blood cells) and the effective direction of the ultrasonic beam, which is normal to the wavefront.

“Doppler velocity” signal refers to a signal whose instantaneous voltage is proportional to the instantaneous Doppler frequency shift, derived by a frequency-to-voltage conversion of the Doppler signal.

“Dipole-dipole interaction” refers to the attraction of the uncharged, partial positive end of a first polar molecule; commonly designated as δ⁺ to the uncharged, partial negative end of a second polar molecule commonly designated as δ⁻. Dipole-dipole interactions are exemplified by the attraction between the electropositive head group, for example, the choline head group, of phosphatidylcholine, and an electronegative atom (e.g., a heteroatom such as oxygen, nitrogen, or sulfur) which is present in a stabilizing material (e.g., a polysaccharide). “Dipole-dipole interaction” also refers to intermolecular hydrogen bonding in which a hydrogen atom serves as a bridge between electronegative atoms on separate molecules, and in which a hydrogen atom is held to a first molecule by a covalent bond and to a second molecule by electrostatic forces.

“Droplet” refers to a spherical or spheroidal entity which may be substantially liquid or which may comprise liquid and solid; solid and gas; liquid and gas; or liquid, solid, and gas. Solid materials within a droplet may be, for example, particles, polymers, lipids, proteins, or surfactants. “Dry,” and variations thereof, refer to a physical state that is dehydrated or anhydrous (i.e., substantially lacking liquid). Drying includes, for example, spray drying, lyophilization, and vacuum drying.

The abbreviation “e.g.” refers to the phrase “for example.”

“Emulsion” refers to a mixture of two or more generally immiscible liquids, and is generally in the form of a colloid (i.e., suspension). The mixture may be of polymers, for example, which may be homogeneously or heterogeneously dispersed throughout the emulsion. Alternatively, the polymers may be aggregated in the form of for example, clusters or layers, including monolayers or bilayers, as embodied by many of the nanocarriers of this specification. Immiscible liquids can sometimes remain mixed by the addition of an emulsifier. An “emulsifier,” also known as a surfactant or emulgent, refers to a substance which stabilizes an emulsion. A wide variety of emulsifiers are used in formulating therapeutics for the patient, such as, for example, propofol, polysorbates, and sorbitan esters, to prepare vehicles, creams, and lotions. Generally, the Bancroft rule applies: Emulsifiers and emulsifying particles tend to promote dispersion of the phase in which they are not very soluble. For example, proteins dissolve better in water than in oil, and so tend to form oil-in-water emulsions (i.e., they promote the dispersion of oil droplets throughout a continuous phase of water). An “encapsulating membrane” refers to a vesicle, in all respects, except for the necessity of an aqueous solution.

The abbreviation “etc.” refers to “et cetera,” which is Latin for “and the others,” and is generally used herein to represent the logical continuation of some sort of series. The abbreviation “et al.” is used in place of “etc.,” in lists of persons, such as authors and inventors of, for example, peer-reviewed research publications and patents, respectively.

“Extracellular” or “extracellular space” generally refers to the area or region outside of an animal cell. This space is usually taken to be outside the plasma membranes and is occupied by fluid. The term is used in contrast to intracellular. The cell membrane is the barrier between the extra- and intracellular regions, and the chemical composition of extra- and intracellular milieu can be radically different. The composition of the extracellular space includes metabolites, ions, proteins, and many other substances that might affect cellular function. For example, hormones act by traveling in the extracellular space toward cell receptors. Other proteins that are active outside of the cell are, for example, digestive enzymes. The term “extracellular” is often used in reference to the extracellular fluid (ECF) which composes approximately 15 liters of the average human body.

“Far field (Frauenhofer zone)” refers to the region of the ultrasound field in which the acoustic energy flow proceeds essentially as though coming from a point source located in the vicinity of the transducer. For an unfocused circular transducer assembly, the far field commonly is ascribed to ranges greater than S/λ, where S is the radiating cross-sectional area of the transducer and λ is the acoustic wavelength in the medium.

“Fast Fourier Transform (FFT)” generally refers to a numerical algorithm used to compute the frequency components present in a periodic function varying with, for example, time. The Doppler signal is an example of such a function; the FFT produces an estimate of the relative amplitude of each frequency component, known as the Doppler spectrum.

“Feedback” generally refers to the process in which part of the output of a system is returned to its input in order to regulate its further output. In addition, feedback, as is the case with the present invention, can include a response, which may be adjustments, corrections, additions, and deletions, elicited from control systems and/or users to any deliverable or deliverable component, such as, for example, properties or characteristics of acoustic energy transmitted to the patient. Feedback may be negative, which tends to reduce output but in amplifiers, stabilizes, and linearizes operation; positive, which tends to increase output; or bipolar, which can either increase or decrease output.

“Filter” refers to a device used for suppressing acoustic or electromagnetic waves of certain frequencies while allowing others to pass.

“Filter, highpass” refers to a device which allows high—but not low—frequency variations to pass through. An example is the electrical filter used in Doppler devices to eliminate low-frequency Doppler shifts caused by clutter. This is also referred to as the “wall” or “thump” filter.

“Filter, lowpass” refers to a device that allows low—but not high—frequency variations to pass through. An example is a stenosis, which has the effect of damping rapid variations in the pressure and flow waveforms.

“Flow phantom” refers to a type of test object comprising a fluid containing ultrasound scatterers which is pumped through a pipe.

“Flow rate” refers to the volume of fluid passing through a given vessel per unit time, measured in milliliters per second or liters per minute.

“Focus, transmit” refers to the point on the axis of an ultrasonic beam where the width of the beam has a minimum value; generally, all the waves passing through the focus are in phase in relation to the surface of the transducer or to the electronic summing point of an electronically focused array. In contrast imaging, the focus is the point at which maximum bubble disruption can be expected to occur.

“Focusing, dynamic” refers to a method for controlling the axial position of the focus of an ultrasonic beam, often realized by phase control of the signals detected by a transducer array.

“For example” refers to an illustrative instance. At no time should the phrase “for example” convey any type of limitation or exclusive enumeration.

“Fourier analysis” refers to a mathematical technique for the representation of a periodic function (e.g., a time-varying waveform) as a sum of sinusoidal functions of different frequencies. Each of these constituent functions has a frequency that is an exact multiple of the same number. Fourier analysis allows the presentation of a Doppler signal in terms of the relative power of the various Doppler shift frequencies of which it is composed.

“Frame” refers to a single ultrasound image.

“Frame averaging” refers to the addition of consecutive frames in real-time imaging to smooth temporal variation; a form of lowpass filtering.

“Framerate” or “frame rate” refers to a number of ultrasound images acquired and displayed per second.

“Frequency, Doppler shift” refers to the difference between the frequencies of the transmitted wave and of the echo received from a moving target.

“Frequency spectrum” refers to the range of frequencies present in a signal recorded over some period of time.

“Fundamental frequency” refers to the natural or resonant frequency of a system. The first harmonic of a system's oscillation.

“Fundamental imaging” generally refers to a term used to describe imaging and Doppler modes in which the detected signal is acquired and processed under the assumption of linear propagation and scattering.

“Genetic material” refers generally to nucleotides and polynucleotides, including deoxyribonucleic acid (DNA) and ribonucleic acid (RNA). The genetic material may be made by synthetic chemical methodology, known to one of ordinary skill in the art, or by the use of recombinant technology, or by a combination thereof. The DNA and RNA may optionally comprise unnatural nucleotides and may be single- or double-stranded. “Genetic material” also refers to sense and anti-sense DNA and RNA; that is, a nucleotide sequence which is complementary to a specific sequence of nucleotides in DNA and/or RNA, including RNA interference (RNAi), small interfering RNA (siRNA), aptamers, and the like.

“Graft copolymer” or “graft polymer” generally refers to a polymer having polymer chains, of one kind chemically bonded onto the sides of polymer chains with a different chemical composition. As with block copolymers, the quasi-composite product has properties of both “components.” Also called comb-type polymers, there are two general methods that have been applied to synthesize graft polymers, according to the properties of backbone and branching. One method refers to the direct copolymerization of two, or more than two, monomers, one of which must already have branching. The other method uses the polymers as a backbone in the presence of polyfunctional active sites, which are used to couple with new branches or to initiate the propagation of branching. The use of graft polymers in delivery vesicles for acoustically mediated drug delivery represents a preferred embodiment of the present invention.

“Harmonic” refers to an oscillation of a system at a frequency that is a simple multiple of its fundamental frequency of sinusoidal motion. The fundamental frequency of a sinusoidal oscillation is usually called the first harmonic. The second harmonic has a frequency twice that of the fundamental, and so on.

“Harmonic imaging” refers to an ultrasound technique in which echoes at higher harmonics—usually the second—of the transmitted fundamental frequency are detected preferentially. These echoes may originate from nonlinear scatterers (e.g., microbubbles of a contrast agent) or from linear scattering of sound which has undergone nonlinear propagation and hence developed harmonics.

“Harmonic power Doppler” generally refers to a contrast specific imaging mode in which a power Doppler imaging is formed from echoes detected at the second harmonic of the transmitted frequency. Because power Doppler is sensitive to echoes which decorrelate with time, this method is most effective at detecting bubbles undergoing disruption. The harmonic filtering helps suppress linear tissue motion (i.e., clutter).

“Hertz” refers to the unit of frequency, defined as one cycle per second. Diagnostic ultrasound imaging is generally performed at frequencies of 1 MHz to 40 MHz.

“HIFU” refers to an acronym for “high-intensity focused ultrasound,” a minimally invasive medical technique used for a variety of procedures, including tumor ablation and destruction. HIFU technology is noninvasive and is used in both inpatient and outpatient facilities. An extracorporeal applicator generates a powerful, converging beam of ultrasound rays, focusing on a very precise point on the exterior or, preferably, inside the body of the patient. When the volume is larger, a sweep is performed with successive, juxtaposed exposures. Depending on conventional instrumentation parameters and other variables, concentrated acoustic energy causes a very rapid rise in temperature at this exact focal point. Outside of this point, the temperature remains normal. Importantly, in the preferred embodiments of the present invention, HIFU is used in primarily mediating intracellular drug delivery in vivo. This is accomplished, generally, by utilizing HIFU for initiating, maintaining, and controlling acoustic cavitation. Importantly, one of the goals in this type of application is little or no overall increase in temperature at the target region associated with said ultrasonic exposure.

“Hybrid” refers to a composite of mixed content or origin.

“Hydrogen bond” refers to an attractive force, or bridge, which may occur between a hydrogen atom which is bonded covalently to an electronegative atom; (e.g., oxygen, sulfur, or nitrogen) and another electronegative atom. The hydrogen bond may occur between a hydrogen atom in a first molecule and an electronegative atom in a second molecule (i.e., intermolecular hydrogen bonding). Also, the hydrogen bond may occur between a hydrogen atom and an electronegative atom, which are both contained in a single molecule (i.e., intramolecular hydrogen bonding).

“Hydrophilic” or “hydrophilic interaction” generally refers to molecules or portions of molecules which may substantially bind with, absorb, and/or dissolve in water. This may result in swelling and/or the formation of reversible gels. “Hydrophobic” or “hydrophobic interaction” generally refers to molecules or portions of molecules which do not substantially bind with, absorb, and/or dissolve in water.

“Hydrophone” refers to a transducer designed for underwater measurement of acoustic fields. The diameter of a hydrophone should be smaller than the wavelength of ultrasound to be measured, and its bandwidth should be large.

The abbreviation “i.e.” refers to the phrases “that is (to say),” “in other words,” “or sometimes,” or “in this case,” depending on the context.

“Impedance, acoustic” refers to the product of speed of sound and density of a medium in which sound is traveling. Changes in acoustic impedance are responsible for the echoes on which ultrasound imaging and Doppler flow detection are based.

“Including” or “includes” refers to enlargement, have as a part, be made up of not of exclusive enumeration, and without limitation of any kind.

“Indicator dilution” refers to a method for measuring flow in which a detectable tracer is injected in the flowstream and its rate of dispersal by transit in the flow system measured.

“Infusion” refers to a steady, usually slow, injection of material. With contrast agents, often achieved by means of a pump.

“Ionic interaction” or “electrostatic interaction” refers to intermolecular interaction among two or more molecules, each of which is positively or negatively charged. Thus, for example, “ionic interaction” or “electrostatic interaction” refers to the attraction between a first, positively charged molecule and a second, negatively charged molecule. Ionic or electrostatic interactions include, for example, the attraction between a negatively charged stabilizing material (e.g., genetic material and a positively charged polymer). “In combination with” refers to the incorporation of, for example, bioactive agents, therapeutics, and/or targeting ligands, in a composition of embodiments of the present invention, including emulsions, suspensions, and vesicles. The therapeutic, bioactive agent, and/or targeting ligand can be combined with the therapeutic delivery system, and/or stabilizing composition(s), including vesicles, in a variety of ways. For example, the therapeutic, bioactive agent and/or targeting ligand may be associated covalently and/or non-covalently with the delivery system or stabilizing material(s). Further, the therapeutic, bioactive agent and/or targeting ligand may be entrapped within the internal void(s) of the delivery system or vesicle. The therapeutic, bioactive agent and/or targeting ligand may also be integrated within the layer(s) or wall(s) of the delivery system or vesicle, for example, by being interspersed among stabilizing material(s) which form, or are contained within, the vesicle layer(s) or wall(s). In addition, it is contemplated that the bioactive agent and/or targeting ligand may be located on the surface of a delivery system or vesicle or non-vesicular stabilizing material. The therapeutic, bioactive agent and/or targeting ligand may be concurrently entrapped within an internal void of the delivery system or vesicle and/or integrated within the layer(s) or wall(s) of the delivery vesicles and/or located on the surface of a delivery vesicle or non-vesicular stabilizing material. In any case, the therapeutic, bioactive agent and/or targeting ligand may interact chemically with the walls of the delivery vesicles, including, for example, the inner and/or outer surfaces of the delivery vesicle, and may remain substantially adhered thereto. Such interaction may take the form of for example, non-covalent association or bonding, ionic interactions, electrostatic interactions, dipole-dipole interactions, hydrogen bonding, van der Waal's forces, covalent association or bonding, cross-linking, or any other interaction, as will be readily apparent to one skilled in the art, in view of the present disclosure. In certain embodiments, the interaction may result in the stabilization of the vesicle. The bioactive agent may also interact with the inner or outer surface of the delivery system or vesicle or the non-vesicular stabilizing material in a limited manner. Such limited interaction would permit migration of the bioactive agent, for example, from the surface of a first vesicle to the surface of a second vesicle, or from the surface of a first non-vesicular stabilizing material to a second non-vesicular stabilizing material. Alternatively, such limited interaction may permit migration of the bioactive agent, for example, from within the walls of the delivery system, vesicle and/or non-vesicular stabilizing material to the surface of the delivery system, vesicle and/or non-vesicular stabilizing material, and vice versa, or from inside a vesicle or non-vesicular stabilizing material to within the walls of a vesicle or non-vesicular stabilizing material, and vice versa.

“Insonate,” and variations thereof, refers to exposing, for example, regions of the patient to ultrasonic waves. The term “interpolymer” refers to a polymer comprising at least two types of monomers and, therefore, encompasses copolymers, terpolymers, and the like.

“Intensity” refers to the intensity (I) of a sound wave which is the rate of energy flux (i.e., power) through a unit area perpendicular to the direction of propagation. The unit of intensity is watts per square meter (W/cm²). The definitions of intensity commonly used in diagnostic ultrasound include “pulse-average intensity,” “spatial-average intensity,” “spatial-average pulse-average intensity,” “spatial-peak pulse-average intensity,” “spatial-peak temporal-peak intensity,” and “temporal-average intensity.”

“Interference” refers to the phenomenon describing the interaction between two waves of the same or different frequencies to produce a resultant wave, the amplitude of which depends on the amplitude and phase relationship of the interfering waves.

“Intracellular” or “intracellularly” refers to the area enclosed by the plasma membrane of a cell including the protoplasm, cytoplasm, and/or nucleoplasm.

“Intracellular delivery” refers to the delivery of a bioactive agent, such as, for example, a therapeutic, into the area enclosed by the plasma membrane of a cell.

“Laboratory” or “lab” generally refers to a place where scientific research and experiments are conducted.

“Laminar flow” refers to the flow in which there is smooth and gradual variation of velocity with position and with time. Flow may be thought of as comprising a series of individual laminae, each moving at one velocity, with viscous cohesion maintaining the flow of adjacent laminae at nearly the same velocity.

“Line density” refers to the number of lines transmitted by the ultrasound transducer per imaging frame. In contrast imaging, low-line density can reduce bubble destruction.

“Linear phased array” generally refer to a linear switched array which, in Doppler mode, operates a subset of its elements as a linear phased array and can thus steer the Doppler beam at a selected angle to the imaging beam; a popular configuration for peripheral vascular scanning.

“Linear scattering” generally refers to scattering, usually from specular reflectors or tissue parechyma, in which the echo is a faithful copy of the incident ultrasound pulse. If, for example, the phase or amplitude of the transmitted sound is altered, the phase or amplitude of the echo will be correspondingly altered. Nonlinear scatterers, such as microbubbles, do not follow these rules.

“Lipid” refers to a naturally occurring synthetic or semi-synthetic (i.e., modified natural) compound which is generally amphipathic. The lipids typically comprise a hydrophilic component and a hydrophobic component. Exemplary lipids include, for example, fatty acids, neutral fats, phosphatides, oils, glycolipids, surface-active agents (i.e., surfactants), aliphatic alcohols, waxes, terpenes, and steroids. The phrase semi-synthetic (i.e., modified natural) denotes a natural compound that has been chemically modified in some fashion. In this application, lipids are differentiated from other amphipathic compounds by having two hydrophobic “chains.” A “lipid bilayer” refers to a eucaryotic (i.e., animal) cell plasma membrane which comprises a double layer of phospholipid/diacyl chains, wherein the hydrophobic fatty acid tails of the phospholipids face each other and the hydrophilic polar heads of each layer face outward toward the aqueous solution. Numerous receptors, steroids, transporters, and the like are embedded within the bilayer of a typical cell. Throughout this specification, the terms “cell membrane,” “plasma membrane,” “lipid membrane,” and “biomembrane” may be used interchangeably to refer to the same lipid bilayer surrounding an animal cell.

A “liposome” refers to a generally spherical or spheroidal cluster or aggregate of amphipathic compounds, composed mainly of phospholipids, typically in the form of one or more concentric layers (e.g., bilayers). They may also be referred to herein as lipid vesicles. The liposomes may be formulated, for example, from ionic lipids and/or non-ionic lipids.

A “liposphere” refers to an entity comprising a liquid or solid oil, surrounded by one or more walls or membranes, with a gaseous central core.

“Loss of correlation imaging” generally refers to a term sometimes applied misleadingly to describe conventional power or color Doppler imaging when used to detect bubble disruption.

“Lyphilized,” or freeze drying, refers to the preparation of a polymer or other composition in dry form by rapid freezing and dehydration in the frozen state, sometimes referred to as sublimation. Lyophilization takes place at a temperature resulting in the crystallization of the composition to form a matrix. This process may take place under a vacuum at a pressure sufficient to maintain the frozen product with the ambient temperature of the containing vessel at approximately room temperature; preferably less than 500 mTorr; more preferably less than approximately 200 mTorr; and even more preferably, less than 1 mTorr.

“Mechanical Index (MI)” generally refers to part of the AIUM/NEMA Real Time Output Display Standard for the labelling of acoustic output on diagnostic ultrasound systems. It is defined as the peak rarefactional pressure—expressed in MPa—when a simple, uniform medium is scanned, divided by the square root of the center frequency of the pulse. The medium is assumed to have an attenuation of 0.3 dB/cm-MHz. In contrast imaging, the MI is the best practical indication of the exposure of a bubble to ultrasound, upon which its behavior depends. Note that the peak MI is estimated by the scanner and only occurs at one point, generally near the transmit focus of the transducer and near the center of the scanned plane. Further, for the purposes of this specification, the MI is a measure of the likelihood of inertial cavitation occurring.

“Megahertz (MHz)” refers to a unit of frequency equivalent to one million “cycles per second” (cps). One Megahertz (1 MHz) equals 1,000,000 cps.

A “membrane” refers to a spatially distinct collection of molecules that defines a 2-dimensional surface in 3-dimensional space, and thus separates one space from another in at least a local sense. Such a membrane must also be semi-permeable to solutes. Said membrane must also be submicroscopic (i.e., less than optical wavelengths of around 500 nm) in thickness, resulting from a process of self-assembly. Said membrane can have fluid or solid properties, depending on temperature and on the chemistry of the amphiphiles from which it is formed. At some temperatures, the membrane can be fluid (i.e., having a measurable viscosity), or it can be solid-like, with an elasticity and bending rigidity. The membrane can store energy through its mechanical deformation, or it can store electrical energy by maintaining a transmembrane potential. Under certain conditions, membranes can adhere to each other and coalesce (i.e., fuse). Soluble amphiphiles can bind to and intercalate within a membrane.

A “micelle” refers to colloidal entities formulated from primarily single-chain amphiphiles; in several preferred embodiments, a micelle is designed for a specified level of acoustic sensitivity. In certain preferred embodiments, the micelles comprise a monolayer, bilayer, or other structure.

A “microbubble” refers to a gaseous ultrasound contrast agent bounded by one or more membranes.

A “mixture” refers to the product of blending or mixing of chemical substances like elements, compounds, and other structures, including the nanocarriers of embodiments of the present disclosure, comprised, for example, of different polymers containing the same or different therapeutics, usually without chemical bonding or other chemical change, so that each ingredient and substance retains its own chemical properties and makeup. While there are frequently no chemical changes in a mixture, physical properties of a mixture may differ from those of its components. Mixtures can usually be separated by mechanical means. The term “mixture” includes solutions, homogeneous mixtures, heterogeneous mixtures, emulsions, colloidal dispersions, suspensions, dispersions, and the like.

“Mole fraction,” “mole percent,” and “mole %” refer to a chemical fraction defined as a part over a whole. Thus, a mole fraction involves knowing the moles of a solute, or component of interest (i.e., a particular copolymer species) over the total moles of all component(s) in a system (i.e., total nanocarrier components in a mixture). Multiplying the fraction calculated with the equation below by 100 yields the “mole percent.”

$${X}_{solute} = \frac{moles of solute}{total moles of all components} \qquad Equation 2$$

“Multiple frame trigger” refers to a trigger, usually from the ECG, that initiates the acquisition of a series of consecutive frames; used in triggered harmonic power Doppler modes to identify motion artifact in contrast perfusion imaging.

“Monitor” or “monitoring,” depending on the context, generally refers to maintaining regular surveillance, or close observation, over a system, process, or method. Monitoring may also refer to conducting a planned sequence of observations or measurements to assess a variety of characteristics associated with a system, process, or method of interest, and possibly creating accurate records of said characteristics for future use in verification and for other purposes. In the context of a device component, monitor refers to a display produced by a device that takes signals and displays them on a computer or other monitor, such as, for example, a television screen.

“Motion discrimination” detector generally refers to a class of signal processing used in color Doppler systems which attempts to distinguish between Doppler shifts from moving blood (i.e., which the system normally seeks to display) and Doppler shifts from moving tissue (i.e., which the system normally seeks to suppress). This processing is especially important when the velocities of moving tissue and blood are similar, such as in the detection of small vessel flow.

A “nanocarrier” refers to a vesicular (i.e., vesicle) embodiment of the present invention that may or may not be acoustically responsive and capable of being disrupted, temporarily, permanently, completely, or in part, with ultrasonic energy, having a diameter generally between 20 nm and 1,000 nm (1 μm).

“Near field (Fresnel zone)” generally refers to the region closest to the transducer. In contrast to the far field, the near field is characterized by inhomogeneity in acoustic pressure. For an unfocused circular transducer assembly, the near field commonly is ascribed to ranges less than S/λ where S is the radiating cross-sectional area of the transducer and λ is the acoustic wavelength in the medium.

“Negative bolus” refers to a term used to describe the exploitation of ultrasound's ability to destroy steadily infused microbubbles at a specific location in the circulation, thus creating a bolus defined by the absence of bubbles, which can be used as an indicator.

“Noise” generally refers in most contexts to random, and usually unwanted, signals. “Electrical Noise” refers to noise signals arising within the electrical circuits.

“Non-covalent bond” or “non-covalent association” refers to intermolecular interaction, among two or more separate molecules, which does not involve a covalent bond. Intermolecular interaction is dependent upon a variety of factors, including, for example, the polarity of the involved molecules and the charge (e.g., positive or negative), if any, of the involved molecules. Non-covalent associations are selected from electrostatics (e.g., ion-ion, ion-dipole, and dipole-dipole), hydrogen bonds, π-π stacking interactions, van der Waal's forces, hydrophobic and solvatophobic effects, and the like, and combinations thereof.

“Nonlinear imaging” refers to an ultrasound imaging designed to detect preferentially nonlinear components of the received echo. Harmonic and pulse inversion imaging are examples of nonlinear imaging methods. Nonlinear imaging is used to detect contrast microbubbles.

“Nonlinear propagation” refers to the distortion of a wavefront propagating in a medium in which the compressional phase moves slightly faster than the rarefactional phase. The result is the conversion of some of the wave energy into higher harmonics of the fundamental frequency. The effect increases strongly with increasing wave amplitude.

“Nonlinear scattering” refers to the formation of an echo from a target undergoing oscillation with components at higher harmonics. In the case of a microbubble in an acoustic field, the oscillation is asymmetric with time, producing echoes with even harmonics.

“Nyquist criterion” generally refers to the criterion that a continuously varying signal can only be unambiguously represented by instantaneous samples if the sampling rate is more than twice the maximum frequency present in the signal.

“Nyquist limit” refers to the highest frequency in a sampled signal that can be represented unambiguously; equal to one-half of the sampling frequency. In Doppler systems, this is one-half of the repetition rate for flows in each direction.

“Opacification” generally refers to the filling of an echo-free area in contrast studies (e.g., a ventricular cavity) with echoes from microbubble contrast.

Generally, “the patient” or “a patient” refers to animals, including vertebrates, preferably mammals, and most preferably humans.

“Peak negative pressure” refers to the peak rarefaction pressure attained during the negative portion of a propagating ultrasound pulse in a medium such as tissue.

“Peak pressure” refers to the maximum pressure of the fluid medium (e.g., tissue) obtained during propagation of an ultrasound pulse.

A “peptosome” generally refers to a vesicle which is assembled from copolypeptides in aqueous or near-aqueous solutions. Peptosomes are composed substantially of amino acid residues or modified amino acids of either natural, synthetic, or semi-synthetic origin. The term “substantially” means that greater than approximately 50 mole percent (%) of the vesicle components are composed of amino acids or modified amino acid residues. If desired, greater than approximately 60%, 70%, 80%, 90%, 95%, or even 100 mole % of the peptosome components are composed of amino acid or modified amino acid residues. Peptosomes may be, for example, supramolecular complexes, stabilized, or otherwise cross-linked.

“Perfluorocarbons” refers to a class of compounds obtained by replacing the hydrogen atoms of hydrocarbons by fluorine atoms. Their stability, inertness, low solubility, and low diffusion constant make them suitable gases for microbubble contrast agents.

“Perfusion imaging” refers to an imaging of flow or blood volume at the capillary level. Because the flow velocities are comparable or lower than tissue velocities, conventional Doppler methods will not suffice.

“Persistence” generally refers to a form of temporal smoothing used in both greyscale and color Doppler imaging in which successive frames are averaged as they are displayed. The effect is to reduce the variations in the image between frames, hence lowering the temporal resolution of the image.

“Phantom” generally refers to a device which simulates some parameters of the human body, allowing measurements of ultrasound system parameters or visualization of simulated anatomical features.

“Phantom, Doppler” refers to a phantom designed to provide an acoustic simulation of biological tissue containing moving scatterers, usually blood.

“Phase quadrature” generally refers to a signal-processing technique depending on an input signal being available both with its original phase and shifted through 90° of phase angle.

“Phased array” generally refers to a transducer configuration which consists of several piezoelectric elements which can be excited independently. Using proper time delays of the excitations, a wavefront of the desired configuration can be synthesized. Phased arrays have been utilized for electronic beam steering and focusing. The most preferred embodiments of the present invention use one or more phased arrays for acoustically mediated drug delivery.

“Photopolymerize” and “photopolymerization” refer to a technique wherein light is used to initiate and propagate a polymerization reaction to form a linear or cross-linked polymer structure. In the context of embodiments of the present invention, this type of system utilizes, for example, a light source, photoinitiators, and photocrosslinkable biopolymer/biodendritic macromolecular structure, including dendritic supramolecular complexes. Photopolymerization can occur via a single- or multi-photon process. In two-photon polymerization, laser excitation of a photoinitiator proceeds through at least one virtual or non-stationary state. The photo-initiator will absorb two near-IR photons, driving it into the S₂ state, followed by decay to the S₁, and intersystem crossing to the long-lived triplet state. When the spatial density of the incident photons is high, the initiator molecule (i.e., in the triplet state) will abstract an electron from, for example, triethylamine (TEA), and thus start the photocrosslinking reaction of the polymer. Indeed, controlled microfabrication via, for example, 2-photon-induced polymerication (TRIP) has been used to develop a variety of biomedical-related polymeric materials.

“Piezoelectric” refers to systems driven by the effect of certain crystals (e.g., lead-zirconate-titanate) and other materials which expand and contract in an alternating (i.e., charged) electrical field.

“Polymer” or “polymeric” refers to a substance composed of molecules which have long sequences of one or more species of atoms, or groups of atoms, linked to each other by primary, usually covalent bonds. Thus, polymers are molecules formed from the chemical union of two or more repeating units. Accordingly, included within the term “polymer” may be, for example, dimers, trimers, and oligomers. The polymer may be synthetic, naturally occurring, or semisynthetic, and linear, networked, or branched. In a preferred form, “polymer” refers to molecules which comprise 10 or more repeating units. In addition, a “polymer” can be synthesized by starting from a mixture of monomers followed by a polymerization reaction, and subsequently functionalized by coupling with suitable compounds or groups. The term “polymer” may also refer to compositions comprising block copolymers or terpolymers, random copolymers or terpolymers, random copolymers, polymeric networks, branched polymers and copolymers, hyperbranched polymers and copolymers, dendritic polymers and copolymers, hydrogels, and the like, all of which may also be grafted and mixtures thereof.

A “polymersome” generally refers to a vesicle which is assembled from polymers or copolymers in aqueous solutions. Polymersomes are composed substantially of synthetic polymers and/or copolymers. Unlike liposomes, a polymersome does not include lipids or phospholipids as its majority component. Consequently, polymersomes can be acoustically, thermally, mechanically, and chemically distinct and, in particular, more durable and resilient than the most stable of lipid vesicles. Polymersomes assemble during processes of lamellar swelling (e.g., by film or bulk rehydration) or through an additional phoresis step, or by other known methods. Like liposomes, polymersomes form by “self-assembly,” a spontaneous, entropy-driven process of preparing a closed, semi-permeable membrane. The choice of synthetic polymers, as well as the choice of molecular weight of the polymer, are important, as these distinctive molecular features impart polymersomes with a broad range of tunable carrier properties. The term “substantially” means that greater than 50 mole percent (%) of the vesicle components are composed of synthetic polymers. If desired, greater than approximately 60%, 70%, 80%, 90%, 95%, or even 100 mole % of the polymersome components are composed of synthetic polymers. Polymersomes may be, for example, supramolecular complexes, stabilized or otherwise cross-linked.

A “polypeptide” generally refers to a single linear chain of amino acids, and the family of short molecules formed from the linking, in a defined order, of various α-amino acids. The link between one amino acid residue and the next is an amide bond, and is sometimes referred to as a peptide bond. Polypeptides do not possess a defined tertiary or quaternary structure.

“Power” generally refers to the energy delivered by a wave or in a signal per unit of time; measured in watts (W) (proportional to the square of the amplitude).

“Power map” refers to the range of colors to which corresponds to the power of the Doppler signal in a power mode display.

“Power mode” refers to a color Doppler mode in which the power of the Doppler signal, rather than its estimated frequency, is mapped to color in the image. Also known as energy mode. Because the power is a scalar quantity, it does not have negative values. Because it is independent of sampling frequency, aliasing is not visible in power mode images. Because power mode plots the quantity enhanced by a contrast agent, it is often preferred in contrast Doppler imaging examination.

“Power modulation imaging” refers to a nonlinear imaging method in which the amplitude and, hence power of every other pulse transmitted into tissue is changed (i.e, doubled). The received echoes from each low amplitude pulse are then amplified more—in this case, by double the gain—so that all echoes from a linear scatterer are equal. Sequential pairs of pulses are then subtracted. Echoes from linear scatterers cancel, but those from nonlinear scatterers (e.g., bubbles) do not cancel. This method forms the basis of the separation of microbubble echoes at low mechanical index, allowing real-time perfusion imaging.

“Power spectrum” generally refers to a graph showing the relative power of each frequency component in a periodic function. For a Doppler signal, the power spectrum gives the distribution of Doppler shift frequencies present in the signal.

A “precursor” to a targeting ligand refers to any material or substance which may be converted to a targeting ligand. Such conversion may involve, for example, anchoring a precursor to a targeting ligand. Exemplary targeting precursor moieties include maleimide groups, disulfide groups, (e.g., ortho-pyridyl disulfide), vinylsulfone groups, azide groups, α-iodo acetyl groups, and the like.

“Pressure” generally refers to a form of potential energy, including acoustic energy, in for example, a fluid or tissue. Pressure is defined as the force acting on each square meter of an imaginary plane facing any direction in a fluid.

A “protein” generally refers to molecules comprising, and preferably consisting essentially of α-amino acids in peptide linkages. Included within the term “protein” are globular proteins such as, for example, albumins, globulins, histones, and fibrous proteins (e.g., collagens, elastins, and keratins). Also included within the term “protein” are “compound proteins,” wherein a protein molecule is united with a nonprotein molecule, such as, for example, nucleoproteins, mucoproteins, lipoproteins, and metalloproteins. The proteins may be naturally occurring, synthetic, or semi-synthetic.

“Pulse-average intensity” refers to the instantaneous intensity at a point in space, averaged over the duration of a single pulse.

“Pulse inversion Doppler” refers to a nonlinear imaging method in which a sequence of pulses is transmitted into tissue whose phase or amplitude is changed from pulse to pulse in an incremental way. The received echoes are detected using fundamental or harmonic Doppler processing. The result is a Doppler spectrum whose Doppler shift frequencies reflect not only target velocity, but whether the target echoes are linear or nonlinear. This method forms the basis of the separation of microbubble echoes at low mechanical index, allowing real-time perfusion imaging.

“Pulse inversion imaging” generally refers to a nonlinear imaging method whereby two pulses are transmitted into tissue, the second an inverted copy of the first. The received echoes are summed, canceling echoes from linear structures and enhancing echoes with even harmonic components.

“Pulse repetition frequency” (“PRF”) generally refers to the repetition of the transmission pulses of a pulse-echo system; the inverse of the pulse repetition period.

“Pulsed” or “pulsed ultrasound” generally refers to ultrasonic or acoustic energy which is produced or transmitted or modulated in short bursts or pulses. As used herein, pulsed ultrasonic energy may contain segments of continuous ultrasonic energy. The use of pulsed ultrasonic energy, with unique and application-specific, customizable sequences for drug delivery purposes, is a major feature of the present invention.

“Range gate” generally refers to an electronic circuit for selecting an ultrasonic signal according to its depth along the ultrasonic beam by gating the signal with an appropriate time delay.

“Rarefaction” generally refers to the reduction in pressure of the medium during the negative portion of the cycle of a traveling acoustic wave.

“Rayleigh Scattering” refers to the name given to the deflection of waves by an ensemble of targets much smaller than the wavelength of the incident radiation. Red blood cells are Rayleigh scatterers to ultrasound. The intensity of ultrasound scattered back to the transducer by the Rayleigh process is proportional to the fourth power of frequency.

“Real-time” generally refers to the acquisition and display of ultrasonic images at a sufficiently rapid rate that moving structure can be “seen” to move at their natural rate. Frame rates of approximately 15 frames per second or greater are considered real-time, though faster rates are typically employed in cardiac imaging.

“Receive gain” refers to the amplification to which a detected echo is subjected by an ultrasound system. In nonlinear imaging, it is important to distinguish the effect of this from change in the transmit power.

“Receptor” generally refers to a molecular structure within a cell, or on the surface of a cell, which is generally characterized by the selective binding of a specific substance. Exemplary receptors include, for example cell-surface receptors for peptide hormones, neurotransmitters, antigens, complement fragments, immunoglobulins, cytoplasmic receptors for steroid hormones, and the like.

“Reflection” generally refers to a change in the direction of propagation of a wave as it encounters an interface between two media across which the acoustic impedance changes. The amplitude of the reflected wave is determined by the magnitude of this difference. “Specular reflection” generally refers to the phenomenon of reflection of a wave by a flat surface large in relation to the wavelength.

“Refraction” refers to a change in the direction of propagation of a wave as it crosses an interface between two media with different speeds of sound. The amount by which the portion of the wave entering the second medium is deviated depends on the difference in propagation velocity between the media and the angle of incidence at their interface.

“Region of a patient” or “region of the patient” refers to a particular area or portion of the patient and, in some instances, to regions throughout the entire patient. Exemplary of such regions are the eye; gastrointestinal region; the cardiovascular region, including myocardial tissue; the renal region as well as other bodily regions, tissues, lymphocytes, receptors, organs, and the like, including the vasculature and circulatory system; as well as diseased tissue, including cancerous tissue, such as in the prostate or breast.

“Region of a patient” includes, for example, regions to be imaged with diagnostic imaging, regions to be treated with a bioactive agent (e.g., a therapeutic), regions to be targeted for the delivery of a bioactive agent, and regions of elevated temperature. The “region of a patient” is preferably internal; although, if desired, it may be external. The term “vasculature” denotes blood vessels, including arteries, veins, and the like. The phrase “gastrointestinal region” includes the region defined by the esophagus, stomach, small and large intestines, and rectum. The phrase “renal region” denotes the region defined by the kidney and the vasculature that leads directly to and from the kidney, and it includes the abdominal aorta. “Region to be targeted,” “targeted region,” “target region,” or “target” refers to a region of the patient where delivery of a therapeutic is desired.

“Region to be imaged” or “imaging region” denotes a region of a patient where diagnostic imaging is desired.

“Resolution (spatial)” refers to a measure of the ability of a system to display distinguishable images of two closely spaced point structures as discrete targets.

“Resolution (temporal)” refers to a measure of the ability of a system to display two closely spaced events in time as discrete entities.

“Resonance” refers to oscillation of a system at its natural frequency of vibration, as determined by the physical parameters of the system. At resonance, large amplitude vibrations will ultimately result from low-power driving of the system. Resonance can occur in microbubbles driven by an acoustic wave. The resonant frequency for a free gas bubble is primarily determined by its size.

“Resuspending” refers to adding a liquid to change a dried physical state of a substance to a liquid physical state. For example, a dried therapeutic delivery system may be resuspended in a liquid such that it has similar characteristics in the dried and resuspended states. The liquid may be an aqueous liquid or an organic liquid, for example. In addition, the resuspending medium may be a cryopreservative. Polyethylene glycol, sucrose, glucose, fructose, mannose, trebalose, glycerol, propylene glycol, sodium chloride, and the like, may be useful as a resuspending medium.

“Reynolds number” refers to a number expressing the balance of inertial and viscous forces acting on a flowing fluid. Reynolds numbers higher than a critical value result in disturbed flow progressing to turbulent flow.

“Reynolds stress” refers to the increased resistance to flow offered by a fluid in turbulence, which has its origin in viscous forces resulting from chaotically oriented velocity gradients.

“Rupture” refers to the act of breaking, bursting, or disassociating, usually in response to specific stimuli, such as, for example, ultrasonic energy; “rupturing” means undergoing rupture.

“Sample volume,” as used in this specification (depending on the context), generally refers to the region of the ultrasound beam—or beams in a CW system—sensitive to the presence of Doppler-shifted echoes. In a pulsed Doppler system, the axial position and extent of the sample volume is determined by the length of the transmitted pulse and the location and length of the range gate, both of which are normally under control of the operator. The sample volume width is determined by the lateral extent of the ultrasound beam.

“Scatterer” refers to a discontinuity small in relation to the wavelength that reradiates ultrasound through an angle rather than in specular fashion.

“Shell” generally refers to, for example, the coating which stabilizes the gas contents of a microbubble within the fluid medium. In ultrasound contrast agents, the shell is made from a lipid, protein, or other biocompatible material.

“Sideband” refers to the components of a signal whose frequencies are either above (i.e., upper sideband) or below (i.e., lower sideband) the frequency of the carrier transmitted signal.

“Signal-Noise Ratio (SNR)” refers to the ratio of the amplitude of a signal to that of noise. The larger the signal-noise ratio, the easier it is to detect and measure a signal. The sensitivity of any device is ultimately limited by the signal-noise ratio. The SNR is usually expressed in decibels.

“Signal-to-Clutter Ratio (SCR)” refers to the ratio of the amplitude of the wanted portion of a Doppler signal to that of its largest clutter component. The larger the signal-clutter ratio, the easier it is to distinguish Doppler shifts due to blood flow from those of other targets. Because the clutter is usually much greater in amplitude than the wanted signal in clinical Doppler examinations, the SCR is a primary determinant of the detectability of flow in a given vessel. It is usually expressed in decibels.

“Sonolysis” “Sonolysis” refers to the disruption of biological cells, either directly or indirectly, through application of ultrasonic energy.

“Sound” generally refers to the vibrational energy that propagates through a medium. Liquids and gases support longitudinal (i.e., compression) waves. Solids support other vibration modes in addition to longitudinal waves.

“Spatial-average intensity” refers to the same as the spatial-average temporal-average intensity. Generally, this parameter is used when specifying the intensity for continuous-wave (CW) ultrasound.

“Spatial-average pulse-average intensity (SAPA)” refers to the pulse average intensity averaged over the beam cross-sectional area.

“Spatial-average temporal-average intensity (SATA)” refers to the temporal average intensity averaged over the beam cross-sectional area in a specified plane.

“Spatial-peak pulse-average intensity (SPPA)” refers to the value of the pulse average intensity at the point in the acoustic field where the pulse average intensity is a maximum, or is a local maximum within a specified region.

“Spatial-peak temporal-peak intensity (SPTP)” refers to the value of temporal peak intensity at the point in the acoustic field where the temporal peak intensity is a maximum, or is a local maximum within a specified region.

“Spectral broadening” refers to the width of the Doppler spectrum on a sonogram display, which corresponds to the range of Doppler shift frequencies present at a given time. Spectral broadening will be seen to increase when this range is increased; one example is the Doppler signal obtained when laminar flow with a blunt flow profile becomes disturbed.

“Spectral Doppler” refers to a name commonly used to refer to the combination of either CW or pulsed Doppler with a spectral display.

“Spectral width” refers to the estimated range of frequencies present in a spectrum, defined as the difference between the upper bandwidth frequency and lower bandwidth frequency.

“Spectrum” refers to a range of values, often continuous (i.e., the range of frequencies in a Doppler-shifted signal).

“Spray drying” refers to drying by bringing an emulsion of surfactant and a therapeutic, or portions thereof, in the form of a spray into contact with a gas (e.g., air) and recovering it in the form of a dried emulsion. A blowing agent, such as methylene chloride, for example, may be stabilized by said surfactant.

“Stabilized” or “stabilization” refers to exposure of materials (e.g., polymers, mixtures, emulsions, and the like) including materials of embodiments of the present invention, to stabilizing materials or stabilizing compounds. “Stabilizing material” or “stabilizing compound” refers to any material which is capable of improving the stability of compositions containing the therapeutics for use with embodiments of the present invention, including targeting ligands and/or other bioactive agents described herein, and including, for example, mixtures, suspensions, emulsions, dispersions, vesicles, and the like. Encompassed in the definition of “stabilizing material” are certain bioactive agents. The improved stability involves, for example, the maintenance of a relatively balanced condition, and may be exemplified, for example, by increased resistance of the composition against destruction, decomposition, degradation, rupture, and the like. In the case of preferred embodiments involving nanocarriers filled with therapeutics and/or bioactive agents, the stabilizing compounds may serve to either form the vesicles or stabilize the vesicles, in either way serving to minimize or substantially including completely prevent the escape of liquids, therapeutics, and/or bioactive agents from the vesicles, until said release is desired. The term “substantially,” as used in the present context of preventing escape of liquids, therapeutics and/or bioactive agents from said nanocarriers, means greater than approximately 50% is maintained entrapped in the nanocarriers until release is desired, and preferably greater than approximately 60%, more preferably greater than approximately 70%, even more preferably greater than approximately 80%, still even more preferably greater than approximately 90%, is maintained entrapped in the nanocarriers until release is desired. In particularly preferred embodiments, greater than approximately 95% of the liquids, therapeutics, and/or bioactive agents maintained entrapped until release is desired. The liquids, therapeutics or bioactive agents may also be completely entrapped (i.e., approximately 100% is maintained entrapped) until release is desired. Exemplary stabilizing materials include, for example, lipids, proteins, polymers, carbohydrates, surfactants, and the like. The resulting mixture, suspension, emulsion, or the like, may comprise walls (i.e., films, membranes, and the like) around the bioactive agent, or may be substantially devoid of walls or membranes, if desired. The stabilizing may, if desired, form droplets. The stabilizing material may also comprise salts and/or sugars. In certain embodiments, the stabilizing materials may be substantially (i.e., including completely) cross-linked. The stabilizing material may be neutral or positively or negatively charged.

“Subharmonic” generally refers to an oscillation of a system at a frequency that is a simple fraction of that its fundamental sinusoidal oscillation. The second subharmonic has a frequency of one half the fundamental frequency, and so on.

“Supramolecular assembly,” “supramolecular complex,” or “supramolecular structure” generally refers to a defined complex of molecules held together by non-covalent bonds and, in several preferred embodiments, is designed for a specified level of acoustic sensitivity. While a supramolecular assembly can be simply composed of two molecules (e.g., a DNA double helix or an inclusion compound), in the embodiments of the present invention, supramolecular assembly refers to larger complexes of molecules that form sphere, rod-like, and/or other vesicles for the delivery of therapeutics or other substances to the patient. The dimensions of supramolecular assemblies can range from nanometers to micrometers. Supramolecular complexes allow access to nanoscale objects using a bottom-up approach, in much fewer steps than a single molecule of similar dimensions. The process by which a supramolecular assembly forms is termed “self-assembly” or “self-organization,” where self-assembly is the process by which individual molecules form the defined aggregate, and self-organization is the process by which those aggregates create higher-order structures. A great advantage to the supramolecular approach in drug delivery is that the larger complexes of molecules will disassociate or degrade back into the individual molecules comprising said assembly, which can be broken down by the patient. Some of the preferred embodiments of the present invention are, for example, supramolecular structures formed from, for example, dendritic polypeptides.

“Surfactant” or “surface active agent” refers to a substance that alters energy relationships at interfaces; such as, for example, synthetic organic compounds displaying surface activity, including inter alia, wetting agents, detergents, penetrants, spreaders, dispersing agents, and foaming agents. The term surfactant is derived from “surface active agent”; surfactants are often organic compounds that are amphipathic and typically are classified into three primary groups: (1) anionic, (2) cationic, and (3) non-ionic.

A “suspension” or “dispersion” refers to a mixture, preferably finely divided, of two or more phases (i.e., solid, liquid or gas), such as, for example, liquid in liquid, solid in solid, gas in liquid, and the like, which preferably can remain stable for extended periods of time.

“Synthetic polymer” refers to a polymer that comprises, in whole or in part, substances that are created by chemical synthesis, rather than produced naturally by an organism. A substance that is a naturally occurring polymer may also be created by chemical synthesis; for example, peptides and nucleotides can be created either naturally or in the laboratory.

“Targeted vesicle” refers to a vesicle, such as, for example, a nanocarrier with a targeting ligand covalently or noncovalently attached to, or anchored within, said vesicle. “Targeting ligand” or “targeting moiety” generally refers to any material or substance which may promote targeting of tissues and/or receptors, in vivo or in vitro, with the compositions of embodiments of the present invention. The targeting ligand may be synthetic, semi-synthetic, or naturally occurring. Materials or substances which may serve as targeting ligands include, for example, proteins, including antibodies, antibody fragments, hormones, hormone analogues, glycoproteins and lectins, peptides, and polypeptides; amino acids; sugars; saccharides, including monosaccharides and polysaccharides; carbohydrates and vitamins; steroids; steroid analogs; hormones; cofactors; bioactive agents; and genetic material including aptamers, nucleosides, nucleotides, nucleotide acid constructs, and polynucleotides. Magnetic compositions are also preferred targeting moieties for use with embodiments of the present invention, and may be used alone or in combination with other targeting moieties, such as, for example, ligands of synthetic, semi-synthetic, or naturally occurring origin.

“Temporal-average intensity” refers to the time average of instantaneous intensity at a point in space; this is equal to the mean value of the instantaneous intensity at the point considered. For scanning systems, the instantaneous intensity is averaged over one or more scan repetition periods for a specified operating mode.

“Temporal peak intensity” generally refers to the peak value of the instantaneous intensity at the point considered. It is given by P²/ρ^{c}, where P is the instantaneous acoustic pressure, ρ is the density of the medium, and c is the speed of sound in the medium.

“Test object, Doppler” generally refers to a device designed to create a reproducible acoustic and physical setting in which one or more aspects of a Doppler system's performance may be tested or calibrated.

“Therapeutic” “drug,” “pharmaceutical,” “pharmacologically active agent,” “permeant,” or “deliverable substance” refers to any pharmaceutical, drug or prophylactic agent which may be used in the treatment, including the prevention, diagnosis, alleviation, or cure of a malady, affliction, disease, or injury of the patient. In addition, “therapeutic” means any chemical or biological material or compound suitable for delivery by the methods previously known in the art, combined with the methods of and/or by the present invention, which induces a desired effect, such as a biological or pharmacological effect, which may include, but is not limited to (1) having a prophylactic effect on the patient, and preventing an undesired biological effect such as, for example, preventing an infection; (2) alleviating a condition caused by a disease (i.e., alleviating pain or inflammation caused as a result of disease); (3) either alleviating, reducing, or completely eliminating the disease from the patient; and/or (4) the placement within the viable tissue layers of the patient of a compound or formulation which can react, optionally in a reversible manner, to changes in the concentration of a particular analyte and, in so doing, cause a detectable shift in this compound or formulation's measurable response to the application of, for example, energy to this area which may be electromagnetic, mechanical, or most preferably acoustic (i.e., ultrasonic). The effect may be local, such as providing for local tissue permeability to, for example, a nucleic acid (e.g., RNAi, siRNA, etc.), or the effect may be systemic. The term “therapeutic” also includes contrast agents and dyes for visualization. Obviously, therapeutically useful peptides, polypeptides, polynucleotides, and other therapeutic macromolecules may also be included within the meaning of the term “pharmaceutical” “drug,” or “therapeutic.”

“Therapeutic macromolecule” refers to a pharmacologically active agent produced either partially, or in full, by modern biotechnological and/or other techniques (e.g., proteins, nucleic acids, synthetic peptides).

“Therapeutic ultrasound” refers to high-intensity focused ultrasound, or HIFU.

“Therapy” refers to the treatment of a disease or disorder by various methods.

“Thermal index” generally refers to the part of the AIUM/NEMA Real Time Output Display Standard for the labeling of acoustic output on diagnostic ultrasound systems. It is defined as the ratio of the power being emitted to the power required to raise the temperature by 1 degree Celsius in a simple, uniform medium insonified by the active transducer. The medium is assumed to have an attenuation of 0.3 dB/cm-MHz.

“Tissue harmonic imaging” refers to the nonlinear imaging mode which detects preferentially echoes from higher harmonics of the fundamental transmitted signal, developed as a consequence of nonlinear propagation in tissue.

“Tissue” refers generally to specialized cells which may perform a particular function. The term “tissue” may refer to an individual cell, or a plurality or aggregate of cells, (e.g., membranes, blood, or organs). The term “tissue” also includes reference to an abnormal cell or a plurality of abnormal cells. Exemplary tissues include myocardial tissue including myocardial cells and cardiomyocites, membranous tissues, including endothelium and epithelium, laminae and connective tissue including interstitial tissue, and tumors.

“Transdermal,” “percutaneous,” “transmembrane,” “transmucosal,” or “transbuccal” refers to passage of a permeant (e.g., a therapeutic) into or through the biological membrane or tissue to achieve effective therapeutic levels of a drug in, for example, blood, tissue, and/or cells, or the passage of a molecule present in the body (i.e, “analyte”) out through the biological membrane or tissue, so that the analyte molecule may be collected on the outside of the body. Disruption of said biological membrane, by the methodologies of this disclosure, may preferably facilitate said passage of said permeant.

“Transient echo,” in contrast imaging, generally refers to an echo of high intensity and short duration associated with disruption of a bubble, following its exposure to an acoustic field.

“Transmit intensity” refers to the intensity of the pulse of sound emitted by the transducer into the body. For contrast imaging, the peak rarefactional pressure is a major determinant of bubble response.

“Transmit power” refers to a common name given to the control on an ultrasound system that determines transmit intensity; the total energy transmitted into tissue by the transducer. In clinical systems, this is monitored by the Mechanical Index (MI), a normalized index of peak transmitted pressure.

“Transit time broadening” refers to the spectral broadening that occurs as a consequence of the movement of scatterers through a Doppler sample volume of finite size. The smaller the sample volume, the more pronounced is the transit time broadening. Also known as geometric or intrinsic spectral broadening.

“Triggered imaging” refers to the control of the acquisition of a single or series of ultrasound images by an external signal. In echocardiography, the trigger is usually derived from the ECG signal.

“Turbulence” generally refers to a disorganized flow with chaotically oriented components in many directions.

“Ultraharmonic” refers to the oscillation of a system at a frequency that is a rational multiple of that of its fundamental sinusoidal oscillation (i.e., 1.5 or 2.5 times the fundamental frequency).

“Ultrasonic” refers to frequencies of sound above normal human hearing, generally accepted to be at 20 KHz to 2 MHZ and above, but also extended down to the 5 KHz to 20 KHz range in certain processing applications; subsonic, supersonic, or transsonic has to do with the speed of sound. As used throughout this specification, “ultrasonic” also refers to any processes, practices, or methods employing ultrasound, either high-intensity focused ultrasound (HIFU), for therapeutic or other purposes; or diagnostic ultrasound, for imaging; or any other use of acoustic energy.

“Ultrasonically sensitive material” or “ultrasonically sensitive materials” is generally used herein to refer to a compound, molecule, drug, therapeutic, polymer, copolymer, and/or other material, including those of synthetic, semi-synthetic, or natural origin; alone or in combination with other materials that may, or may not be ultrasonically sensitive; which are sensitive to mechanical rectification or other aspects of exposure to high-intensity ultrasound, high-intensity focused ultrasound (HIFU), or ultrasound (i.e., said material changes shape, conformation, and/or chemical reactivity, etc., in response to ultrasound). These ultrasonically sensitive materials can be used in various applications of the present teachings, and several types of ultrasonically sensitive material may be employed, either alone or in combination with other compounds. A most preferred embodiment is the disassociation of therapeutic-containing nanocarriers of embodiments of the present invention by ultrasound, at a treatment site of the patient, releasing said therapeutic(s). Various other embodiments of ultrasonically sensitive materials include pharmaceutical agents complexed with ultrasonically sensitive materials, either covalently or through non-covalent interactions. For example, ultrasonically sensitive materials can be switchable to release a pharmaceutical agent. In some embodiments, molecules sensitive to asymmetrical waveforms prevalent due to nonlinear propagation of ultrasonic waveforms may be used. With such waveforms, the peak positive pressure can be an order of magnitude, or more, greater than the peak negative pressure. Further, a compressible material, or part of a material, can act as an effector by changing its shape considerably during ultrasound exposure, thus triggering a specific event or process, including drug release, formation of a gaseous contrast microbubble for imaging, enhancing acoustic cavitation, etc. In addition, “ultrasonically sensitive materials” can enhance chemical reactivity, thereby having a direct pharmacological effect, or said materials can enhance the pharmacological effect of other therapeutics and/or prodrugs. Likewise, ultrasonically sensitive materials include molecules that are sensitive to peak negative or positive pressures and/or ultrasonic intensities.

Additional embodiments of “ultrasonically sensitive materials” include use of molecules, polymers, therapeutics, and the like, that are sensitive to free radical concentration. For example, acoustic cavitation can generate free radicals that may be used as a trigger, causing the molecules to become effectors. Moreover, since free radicals are part of the natural inflammation process, such free radical sensitive molecules can be useful effectors even without an ultrasound trigger, thus allowing more pharmacological control of the inflammation process. These free radical detecting molecules can also be used for cavitation detection, in vivo, as inflammation detectors. Further, the term “ultrasonically sensitive materials” includes molecules designed to generate or process dissolved gasses so as to form free gas bubbles in response to many different triggering events or sensing environments. For example, when bound to a tumor-specific antigen, the molecule can change functionality and produce a gas bubble. This gas bubble would then be useful as a contrast agent for diagnostic detection or as a nucleus for acoustic cavitation. First, cardiac infarction or stroke produces ischemic tissue and/or inflammation which, in turn, damages affected tissues by free radical formation. A free radical sensitive molecule can release drugs comprising contrast agents, thereby allowing quicker diagnosis and/or treatment. Second, a molecule reacting to some aspect of an ultrasonic exposure, such as pressure, intensity, cavitation asymmetric waveforms due to nonlinear propagation, cavitation, and/or free radical formation due to cavitation, can be an ideal candidate as a drug carrier, contrast agent delivery vehicle, nuclei for therapeutic cavitation, etc. Third, ultrasonically sensitive molecules that change in response to ultrasound exposure, by any of the mechanisms mentioned herein, can have biological effectiveness by many different mechanisms, including switchable enzymatic activity; switchable water affinity (e.g., change from hydrophobic to hydrophilic); switchable buffer modulating local pH; switchable chemical reactivity allowing remote ultrasound control of an in vivo chemical reaction, perhaps, for example, producing a drug in situ or modulating drug activity; switchable conformations of a smart molecule, allowing the covering or uncovering (i.e., presentation) of an active site which could bind with any designed binding specificity (e.g., a drug which was inactive [inert] until triggered locally by ultrasound). Fourth, ultrasonically sensitive molecules that are switchable free radical scavengers can be activated by ultrasound for tissue protection, for example, following a stroke or cardiac infarction.

Other embodiments of “ultrasonically sensitive materials” can be directly or indirectly affected by free radical generators and scavengers as cavitation modulators. Additional “ultrasonically sensitive materials” can work with changes in localized concentration of many other reagents, molecules, drugs, etc., to protect some regions of the patient and to predispose others to, for example, penetration of biological barriers by a variety of molecules, compounds, or other structures. Exemplary applications include modulating cavitation nuclei, either naturally or by some ultrasonically sensitive molecules designed to act as cavitation nuclei or a processor of cavitation nuclei, and which are controlled in their activity by ultrasonically induced changes in free radical concentrations, pH, etc.

“Vacuum drying” refers to drying under reduced air pressure, resulting in drying at a lower temperature than required at full pressure.

“Van der Waal's forces” refers to dispersion forces between nonpolar molecules that are accounted for by quantum mechanics. Van der Waal's forces are generally associated with momentary dipole moments which are induced by neighboring molecules, involving changes in electron distribution.

“Variance map” refers to a color Doppler display in which the saturation of a color corresponds to the estimated variance of the Doppler signal. This is often combined with the velocity map by using a different hue, so that the combination of the two quantities can be used for the detection of turbulence.

“Vector” and “cloning vehicle” generally refers to non-chromosomal double stranded DNA comprising an intact replicon such that the vector is replicated when placed within a unicellular organism (e.g., a bacterium), for example, by a process of transformation.

“Velocity,” as used herein generally refers to a vector describing the rate of change of position with time. Also used for the magnitude of the velocity vector, although this quantity is really the flow “speed.”

“Velocity gradient” generally refers to the rate of change of velocity with position. With steady laminar flow in a round vessel, this gradient is usually in a radial direction.

“Velocity profile” generally refers to the variation of velocity with radial position for flow in a vessel. “Blunted velocity profile” refers to a modification of the parabolic flow profile that is commonly encountered in physiological circumstances. The central laminae move at almost one velocity. “Parabolic velocity profile” refers to the form of the velocity profile found with steady flow in a round vessel that exhibits flow resistance only. The parabolic flow profile has the special property that the average velocity across the vessel is exactly one-half of the maximum velocity in the centre stream (also called Poiseuille flow). “Critical velocity” refers to the flow velocity at which the Reynolds number attains its critical value and the transition of disturbed flow to turbulence occurs.

“Viral vectors” include retroviruses, adenoviruses, herpesvirus, papovirus, or otherwise modified naturally occurring viruses. Vector also means a formulation of DNA, with a chemical or substance, which allows uptake by cells. In addition, materials could be delivered to inhibit the expression of a gene. Approaches include antisense agents, such as synthetic oligonucleotides, which are complementary to RNA or the use of plasmids expressing the reverse complement of a gene; catalytic RNAs or ribozymes which can specifically degrade RNA sequences by preparing mutant transcripts lacking a domain for activation; or over-expressed recombinant proteins which antagonize the expression, or function, of other activities. Advances in biochemistry and molecular biology, in recent years, have led to the construction of recombinant vectors in which, for example, retroviruses and plasmids are made to contain exogenous RNA or DNA respectively. In particular instances, the recombinant vector can include heterologous RNA or DNA, by which is meant RNA or DNA which codes for a polypeptide not produced by the organism (e.g., the patient) susceptible to transformation by the recombinant vector. The production of recombinant RNA and DNA vectors is well understood in the prior art, and need not be summarized here.

“Vesicle” generally refers to an entity which is usually characterized by the presence of one or more walls or membranes which form one or more internal voids. Vesicles, such as the nanocarriers of embodiments of the present invention, may be formulated, for example, from a stabilizing material such as a copolymer, including the various polymers described herein, especially “block copolymers,” a proteinaceous material, including the various polypeptides described herein, and a lipid. As discussed herein, vesicles may also be formulated from carbohydrates, surfactants, and other stabilizing materials, as desired. The proteins, polymers, copolymers, and/or other vesicle-forming materials may be natural, synthetic, or semi-synthetic. Preferred vesicles are those which comprise walls or membranes formulated from polymers, dendritic polymers, copolymers, polypeptides, copolypeptides, etc. The walls or membranes may be concentric or otherwise. The stabilizing compounds may be in the form of one or more monolayers or bilayers. In the case of more than one monolayer or bilayer, the monolayers or bilayers may be concentric. Stabilizing compounds may be used to form a unilamellar vesicle, comprised of one monolayer or bilayer; an oligolamellar vesicle, comprised of approximately two or three monolayers or bilayers; or a multilamellar vesicle, comprised of more than approximately three monolayers or bilayers. The walls or membranes of vesicles may be substantially solid (i.e., uniform), or they may be porous or semi-porous. The vesicles described herein include such entities commonly referred to as, for example, microspheres, hydrogels, microcapsules, microbubbles, particles, nanocarriers, nanoparticles, nanovesicles, micelles, bubbles, microbubbles, polymer-coated bubbles, and/or protein-coated bubbles, polymer matrixes, microbubbles and/or microspheres, nanospheres, microballoons, aerogels, clathrate-bound vesicles, and the like. The internal void of the vesicles may be filled with a wide variety of materials including, for example, water, oil, liquids, therapeutics, and bioactive agents, if desired, and/or other materials. The vesicles may also comprise one or more targeting moieties, if desired.

“Vesicle stability” refers to the ability of vesicles to retain the therapeutic or bioactive agents entrapped therein, after being exposed, for approximately one minute, to a pressure of approximately 100 millimeters (mm) of mercury (Hg). Vesicle stability is measured in percent (%), this being the fraction of the amount of gas which is originally entrapped in the vesicle and which is retained after release of the pressure. Vesicle stability also includes “vesicle resilience,” which is the ability of a vesicle to return to its original size after the release of said pressure.

“Wall filter” refers to a highpass filter designed to exclude low-frequency, high-amplitude Doppler signals from moving solid tissue, such as a vessel wall. Wall filter performance is critical to the success of a color Doppler system.

“Wall thump” refers to a strong, low-frequency clutter signal tending to obscure the Doppler frequency spectrum of interest, often arising from motion of the walls of a blood vessel.

Further, it must be noted that as used in this specification and claims, the singular forms “a”/“an” and “the” include plural referents unless the context clearly dictates otherwise. In addition, specific ranges recited are intended to be inclusive of the parameters bounding the range unless the context clearly dictates otherwise.

The following is a detailed description of illustrative embodiments of the present invention. As these embodiments are described with reference to the aforementioned drawings and definitions, various modifications or adaptations of the methods and/or specific structures described herein may become apparent to those skilled in the art. All such modifications, adaptations, or variations that rely on the teachings of this disclosure, and through which these teachings have advanced the art, are considered to be within the spirit and scope of this specification.

### Overview of the Preferred Embodiments

Therapeutic macromolecules such as, for example, antisense oligonucleotides, small interfering RNA (siRNA), and plasmid DNA, show enormous potential in the treatment of for example, a wide variety of inherited and acquired genetic disorders, viral infections, and cancer. Gene therapy aims to deliver these nucleic acids to specific cells to, for example, introduce novel genes and/or repair malfunctioning ones. However, delivery of said genetic and other therapeutic materials to cells, most with the requirement of intracellular delivery, provides multiple challenges which many preferred embodiments of the present invention are designed to overcome.

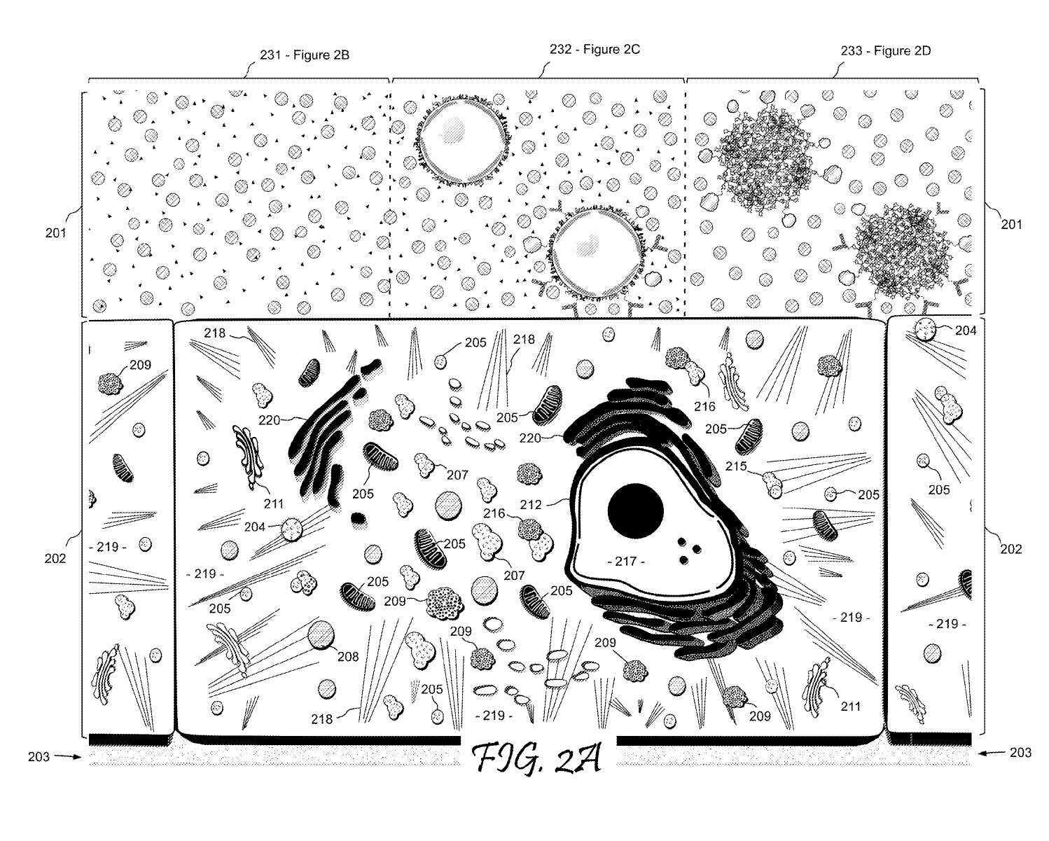

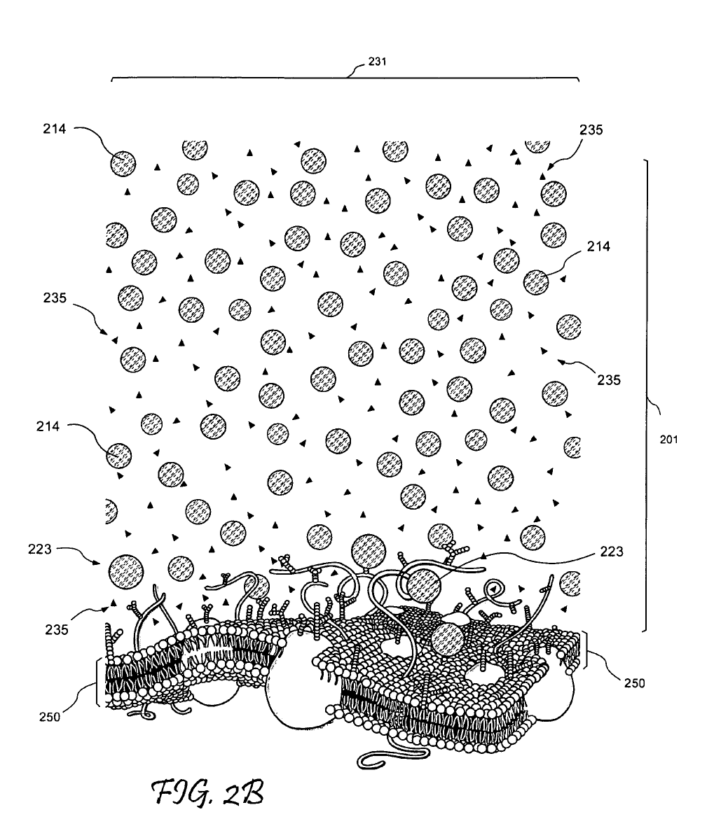

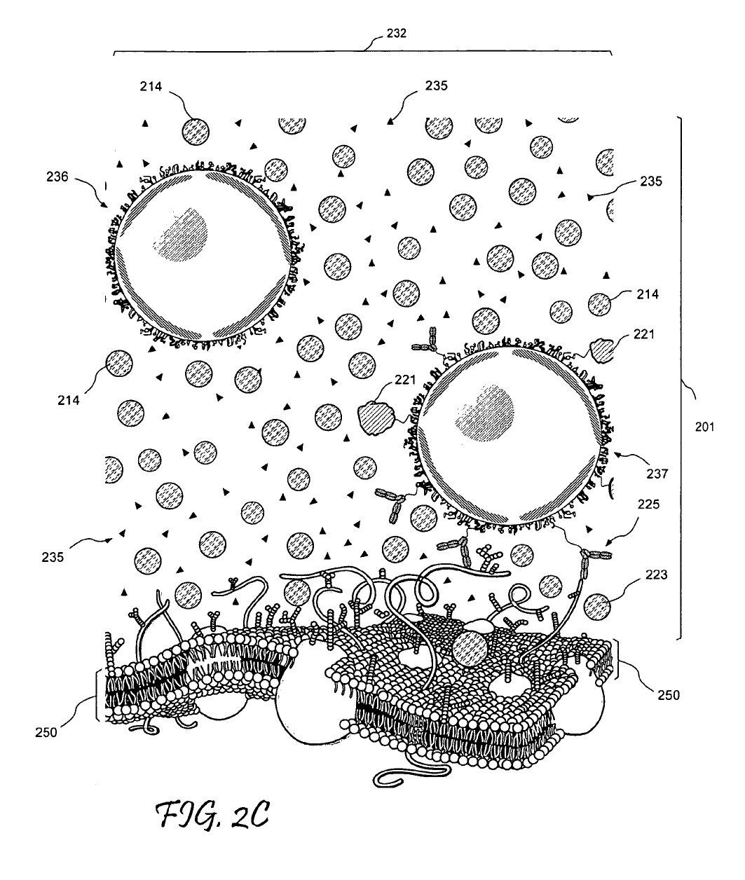

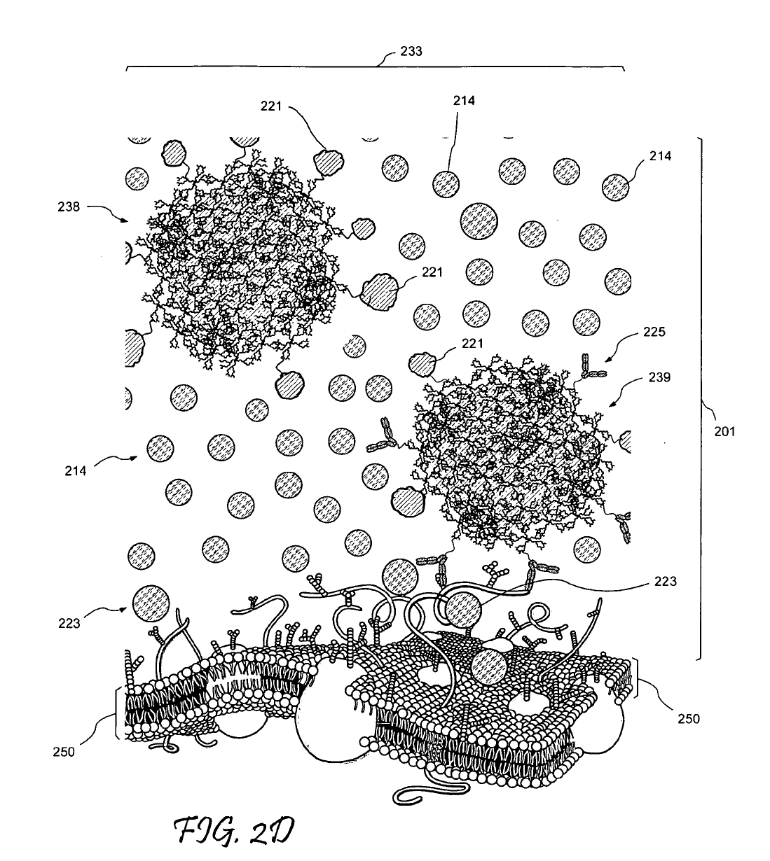

As discussed herein, to exert efficiently its activity without toxic effects, a drug must reach its pharmacological site(s) of action within the body. This may be inside the cell cytoplasm (FIG. 2A [219]) or into the nucleus (217) or other specific organelles, such as lysosomes (209), mitochondria (205), golgi apparatus (206), and/or the endoplasmic reticulum (220). Example pharmaceuticals requiring intracellular delivery include preparations for gene, antisense, and other therapeutic approaches, many of which must reach the cell nuclei (217); proapoptotic drugs, which target mitochondria (205); lysosomal enzymes which must reach the lysosomal compartments (209); and many others. Thus, the intracellular transport of different biologically active molecules and macromolecules is currently one of the key problems in drug delivery.

As will be reviewed in greater detail below, intracellular membrane barriers exist both due to the cell membrane itself (FIGS. 2A-2D [250]) and a variety of membrane-bounded intracellular vesicles (FIG. 2A) including, for some therapeutics, the nuclear membrane (212). In addition, the cytoplasm may constitute a significant diffusional barrier to gene transfer to the nucleus, depending primarily on therapeutic size. Embodiments of the present invention represent many new materials, methods, systems, and strategies to overcome these biological barriers and other challenges so troublesome to efficient intracellular drug delivery, especially for the delivery of therapeutic macromolecules.

FIG. 2A illustrates a section of a continuous blood vessel endothelium (202) of a patient. Following parenteral administration of free therapeutic (235) and/or parenteral administration the drug-carrying vesicles (236, 237, 238, and 239) of this disclosure, these components will be present at specific concentrations in the blood vessel lumen (201), along with, in this example, coadministered gaseous contrast agents (i.e., microbubbles; 214). The exact location of said therapeutics and/or vesicles will be dependent on a large variety of variables, including their size, surface charge, hydrophobicity, hydrophilicity, and many other characteristics and variables.

FIG. 2B illustrates a magnified view of a small portion of FIG. 2A (231). In this embodiment, free therapeutic has been administered to the patient, such as, for example, a stabilized protein (235). Contrast agents are also present (214) surrounding target tissues. Said microbubbles are at predefined concentrations so they may be, in many embodiments, at high densities (223), close to target cell membranes (250) and otherwise FIG. 2B. In additional embodiments, not illustrated, contrast agents (214) may be labeled with one or more targeting ligands, which may or may not be attached to said contrast agents via tethers.

FIG. 2C illustrates a magnified view of a small portion of FIG. 2A (232). Fully assembled, therapeutic-containing nanocarriers (e.g., polymersomes [236 and 237]) are illustrated along with unencapsulated free therapeutic (XXX) coadministered with said nanocarriers, where said nanocarriers (213) have reached the plasma membrane (201) of a target cell/tissue. In this embodiment (FIG. 2B) targeting ligands (i.e., antibody fragments [225] and components useful in magnetically targeting said vesicle [221]) are attached to one nanocarrier (237), again in this example, using tethers comprised of, for example, polyethylene glycol, where said ligands have actively guided the vesicle to its target.

In additional embodiments, nanocarriers may be passively targeted. Contrast agents are also present (214) surrounding the nanocarrier, and in this example, filling the extracellular spaces surrounding target tissues. Again, said microbubbles are at predefined concentrations so they may be, in some embodiments, at high densities (223), close to target cell membranes (250) and otherwise FIG. 2B. Further, in other embodiments not illustrated, contrast agents (214) may be labeled with one or more targeting moieties.

FIG. 2D illustrates a magnified view of a small portion of FIG. 2A (233). In this embodiment, therapeutic is encapsulated within nanocarriers comprised substantially of dendritic polymers (238 and 239), wherein said nanocarriers are in close proximity to the plasma membrane (250) of a target cell/tissue. The use of dendritic polymers are especially preferred in carrier vesicles for acoustically mediated drug delivery because their highly branched, monodisperse characteristics in the nanometer size range offer the control that this type of embodiment requires. This includes control of the chemical nature of the carrier, control of molecular weight, control of the surface and internal structure/character, and a variable vital in cell and tissue-specific targeting, control of dimensions. Most importantly, dendritic polymer architecture offers the ideal characteristics to adjust and control the acoustic responsiveness of drug carrying vesicles, and thus represents an optimal nanocarrier embodiment. As such, dendritic polymers are the latest evolutionary stage of polymer chemistry and present significant opportunities in a variety of disciplines including, as exemplified in the many embodiments of the present invention, radically new applications in drug and gene delivery. Also in this embodiment FIG. 2D, targeting ligands (i.e., antibody fragments [225] and components useful in magnetically targeting said vesicle [221]) are attached to said nanocarrier (239), again in this example, using tethers comprised of, for example, polyethylene glycol, where said ligands have actively guided the vesicle to its target. In additional embodiments, nanocarriers may be passively targeted. Additional preferred embodiments, not illustrated, include enclosing the therapeutic-containing nanocarriers or free therapeutic in an acoustically responsive or other drug-delivery polymer matrix, such as a hydrogel.

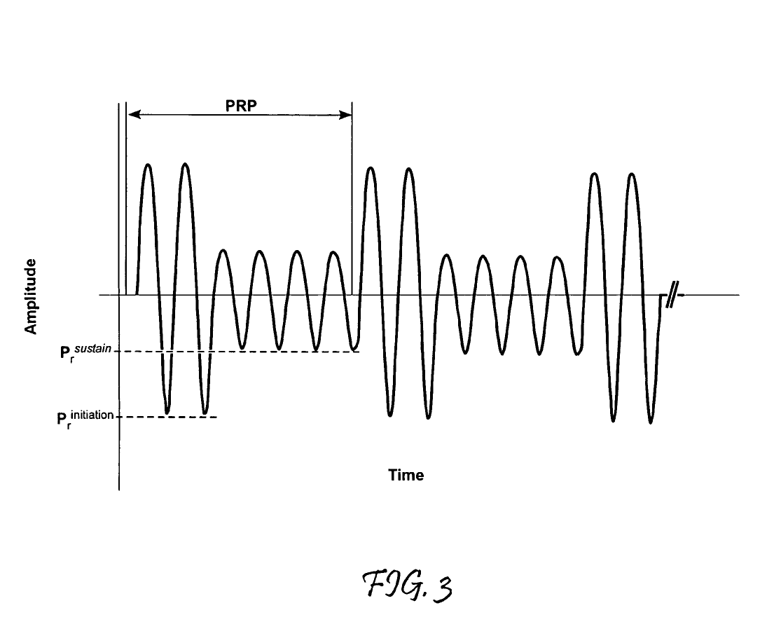

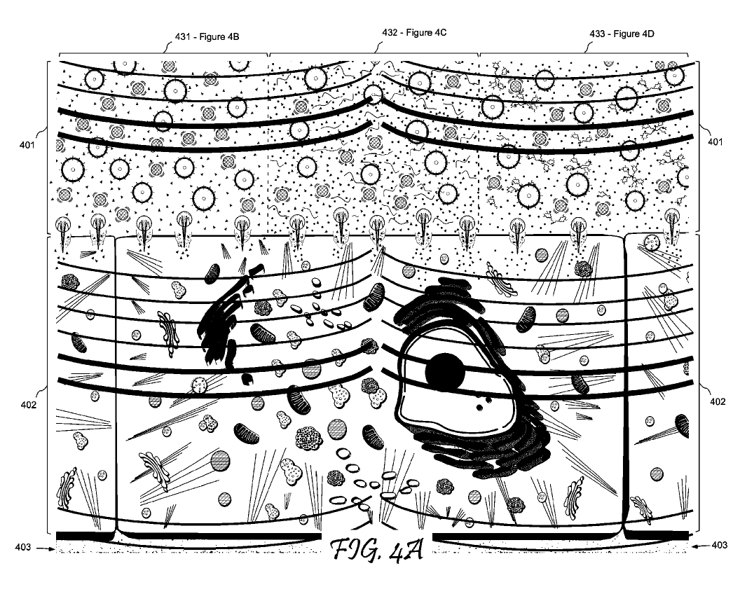

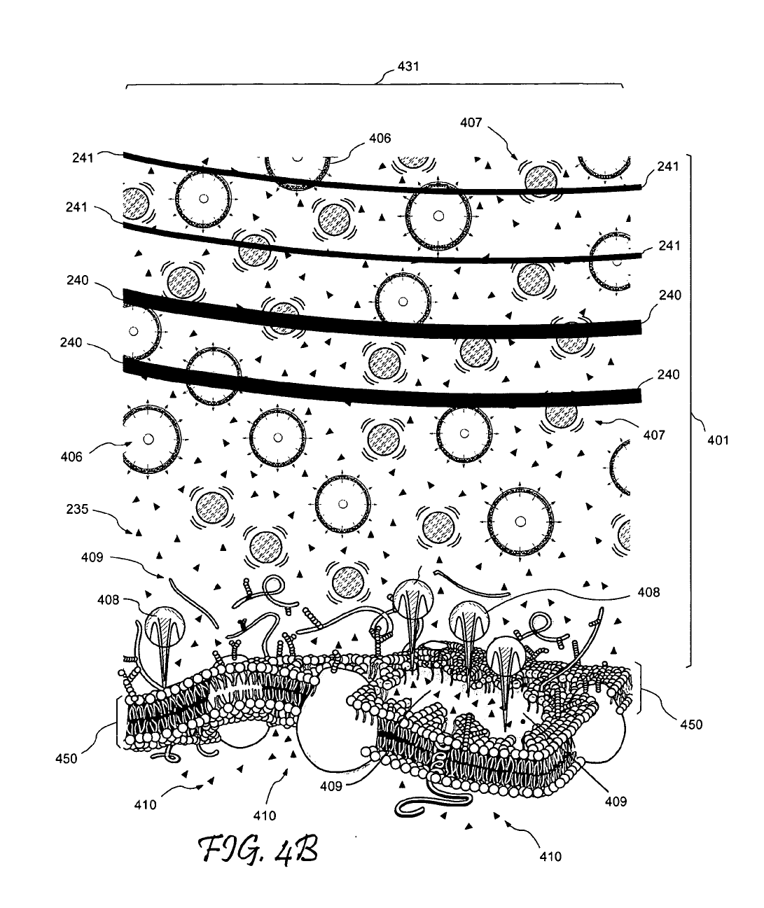

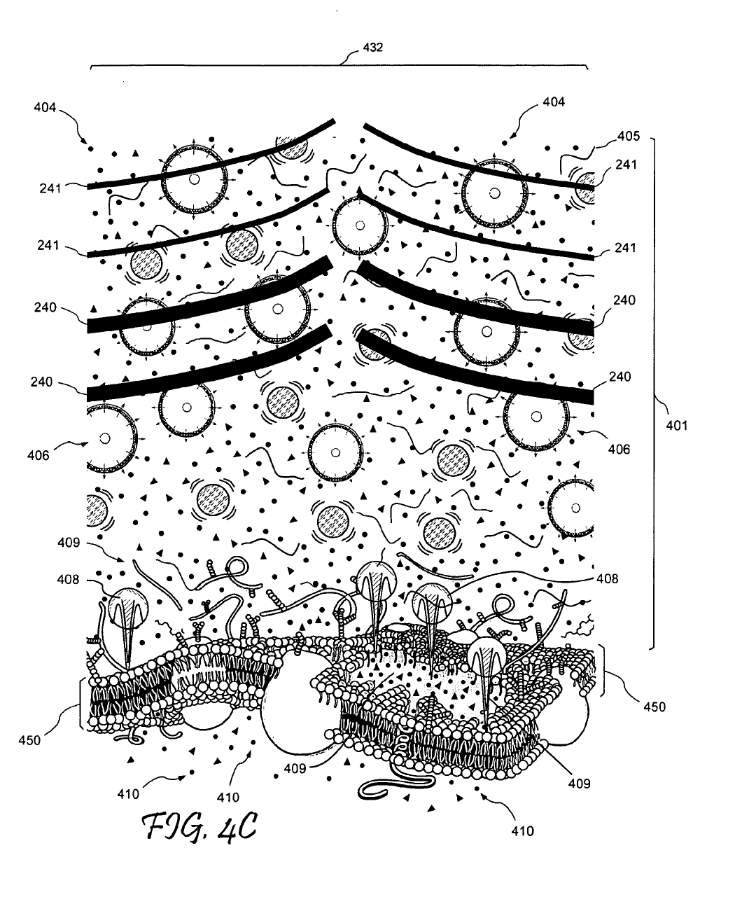

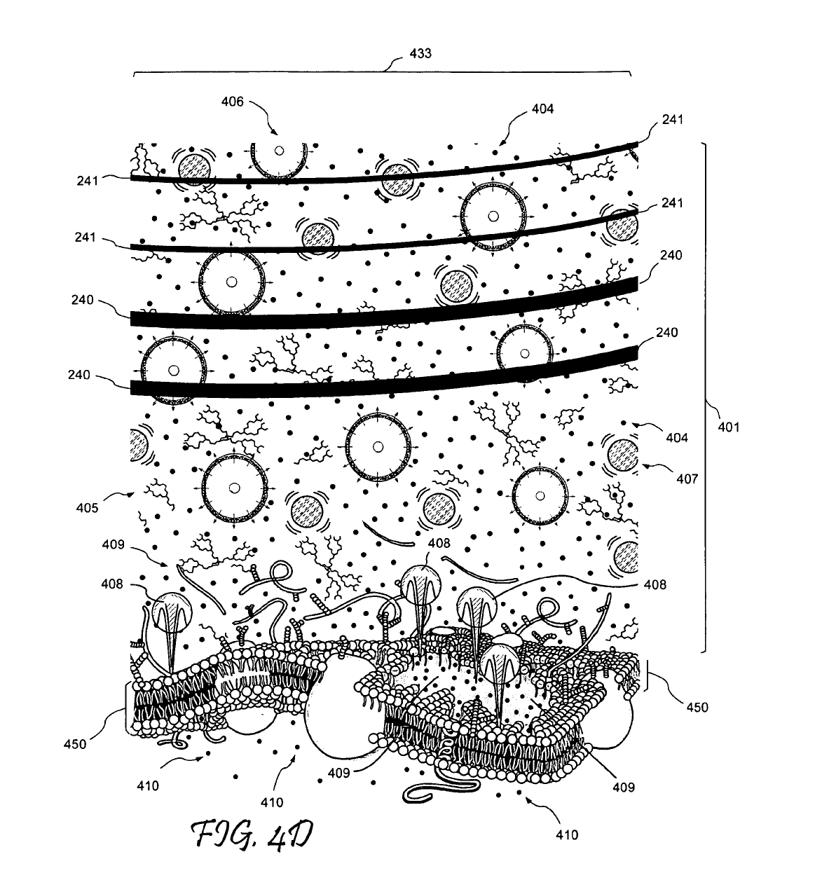

Once free therapeutic and/or therapeutic-containing nanocarriers have reached their target (FIG. 2A-2D), therapeutic release is initiated by exposure of the target region to preferably, pulsed, high-intensity focused ultrasonic energy (FIG. 3). FIGS. 4A-4D illustrate the regions described in FIGS. 2A-2D during this ultrasonic energy exposure, for example, the pulse sequences illustrated in FIG. 3. The ultrasonic energy used in the present teachings will have different characteristics, depending on the drug delivery application. In one embodiment, ultrasonic energy is applied at, for instance, a center frequency of 1 MHz, with energy levels varying from approximately 0.75 Watt (W) per square centimeter (cm²) to approximately 2.75 W/cm², where said pulsed ultrasonic energy is optimally comprised of cavitation initiating (240) and sustaining sequences (241) (FIG. 3).

In other embodiments, the frequency of the ultrasonic energy used may vary from approximately 0.025 MHz to approximately 10 MHz. In general, frequency for ultrasonic drug delivery preferably ranges between approximately 0.75 MHz and approximately 3 MHz in most applications, with from approximately 1 MHz and approximately 2 MHz being normally preferred. In addition, energy levels may vary from approximately 0.5 Watt (W) per square centimeter (cm²) to approximately 5.0 W/cm² in most circumstances, with energy levels from approximately 0.5 to approximately 2.5 W/cm² normally being preferred. Energy levels for ultrasonic treatments causing hyperthermia are generally from approximately 5 W/cm² to approximately 50 W/cm², and in most circumstances, with the present teachings, should be avoided. When very high frequencies are used, for example, greater than approximately 10 MHz, the sonic energy will generally penetrate fluids and tissues to a limited depth only.

FIG. 3 illustrates one of the large number of possible ultrasonic pulse sequences that may be used in practicing the methods of this specification, with the characteristics of said sequences and ultrasonic energy customized to a particular application (e.g., focus, frequency, pulse length, pulse repetition frequency, pulse repetition period [PRP]). Each ultrasonic energy pulse in this example has three primary functions at the target site:

1. To initiate and/or sustain acoustic cavitation,

2. A small fraction of the desired tissue permeation results, and

3. Said energy predisposes tissue to permeation initiated and/or sustained by subsequent pulses.

In addition, as detailed later, a set of multiple parameters are created, including but not limited to ultrasonic intensity, peak negative pressure, peak positive pressure, time of arrival, duration, and frequency, allowing for several feedback, optimization, and real-time monitoring opportunities. For example, by altering the characteristics of the therapeutic acoustic pulses at biological barriers in the target area, membranes and obstructions to drug transport are broken down systematically and controllably, facilitating enhanced therapeutic transport and diffusion across said barriers, and minimizing tissue damage.

By assembling a known, and/or optimally sized distribution of microbubbles in the tissue volume under treatment (FIGS. 2A-2D and FIGS. 4A-4D), lower ultrasonic energy intensities may be utilized, thereby avoiding excessive tissue heating, and in some cases, allowing sound propagation through intervening bone, such as the rib cage or skull. Moreover, in a preferred embodiment of the invention, by the proper pre-sizing of for example, magnetically targeted microbubble ensembles, the therapeutic sound field need not be focused or localized if the therapy volume is the only tissue with said microbubbles that are properly “tuned” to the incident ultrasound frequency. Cavitation also has interesting chemical effects on drugs, which can enhance their intended effect (e.g., effective activation of anticancer drugs). Finally, in the methods outlined herein, feedback of the permeation process can be accomplished during tissue alteration and drug delivery treatment; either continuously or at intervals.

Each individual pulse produces little tissue disruption and permeation on its own; rather, many thousand and possibly more than one a million pulses are required to produce the desired effect. In addition to microbubbles that may be administered to the patient, either systemically or locally at the target site, each pulse may also assist in producing a bubble cloud at said site. These bubble ensembles can be easily seen by ultrasound imaging scanners or by special transducers used for techniques such as, for example, Active Cavitation Detection (i.e., ultrasound backscatter detection; ACD). In the case of existing imaging systems, said bubbles show up as bright spots on the image, which may be localized to the target region on said image by moving the therapeutic transducer focus either mechanically, or in another preferred embodiment, by phased array electronic focus scanning.

Without wishing to be bound by any particular theory, when an ultrasound wave propagates in tissue (FIGS. 4A-4D), a mechanical strain is induced, where strain refers to the relative change in dimensions or shape of the material that is subjected to stress, and where said strain may be especially significant near gas or vapor bubbles. Depending on a variety of parameters, acoustic cavitation may result, a most important phenomenon for the application of the present teachings. FIG. 4B is a magnified view of a region of FIG. 4A (431), FIG. 4C is a magnified view of another region of FIG. 4A (432), and FIG. 4D is a magnified view of yet another portion of FIG. 4A (433). Both initiation (240) and sustaining (241) sequences of pulsed ultrasonic energy are illustrated, inducing acoustic cavitation in the lumen (401) of the blood vessel section illustrated in FIGS. 4A-4D.

Cavitation, in a broad sense, refers to ultrasonically induced activity occurring in a liquid or liquid-like material that contains bubbles or pockets containing gas or vapor. These bubbles originate at locations termed “nucleation sites,” the exact nature and source of which are not well understood in a complex medium such as tissue. Or, as in preferred embodiments of this disclosure, these bubbles may also be introduced into the insonated area FIGS. 2A-2D (214), either directly or indirectly, in the form of for example, gaseous contrast agents (i.e., microbubbles) (214). Under ultrasonic stimulation FIGS. 4A-4D, with the appropriate parameters (e.g., focus, frequency, pulse length, pulse repetition frequency, etc.), said bubbles oscillate (407), creating a circulating fluid flow—called microstreaming—around the bubble, with velocities and shear rates proportional to the amplitude of the oscillation. At high amplitudes, the associated shear forces are capable of shearing open cells and synthetic vesicles such as those of this disclosure. Further, said bubbles may collapse, sending out, for example, shockwaves in their immediate vicinity (406). These shockwaves and other disturbances at the target site also result in, for example, nanocarrier disruption (405) and therapeutic release (404). In addition, bubbles close to surfaces while undergoing inertial cavitation (408), in an optimal embodiment, may emit membrane-piercing microjets (408). If properly controlled, said microjets (408) preferably function in the present disclosure, for example, in permeabilizing cell membranes at the target, and in some cases tearing pieces of membrane (409) from target cells and tissues. Emitted shock waves (406) from collapsing bubbles probably also contribute to membrane disruption. Thus, cavitation is a potentially violent event, effectively concentrating ultrasonic energy into a small volume.

Said oscillating bubbles can also result in acoustic pressure, a net force acting on other suspended bodies in the vicinity of an oscillating bubble. If the body is more dense than the suspending liquid, the body is pushed toward the oscillating bubble; if less dense, the body is repelled (Nyborg, 2001). Many of the drug-carrying vesicles of this disclosure are more dense than water, and thus will be convected toward the bubble, thus increasing the dispersive transport of the drug carrier, particularly if the vesicle is drawn into the microstreaming field around said bubble and is sheared open by the high shear rate, thus releasing therapeutic. If the vesicle is another microbubble, it will be dispersed away from the primary oscillating bubble because it is less dense. Thus, a field of microbubbles, such as those coadministered with the nanocarriers described herein, will tend to spread itself in the ultrasonic field, and at the same time, attract and shear more dense vesicles such as suspended cells or the nanocarriers of the present teachings. In theory, said nanocarriers will not be acoustically active since they contain no gas. However, these nanocarriers should be drawn toward and then sheared open by the action of the surrounding cavitating bubbles, as long as said bubbles are at sufficient densities.

In addition to the many mechanical stresses summarized above, cavitation may affect a biological system by virtue of a temperature increase and/or free radical production. While cavitation can produce extremely high temperatures immediately close to the nucleation site, it is traditionally referred to as a non-thermal mechanism of tissue damage (O'Brien, 2007). Indeed, for the purposes of this specification, temperature increases at the target must be minimized. This is accomplished, in part, by selecting the most appropriate ultrasound parameters for a given application. The occurrence of cavitation, and its behavior, depends on many variables, including the ultrasonic pressure, whether the ultrasonic field is focused or unfocused, continuous or pulsed, or combinations thereof; to what degree there are standing waves (i.e., energy reflecting back onto itself); the nature and state of the material and its boundaries; as well as many other variables. Thus, the ultrasonic energy utilized by the present teachings must have its properties tailored to specific drug delivery applications. The major goal in many of said applications is to control the amount and extent of acoustic cavitation at the target site. If properly controlled, the nanocarriers of this disclosure will be effectively disrupted (FIGS. 4C-4D [405]) and the enclosed therapeutic(s) freed into the surrounding medium (404). Said cavitation activity also results in tissue permeation allowing extravasation and therapeutic entry; importantly in the case of therapeutic macromolecules, avoiding entry by the usually destructive endocytic pathway, reviewed below, while minimizing permanent and long-term damage (i.e., sonolysis and cytotoxicity) to the patient.

Because many eucaryotic cells inhabit mechanically stressful environments, their plasma membranes are frequently disrupted. Survival requires that the cell rapidly repair or reseal said disruption (McNeil et al., 2003). Obviously, this phenomena is critical for the successful application of the present teachings. Rapid membrane resealing is an active and complex structural modification that employs endomembrane, as its primary building block (i.e., literally a “patch”), and cytoskeletal and membrane fusion proteins as its catalysts. Endomembrane is delivered to the damaged plasma membrane through exocytosis, a ubiquitous Ca²⁺-triggered response to disruption. Tissue and cell level architecture may prevent disruptions from occurring, either by shielding cells from damaging levels of force or, when this is not possible, by promoting safe force transmission through the plasma membrane via protein-based cables and linkages (McNeil et al., 2003). Therefore, membrane damage and its subsequent repair is a normal process occurring in the patient; embodiments of the present invention take advantage of these repair mechanisms for assisting in safe and effective intracellular drug delivery.

Without wishing to be bound by any particular theory, in order to more fully illustrate the novelty and importance of the present teachings, some of the preferred embodiments of this specification will be discussed in the context of currently accepted biological structures and how said embodiments may overcome barriers to conventional drug delivery know in the art. These biological barriers can be broadly categorized into extracellular and intracellular barriers.

Extracellular barriers. To protect, for example, therapeutic nucleic acids from degradation while transferring extracellular spaces before reaching their target, said therapeutics may be enclosed within a drug-carrying vesicle (e.g., a nanocarrier comprised preferably of biodegradable polymers, or mixtures thereof). Said vesicles must travel through extracellular barriers (e.g., blood) before they reach their target tissue, which may be situated near, as well as far away from, the site of administration. Systemic, parenteral administration of free therapeutic(s) and/or the nanocarriers of this disclosure is strongly preferred, as it may allow the distribution of said carriers via the bloodstream to tissues that are otherwise difficult to reach via more localized application. However, the blood forms a major barrier to said therapeutics and vesicles, as biomolecules (e.g., albumin) are known to extensively bind to cationic carriers, causing a neutralization or reversion of their surface change. Neutralization of said nanocarriers by albumin or other biomolecules abolishes the electrostatic repulsion that exists between said carriers, allowing them to come into close contact upon collision. When such close contact happens, for example, Van der Walls forces may take place and hold the vesicles together, resulting in aggregates. In addition, the binding of such negatively charged biomacromolecules to, in an optimal embodiment, self assembled, acoustically responsive nanocarriers, may subsequently deassemble said vesicles. These difficulties and complications will be largely dependent on nanocarrier composition and solved in a variety of ways, some of which are described herein.

Other major extracellular barriers that must be overcome include endothelial cells and basement membranes. Indeed, the nanocarriers of this disclosure have to extravasate before they can reach tissues localized outside of the bloodstream. With conventional drug-containing vesicles, extravasation is mainly determined by the vesicle's size and the permeability of the capillary walls, a characteristic that greatly varies between tissues (Takakura et al., 1998). Based on the morphology of the endothelial and basement membrane, capillary endothelium can be divided into continuous, fenestrated, and discontinuous endothelium (Simionescu, 1983). The continuous endothelium, which is found in all types of muscular tissues and lung, skin, and subcutaneous tissues, is the tightest and prevents the passage of materials greater than approximately 2 nm. The brain endothelium offers an even stronger barrier; only small hydrophobic molecules can cross the blood-brain barrier. Fenestrated endothelia, which occur in the intestinal mucosa, the kidney, and the endocrine and exocrine glands, contain openings of approximately 40 nm to 60 nm in diameter. However, the continuous basement membrane surrounding these capillaries prevents the passage of macromolecules larger than approximately 11 nm. Discontinuous capillaries or sinusoidal capillaries are found in the liver, spleen, and bone marrow. These capillaries have endothelial junctions of approximately 150 nm or even up to approximately 500 nm, and contain either no (e.g., the liver) or a discontinuous basement membrane (e.g., the spleen and bone marrow). Leaky capillaries are also found at sites of inflammation and in tumors (Baban et al., 1998). Extravasation of particles with a diameter of up to 400 nm in certain tumors has been reported (Yuan et al., 1995). Although, other reports found no extravasation of particles larger than 100 nm in tumors (Kong et al., 2000). Thus, the size of the nanocarriers of this disclosure, like many conventional drug-carrying vesicles, may be largely dependent upon their intended use. However, it is contemplated that acoustically disrupting and modifying the permeability of target and possibly other tissues, using the methods and techniques of this specification, may allow much greater-sized vesicles to extravasate, with higher therapeutic-containing payloads, and cross other biological barriers in a far more efficient manner when compared to current drug delivery vesicles and methods known in the art.

Ocular drug and gene therapy may offer new hope for severe eye diseases such as, for example, retinitis pigmentosa and age-related macular degeneration (AMD). Many of these ocular diseases are due to a gene defect in the retina, a multilayered sensory tissue that lines the back of the eye. The blood-retinal barrier and the sclera prevent large molecules, such as many conventional drug-carrying vesicles, from accessing the retina after systemic or topical applications (Duvvuri et al., 2003). However, by using the methods of the present teachings, the blood-retinal barrier and sclera may be temporarily and safely disrupted, allowing the nanocarriers of this disclosure to effectively pass said structures. In addition, intravitreal injection, which is less invasive than subretinal injection, may also be a route for ocular drug delivery using the present teachings. However, before free therapeutic and/or the drug-carrying vesicles of this specification can reach the retina, they must travel through the vitreous, a gel-like material built up from collagen fibrils bridged by proteoglycan filaments that contain negatively charged glycosaminoglycans (Bishop, 1996). This biopolymer network may immobilize many conventional carriers with, for example, glycosaminoglycans, further binding and impeding said vesicles. Indeed, recent studies have confirmed this, where a thin layer of vitreous on top of retinal cells almost completely blocked the gene expression of cationic polyplexes and lipoplexes (Pitkanen et al., 2003). Further, cationic lipoplexes have been shown to be severely aggregated when mixed with vitreous (Peeters et al., 2005). This aggregation is most likely due to the binding of negatively charged biopolymers in the vitreous, such as, for example, charged glycosaminoglycans, to the cationic lipoplexes, neutralizing their surface charge, thus leading to aggregation. These aggregated carriers may become completely immobilized in the vitreous gel, having little chance to reach retinal cells. In addition, binding of glycosaminoglycans to lipo- and polyplexes may also impede the intracellular processes which lead to successful gene expression (Ruponen et al., 2001). By following the teachings of this disclosure, optimal embodiments of the present invention may allow effective intracellular drug delivery to both the anterior and posterior portions of the eye by, for example, temporarily disrupting said structures, assisting free therapeutic and/or the nanocarriers of this specification to effectively travel through the vitreous, breaking up and limiting free therapeutic and/or nanocarrier aggregation, and assisting in crossing formidable membrane barriers.

Some of these extracellular barriers may be avoided, at least partially, by coating the nanocarriers of this disclosure with compounds, such as, for example, polyethylene glycol (PEG), derivatives of PEG, and other polymers and materials which may prevent aggregation, reduce toxicity, prevent uptake by the mononuclear phagocytic system, enhance the circulation time in the bloodstream and improve the journey of said nanocarriers through extracellular matrices like serum, sputum, and vitreous. The vesicles of this disclosure can be shielded with polymers, for example, by using a cationic nanocarrier with DNA covalently coupled to the shielding polymer, and subsequently mixed with the DNA. During the self-assembly of said vesicle, the DNA and the cationic carrier interact with each other creating a slightly hydrophobic core that is surrounded by a shield of hydrophilic polymers. This method has the disadvantage that the shielding polymers can hinder the self-assembling process between the cationic carrier and the anionic DNA, especially when high amounts of shielding polymer are used. Therefore, post-shielding may also be utilized, involving the physical incorporation or covalent attachment of the shielding polymer, or other compound, to preformed nanocarriers. Further, the cationic surface of a preassembled nanocarrier also allows ionic coating by negatively charged polymers. In addition, anionic polymers, such as, for example, poly (propylacrylic acid) (PPAA) may be utilized, as discussed in greater detail later in this disclosure.

The presence of hydrophilic polymers on the surface of the nanocarriers, described herein, also prevents aggregation by avoiding vesicles that can come in close proximity to each other during collision. In addition, when present in sufficient amounts, these dangling polymers protruding on the surface of said nanocarriers also avoid macromolecules that can reach, in some embodiments, the charged core of the nanocarrier. Unfortunately, they can also prevent close interactions between the nanocarriers and cell membranes. This may be overcome by reversible shielding of said vesicles with, for example, polyethylene glycol (PEG), which implies that the vesicles lose their protective shield at or in the target cells. In addition, there are normally gaps between the polymer chains that may allow small charged molecules to reach the surface of the nanocarriers. The size of these gaps depends on the degree of shielding and the chain length of the polymers. However, long PEG chains (>10 kDa) may entangle in the biopolymer network of biogels. Therefore, particles containing such long, for example, PEG chains may become immobilized in mucus or vitreous. Alternative methods for shielding include the use of synthetic polypeptides, poly(propylacrylic acid), polysaccharides, and the like, either alone, or along with PEG or a derivative of PEG. Importantly, in an optimal embodiment, ends of the shielding polymers may be used to provide the nanocarriers of this disclosure with a variety of different targeting moieties.

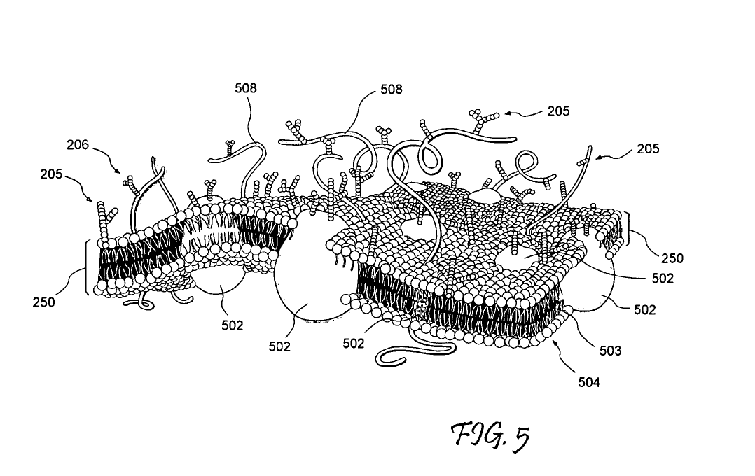

Intracellular barriers. Even though parenteral administration of pharmaceuticals ensures delivery to the systemic circulation, a drug still must traverse the semipermeable, plasma membrane bordering target cells, before reaching the interior of the cells of target tissues as well as, possibly, intracellular membranes depending primarily on the therapeutic and its intended site of use. These membranes are biologic barriers that selectively inhibit the passage of larger drug molecules, (e.g., therapeutic macromolecules) and are composed primarily of a bimolecular lipid matrix, containing mostly cholesterol and phospholipids (FIG. 5). The lipids provide stability to the membrane and, a most important characteristic for the present teachings, determine its permeability characteristics. Globular proteins of various sizes and compositions are embedded in the matrix; they are involved in transport and function as receptors for cellular regulation. An illustration of the Fluid Mosaic Model of plasma membranes developed by Singer et al. in 1972 is shown in FIG. 5. All current evidence in modern biology is compatible with the Fluid Mosaic Model, and this embodiment is broadly accepted among scientists from multiple disciplines.

According to the Fluid Mosaic model (FIG. 5), a membrane is a liquid in two dimensions, but an elastic solid in the third dimension (250). Importantly, and for the successful application of the present teachings, this elasticity is critical and contributes substantially to the self-healing properties of biological membranes. In this model, proteins float freely in the fluid bilayer (502), and are also held in place by their lipophilic sections which are attracted to the fatty middle layer of the membrane (503), accounting for their high mobility in biological membranes. According to the Fluid Mosaic Model (FIG. 5), the basic structure of the membrane is provided by the phospholipid molecules. For example, unsaturated fatty acid tails make a membrane more liquid, while the addition of cholesterol to the fatty layer makes said membrane more viscous and more repellent to water. Proteins are responsible for many of the special characteristics of different types of membranes (502) controlling the ability of cells to transport molecules, receive chemical messages, and attach to adjacent cells, as well as many other characteristics and processes (502). Because lipid molecules are small when compared to proteins, there are typically many more lipid molecules than protein molecules in biological membranes—approximately 50 lipid molecules for each protein in a membrane that is 50% protein by mass. Like membrane lipids (505), membrane proteins often have oligosaccharide chains attached to them on the portion of the molecule that faces the cell exterior (506). Thus, the surface the cell presents to the exterior is rich in carbohydrate, essentially forming a cell coat. As described later in this specification, these external structures on cell membranes may represent important targeting structures for the drug carrying vesicles (i.e., nanocarriers) of this specification (FIG. 5).

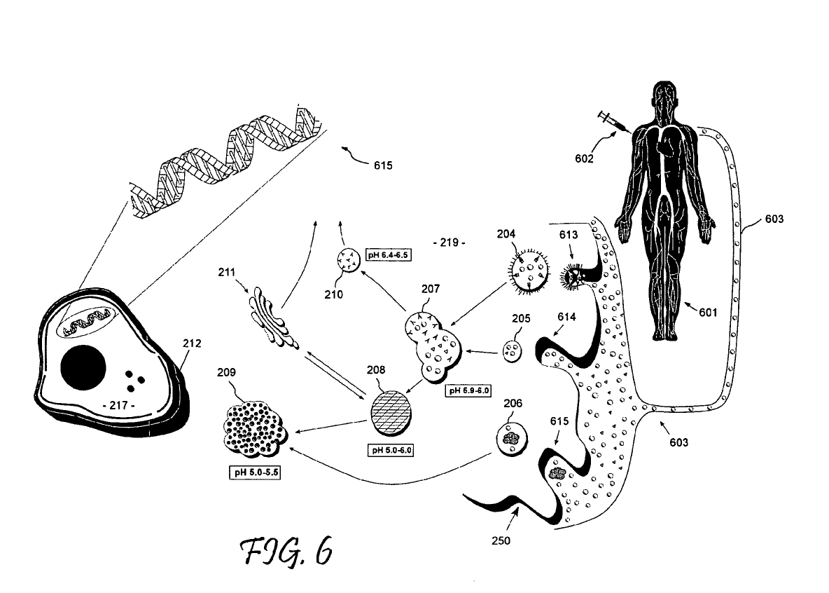

Especially troublesome intracellular barriers for the delivery of for example, therapeutic macromolecules, are the vesicles of the endocytic pathway. FIG. 6 illustrates a schematic representation of the biological distribution of many currently used colloidal therapeutic carriers (603) following parenteral administration (602) to a patient (601). These conventional carriers are usually required to circulate in the bloodstream (603), and as reviewed previously, escape recognition by the reticuloendothelial system (RES) and avoid hepatic clearance, glomerular excretion, etc. Receptor-mediated targeting may be achieved by installing pilot/targeting moieties on the surface of these carriers using, for example, end-functionalized block copolymers. Without wishing to be bound by any particular theory, many conventional macromolecular carriers, such as, for example, nanospheres, micelles, liposomes, and the like, usually enter target cells by endocytosis (FIG. 6) where endosomes with encapsulated carriers are separated from the cell membrane by a process of inward folding (613, 614, 615). These vesicles have an increasingly acidic pH as the endosomes move toward a final destination of cellular lysosomes (209), where nearly all materials, including the endocytosed therapeutic macromolecules, will be destroyed (i.e., hydrolyzed). Thus, membranes inside the cell, including the nuclear membrane (212), may represent additional biological barriers for the delivery of therapeutic macromolecules (FIG. 6) as discussed in greater detail below. For the purposes of more clearly understanding the present teachings, endocytosis may be divided simplistically into three major processes: (1) receptor-mediated endocytosis, (2) pinocytosis, and (3) phagocytosis.

Receptor-mediated endocytosis (FIG. 6 [613]) is typically prompted by the binding of a large extracellular molecule—such as a protein—to a receptor on the cell membrane. Many conventional intracellular drug delivery vesicles utilize receptor-mediated endocytosis for entry into the cell cytoplasm. The receptor sites utilized by this process are commonly grouped together along coated pits, in the membrane, which are lined on their cytoplasmic surface with bristle-like coat proteins (613) (i.e., clathrin chains with the AP-2 adaptor complexes). The coat proteins are thought to play a role in enlarging the pit and forming a vesicle (204). When the receptors bind their target molecules, the pit deepens (613) until a protein-coated vesicle is released into the cytosol (204). Through receptor-mediated endocytosis, active cells are able to take in significant amounts of particular molecules (e.g., ligands), including ligand-labeled, drug-containing vesicles that bind to the receptor sites, extending from the cytoplasmic membrane into the extracellular fluid surrounding the cell. However, vesicles produced via receptor-mediated endocytosis may internalize other molecules in addition to ligands, although the ligands are usually brought into the cell in higher concentrations (FIG. 6).

By the mechanisms of pinocytosis, a cell is usually able to ingest droplets of liquid from the extracellular fluid (FIG. 6). This is a constant process with the rate varying from cell to cell; for example, a macrophage internalizes approximately 25% of its volume every hour. All solutes found in the medium outside the cell (603) may become encased in the vesicles formed via this process (205). Those present in the greatest concentration in the extracellular fluid are likely to be the most concentrated in the membrane vesicles (205). Pinocytic vesicles tend to be smaller than vesicles produced by other endocytic processes (205), with the major purpose of pinocytosis being to take in a wide range of extracellular molecules and atoms including minerals. Entry of, for example, many smaller therapeutic nucleic acids by conventional methods of administration, is believed to be almost entirely by pinocytosis (Akhtar et al., 2007). Also, cholesterol-containing particles called LDLs, composed of cholesterol and proteins, are taken up by pinocytosis. The other purpose of pinocytosis is also important, but less obvious. In order for cells to communicate with each other, they must have the ability to constantly secrete hormones, growth factors, neurotransmitters for nervous system function, etc. As mentioned previously, secretion of membrane is also critical for repairing cell membrane damage. While some of this membrane is synthesized and stored inside the cell, much comes from the cell surface that is continually internalized by pinocytosis (614 and 205). Therefore, this constant process means there is a considerable amount of membrane internalized to quickly recycle to the surface for secretion, and most importantly for the present teachings, a ready source of renewable materials for membrane repair.

Phagocytosis is the process by which cells ingest large objects (FIG. 6 [615]) (e.g., such as prey cells or chunks of dead organic matter) and is probably the most well-known manner in which a cell imports materials from the extracellular fluid. This debris is then sealed off into larger vacuoles (206). Lysosomes (209) then merge with this vacuole, turning it into a digestive chamber. The products of the digestion are then released into the cytosol. Macrophages are cells of the immune system that specialize in the destruction of antigens (e.g., bacteria, viruses, and other foreign particles) by phagocytosis. With all three endocytic mechanisms, the vessels formed can be broadly termed endosomes (FIG. 6).

Typically, once endosomal vessels have formed and gained entrance to the cell cytoplasm, some of the ingested molecules are selectively retrieved and recycled to the plasma membrane (210), while others pass on into late endosomes (FIG. 6 [208]). This is the first place that endocytosed molecules usually encounter caustic enzymes (i.e., primary hydrolases). The interior of the late endosomes is mildly acidic, allowing for the beginning of hydrolytic digestion. Once freed into the cytoplasm, several small vesicles produced via endocytosis may come together to form a single entity (207). This endosome generally functions in one of two ways. Most commonly, endosomes transport their contents in a series of steps to a lysosome (209), which subsequently digests the materials. In other instances, however, endosomes are used by the cell to transport various substances between different portions of the external cell membrane. An endosome that is destined to transfer its contents to a lysosome generally goes through several transformations along the way. In its initial form, when the structure is often referred to as an early endosome, the specialized vesicle contains a single compartment. Over time, however, chemical changes in the vesicle take place and the membrane surrounding the endosome folds in upon itself in a way that is similar to the invagination of the plasma membrane. In this case, however, the membrane is not pinched off. Consequently, a structure with multiple compartments, termed a multivesicular endosome, is formed (207). The multivesicular endosome (207) is an intermediate structure in which further chemical changes, including a significant drop in pH, take place as the vesicle develops into a late endosome (308). Though late endosomes (308) are capable of breaking down many proteins and fats, a lysosome (309) is needed to fully digest all of the materials contained within multivesicular and late endosomes. Therefore, the necessity of escape of endocytosed therapeutic macromolecules from these endosomal and other vesicles is a key problem in intracellular drug delivery. The present teachings represent broadly applicable methodologies to solve these and other critical challenges.

Cytosolic sequestration and degradation is yet another problem especially for, for example, nucleic acid macromolecules (FIG. 6 [615]) that require entry into the cell nucleus for therapeutic efficacy. The cytoplasm (FIG. 2A [219]) is composed of a network of microfilamental and microtubule systems and a variety of subcellular organelles bathing in the cytosol. The cytoskeleton (218) is responsible for the mechanical resistance of the cell, as well as the cytoplasmic transport of organelles and large complexes. The mesh-like structure of the cytoskeleton (218), the presence of organelles, and the high protein concentration impose an intensive molecular crowding of the cytoplasm which limits the diffusion of large-sized macromolecules (Luby-Phelps, 2000). Indeed, the cytosol (204) probably constitutes a significant diffusional barrier to gene transfer to the nucleus. Embodiments of the present invention may be of assistance in increasing cytoplasmic diffusion of therapeutic and molecules and macromolecules inside target cells and tissues by methods including those described previously.

Sequence-specific gene silencing, using small interfering RNA (siRNA), is now being evaluated in clinical trials, and is a Nobel prize-winning technology with considerable therapeutic potential. However, efficient intracellular siRNA delivery to specific target sites in the body following systemic administration is the most important hurdle for widespread use of RNAi in the clinic (Akhtar et al., 2007). At present, it is widely thought that cellular uptake of siRNA occurs via pinocytosis, most likely in a manner similar to that observed for other gene-silencing molecules (e.g., oligonucleotides and ribozymes) (Akhtar et al., 2007). Thus, preferred embodiments of the present invention should offer ideal systems and methods for successful siRNA delivery. In order for these applications to be successful clinically, the RNA duplex structure may be modified chemically, such as, for example, modifications to the backbone, base, or sugar of the RNA. In addition, transfection conditions will need to be optimized for each particular application, including, for example, the duplex siRNA (e.g., chemistry, length, and charge), the nature of the target gene/gene product. In particular, suspending the siRNA in some type of gel or polymer matrix, such as, for example, a hyaluronic acid gel or cationic polymer such as polyethyleneimine, previous to being encapsulated in a vesicle, with said suspended therapeutic then delivered by methods of the present teachings. Following diffusion of the suspended siRNA through the target cell membrane, the suspending medium then slowly dissolves, allowing “timed-released” siRNA delivery and other therapeutics (e.g., oligonucleotides) directly into the target cell cytoplasm over extended periods.

The nucleus (FIG. 2A [217]) is surrounded by a double membrane (212) which compartmentalizes nuclear and cytoplasmic reactions. Besides its key role in regulating nucleocytoplasmic transport, the nuclear membrane provides a structural support for the attachment of other macromolecular structures (e.g., the nuclear lamina, nucleoskeleton, cytoskeleton, and chromatin). The nuclear envelope is the ultimate obstacle to the nuclear entry of, for example, therapeutic plasmid DNA, where the inefficient nuclear uptake of said plasmid from the cytoplasm was recognized decades ago (Capecci, 1980). Indeed, no more than 0.1-0.001% of cytosollically injected plasmid DNA could be successfully transcribed (Capecci, 1980). However, nucleocytoplasmic transport of macromolecules through the nuclear membrane is a fundamental process for the metabolism of eucaryotic cells and involves nuclear pore complexes (NPCs) that form an aqueous channel through the nuclear envelop (not illustrated; Laskey, 1998). While molecules smaller than ˜40 kilodalton (kDa) can diffuse through the NPC passively, plasmids and other macromolecules larger than ˜60 kDa usually comprise a specific targeting signal, the nuclear location sequence (NLS), to transverse the NPC successfully in an energy-dependent manner (Talcott et al., 1999). Now it is widely accepted that the size of expression cassettes constitutes a major impediment to nuclear targeting. DNA fragments diffuse passively into the nucleus if their size is small enough (i.e., a 20 base pair double stranded oligomer is approximately equivalent in size to a 13 kDa polypeptide), where oligonucleotides efficiently escape the transport barrier of the cytoplasm and nuclear envelope (Lechardeur et al., 2002). While larger, for example, plasmid DNA and DNA fragments require nuclear localization sequences or other methods that facilitate active transport to the nucleus and through its membrane. Embodiments of this disclosure may be of assistance in nuclear entry of a variety of compounds by cavitation-mediated energy, as well as other mechanisms.

The aforementioned examples are only representative of the many potential embodiments of this disclosure. Most importantly, these embodiments are intended to be exemplary only, and therefore non-limiting to the present specification. A plethora of variables can be altered with each of these examples, as well as many other embodiments; therefore, a wide variety of techniques, materials, and other properties are available for the preparation of targeted and non-targeted nanocarriers for acoustically mediated drug delivery, as well as the administration and activation procedures of said components. Optimally, said therapeutics and/or materials will be designed and engineered for specific levels of acoustic sensitivity.

The following is a detailed description of illustrative embodiments of the present invention. As these embodiments of the present invention are described with reference to the aforementioned drawings and definitions, various modifications or adaptations of the methods and or specific structures described herein may become apparent to those skilled in the art. All such modifications, adaptations, or variations that rely upon the teachings of the present invention, and through which these teachings have advanced the art, are considered to be within the spirit and scope of the present invention.

### Acoustic Cavitation, the Present Invention, and Tissue Permeation

Situations and applications involving liquids often arise where properties, structures, or processes are affected or disturbances produced by the presence of a gaseous or vaporous phase in the fluid. Among many non-acoustic examples are bubbling and filling processes used in industry, erosion of ship propellers, and decompression hazards encountered by divers and aviators. Acoustic examples include noise generation from bubbles at the sea surface, and the diverse physical, chemical, and biological effects produced by sound, especially ultrasound. In all of these situations, the observations can be attributed to some form of cavitation.

Cavitation activity is a response to a change in pressure at some location in a liquid. Under special conditions, a cavity containing vapor or gas can be created in a homogeneous liquid (i.e., away from boundaries) and become a site for cavitation (i.e., a cavitation nucleus). Much more commonly, the nuclei for cavitation are preexisting gas-filled cavities; they are often of microscopic size, stabilized in some way against dissolution, and special means are required to detect and characterize them. Acoustical, optical, electrical, and other methods for determining the number and size of these small cavities are know in the art.

Later in this specification, accepted methods known in the prior art are summarized for detecting and/or characterizing the cavitation activity itself, many of which are important to the teachings of the present specification. In its simplest form, basic cavitation activity may consist of spherically symmetrical vibrations of one or more gas-filled bubbles. In many applications, including those of the present teachings, it involves motions that are more complex; these include the formation of microbubbles or liquid jets, radiated shock waves, bubble coalescence, streaming of fluids within bubbles or external to them, and movements of the bubbles themselves. The vibrational motion can be studied by optical methods, or by methods embodied herein, such as acoustical methods for analyzing the spectra of sound generated by, and in response to, the cavitation.

In solutions or suspensions, cavitation produces any of a wide variety of physical, chemical, and biological effects whose results can be assessed and used as indices of cavitation activity. In organized biological tissues, known gas-filled cavities include respiratory channels, lung alveoli, and intercellular channels; sound may cause these to be activated with consequences that can be assessed biologically or by measurements of acoustic emissions. There is evidence that other cavities exist naturally in animal tissues, but little is known about their distribution or about effects resulting from their activation. In addition, as exemplified by the methods and embodiments of the present invention, in modern medical procedures utilizing diagnostic ultrasound, small gaseous bodies are introduced into the bloodstream of patients to increase the contrast in images or to add information obtained with Doppler methods. As described in detail herein, these externally introduced gaseous bodies, either alone or in combination with other agents, can be used effectively, either directly or indirectly, for drug delivery purposes.

The prior art includes many materials that are important to the teachings of the present disclosure, these publications include Acoustics: An Introduction to its Physical Principles and Applications (Pierce, 1989); Acoustic Characterization of Contrast Agents for Medical Ultrasound Imaging (Hoff, 2001); Basic Acoustics (Hall, 1993); Ultrasonic Exposimetry (Ziskin and Lewin, 1993); The Acoustic Bubble (Leighton, 1994); Nonlinear Acoustics (Research Trends in Physics) (Naugol'Nykh, 1994); Cavitation and Bubble Dynamics (Oxford Engineering Science Series) (Brennen, 1995); Sonochemistry and Cavitation (Margulis, 1995); Ultrasound in Medicine (Duck et al., 1998); Fundamentals of Acoustical Oceanography (Applications of Modern Acoustics) (Medwin et al., 1998); Cavitation Reaction Engineering (The Plenum Chemical Engineering Series) (Shah et al., 1999); Fundamentals of Physical Acoustics (Blackstock, 2000); Fundamentals of Acoustics (Kinsler, 2000); Applied Sonochemistry: Uses of Power Ultrasound in Chemistry and Processing (Mason et al., 2001); Acoustical Imaging Volume 27 (Acoustical Imaging) (Arnold, 2004); Suspension Acoustics: An Introduction to the Physics of Suspensions (Temkin, 2005); and The Science and Applications of Acoustics (Raichel, 2006). Peer-reviewed research publications important to the present teachings include (Flynn, 1975a; Flynn, 1975b; Coakley, 1978; Apfel, 1981a; Apfel, 1981b; Apfel, 1982; Morton et al., 1983; Apfel, 1986; Atchley et al., 1988; Apfel et al., 1991; Greenleaf et al., 1998; Hilgenfeldt et al., 2000; Cochran et al., 2001; Guzman et al., 2001a; Guzman et al., 2001b; Chen et al., 2002; Guzman et al., 2002; Guzman et al., 2003; and Thomas et al., 2005). For enablement and all other purposes, the disclosures of each of the foregoing publications in this paragraph [0314] are hereby incorporated by reference herein in their entirety.

As described, the present invention utilizes ultrasonic energy for the safe and effective permeation of patient tissues for mediating intracellular drug delivery, in both in vitro and in vivo applications. Typically, with nearly all therapeutic ultrasound applications in the prior art, acoustic cavitation was avoided because the effect on tissues was so unpredictable, especially regarding the location of the tissue alterations produced, and the thresholds thereof. However, in the present disclosure, microbubbles, both in the form of contrast agents and/or other active agents infused into the patient, and/or bubbles formed from previous ultrasound exposure, allow for predictable cavitation thresholds, requiring much lower incident ultrasound intensities for treating tissue. In addition, by following the present teachings, much more spatially regular areas of controlled tissue permeability are produced, limiting cytotoxicity and sonolysis, and maximizing intracellular drug delivery. Moreover, by using pulsed ultrasound in the preferred embodiments of the invention, a large number of parameters are created, which provided the appropriate monitoring and feedback mechanisms are present, allow the use of a diversity of parameter optimizations and control systems for “fine tuning” the system for a given drug delivery application.

### Bubble Cloud Activation and the Cavitation-Mediated Ultrasonic Drug Delivery Process

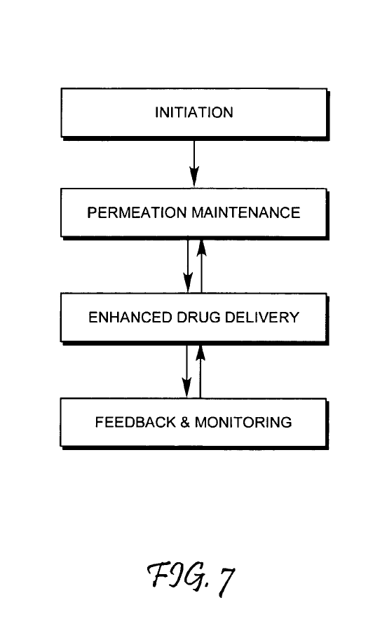

In its most preferred embodiments, bubble cloud activation during the cavitation-mediated ultrasonic drug delivery process, using the methods and systems described herein, may be categorized into four major subprocesses (FIG. 7):
1. Initiation—Cavitation nuclei are placed, generated, or seeded into the target region of the patient where tissue permeation for drug delivery purposes is sought. The presence of said cavitation nuclei reduces the threshold for cavitation by subsequent therapeutic pulses, and without initiation, the disruption and permeation process will not proceed. Initiation assures that the process will progress until it spontaneously, or through active intervention, extinguishes. Importantly, the initiation step can be terminated or cancelled by employing the opposite process, namely the active removal (i.e., deletion) of cavitation nuclei in parts of the target tissue volume. In order to protect certain tissue volumes or structures from damage, ultrasonic pulses can be used to locally cancel cavitation.
2. Permeation maintenance—The presence of micro-nuclei in the therapy volume is actively maintained, assuring that subsequent pulses produce a desired tissue effect. In some embodiments, an appropriate tissue effect might include at least a portion of the final, desired tissue disruption and permeation. While not wishing to be bound by any particular theory in this step, cell membrane disruption and increased permeation result primarily from shockwaves produced by oscillating, as well as some collapsing microbubbles. Thus, intracellular drug delivery begins here, as biological barriers are partially permeated. Increased therapeutic movement in the target region, as well as increased therapeutic diffusion into cells, is a result of convective microstreaming and other processes reviewed herein. The opposite of permeation maintenance would be to actively extinguish the process, perhaps by removing (i.e., deleting) microbubbles or by manipulation of microbubble size, density, or some other property, protecting, for example, a desired tissue volume in the target region from disruption.
3. Enhanced drug delivery—Micro-cavitation nuclei (i.e., small microbubbles) that have been properly initiated and maintained by the preceding processes can be impinged upon by additional ultrasonic pulses to produce acute (i.e., inertial or transient) cavitation. This results in the generation of microjets, enhanced microstreaming, and significant tissue disruption and membrane permeation. Each therapy pulse can produce just a small part of the overall therapy effect, which in some embodiments may include mechanical fractionation to various degrees depending primarily on the drug delivery application.
4. Feedback & parameter monitoring—Each of the prior subprocesses can be monitored as well as the overall progress of therapy. The feedback and monitoring step allows for various parameters of the pulsed cavitational ultrasound drug delivery process to be varied in real time or in stages, if desired, permitting precise, controlled administration of the ultrasonic drug delivery process. For example, said process can be terminated, the extent of therapy measured, and said process reinitiated. In particular, the feedback subprocess enables adjustment and fine tuning of the ultrasonic drug delivery process.
   During the enhanced drug delivery step, the properly initiated and maintained micronuclei may be impinged upon by a therapy pulse that produces acute cavitation and tissue permeation. Each therapy pulse can produce just a small part of the overall therapy effect, which in addition to cell membrane permeation, might include mechanical disruption depending primarily on the intensity of the cavitation produced per unit volume of tissue.

In the simplest process (FIG. 7), the therapeutic transducer initiates, maintains, and produces the desired therapy effect, mediating drug delivery. Thus, for example, a series of higher intensity pulses (FIG. 3 [XXX]) are focused onto the target region sufficient to initiate bubble cloud activation. The intensity of the pulses can then be decreased to an intermediate intensity below a value that would otherwise not initiate the process. This intermediate intensity is sufficient to sustain the process, otherwise, the process can be reinitiated, if necessary, to produce adequate tissue permeation. As will be described herein, feedback on the presence or absence of a bubble cloud can be obtained by, for example, monitoring the therapeutic pulse backscatter from the bubble cloud, where the absence of said backscatter indicates an extinguished cavitation process. In one embodiment, the backscatter is monitored by the therapeutic transducer—or subset of therapeutic transducer array elements—in the receive mode, or in another preferred embodiment, by a simple—and separate—imaging transducer. In additional embodiments, multiple transducers may be employed for feedback and parameter monitoring.

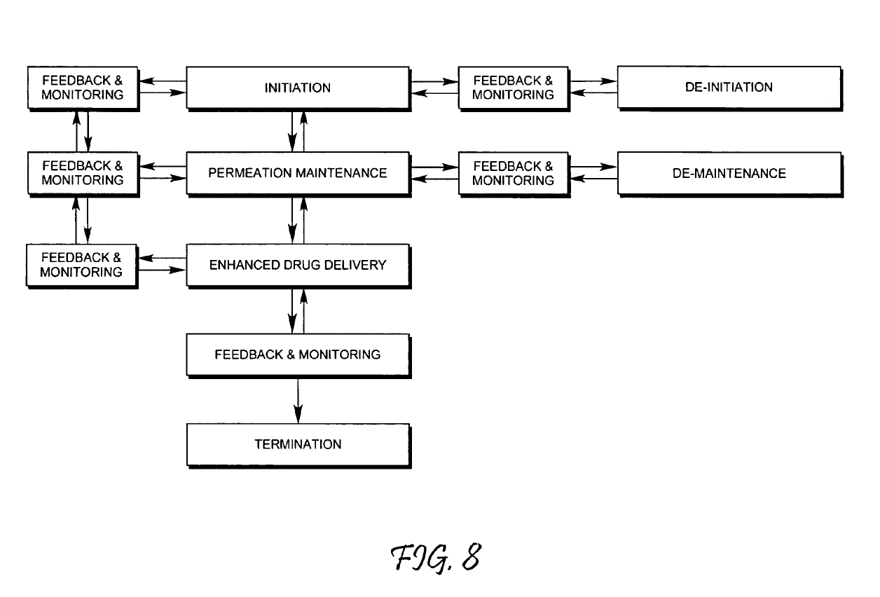

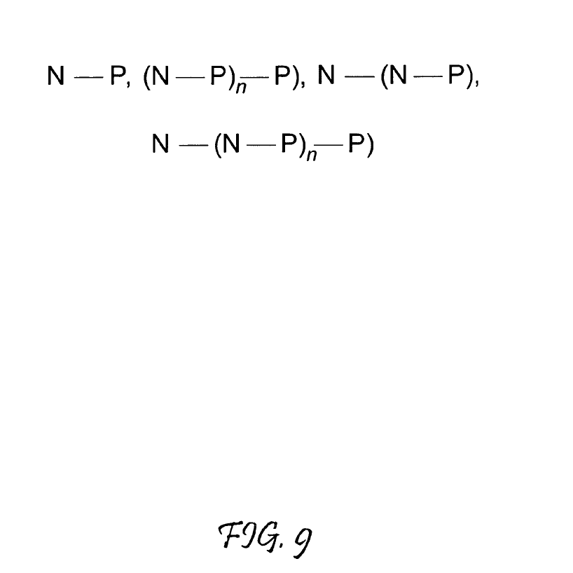

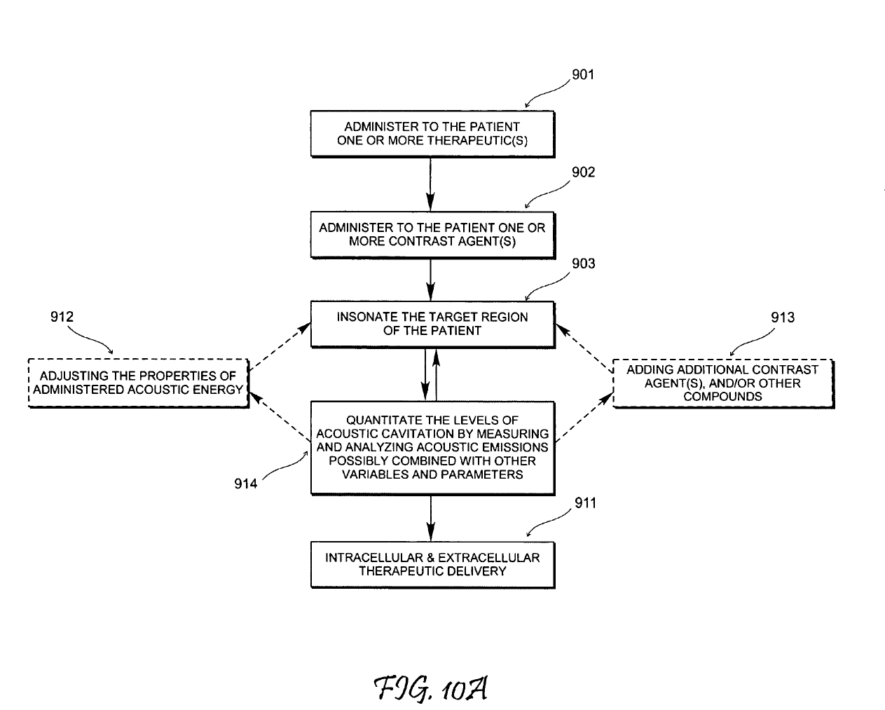

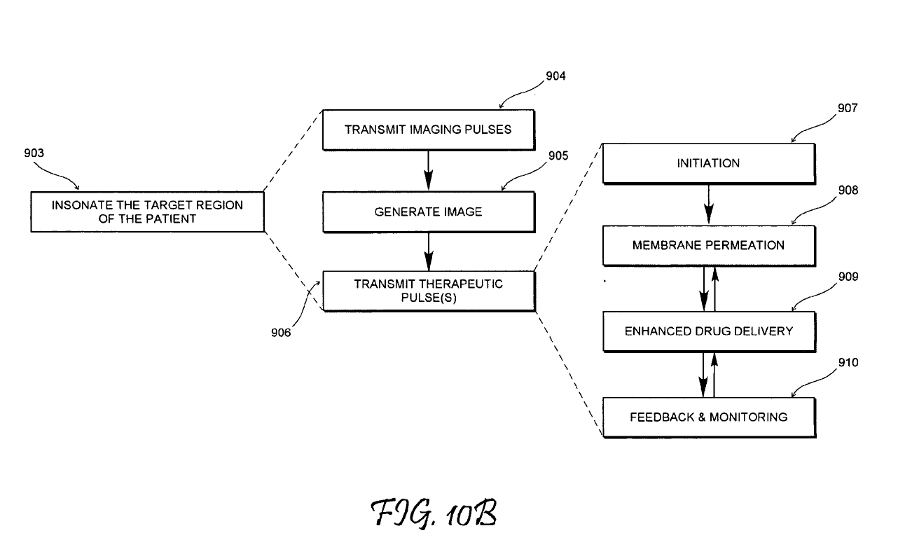

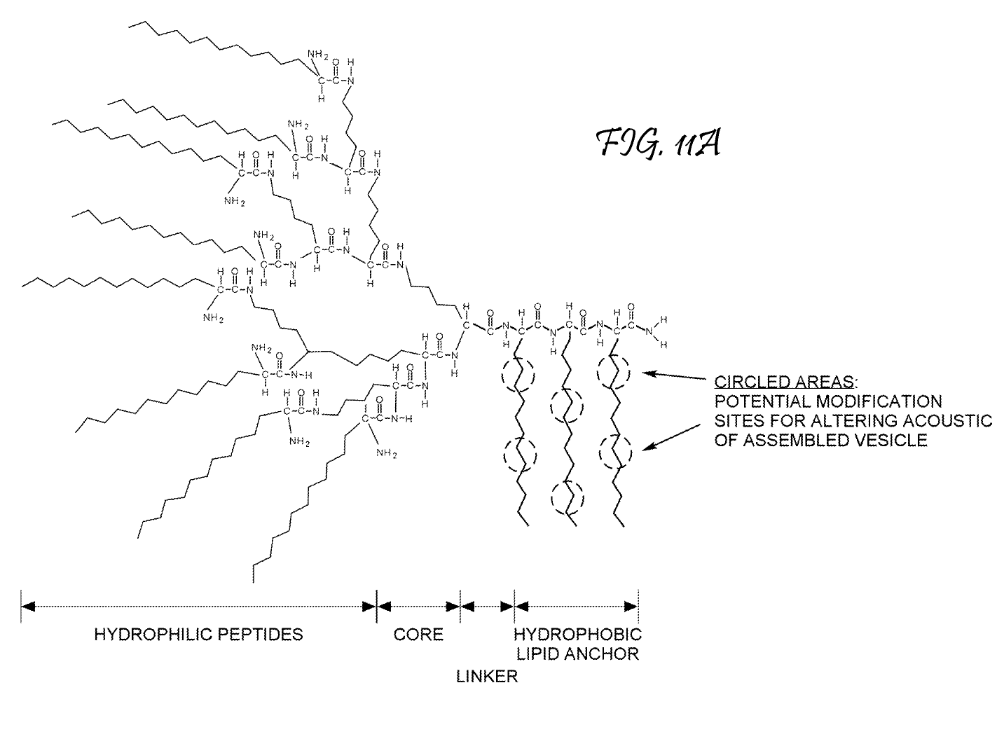

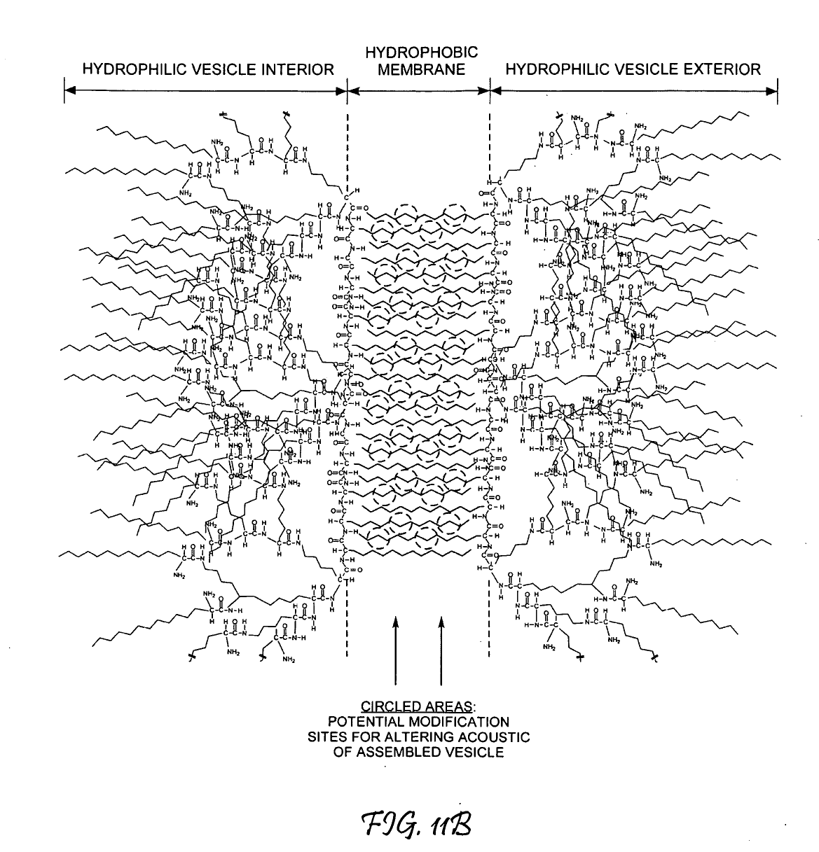

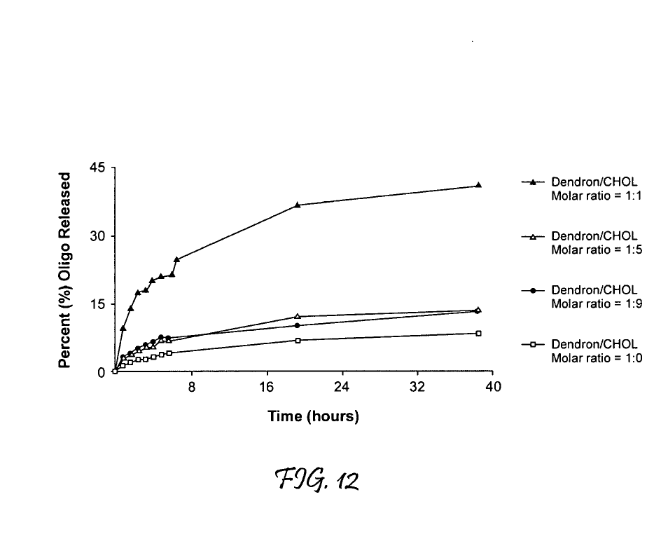

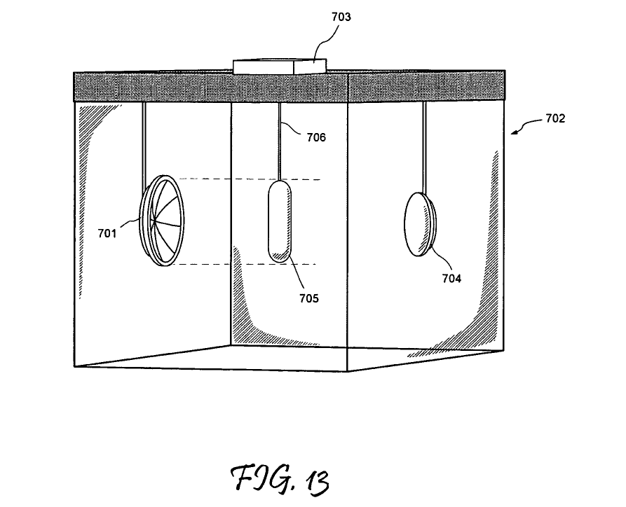

During the feedback step, each of the four major subprocesses may be monitored to evaluate the progression of therapy. Further, the feedback and monitoring step allows for a variety of parameters of the pulsed cavitational ultrasound process to be varied in real time or in stages, if desired, permitting controlled administration of the ultrasonic drug delivery process. For example, the cavitation process can be terminated, the extent of therapy measured, and the process reinitiated (FIG. 8).

Importantly, the methods of the present teachings can include variations where each of the four major subprocesses described herein can use different methods of energy delivery with different energy sources and feedback schemes. Additional details of the various embodiments of each major subprocess are as follows.

### Initiation

Initiation can comprise an initiation pulse sequence, which is also referred to herein as an initiation sequence or pulse, or initiation. Initiation activates threshold-reducing cavitation nuclei and can be accomplished with a therapeutic transducer using acoustic energy of usually high-intensity pulses, at the same frequency as the sustaining processes. However, initiation can be accomplished by other forms of energy including high intensity laser (or optical) pulses that create a vapor cloud or even a plasma cloud, or x-rays (i.e., the ionizing radiation bubble chamber effect). In a most preferred embodiment, cavitation nuclei are injected intravascularly, or can be injected, or shot (i.e., mechanically jetted) into the therapy volume. As described later in this specification, microbubbles or proto-bubble droplets (e.g., perfluorocarbon droplets) can be targeted to a therapy volume by molecular or other recognition mechanisms, for example, antibody against tumor antigens conjugated to nuclei (or proto-nuclei) that would concentrate in or near a tumor. Initiation can also occur via mechanical stimulation sufficient to generate cavitation or cavitation nuclei. Further, in some embodiments, initiation can be accomplished by an ultrasound imaging transducer whose additional role is obtaining feedback information on the drug delivery process.

Preferred systems for cavitation-mediated drug delivery may use separate acoustic transducer(s), which can be an array or a plurality of transducers, to initiate cavitation, and then use the therapeutic transducer for permeation maintenance and enhanced drug delivery subprocesses. This would enable the use of high-frequency ultrasound for initiation, thereby making use of the higher resolution of high-frequency transducers or arrays. In this embodiment, initiation may aid in highlighting the target area for drug delivery with high spatial resolution, and drug delivery may progress at lower frequencies using the therapeutic transducer or an array of transducers. For example, lower frequencies might propagate through some bone and air. Thus, methods can include predisposing (i.e., initiating) with high resolution and disposing (i.e., providing therapy) at a lower frequency that can cover the entire therapy volume. Because lower frequency sound propagates more easily through, for example, bone and air, methods of the present teachings may thus be applied to sites beyond said and other structures. In addition, lower frequency sound has lower thermal absorption, reducing heat generation.

Further, it can be useful to use de-initiation as an aid in the protection of certain regions in or near the target site (FIG. 8). De-initiation can, for example, remove or delete microbubbles, or cavitation nuclei, greatly increasing the cavitation threshold in these sub-volumes and protecting the tissue therein. For example, in delivering drugs to the prostate using the present invention, the neurovascular bundle just outside the outer capsule of the prostate could be de-initiated (i.e., cavitation nuclei deleted or removed) prior to treatment, thus, protecting this zone and preventing subsequent sequelae such as impotence and incontinence in treated patients. The de-initiation could be introduced by the therapeutic transducer, with a special pulse sequence, or could be accomplished by a separate transducer similar to the multi-transducer initiation scheme discussed herein. De-initiation may also be introduced by other energy sources (laser, microwaves, thermal, etc.; [FIG. 8]).

Feedback is important in determining if initiation has occurred because the cavitation-mediated drug delivery process will not progress without initiation (FIG. 8). In some embodiments, feedback can include monitoring the backscattered signal from the therapeutic pulses. If no significant backscatter occurs, initiation has not been successful or the process has extinguished and needs to be re-initiated. In some embodiments, feedback can employ one or more of the following: an ultrasound imaging modality that would detect the microbubbles as a hyperechoic zone; a separate transducer to ping (i.e., send an interrogation pulse or pulses), and a transducer to receive it; optical processes wherein optical scattering from the microbubbles, when initiated, are detected; MRI imaging to detect the microbubbles; and low-frequency hydrophones which can detect the low frequency sound produced when bubble clouds expand and contract.

In some embodiments, the feedback scheme can determine the parameters of existing cavitation nuclei in the target area and the dynamic changes, of said bubbles, with sufficient precision to predict the optimum characteristics or parameters for the next therapeutic pulse (e.g., intensity, peak negative pressure, peak positive pressure, time of arrival, duration, frequency, etc.).

### Permeation Maintenance

Membrane permeation maintenance can comprise a sustaining pulse sequence (FIG. 3) which is also referred to herein as a sustaining sequence, sustaining or maintenance pulse, or maintenance. Permeation maintenance can follow initiation as well as being a part of initiation. Generally, once initiated, the cavitation process must be maintained or it will spontaneously extinguish. For example, cavitation can be extinguished when the next therapy pulse does not encounter sufficient nuclei to effectively cavitate at least a portion of the microbubbles in the target area. In various embodiments, permeation maintenance is accomplished by the next therapeutic pulse that activates/creates a bubble cloud, leaving behind sufficient nuclei for the subsequent pulse(s).

Permeation maintenance can also be accomplished by a separate sustaining transducer producing ultrasound to maintain (i.e., sustain) the appropriate nuclei characteristics and population. Thus, the separate transducer(s) described herein for initiation can also maintain (i.e., sustain) the nuclei. Likewise, in some embodiments, maintenance can be continued by optical means, x-rays (i.e., ionizing radiation), mechanical stimulation, or thermal means. In some embodiments, permeation maintenance can be accomplished by a feedback ultrasound imaging transducer. For example, if a slow therapy pulse repetition frequency is desired (e.g., to prevent tissue heating), sustaining sequences or pulses—of lower intensity, for example—can be interleaved between the therapeutic pulses to sustain the microbubble or nuclei population and characteristics necessary to allow the next therapy pulse to be effective (FIG. 3). These interleaved sustaining sequences can be applied by the various means enumerated herein for permeation maintenance or initiation.

The schemes outlined herein for deleting or canceling cavitation nuclei can only be used in certain volumes of the target area protecting or shielding certain tissues from permeation and drug delivery. Processes described herein can be applied to effectively delete nuclei while maintaining other parts of the therapy volume. Thus, in some embodiments, active de-maintenance procedures (FIG. 8) can be instituted using the various energy modalities outlined herein, where some therapeutic volume(s) are maintained for therapy progression while other volumes in the target area are actively protected or the cavitation nuclei in these volumes can be allowed to extinguish passively. Maintenance feedback and monitoring can be similar to the initiation feedback step outlined herein, except in some embodiments, lower pulse intensities can be used compared to pulses employed in the initiation step.

### Enhanced Drug Delivery

Enhanced drug delivery can comprise a therapeutic pulse sequence, which is also referred to herein as a therapeutic sequence, therapeutic pulse, or therapy. The enhanced drug delivery process is the interaction of ultrasound on existing cavitation nuclei to produce sufficiently vigorous cavitation to mechanically disrupt cell membranes and other physiological tissues in the target area, making said membranes and structures highly susceptible to intracellular therapeutic entry by, for example, diffusion and passive diffusion. The transducer or transducers used in delivering ultrasonic energy for enhanced drug delivery can be either single-focus, or multi-focus, or phased arrays where the focus can be scanned in 1, 2, or 3 dimensions. These therapy transducer(s) can be contiguous spatially or can be separated spatially. Further, the transducers can also operate at different frequencies individually or as an overall ensemble of therapeutic transducers. These transducer(s) can also be mechanically scanned to generate larger drug delivery zones, and/or a combination of mechanically and electronically (i.e., phased array) scans can be used. In addition, the therapeutic transducer(s) can also be used, as outlined herein, as sources of initiation and/or permeation maintenance processes and procedures. The therapeutic transducer(s) can be intimately involved in the feedback processes and procedures, such as, for example, as sources of interrogation sequences or as receivers or even imagers. Thus, in some embodiments, the therapeutic ultrasonic pulses or sequences can initiate and maintain cavitation, permeate membranes, and initiate enhanced drug delivery.

The use of a multiplicity of transducers enables various embodiments where one of the therapy transducers could operate at a significantly lower frequency from the other(s). For example, the higher frequency transducer can initiate (i.e., predispose) microbubbles to cavitation and the lower frequency transducer can actively permeate membranes by initiating and maintaining cavitation.

In some embodiments, one or more low-frequency transducers can act as a pump with the other transducer(s) sending pulse sequences propagating along with the low-frequency pump. For example, if a higher frequency, short therapeutic pulse arrives in the target area in a particular orientation to the phase of the low-frequency pump pulse, multiple effects can be obtained depending on this relative phase relationship of said pulses. If the higher frequency pulse rides on the peak rarefactional (i.e., negative pressure) portion of the pump, the peak negative (i.e., rarefactional) pressure of the high-frequency pulse can be increased to enhance its ability to cavitate available nuclei. Thus, the pump acts as a significant enhancer of membrane permeability. The same arrangement can be employed to enhance initiation.

If the higher-frequency pulse arrives at the therapeutic volume on the peak positive pressure of the pump, the cavitational effect is reduced, but can enhance the ability of said high-frequency waveform to delete cavitational nuclei. Thus, said high-frequency pulse can have a de-initiation or cancellation function. Also, if the pump and therapeutic pulse arrive at different propagation angles, it can serve to spatially sharpen the effective focus of the therapeutic pulse. The maximum sharpening effect occurs when the pulses arrive at the target after being propagated in opposite directions or 90° from each other.

The therapy transducers (i.e., high- and low-frequency) can also operate in conjunction with the feedback transducers to enhance various treatment and/or imaging effects. For example, if an imaging transducer is used for feedback on initiation, permeation maintenance, or enhanced drug delivery, said transducer can be used in a similar way, as discussed herein, to enhance the detection of microbubbles or nuclei. That is, if the imaging pulse arrives in the imaging tissue volume on the rarefactional trough of the pump pulse, the bubbles will have expanded and will be relatively hyperechoic. If the imaging pulse arrives on the peak positive pressure, the nuclei or microbubbles will be smaller in size (i.e., compressed) and the image in this interaction zone will be relatively hypoechoic. Thus, by using a “difference image,” only activated microbubble activity can be observed as the other tissue echoes will be constant (i.e., the same) in both images.

In some embodiments, the therapeutic pulse can be used as a pump and the imaging pulse can be propagated therewith. If one or more therapeutic pulses are focused on the target area, or portion of the target, the intensity is usually greater at the focal point of the therapeutic pulse. Therefore, the effect on microbubbles will be greater in the focused therapeutic volume, and less away from the focused therapeutic volume. By co-propagating the imaging and therapy pulse alternately, with the imaging pulse riding on the peak rarefactional pressure of the therapeutic pulse and the peak positive pressure of the therapy pulse, a difference image will show the greatest difference near the focused therapeutic pulse(s). The difference will be less away from said therapeutic pulse(s). Thus, this scheme allows direct imaging of the therapeutic pulse beam pattern. This can also be used to identify and locate where the maximum tissue permeation will occur in the target region before treatment.

### Feedback and Parameter Monitoring

In several preferred embodiments, feedback enables assessment of parameters related to successful intracellular drug delivery in vivo. These feedback methods and devices depend on the therapeutic effect of cavitation being progressive over multiple ultrasonic pulse sequences, where the exposed cells and tissues are changed physically over a specific time period. Further, embodiments of the present teachings make it possible to monitor the therapeutic effectiveness, both during and after the drug delivery process, where this type of feedback monitoring has been unobtainable in previous noninvasive “active” drug delivery procedures known in the art.

In some embodiments, feedback and monitoring can include evaluating changes in backscatter from bubble clouds, speckle reduction in backscatter, backscatter speckle statistics, shear wave propagation, acoustic emissions, and electrical impedance tomography, and combination thereof as described below. Also, additional methods may be employed for feedback and monitoring, when said methods assist in directly or indirectly measuring the amount and extent of bubble cloud activation and/or acoustic cavitation at the target site.

Backscatter from bubble clouds. This feedback method can determine immediately if the cavitation process has been initiated, is being properly maintained, or even if it has been extinguished. For example, this method enables continuous monitoring of the drug delivery process in real time. The method can also provide feedback, permitting the pulsed cavitational drug delivery process to be initiated at a higher intensity and then maintained at a much lower intensity. For example, backscatter feedback can be monitored by any transducer or ultrasonic imager. By measuring feedback for the therapy transducer, an accessory transducer can send out interrogation pulses. Moreover, the nature of the feedback received can be used to adjust acoustic parameters—and associated system parameters—to optimize the drug delivery and/or tissue permeation process.

Backscatter speckle reduction. Progressively permeated tissue results in changes in the size and distribution of acoustic scatter. At some point in the process, the scattering particle size and density are reduced to levels where little ultrasound is scattered, or the amount scattered is reduced significantly. This results in a significant reduction in speckle, which is the constructive and destructive interference patterns of light and dark spots seen on images when coherent sources of illumination are used; in this case, ultrasound. After some treatment time, the speckle reduction results in a darkened area in the therapy volume. Thus, treatment can proceed until a desired speckle reduction level has been reached and is easily seen and evaluated on standard ultrasound imaging systems. Specialized transducers and systems can also be used to evaluate the backscatter changes.

Backscatter, changes in speckle statistics. Speckle in an image persists from frame to frame, and changes little as long as the scatter distribution does not change, and there is no movement of the imaged object. However, long before the scatters are reduced enough in size to cause visible speckle reduction, they may be changed sufficiently where said change can be detected by signal processing and other methods. This family of techniques can operate as detectors of speckle statistical changes. For example, the size and position of one or more speckles in an image will begin to decorrelate before observable speckle reduction occurs. Speckle decorrelation, after appropriate motion compensation, can be a sensitive measure of tissue permeation, and thus, ultimately an indirect measure of drug delivery efficacy. This feedback and monitoring technique permits early observation of changes, and thus identifies changes in target cells and tissue before substantial or complete sonolysis or cytotoxicity occurs. Thus, changes in speckle statistics represent a most preferred methodology for use in feedback and monitoring of the cavitation-mediated drug delivery process.

Shear wave propagation changes. The permeation of tissues makes the tissue more fluid and less solid, and fluid systems generally do not propagate shear waves. Thus, the extent of tissue fluidization provides opportunities for feedback and monitoring of the cavitation-mediated drug delivery process. For example, ultrasound and MRI imaging systems can be used to observe the propagation of shear waves. The extinction or significant reduction of such waves in a treated volume is used as a measure of tissue permeation or disruption. Moreover, dedicated instrumentation can be used to generate and measure the interacting shear waves.

Acoustic emissions. As a tissue volume is permeated, its effect on microbubbles is changed. For example, bubbles may grow larger and collapse and/or have a different lifetime or other characteristics in intact versus fluidized tissue. Bubbles may also move and interact after tissue is subdivided, producing larger bubbles or cooperative interaction among bubbles, all of which can result in changes in acoustic emissions. These emissions can be heard during treatment, and said emissions can change during treatment. Thus, analysis of these changes, and their correlation to efficacious drug delivery procedures, enable monitoring of the progress of therapy and represent a most preferred method for feedback and monitoring of embodiments of the present invention. A detailed example illustrating the monitoring of acoustic emissions during the cell membrane permeation process is presented later in this specification.

Electrical impedance tomography. An impedance map of a therapy site can be produced based on the spatial electrical characteristics throughout the target. Imaging of the conductivity or permittivity of the target site of a patient can be inferred from taking skin surface electrical measurements. Conducting electrodes are attached to a patient's skin, and small alternating currents are applied to some or all of the electrodes. One or more known currents are injected into the surface, and the voltage is measured at a number of points using the electrodes. The process can be repeated for different configurations of applied current. The resolution of the resultant image can be adjusted by changing the number of electrodes employed. A measure of the electrical properties of the therapy site within the skin surface can be obtained from the impedance map, and changes in and location of the bubble cloud, and the cavitation-mediated drug delivery process can be monitored using this methodology.

### Adjusting the Cavitation-Mediated Drug Delivery Process

In some embodiments of the present teachings, opportunities exist to adjust or customize the drug delivery process for particular applications, and said adjusting and customizing are considered a part of the feedback and monitoring step(s) (FIG. 7 and FIG. 8). By changing various parameters, the process can be initiated by high-intensity pulses and maintained by low-intensity pulses, therapy intensity can be varied, and changes in maintenance (i.e., sustaining) pulses can be produced (FIG. 3). The aforementioned feedback and monitoring methods readily allow these directed parameter adjustments, and the effects thereof to be observed during the drug delivery process, in real time, and/or permit therapy progress measurement in stages, where therapy can be reinitiated as desired or as necessary.

In some embodiments, cavitation-induced soft tissue permeation can be enhanced by a process in which a short, high-intensity sequence of pulses is used to initiate permeation, and lower-intensity pulses are employed to sustain the process. This strategy generates cavitation nuclei using high-intensity pulses which provide seeds for the subsequent lower-intensity pulses to sustain said cavitation and permeation (FIG. 3). If lower-intensity pulses are used for permeation, but instantaneous initiation is ensured by a short higher-intensity sequence, the energy infused into the target region before initiation can be retained and thermal complications associated with subsequent pulses reduced. Indeed, by using the high-intensity initiating sequence strategy (FIG. 3), permeation can be sustained at a much lower average intensity and with less overall transmitted energy into the target region. In addition to minimizing thermal damage and around the treated region, the probability of thermal damage to the therapeutic transducer is also lowered.

In some embodiments, a high-intensity initiating sequence can help to increase the probability of permeation at lower intensities with only a slight increase in total propagated energy. Consequently, the intensity threshold for generating permeation is significantly lower using such an initiating sequence. Further, the initiating sequence increases the permeation rate by ensuring instantaneous initiation and permeation maintenance with subsequent energy pulses. Therefore, little energy is wasted on acoustic pulses preparing for initiation, but producing no tissue disruption and permeation.

Without wishing to be bound by any particular theory, the increased probability of permeation, when using one or more high-intensity initiation pulses, followed by lower-intensity pulses (FIG. 3), is likely a result of the following mechanism. A cloud of activated microbubbles is generated by the initiating sequence, providing a set of cavitation nuclei for the lower-intensity pulses. Thus, cavitation nuclei are generated only at the desired location, minimizing damage to cells and tissues outside of the target region. However, cavitation will likely last for shorter durations after each successive initiation, suggesting either a depletion of certain essential components to sustain cavitation (e.g., cavitation nuclei) over time, or increased interferences (e.g., shadowing from larger bubbles). Thus, cavitation appears to be a threshold phenomenon which only occurs when the density or population of microbubbles within a certain size range exceeds said threshold. Thus, a variety of variables can be adjusted/altered to affect this threshold including, for example, infusing additional microbubbles into the target region or the entire patient systemically, before and/or at times during therapy.

In addition, the duration of active cavitation will probably not depend on the number of pulses within the initiating sequence. An initiating sequence containing additional pulses will also likely provide longer active cavitation initiation or more tissue permeation. Therefore, only the minimum number of high-intensity pulses (i.e., the minimum activation energy) is required for the cavitation-mediated drug delivery process.

Once cavitation is extinguished, active cavitation is unlikely to reinitiate spontaneously, and can be shorter in duration if reinitiation does occur. A feedback strategy can be formed where the high-intensity initiating sequence is used to initiate cavitation, lower-intensity pulses are used to maintain it, and the initiating sequence used again, when necessary, to reinitiate the process when extinction is detected. This strategy can accomplish tissue permeation with lower propagated energy, reducing heating of overlying tissue, as well as the transducer, which is a concern for any ultrasound therapy.

Tissue inhomogeneities may also affect the cavitation-induced permeation process. For example, atrial septum and atrial wall tissues both consist of two layers of membrane tissue with soft muscle in between. Membrane can be harder to permeate than soft muscle tissue, requiring a higher intensity. An efficient strategy is to permeate, for example, membrane tissue with higher-intensity pulses and permeate the soft tissue with lower-intensity pulses. Thus, acoustic parameters can be chosen specifically for the tissue type, as well as the drug delivery application, maximizing the efficiency of the process. Further, intensity thresholds of cavitation-mediated drug delivery can also be varied, as needed. The feedback and monitoring methods of the present disclosure allow changes in intensity to be observed in real time or in stages, as desired, and said changes can identify and tune intensity thresholds for ultrasound-induced tissue permeation, optimizing local tissue disruption and permeation in the target region. Additional parameter adjustments can affect the structure of tissue alternations produced by the cavitational drug delivery process. For example, adjustment of specific acoustic parameters (e.g., such as pulse sequence repetition frequency [PRF], and sustaining pulse amplitude) can result in marked effects on the physical characteristics of resulting tissue changes.

### EXAMPLE SYSTEMS FOR CAVITATION-MEDIATED ULTRASONIC DRUG DELIVERY

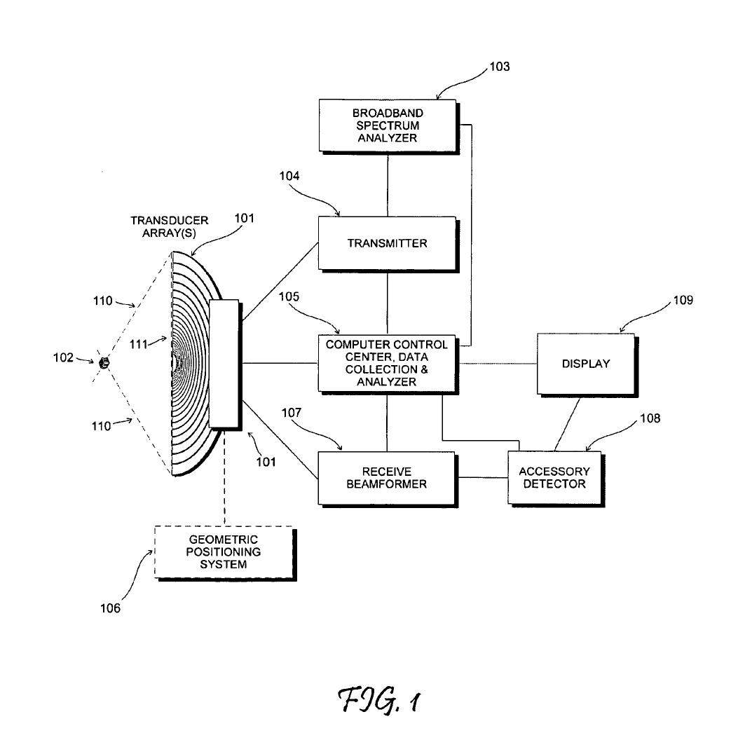

FIG. 1 illustrates one exemplary apparatus for performing the cavitation-mediated ultrasonic drug delivery process, designed in accordance with the teachings of the present specification. This system is a single preferred embodiment of the invention; importantly, neither FIG. 1 nor this specification are definitions of the invention.

The apparatus comprises one or more transducers [101] for focusing imaging and therapeutic acoustic transmissions on one or more targets [102], a broadband spectrum analyzer [103], a transmit beamformer [104], a computer control center, data collector, and analyzing system [105], one or more mechanical rotation or position tracking devices [106], a receive beamformer [107], a processor or detector [108], and a display [109] electrically connected as shown (FIG. 1). Additional, different, or fewer components may be provided for the illustrated system, and said components may be connected differently. In a preferred embodiment, said system comprises a commercial ultrasound system from one of the manufacturers listed herein, or another manufacturer.

The transducer(s) [101] comprise, for example, one or more piezoelectric or a capacitive microelectromechanical ultrasound transducer. The transducer [101] has one or more elements for transducing between electrical and acoustical energies. In one embodiment, the transducer [101] includes only a single linear array of elements, (e.g., a flat linear array or most preferably a curved linear array). In other embodiments, the transducer comprises a two-dimensional array, a 1.5 dimensional array, or other multidimensional configurations of elements. The array of elements are configured for insertion into a patient or used preferably external to a patient without, but preferably with, mechanical rotation or position tracking devices [106].

The transducer(s) [101] contain a standard imaging transducer, such as a transducer associated with halfwavelength spacing of elements sandwiched between a backing block for absorbing acoustic energy and matching layers for matching the acoustic impedance of the elements to a patient.

In alternative embodiments, a transducer is modified for heat dissipation. For example, a copper foil or copper braid is connected with a lens of a transducer [101] for dissipating heat from the lens, or said transducer(s) may be liquid cooled or with some type of refrigerant. Different piezoelectric materials or matching layers may be optimized for providing a better acoustic or electrical impedance match, reducing an amount of heat generated by the transducer. In one embodiment, multiple layers of piezoelectric or microelectromechanical material separated by electrodes are provided for each element. The multiple layers provide better electrical impedance matching of the transducer to the cable impedance, lowering the generation of heat. In another embodiment, a lensless array or a piezoelectric material shaped to provide elevation focus without a lens focus is provided to reduce the heating of the transducer [101]. Reduced heating or more efficient heat dissipation allows for better penetration of acoustic energy and higher power transmissions, such as associated with color Doppler, or therapeutic acoustic energy.

The transducer(s) [101] are designed for operation within a frequency band. Typically the frequency band is associated with transmission and reception of both imaging and therapeutic pulses having a same or similar center frequency. In alternative embodiments, the transducer(s) [101] are associated with wide band operation, such as operating to transmit at a fundamental frequency and receive at a second- or third-order frequency. The imaging and therapeutic pulses may also be provided at substantially different center frequencies, such as associated with a −6 dB down spectral bandwidth that does not overlap.

The transmitter [104] is a transmit beamformer, waveform generator or other source of electrical excitations for imaging and therapeutic transmissions. In one embodiment, the transmitter [104] is a transmit beamformer that generates waveforms for each of a plurality of channels or transducer elements, such as 128 waveforms, separately delayed and apodized for focusing transmissions along scan lines [110]. Based on the delays and apodization, multiple transmissions may be sequentially scanned across substantially parallel scan lines in the entire field of view [111]. The field of view [111] is formed in response to the scan pattern, such as a linear, sector, or Vector scan patterns.

The computer control center [105] is at the heart of said system and may be comprised of commercially available systems or preferably those that are custom designed and constructed for systems used in practicing the present teachings.

The transmitter [104] includes a large power supply, large capacitors, or other source of energy for generating high-power acoustic transmissions. For example, larger capacitors sufficient to provide 50-200 transmit beams of acoustic energy of 50-200 cycles each at a maximum amplitude (e.g., 50-140 volt), with minimal droop or drain, should be provided. For example, only 10% droop allows for ongoing delivery of high power transmit waveforms. Other systems may have different maximum voltages. Alternatively or additionally, an efficient source of providing high power transmit waveforms for 50-200 pulses of multiple cycles is provided. Other transmitters [104] capable of other maximum amplitudes, numbers of cycles, or numbers of pulses may be used.

The transmitter [104] electrically connects with the transducer [101] for generating transmissions of acoustic energy or transmit pulses in response to the electrical signals from the transmitter [104]. The acoustic energy transmitted includes one of imaging or therapy pulses. Imaging pulses are transmissions adapted for generating an image of the field of view [111], such as sequential transmissions of narrow beams sequentially focused along a plurality of scan lines [110]. Therapy pulses include transmissions adapted for enhancing drug delivery. Therapy pulses or transmissions are operable to force a change in tissue or fluid, such as causing cavitation.

The receive beamformer [107] generates receive beams for imaging. The receive beamformer [107] applies various delays and apodization to electrical signals received from elements of the transducer [101] and sums the signals to generate a receive beam representing a scan line [110] in response to each of the transmissions.

The processor or detector [108] comprises one or more of an application-specific integrated circuit, general processor, digital signal processor, other digital circuitry, analog circuitry, a combination thereof, or other devices for detecting information from the received, beamformed signals for imaging. In one embodiment, the processor [108] comprises a B-mode or Doppler detector. For example, the amplitude of an envelope associated with the received signals is detected. As another example, a frequency shift or velocity, magnitude of a Doppler signal or energy, or variance is detected by Doppler or correlation processing for flow or tissue motion imaging. Other processors for one-dimensional, two-dimensional, or three-dimensional imaging may be used.

### Imaging

In a preferred embodiment, a standard ultrasound system is used for imaging are used with little or no modification. This would include, for example, the Antares System manufactured by Siemens Medical Solutions USA, Inc. Ultrasound Group or the Sequoia System manufactured by Acuson-A Siemens Company. A variety of imaging pulses can be transmitted, for example, pulses for B-mode or Doppler imaging. For B-mode imaging, a 1-5 cycle pulse is transmitted along each of the scan lines within the field of view. For Doppler imaging, a plurality of transmit pulses for determining a Doppler coefficient, correlation or flow characteristic are transmitted along each scan line. Other imaging pulses are possible (e.g., pulses for acoustic radiation force impulse imaging). The transmit pulses have a transmit power determined from the number of cycles, amplitude and pulse repetition frequency of the transmit pulses. The transmit pulse pressure is limited by the Food and Drug Administration to particular mechanical indexes within the field of view. Typically, ultrasound systems provide a transmit pressure near the maximum mechanical index.

In response to the imaging pulses, an image of a field of view is generated. The field of view is determined by the position of the imaging transducer, the steering of the imaging transmissions, and the selected depths of viewing. The field of view is optimized to view a potential region, such as the target region and surrounding tissue. In response to a single image or a sequential set of images, a user selects a region of interest within the field of view. Alternatively, the imaging system automatically determines the region of interest within the field of view.

Further details of suitable imaging systems which may be modified to execute the methods of the present teachings and incorporated into the systems described herein are described, for example, in U.S. Pat. Nos. 6,231,834; 6,457,365; 6,437,946; 6,985,430; 7,041,058; 7,123,450; 7,212,608; and 7,212,609, U.S. patent application Ser. No. 11/070,371, filed Mar. 2, 2005; the disclosures of all of which are incorporated by reference herein in their entirety for all purposes.

### Transducers

As reviewed herein, ultrasound transducers generate ultrasound waves for imaging and therapeutic purposes. A typical ultrasound transducer comprises piezoelectric materials (e.g., PZT ceramics, electrodes, matching layers, and backing materials). The present invention provides ultrasound transducer apparatus(es) specially designed for cavitation-mediated ultrasonic drug delivery, comprising a generally concave array of ultrasound transducer elements. The apparatus(es) enable a reduced number of transducer elements and a larger pitch size compared to that used for the elements in a traditional linear array of transducer elements. Reducing the number of elements also lowers the required number of connection cables and control channels. While providing the same performance, the concave array system is much simpler and less costly than a conventional linear-phased array system. The concave geometry also requires smaller phase differences between transducer elements, thus reducing cross-talk and heating in kerf fills between elements. The geometry also reduces the effect of grating lobe problems during the beam-forming process.

To provide both imaging and therapy functions, in a preferred embodiment, the invention includes circuitry to rapidly switch between low and high Q-factors. Alternatively, in another preferred embodiment, the invention includes one transducer array for imaging and another transducer array for therapy, enabling one of the arrays to selectively act on the target site. For example, the imaging transducer array and therapeutic transducer array may be attached to opposite sides of a rotatable carriage, and alternately, directed to the target site as the carriage rotates.

To control a location of a focus point of the transducer array, in a preferred embodiment, the invention includes one or more geometric (3-axis) positioning systems and/or beam steering mechanisms and/or controllers to adjust the phases or the delays of signals that drive the transducer elements. To increase the transducer bandwidth for better image resolution, an electrical damping circuit can be included to provide the equivalent of a mechanical backing. One or more material acoustic matching layers and/or air backing can optionally be included to improve transducer efficiency and bandwidth. In addition, the present invention may optionally include one or more metal matching layers to improve heat dissipation by the transducer.

Flexible transducer array(s) are preferably provided to control the location of the focus point. Flexible outer layers and kerf fills between transducer elements enable the array(s) to bend in different curvatures. As with a fixed curvature array, the flexible array(s) reduce the number of required transducer elements. However, the flexible array embodiment also enables a user to adjust the imaging field of view (FOV) and simplifies control of treatment focusing, by changing the geometric shape of the array.

To assist in facilitating these capabilities, the suitable systems of this specification include one or more geometry control mechanisms. Preferably, the control mechanisms and flexible transducer array(s) comprise applicator(s) in which a linear actuator translates one end of the flexible transducer array (s) relative to an opposite fixed end, causing the transducer array(s) to flex into a desired curved shape. The actuator alternatively comprises either a manual adjustable shaft, or preferably, a motor-driven threaded shaft, shuttle block, push rod, or the like. Another embodiment includes position stops or a position template to guide the curvature of the array, so that the array matches the profile of said position stops or template. Further, said position stops or template may be preset or adjustable. The geometry control mechanism may also be independently applied to one transducer array that is dedicated to one of the functions of imaging or therapy, while another transducer array is dedicated to the other function. For example, the control mechanism may be applied to a therapy transducer array connected to a rotational carriage, while an imaging transducer array is attached to the opposite side of the rotational carriage and is not provided with any control mechanism.

Another embodiment of the invention includes a plurality of transducer arrays, each directed toward a common focus point. Using multiple transducer arrays enables each array to contain fewer transducer elements and provides a relatively wide imaging and treatment field. Each transducer array may also be allowed to pivot about a pivot point, such that controlled pivoting of the multiple transducer arrays controls the location of the common focus point. This enables controlled movement of the common focus point in at least two directions.

Further details of suitable transducers which may be modified and incorporated into the systems described herein to execute the methods of the present teachings are described in U.S. Pat. Nos. 6,428,477, 6,461,303; 6,515,402; 6,589,180; 6,641,534; 6,780,153; 6,780,157; 6,945,937; 6,972,510; 7,226,417; and 7,364,007, the disclosures of each of which are incorporated by reference herein in their entirety for all purposes.

### Spectrum Analyzer

A spectrum analyzer is a device used to examine the spectral composition of some electrical, optical, or in the case of the present invention, acoustic waveform. The digital spectrum analyzer, most suited for use with the systems of the present specification [FIG. 1 (103)] computes, for example, the Fast Fourier transform (FFT), a mathematical process that transforms an acoustic waveform into the components of its frequency spectrum. A variety of analyzers are available commercially that can be readily incorporated into the systems of the present invention. For example, the E4443A PSA Spectrum Analyzer from Agilent Technologies is one such device.

Further details of suitable spectrum analyzers which may be modified and incorporated into the systems described herein to execute the methods of the present teachings are described in U.S. Pat. Nos. 4,599,892; 4,770,184; 5,172,597; and 6,822,929, the disclosures of each of which are incorporated by reference herein in their entirety for all purposes.

### Positioning System

Further details of suitable positioning systems which may be modified and incorporated into the systems described herein to execute the methods of the present teachings are described in U.S. Pat. Nos. 5,769,790; 6,330,300, 6,437,946; 6,782,287; 6,985,430; 7,123,450; 7,154,991; 7,212,608; 7,212,609; 7,260,426; 7,327,865, and U.S. patent application Ser. Nos. 10/881,315, filed on Jun. 30, 2004; Ser. No. 11/070,371, filed Mar. 2, 2005; and Ser. No. 11/890,881 filed on Aug. 7, 2007, the disclosures of each of which are incorporated by reference herein in their entirety for all purposes.

The aforementioned paragraphs [0348] to [0372] describe only a few preferred embodiments of systems for cavitation-mediated ultrasonic drug delivery, designed specifically for the methods described herein. While these systems have been described with reference to specific embodiments, it will be understood by those skilled in the art that various, sometimes significant changes may be made and equivalents may be substituted for elements thereof without departing from the true spirit and scope of the invention. In addition, modifications may be made without departing from the essential teachings of the invention.

### Alternative Instruments for Cavitation-Mediated Ultrasonic Drug Delivery

The optimal system to be employed with the methods of the present teachings, is an instrumentation system designed to measure, monitor, and control the amount and extent of acoustic cavitation induced by acoustic energy transferred to the patient, at a specific region of said patient, etc., as described in detail throughout this specification (FIG. 1). However, existing diagnostic and HIFU instrumentation may be employed in an attempt to practice the methods of the present teachings. For example, any of the various types of diagnostic ultrasound imaging devices may be employed.

Further details concerning suitable HIFU systems which may be modified and incorporated into the systems for executing the methods of the present teachings are disclosed in U.S. Pat. Nos. 4,084,582; 4,207,901; 4,223,560; 4,227,417; 4,248,090; 4,257,271; 4,317,370; 4,325,381; 4,586,512; 4,620,546; 4,658,828; 4,664,121; 4,858,613; 4,951,653; 4,955,365; 5,036,855; 5,054,470; 5,080,102; 5,117,832; 5,149,319; 5,215,680; 5,219,401; 5,247,935; 5,295,484; 5,316,000; 5,391,197; 5,409,006; 5,443,069, 5,470,350, 5,492,126; 5,573,497, 5,601,526; 5,620,479; 5,630,837; 5,643,179; 5,676,692; 5,840,031; 5,762,066; 6,626,855; 6,685,640, and U.S. patent application Ser. No. 11/070,371, filed Mar. 2, 2005; the disclosures of all of which are incorporated by reference herein in their entirety for all purposes.

Alternative ultrasound devices may be more optimally used with the present teachings, for example, by employing some of the following instrumentation parameters. In general, devices for therapeutic ultrasound should employ from approximately 10% to approximately 100% pulse durations, depending on the area of tissue to be treated. A region of the patient which is generally characterized by larger amounts of muscle mass, for example, the back and thighs, as well as highly vascularized tissues, such as heart tissue, may require a larger duty factor, for example, up to approximately 100% (i.e., continuous). In therapeutic ultrasound, continuous wave ultrasound is typically used to deliver higher energy levels. For rupturing the nanocarriers of the present invention, continuous wave ultrasound may, in some circumstances, be preferred, although the sound energy is usually pulsed, especially in order to optimize acoustic cavitation and minimize temperature increases at the target site. If pulsed sound energy is used, the sound will generally be pulsed in echo train lengths of approximately 8 to approximately 20 or more pulses at a time.

In addition to the pulsed method, continuous wave ultrasound (e.g., Power Doppler) may be applied. This may be particularly useful where rigid vesicles (i.e., nanocarriers that are cross-linked) are employed. In this case, the relatively higher energy of the Power Doppler may be made to resonate ultrasound contrast agents coadministered with the nanocarriers of the present teachings, thereby promoting their rupture. Indeed, as described herein, this can create acoustic emissions which may be in the subharmonic or ultraharmonic range or, in some cases, in the same frequency as the applied ultrasound. Generally, the levels of energy from diagnostic ultrasound should be insufficient to promote the rupture of vesicles, and to facilitate release and cellular uptake of any bioactive agents. As noted previously, diagnostic ultrasound may involve the application of one or more pulses of sound. Pauses between pulses permit the reflected sonic signals to be received and analyzed. Thus, the limited number of pulses used in diagnostic ultrasound limits the effective energy which is delivered to the tissue under treatment.

Higher-energy ultrasound (i.e., ultrasound which is generated by therapeutic ultrasound equipment) is usually capable of causing rupture of embodiments of the present invention. The frequency of the sound used may vary from approximately 0.025 MHz to approximately 10 MHz. In general, frequency for therapeutic ultrasound preferably ranges between approximately 0.75 MHz and approximately 3 MHz, with from approximately 1 MHz and approximately 2 MHz being more preferred. In addition, energy levels may vary from approximately 0.5 Watt (W) per square centimeter (cm²) to approximately 5.0 W/cm²; with energy levels from approximately 0.5 W/cm² to approximately 2.5 W/cm² being preferred. Energy levels for therapeutic ultrasound causing hyperthermia are generally from approximately 5 W/cm² to approximately 50 W/cm². For small vesicles (i.e., vesicles having a diameter of less than approximately 0.5 μm) higher frequencies of sound are generally preferred because smaller vesicles are capable of absorbing sonic energy more effectively at higher frequencies of sound. When very high frequencies are used, for example, greater than approximately 10 MHz, the sonic energy will generally penetrate fluids and tissues to a limited depth only. Thus, external application of the sonic energy may be suitable for skin and other superficial tissues. However, it is generally necessary for deep structures to focus the ultrasonic energy so that it is preferentially directed within a focal zone. Alternatively, the ultrasonic energy may be applied via interstitial probes, intravascular ultrasound catheters, or endoluminal catheters.

For therapeutic drug delivery, after the compositions described herein have been administered to, or have otherwise reached the target region (e.g., via delivery with targeting ligand), the rupturing of the therapeutic-containing nanocarriers of this specification is carried out by applying ultrasound of a certain total exposure time, duty factor, pulse length, and peak incident pressure and frequency, to the region of the patient where therapy is desired. Specifically, when ultrasound is applied at a frequency corresponding to the peak resonant frequency of, for example, gaseous ultrasound contrast agents (i.e., microbubbles) coadministered with said therapeutic-containing nanocarriers, the vesicles should rupture and release their contents at the target area in part because of shockwaves produced by said cavitating microbubbles, as described herein. The peak resonant frequency can be determined either in vivo or in vitro, but preferably in vivo, by exposing the compositions to ultrasound, receiving the reflected resonant frequency signals and analyzing the spectrum of signals received to determine the peak, using conventional means. The peak, as so determined, corresponds to the peak resonant frequency, or second harmonic, as it is sometimes termed.

The therapeutic-containing nanocarriers should also rupture when, for example, coadministered ultrasound contrast agents, are exposed to non-peak resonant frequency ultrasound in combination with a higher intensity (i.e., wattage) and duration (i.e., time). This higher energy, however, results in greatly increased heating and tissue damage. By adjusting the frequency of the energy to match the peak resonant frequency of, for example, the gaseous contrast agents coadministered with said therapeutic-containing nanocarriers, the efficiency of therapeutic rupture and release should be improved, and appreciable tissue heating should not occur (i.e., no increase in temperature above approximately 2° C.); because less overall energy is ultimately required for the release of said therapeutic.

With the present teachings, a therapeutic ultrasound device may be used which employs two frequencies of ultrasound. The first frequency may be x, and the second frequency may be, for example, 2x. It is contemplated such a device might be designed such that the focal zones of the first and second frequencies converge to a single focal zone at the target area. The focal zone of the device may then be directed to the targeted compositions, for example, targeted vesicle compositions, within the targeted tissue. This ultrasound device may provide second harmonic therapy with simultaneous application of the x and 2x frequencies of ultrasonic energy. In the case of ultrasound involving vesicles, it is contemplated that this second harmonic therapy may provide improved rupturing of vesicles as compared to ultrasonic energy involving a single frequency. Lower energy may also be used with this dual frequency therapeutic ultrasound device, resulting in less sonolysis and cytotoxicity in the target area.

Preferably, the ultrasound device used in the practice of the present invention employs a resonant frequency (RF) spectral analyzer. The transducer probes may be applied externally or may be implanted. Ultrasound is generally initiated at lower intensity and duration, and then intensity, time, and/or resonant frequency are increased. Although application of these various principles will be readily apparent to one skilled in the art, in view of the present disclosure, by way of general guidance, the resonant frequency will generally be in the range of approximately 1 MHz to approximately 10 MHz. Using the 7.5 MHz curved array transducer as an example, adjusting the power delivered to the transducer to maximum and adjusting the focal zone within the target tissue, the spatial peak temporal average (SPTA) power will then be a maximum of approximately 5.31 mW/cm² in water. This power should cause some release of therapeutic agents from nanocarriers in close proximity to gas-filled microbubbles, with much greater release being accomplished by using a higher power.

As described in detail herein, the present teachings function most optimally primarily because of the phenomena of inertial cavitation in rupturing the nanocarriers of this disclosure, releasing and/or activating the bioactive agents within said vesicles. Thus, lower frequency energies may be used, as cavitation occurs more effectively at lower frequencies. Using a 0.757 MHz transducer driven with higher voltages (i.e., as high as 300 volts), cavitation of solutions of gas-filled ultrasound contrast agents will occur at thresholds of approximately 5.2 atmospheres. The ranges of energies transmitted to tissues from diagnostic ultrasound on commonly used instruments is known to one skilled in the art and described, for example, by Carson et al. (1978), the disclosure of which is hereby incorporated herein by reference in its entirety for all purposes. In general, these ranges of energies employed in pulse repetition are useful for diagnosis and monitoring compositions, but should be insufficient to rupture most of the nanocarriers of the present invention.

Either fixed frequency or modulated frequency ultrasound may be used in practicing the present invention. Fixed frequency is defined wherein the frequency of the sound wave is constant over time. A modulated frequency is one in which the wave frequency changes over time, for example, from high to low (i.e., PRICH) or from low to high (i.e., CHIRP). For example, a PRICH pulse with an initial frequency of 2.5 MHz of sonic energy is swept to 50 kHz with increasing power from 1 watt to 5 watts. Focused, frequency-modulated, high-energy ultrasound may increase the rate of local gaseous expansion within ultrasound contrast agents coadministered with the compositions described herein; thereby rupturing said nanocarriers to provide local delivery of therapeutics. A plethora of variables can be altered with ultrasonic energy delivery devices for use with the present invention; therefore, a wide variety of materials, transducers, energy generation systems, and other devices and systems are available for use with the present teachings in acoustically mediated intracellular drug delivery in vivo.

### Preferred Drug-Carrying Vesicles

Vesicles for use with the methods and systems of the present teachings are comprised materials from 6 major families: (1) biodegradable triblock polymers, (2) branched-chain polymers, (3) dendritic polymers, (4) peptosomes, (5) polymersomes, and (6) supramolecular assemblies. Optimal embodiments of the nanocarriers of the present teachings include vesicles that are specifically engineered for acoustically mediated drug delivery (i.e., said nanocarriers have a specific level of acoustic sensitivity). Methods for the synthesis of individual nanocarrier components, their assembly for general drug delivery purposes, and techniques (e.g., toxicity analysis) are readily available to those skilled in the art.

### Biodegradable Triblock Copolymers

Preferred triblock copolymers of the present invention are derived from water-soluble, hydrophilic, and nontoxic polymer end-blocks, and a hydrophobic polyarylate oligomer middle block of a biocompatible, nontoxic aliphatic or aromatic diacid; and a derivative of a tyrosine-derived diphenol. Thus, according to one aspect of the present teachings, polyarylate triblock copolymers are provided having an A-B-A structure, wherein each A end-block is a water-soluble, hydrophilic, and nontoxic polymer end-block; and the B middle block is an polyarylate oligomer with the same as or different repeating units having the structure of Scheme 1, wherein Z is between 2 and to approximately 100; R₁ is CH═CH or (CH₂)ₙ, wherein n is from 0 to 18, inclusive; R₂ is selected from hydrogen and straight and branched alkyl and alkylaryl groups containing up to 18 carbon atoms; and R is selected from a bond or straight and branched alkyl and alkylaryl groups containing up to 18 carbon atoms.

The endblocks are preferably poly(alkylene oxides) having the structure R₃—[(CH₂—)ₐCH₃—O—]ₘ—Wherein m for each A end-block is independently selected to provide a molecular weight for each A between approximately 1,000 and approximately 15,000 and R₃ for each A, and within each A, is independently selected from hydrogen and lower alkyl groups containing from one to four carbon atoms; and a is an integer greater than or equal to one. In a preferred embodiment, the end-blocks have the structure CH₃O—[CH₂CH₂O—]ₘ.

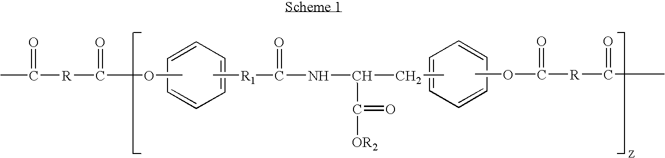

The preferred embodiments of the present teachings provide nanocarrier-encapsulated biologically or pharmaceutically active compounds, wherein the therapeutic-containing nanocarriers are present in an amount sufficient for effective, acoustically mediated, site-specific delivery. The carrier may be an aqueous solution or a polymeric drug delivery matrix. In a most preferred embodiment, nanocarriers that are either targeted or untargeted are utilized for site-specific, intracellular delivery of said active compounds, by administering to the patient an effective amount of said compounds encapsulated by the polymer nanocarriers of the present invention, and acoustically disrupting said nanocarrier.

The triblock copolymers degrade by hydrolysis into the original starting materials (i.e., the tyrosine-derived diphenols; the dicarboxylic acids; and the water-soluble, hydrophilic, and nontoxic oligomer end-blocks). The inventive copolymers are highly hydrophilic, which is advantageous for the nanocarrier drug delivery systems of the present invention. However, the hydrophilic:hydrophobic balance of the copolymers, which is the characteristic likely to determine their acoustic sensitivity, can be varied in several ways. The ester of the pendant chain of the diphenol can be changed, with longer-chain ester groups resulting in increased hydrophobicity. Increasing the molecular weight of the A end-blocks, for example, by increasing the number of carbons in the alkylene group of a poly(alkylene oxide) will also increase hydrophobicity. Changing the dicarboxylic acid will also change the hydrophilic:hydrophobic balance.

While certain of the above-mentioned polymer properties are individually well-known in the prior art, the combination of properties within a single composition, especially when used in acoustically mediated drug delivery, is new and represents a significant technological advance that has broad utility in the fields of drug and gene delivery. Specifically, the family of triblock copolymers described herein has at least three major distinguishing advantages over other triblock copolymers:
1. The family of triblock copolymers is fully resorbable after being introduced into the patient. As the compositions are derived exclusively of nontoxic building blocks, the triblock copolymers themselves as well as the expected degradation products, in vivo, are noncytotoxic and biocompatible.
2. The family of triblock copolymers self-assembles to form hollow nanocarriers with the above-mentioned low critical micelle concentration (CMC) and remains stable even under very high dilution, and is acoustically sensitive depending on the composition of said triblock copolymers.
3. The family of triblock copolymer provides a wide range of structural parameters which can be changed by those skilled in organic synthesis to derive triblock copolymers that are closely related to each other in chemical structure, while allowing the tailoring of key properties (e.g., the rate of bioresorption, the physical characteristics of the nanocarriers formed, and the release profiles obtained for the encapsulated therapeutic). Most importantly for use with the present teachings, these structural parameters can be optimized yielding polymers that form aggregates with specific sensitivity to disruption by acoustic energy for both in vitro and in vivo applications.

### Exemplary Biodegradable Triblock Copolymer Compositions

The copolymers of the present invention are A-B-A type triblocks, where the A end-blocks are water-soluble, hydrophilic, and nontoxic, preferably selected from poly(alkylene oxides), and the hydrophobic middle B block is either a polyarylate or polycarbonate. In a preferred polyarylate embodiment, the mid-block is copolymerized from a tyrosine-derived diphenol and a diacid, linked together by an ester bond between the phenolic hydroxyl group of the tyrosine-derived diphenol and the carboxylic acid group of the diacid. In another preferred embodiment, the polycarbonate mid-block is copolymerized from the same dihydroxy monomers.

Among the more preferred poly(alkylene oxides) end-blocks are polyethylene glycol, polypropylene glycol, polybutylene glycol, Pluronic® polymers, and the like. Polyethylene glycols are especially preferred.

The polyarylate middle blocks of the present invention are prepared by condensation of a diacid with a diphenol according to the method described by U.S. Pat. No. 5,216,115, in which diphenol compounds are reacted with aliphatic or aromatic dicarboxylic acids in a carbodiimide-mediated direct polyesterification using 4-(dimethyl-amino)-pyridinium-p-toluene sulfonate (DPTS) as a catalyst. The disclosures of U.S. Pat. No. 5,216,115 are incorporated herein by reference in its entirety for all purposes. Bis-diacids are selected as the polyarylate middle blocks permitting the A end-blocks to be coupled at each end of the copolymer.

The diphenol compounds are tyrosine-derived diphenol monomers of U.S. Pat. Nos. 5,587,507 and 5,670,602; both of which are incorporated herein by reference in their entirety for all purposes. The polyarylates are prepared using tyrosine-derived diphenol monomers having the structure of Scheme 2: wherein R₁ and R₂ are the same as described above with respect to Scheme 1. The preferred diphenol monomers are desaminotyrosyl-tyrosine carboxylic acids and esters thereof, wherein R₁ is —CH₂—CH₂—, which are referred to as DT esters. For purposes of the present invention, the ethyl ester (R₂=ethyl) is referred to as DTE, the benzyl ester (R₂=benzyl) as DTBn, and so forth. For purposes of the present invention, the desaminotyrosyl-tyrosine free carboxylic acid (R₂=hydrogen) is referred to as DT.

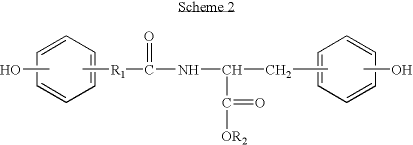

The polyarylate dicarboxylic acids have the structure HOOC—R—COOH (Scheme 3) wherein R is the same as described above with respect to Scheme 2, and preferably contains up to 12 carbon atoms. R is preferably selected so that the dicarboxylic acids employed as starting materials are either important naturally occurring metabolites, or highly biocompatible compounds. Preferred Scheme 3 dicarboxylic acids therefore include the intermediate dicarboxylic acids of the cellular respiration pathway known as the Krebs cycle. These dicarboxylic acids include alpha-ketoglutaric acid, succinic acid, fumeric acid, malic acid, and oxaloacetic acid, for which R is —CH₂—CH₂—C(═O)—, —CH₂—CH₂—, —CH═CH═, —CH₂—CH(—OH)—, and —CH₂—C(═O)—, respectively.

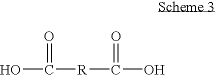

Another naturally occurring, preferred dicarboxylic acid is adipic acid (R═(—CH₂—)₄), found in beet juice. Other preferred biocompatible dicarboxylic acids include oxalic acid (no R), malonic acid (R═—CH₂—), glutaric acid (R═(—CH₂—)₃, pimellic acid (R═(—CH₂—)₅), suberic acid (R═(—CH₂—)₆), and azalaic acid (R═(—CH₂—)₇). In other words, among the dicarboxylic acids suitable for use in the present invention are compounds in which R represents (—CH₂—)_{z}, wherein z is an integer between 0 and 12, inclusive. A preferred class of highly biocompatible aromatic dicarboxylic acids is the bis(p-carboxyphenoxy) alkanes (e.g., bis[p-carboxyphenoxy]propane).

The triblock copolymers of the present invention may be iodine- and bromine-substituted, which renders the copolymers radio-opaque. These copolymers and their methods of preparation are disclosed by U.S. Pat. No. 6,475,577; the disclosures of which are incorporated herein by reference in their entirety for all purposes. Radio-opaque copolymers include repeating structural units in which one or more hydrogens of an aromatic ring, an alkylene carbon, or both, are replaced with an iodine or bromine atom. The triblock copolymers of the present invention may be similarly iodine- and bromine-substituted. Copolymers, according to the present invention comprising the repeating structural units of Scheme 2, are radio-opaque when copolymerized with radio-opaque monomers so that the copolymers also contain radio-opaque repeating structural units, preferably one or more of the A or B blocks in which one or more hydrogens of an aromatic ring, an alkylene carbon, or both, have been replaced with an iodine or bromine atom.

The molecular weights of the triblock copolymers can be controlled either by limiting the reaction time or the ratios of the components. Molecular weights can also be controlled by the quantity of the carbodiimide coupling reagent employed.

Preferred polyarylate oligomers have weight average molecular weights between approximately 1,000 daltons and 50,000 daltons, preferably between approximately 3,000 daltons and 25,000 daltons, and more preferably between approximately 5,000 daltons and 15,000 daltons. Molecular weights are calculated by gel permeation chromatography relative to polystyrene standards in tetrahydrofuran without further correction. The triblock copolymers thus have weight average molecular weights between approximately 2,500 and 75,000 daltons, preferably between approximately 5,000 daltons and 50,000 daltons, and more preferably between approximately 10,000 daltons and 25,000 daltons.

Preferred polycarbonates for use with the present invention have weight-average molecular weights ranging between approximately 1,000 daltons and 100,000 daltons, preferably between approximately 3,000 daltons and 50,000 daltons, and more preferably between approximately 10,000 daltons and 25,000 daltons. The triblock copolymers thus have weight average molecular weights between approximately 2,500 daltons and 130,000 daltons, preferably between approximately 5,000 daltons and 80,000 daltons, and more preferably between approximately 10,000 daltons and 50,000 daltons.

The triblock copolymers are prepared by the reaction of a non-functionalized poly(alkylene oxide) mono-alkyl ether with an excess of the dicarboxylic acid (mediated by a coupling agent [e.g., dicyclohexyl carbodiimide]). The following is a specific example of this general design (Scheme 4), illustrating the synthesis of PEG-oligo-(DTO suberate)-PEG, the molecular weights of the triblock copolymers can be controlled either by limiting the reaction time or the ratios of the components. Molecular weights can also be controlled by the quantity of the carbodiimide coupling reagent employed.

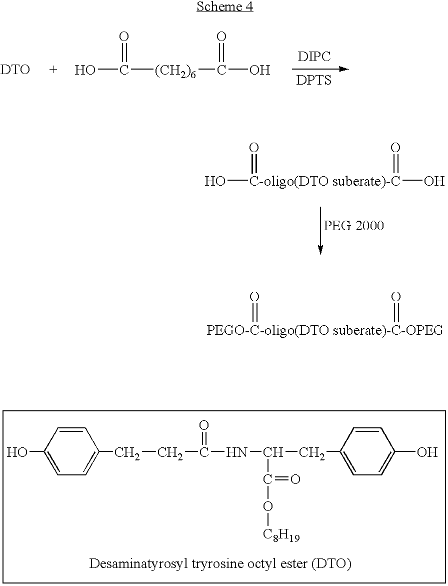

As described herein, the triblock copolymers degrade by hydrolysis into the original starting materials (i.e., the tyrosine-derived diphenols; the dicarboxylic acids; and the water-soluble, hydrophilic, and nontoxic polymer end-blocks). The inventive copolymers are highly hydrophilic, which is advantageous for nanocarrier drug delivery systems. However, the hydrophilic:hydrophobic balance of the copolymers can be varied in several ways. The ester of the pendant chain of the diphenol can be changed, with longer-chain ester groups increasing the hydrophobicity. Increasing the molecular weight of the A end-blocks (e.g., by increasing the number of carbons in the alkylene group of a poly[alkylene oxide]), will also increase hydrophobicity. Changing the dicarboxylic acid will also change the hydrophilic:hydrophobic balance.

### Branched-Chain Polymers

Preferred nanocarriers comprised substantially of branched-chain polymers include polymers consisting of thermosensitive poly((N-(2-hydroxypropyl) methacrylamide mono/dilactate) (poly(HPMAm-mono/dilactate)), N-(2-hydroxyethyl)methacrylamide-oligolactates (HEMAm-Lacₙ). Additional preferred embodiments include acoustically responsive block copolymers with poly(ethylene glycol) (PEG), polyethylenimine (PEI), poly(ethylene oxide) (PEO) composing the hydrophilic block of, for example, a thermosensitive hydrophobic block containing a polylactide, polyglycolide, poly(lactide-co-glycolide), poly(propylene oxide), poly(caprolactone), poly(benzyl aspartate); and more preferably PEG-block-(pNIPAm-co-(N-(2-hydroxypropyl)methacrylamide-dilactate) (HPMAm-Lac₂)) (PEG-b-(pHPMAm-Lac₂)), and PEG-block-(poly(N-(2-hydroxylethyl)methacrylamide-oligolactates) (HEMAm-Lacₙ)) (PEG-b-(pHEMAm-Lacₙ)).

Thermally or acoustically responsive polymers are obtained by choosing the properties of the monomers such that upon incubation, the functionality of the monomers changes (i.e., the solubility); and/or the temperature dependency of the solubility; and/or most preferably, the acoustic sensitivity of the entire polymer changes. In one embodiment, the monomers are chosen so that their hydrophilicity and acoustic sensitivity changes upon incubation. As a result, the hydrophilicity and acoustic sensitivity of the entire polymer changes.

Suitable monomers are, for example, selected from the group comprising ethylene glycol, lactic acid, acrylamide, methacrylamide, acrylic acid, and derivatives and substituted species thereof. These and/or other monomers are then reacted under suitable conditions to form homopolymers from the monomers or copolymers, terpolymers, or other polymers of two or more monomers. Preferred monomers include N-isopropyl acrylamide (NIPAm), 2-hydroxyethyl methacrylate (HEMA), 2-hydroxyethyl acrylate (HEA), acrylamide (Am), glyceryl methacrylate or glycidyl methacrylate (GMA), glyceryl acrylate or glycidyl acrylate (GA), hydroxypropyl methacrylamide (HPMAm), dimethyl-aminoethyl methacrylate (DMAEMA), and dimethylaminoethyl acrylate (DMAEA).

In a more preferred embodiment, the change of solubility characteristics is affected by hydrolysis of a group present on at least one of the monomers that form the polymer. Such a group is preferably chosen from ester, amide, carbonate, carbamate, and anhydride group. Even more preferably, such a group comprises a lactate unit (e.g., a monolactate, a dilactate, or an oligolactate group). For the in vivo applications of the present invention, such a group can advantageously be an enzymatically or chemically hydrolyzable group. The ester groups are introduced in the polymer by choosing suitable monomers as a starting material, such as, for example, 2-hydroxyethyl methacrylate-monolactate. The monomers can be readily provided with ester groups by techniques known to persons skilled in the art.

### Dendritic Polymers

Preferred nanocarriers comprised substantially of dendritic polymers are mostly characterized by a relatively high degree of branching, which is defined as the number average fraction of branching groups per molecule (i.e., the ratio of terminal groups plus branch groups to the total number of terminal groups, branched groups and linear groups). For ideal dendrons and dendrimers, the degree of branching is 1, whereas, for linear polymers, the degree of branching is 0. Hyperbranched polymers have a degree of branching that is intermediate to that of linear polymers and ideal dendrimers, preferably of at approximately 0.5 or higher. The degree of branching is expressed as follows:

$${f}_{br} = \frac{{N}_{t} + {N}_{b}}{{N}_{t} + {N}_{b} + {N}_{1}} \qquad Equation 1$$

where Nₓ is the number of type x units in the structure. Both terminal (i.e., type t) and branched (i.e., type b) units contribute to the fully branched structure while linear (i.e., type 1) units reduce the branching factor. Therefore, 0≦f_{br}≦1, where f_{br}=O represents the case of a linear polymer and f_{br}=1 represents the case of a fully branched macromolecule.

Further, preferred “dendritic polymers” for comprising the nanocarriers of this disclosure also include macromolecules commonly referred to as cascade molecules, arborols, arborescent grafted molecules, tectodendrimers, and the like. Suitable dendritic polymers also include bridged dendritic polymers (i.e., dendritic macromolecules linked together either through surface functional groups or through a linking molecule connecting surface functional groups together) and dendritic polymer aggregates held together by physical forces. Also included are spherical-shaped dendritic polymers (e.g., U.S. Pat. Nos. 4,507,466; 4,568,737; 4,587,329; 4,631,337; and 4,737,550) and rod-shaped dendritic polymers (e.g., U.S. Pat. No. 4,694,064) grown from a polymeric core, the disclosures of which are all incorporated herein by reference in their entirety for all purposes. Additional dendritic polymers, suitable for comprising the nanocarriers of this disclosure, include all of the basic structures where specific chelating groups or moieties are either in the central core of the dendrimer, and/or located within the interior structure on the dendron arms, and/or located on the surface of the dendrimer. Importantly, all of the aforementioned dendritic species are included within the term “dendritic polymers.”

Additional examples of “dendritic polymers” suitable for use with embodiments of the present invention include poly(ether) dendrons, dendrimers and hyperbranched polymers, poly(ester) dendrons, dendrimers, and hyperbranched polymers; poly(thioether) dendrons, dendrimers, and hyperbranched polymers; poly(amino acid) dendrons, dendrimers, and hyperbranched polymers; poly(arylalkylene ether) dendritic polymers; and poly(propyleneimine) dendrons, dendrimers, and hyperbranched polymers. The most preferred dendritic polymers and copolymers for use with embodiments of the present invention are comprised of polyesters and polyamino acids, polyethers, polyurethanes, polycarbonates, and polyamino alcohols, which can be chemically modified. In addition, dendritic polymers and copolymers of, for example, polyesters and polyamino acids with improved properties such as biodegradability, biocompatibility, and acoustic sensitivity, are also preferred embodiments. Additional embodiments include dendritic polymers that can be derivatized to include functionalities such as, for example, peptide sequences or growth factors to improve the interaction of the polymer with cells, tissues, or bone. Further, preferred embodiments of the dendritic polymers for use with the present invention include biocompatible dendrimers based on a core unit and branches, which are composed of glycerol and lactic acid; glycerol and glycolic acid; glycerol and succinic acid; glycerol and adapic acid; and glycerol, succinic acid, and polyethylene glycol.

### Peptosomes

Preferred embodiments of the peptosomes of the invention may comprise a single amphiphilic block copolypeptide. In other embodiments, more than one amphiphilic block copolypeptide may comprise the peptosome. In certain embodiments, amphiphilic block copolypeptides comprise one hydrophobic polymer and one hydrophilic polymer. In other embodiments, the amphiphilic block copolypeptide is a triblock polymer comprising terminal hydrophilic copolypeptide and a hydrophobic copolypeptide. Other amphiphilic block copolypeptides are tetrablock polymers comprising two hydrophilic copolypeptides blocks, and two hydrophobic copolypeptides blocks. Certain tetrablocks have terminal hydrophilic copolypeptide blocks and internal hydrophobic copolypeptide blocks. Other amphiphilic block copolypeptides are a pentablock polymer comprising two hydrophilic copolypeptide blocks and three hydrophobic copolypeptide blocks. In addition, pentablocks having three hydrophilic copolypeptide blocks and two hydrophobic copolypeptide blocks are preferred, yet other pentablocks having four hydrophilic copolypeptide blocks and one hydrophobic copolypeptide block are also embodiments. In yet other embodiments, the amphiphilic block copolypeptide comprises at least six blocks, at least two of which are hydrophilic polymer blocks.

In a preferred embodiment, block copolypeptides for use in the present invention contain greater than 100 monomer units (i.e., residues); and the distribution of chain-lengths in the block copolymer composition is at least approximately 1.01<M_{w}/Mₙ<1.25, where M_{w}/Mₙ=weight average molecular weight divided by number average molecular weight. In one preferred embodiment of the present invention, the block copolypeptide has 10 consecutive identical amino acids per block. In another preferred embodiment, the block copolypeptide is composed of amino acid components g-benzyl-L-glutamate and e-carbobenzyloxy-L-lysine. In another preferred embodiment of the present invention, the copolypeptide is a poly(e-benzyloxycarbonyl-L-lysine-block-g-benzyl-L-glutamate), PZLL-b-PBLG, diblock copolymer. In yet another preferred embodiment, the copolypeptide is a poly(g-benzyl-L-glutamate-block-e-benzyloxycarbonyl-L-lysine-block-g-benzyl-L-glutamate) triblock copolymer. In related embodiments, the number of consecutive monomer units (i.e., residues) in the block copolypeptide is greater than approximately 50, or 100, or 500, or even 1000. In another related embodiment, the total number of overall monomer units (i.e., residues) in the block copolypeptide is greater than approximately 200, or greater than approximately 500, or greater than approximately 1000.

Preferred embodiments of the present invention include diblock copolymers composed of amino acid components g-benzyl-L-glutamate and e-carbobenzyloxy-L-lysine. The polymers are prepared by addition of Lys-NCA to bipyNi(COD) in N,N-dimethylformamide (DMF) to afford living poly(e-carbobenzyloxy-L-lysine), PZLL, chains with organometallic end-groups capable of further chain growth. Glu-NCA is added to these polymers to yield the PBLG-PZLL block copolypeptides. The evolution of molecular weight through each stage of monomer addition is analyzed using gel permeation chromatography (GPC). Molecular weight is found to increase as expected upon growth of each block of copolymer, while polydispersity should remain low, indicative of successful copolymer formation.

Preferred block copolymerizations are not restricted to the highly soluble polypeptides PBLG and PZLL. Copolypeptides containing L-leucine and L-proline, both of which form homopolymers which are insoluble in most organic solvents (e.g., DMF), can also be prepared. Because of the solubilizing effect of the PBLG and PZLL blocks, all of the products are soluble in the reaction media, indicating the absence of any homopolymer contaminants. The block copolymers containing L-leucine are found to be strongly associating in 0.1 M LiBr in DMF, a good solvent for PBLG and PZLL. Once deprotected, the assembly properties of these materials are expected to make them useful as drug carriers.

Other methodologies include adding amino acid-N-carboxyanhydrides (NCAs) to polyaminoacid chains by exposing the NCA to solutions containing polyaminoacid chains having an amido amidate metallacyle end group, reacting the NCA with the amido amidate metallacyle end group so that the NCA is added to the polyaminoacid chain. Additionally, methods of controlling the polymerization of amino acid-N-carboxyanhydrides, by reacting NCAs with initiator molecules, and allowing initiator complexes to regioselectively open the ring of the NCAs through oxidative addition across the O—C₅ or O—C₂ anhydride bonds, resulting in a controlled polypeptide polymerization, are described in some of the aforementioned publications.

### Self-Assembling Amphiphilic Block Copolypeptides

A most preferred embodiment of the present invention entails the synthesis of amphiphilic block copolypeptides, which contain at least one water-soluble block polypeptide (“soluble block”) conjugated to a water-insoluble polypeptide domain (“insoluble block”). The overall mole percent (%) composition of the insoluble block(s) can range from 3%-60% of the total copolymer. Preferably, the soluble block has approximately 30 mole % to 100 mole % identical amino acid residues, having either charged or oligo(ethyleneglycol)-conjugated side chains.

The amphiphilic block copolypeptides of the present invention, for use in acoustically mediated in vivo drug delivery, contain one or more “soluble blocks.” The soluble block(s) of the copolymers can contain some finite fraction of amino acid components with charged side-chains, with the amino acids belonging to the group (e.g., glutamic acid, aspartic acid, arginine, histidine, lysine, or ornithine). They can also contain up to a maximum 99 mole % of the amino acids oligo(ethyleneglycol). The soluble block includes oligo(ethyleneglycol) terminated amino acid (EG-aa) residues. Preferred oligo(ethyleneglycol) functionalized amino acid residues include EG-Lys, EG-Ser, EG-Cys, and EG-Tyr. A most preferred soluble block consists of oligo(ethyleneglycol) terminated poly(lysine).

The amphiphilic block copolypeptides of the invention also contain at least one “insoluble block,” which is covalently linked to the soluble block. The insoluble block can contain a variety of amino acids residues or mixtures thereof, including the naturally occurring amino acids, ornithine, or blocks consisting entirely of one or more D-isomers of the amino acids. However, the insoluble block will typically be composed primarily of nonionic amino acid residues, which generally form insoluble high molecular weight homopolypeptides. In preferred embodiments, approximately 60 mole % to approximately 100 mole % of the insoluble block is comprised of nonionic amino acids. Such nonionic amino acids include, but are not limited to phenylalanine, leucine, valine, isoleucine, alanine, serine, threonine, and glutamine. In another preferred embodiment, any given insoluble block will usually contain 2-3 different kinds of amino acid components in statistically random sequences with mixtures of leucine/phenylalanine and leucine/valine being preferred. In these preferred copolymers, the composition of leucine in the insoluble domain ranges from 25 mole % to 75 mole %, when mixed with phenylalanine, and ranges from 60 mole % to 90 mole %, when mixed with valine.

In some embodiments, the multiblock copolypeptides that comprise the acoustically responsive peptosome membrane can be crosslinked. In other embodiments, biological entities are incorporated within the interior core of the peptosome, including polymers, cytoskeletal molecules, signaling molecules that can induce phosphorylation, dephosphorylation, amidization, acetylation, enolization, and enzymes that can cause chemical transformations of other biological molecules.

Certain peptosomes can comprise an amphiphile that is not a block copolymer, including: lipids, phospholipids, steroids, cholesterol, single-chain alcohols, nucleotides, saccharides, or surfactants.

Some peptosomes contain an amphiphilic copolymer, which is made by attaching two strands comprising different monomers. In some embodiments, the amphiphilic copolymer comprises polymers made by such techniques as, for example, free radical initiation and anionic polymerization.

### Polymersomes

Polymersome-preferred embodiments may comprise a single amphiphilic block copolymer. In other embodiments, more than one amphiphilic block copolymer may comprise the polymersome. In certain embodiments, amphiphilic block copolymer comprises one hydrophobic polymer and one hydrophilic polymer. In other embodiments, the amphiphilic block copolymer is a triblock polymer comprising terminal hydrophilic polymers and a hydrophobic polymer. Other amphiphilic block copolymers are tetrablock polymers comprising two hydrophilic polymer blocks, and two hydrophobic polymer blocks. Certain tetrablocks have terminal hydrophilic polymer blocks and internal hydrophobic polymer blocks. Other amphiphilic block copolymers are a pentablock polymer comprising two hydrophilic polymer blocks and three hydrophobic polymer blocks. In addition, pentablocks having three hydrophilic polymer blocks and two hydrophobic polymer blocks are also embodiments. Yet other pentablocks having four hydrophilic polymer blocks and one hydrophobic polymer block. In yet other embodiments, the amphiphilic block copolymer comprises at least six blocks, at least two of which are hydrophilic polymer blocks. In some preferred embodiments, the polymersome is biodegradable or bioresorbable, while in other embodiments, the polymersome contains block polymer components approved by the United States Food and Drug Administration (FDA) for use in vivo.

In some preferred embodiments, the hydrophilic polymer is substantially soluble in water. Preferred hydrophilic polymers include poly(ethylene oxide) and poly(ethylene glycol).

Some polymersomes of the invention comprise an amphiphilic copolymer where the hydrophilic polymer comprises polymerized units selected from ionically polymerizable polar monomers. In certain of these polymersomes, the ionically polymerizable polar monomers comprise an alkyl oxide monomer. In some embodiments, the alkyl oxide monomer is ethylene oxide, propylene oxide, or any combination thereof. In some preferred embodiments, the hydrophilic polymer comprises poly(ethylene oxide). In yet other preferred embodiments, the volume fraction of the hydrophilic polymers in the plurality of amphiphilic block copolymers is less than or equal to 0.40.

Biodegradable polymersomes. Additional preferred embodiments include block copolymers comprising a cationic polymer and a biodegradable polymer. More particularly, block copolymers comprising cationic polyethylenimine (PEI) as a hydrophilic block and biodegradable aliphatic polyester as a hydrophobic block are especially preferred. Further, the present invention provides self-assembled polymer aggregates formed from said block copolymers in an aqueous solution.

Said biodegradable aliphatic polyester employed as a hydrophobic block may be one selected from the group consisting of poly(L-lactide), poly(D,L-lactide), poly(D-lactide-co-glycolide), poly(L-lactide-co-glycolide), poly(D,L-lactide-co-glycolide), polycaprolactone, polyvalerolactone, polyhydroxybutyrate, polyhydroxyvalerate, poly(1,4-dioxan-2-one), polyorthoester, and copolymers there between.

Poly(D,L-lactide-co-glycolide) (PLGA) may be preferably selected, because biodegradable polymers having various degradation rates can be obtained by controlling the monomer ratio of lactic acid and glycolic acid, and/or by controlling polymerization conditions.

Further, said block copolymer can be obtained by a covalent bond between polyethylenimine and aliphatic polyester (e.g., an ester bond, anhydride bond, carbamate bond, carbonate bond, imine or amide bond, secondary amine bond, urethane bond, phosphodiester bond, or hydrazone bond). In addition, said block polymer may be an A-B type of diblock polymer, wherein A is said hydrophobic block of aliphatic polyester and B is said hydrophilic block of polyethylenimine.

In said block copolymer, the weight ratio of aliphatic polyester and polyethylenimine may be preferably in a range of 100:1˜1:10. If the amount of polyester is in excess, the block copolymer cannot form stable aggregates and thus precipitate. If the amount of polyethylenimine is in excess, the therapeutic containing the core component of the biodegradable polymersome decreases. Accordingly, it may be preferable to limit the ratio in said range.

### Supramolecular Assemblies

A most preferred embodiment of the present invention are acoustically responsive supramolecular complexes, specifically developed for ultrasound-mediated intracellular drug delivery in vivo. In several preferred embodiments, constituents in such a complex include
1. Block copolymers, having at least one nonionic, water soluble segment, and at least one polyionic segment; and
2. At least one charged surfactant having hydrophobic groups, with the charge of the surfactant being opposite to the charge of the polyionic segment of the block copolymer.

The constituents of the complex are bound by interaction between the opposite charges thereof and between surfactant hydrophobic groups. However, in compositions of the present invention comprising an anionic surfactant having biological activity, such an anionic surfactant has a net charge of no more than approximately 10, preferably 5.

The polyionic segment of the block copolymer may be polyanionic, in which case the surfactant is a cationic surfactant, or the polyionic segment of the block copolymer may be polycationic, in which case the surfactant is an anionic surfactant.

Further, a method is provided for preparation of the above-described compositions in the form of vesicles for acoustically mediated in vivo drug delivery of a variety of therapeutics, as listed herein. In preparing the present invention, a block copolymer having at least one nonionic water soluble segment and at least one polyionic segment, is mixed with a charged surfactant having hydrophobic groups, with the charge of the surfactant being opposite to the charge of the polyionic segment of the block copolymer. Further, the ratio of the net charge of the surfactant to the net charge of the polyionic segment present in the block copolymer is between approximately 0.01 and approximately 100.

In a preferred embodiment, the block copolymer is selected from the group consisting of polymers of formulas illustrated in FIG. 9, wherein N is a nonionic, water-soluble segment (“N-type segment”), P is polyionic segment (“P-type segment”) and n is an integer from 1 to 5,000 (FIG. 9). Preferably, the degrees of polymerization of N-type and P-type segments are from approximately 3 to approximately 50,000, more preferably from approximately 5 to approximately 5,000, still more preferably from approximately 20 to approximately 500. If more than one segment of the same type comprises one block copolymer, then these segments may all have the same lengths or may have different lengths (FIG. 9).

The preferred polyanion P-type segments (FIG. 9) include, but are not limited to, those such as polymethacrylic acid and its salts, polyacrylic acid and its salts, copolymers of methacrylic acid and its salts, copolymers of acrylic acid and its salts, heparin, poly(phosphate), polyamino acid (e.g., polyaspartic acid, polyglutamic acid, and their copolymers containing a plurality of anionic units), polymalic acid, polylactic acid, polynucleotides, carboxylated dextran, and the like. Particularly preferred polyanion P-type segments are the products of polymerization or copolymerization of monomers which polymerize to yield a product having carboxyl pendant groups. Representative examples of such monomers include acrylic acid, aspartic acid 1,4-phenylenediacrylic acid, citraconic acid, citraconic anhydride, trans-cinnamic acid, 4-hydroxy-3-methoxy cinnamic acid, p-hydroxy cinnamic acid, trans-glutaconic acid, glutamic acid, itaconic acid, linoleic acid, linolenic acid, methacrylic acid, maleic acid, maleic anhydride, mesaconic acid, trans-β-hydromuconic acid, trans-trans muconic acid, oleic acid, ricinoleic acid, 2-propene-1-sulfonic acid, 4-styrene sulfonic acid, trans-traumatic acid, vinylsulfonic acid, vinyl phosphonic acid, vinyl benzoic acid, and vinyl glycolic acid.

Preferred polycation P-type segments (FIG. 9) include, but are not limited to, polyamino acid (e.g., polylysine), alkanolamine esters of polymethacrylic acid (e.g., poly-(dimethylammonioethyl methacrylate)), polyamines (e.g., spermine, polyspermine, polyethyleneimine), polyvinyl pyridine, and the quaternary ammonium salts of said polycation segments.

Preferably, nontoxic and non-immunogenic polymers-forming N-type and P-type segments should be used (FIG. 9). Because of elevated toxicity and immunogenicity of cationic peptides, the non-peptide P-type segments are particularly preferred.

In the case of block copolymers having at least one polyanionic segment, the nonionic segment may include, without limitation, polyetherglycols (e.g., poly(ethylene oxide), poly(propylene oxide)) copolymers of ethylene oxide and propylene oxide; polysaccharides (e.g., dextran); products of polymerization of vinyl monomers (e.g., polyacrylamide), polyacrylic esters (e.g., polyacroloyl morpholine); polymethacrylamide, poly(N-2-hydroxypropyl)methacrylamide; polyvinyl alcohol; polyvinyl pyrrolidone; polyvinyltriazole, N-oxide of polyvinylpyridine); polyortho esters; polyamino acids; polyglycerols (e.g., poly-2-methyl-2-oxazoline, poly-2-ethyl-2-oxazoline) and copolymers; and derivatives thereof.

Block copolymers comprising at least one polycationic segment may be similarly formulated using nonionic segments such as polyetherglycols (e.g., polyethylene glycol) or copolymers of ethylene oxide and propylene oxide.

The charged surfactants suitable for use in the practice of the present invention are broadly characterized as cationic and anionic surfactants having hydrophobic/lipophilic groups, (i.e., the groups poorly soluble in water) and/or revealing an ability to absorb at a water-air interface, and/or solubilize in organic solvents with low polarity and/or self-assemble in aqueous media to form a nonpolar microphase. The use of such compounds is an important feature of the present teachings. The interactions of hydrophobic groups of surfactant molecules with each other contribute to cooperative stabilization of the ionic complexes formed, with said complexes formed between the block copolymers and surfactants of the opposite charge in the compositions of the present teachings, as will be further described below. These include single-, double-, and triple-tail surfactants. Typically, the cationic surfactants will be lipophilic quaternary ammonium salts, lipopolyamines, lipophilic polyamino acids or a mixture thereof, particularly those proposed heretofore as a constituent of cationic lipid formulations for use in nucleic acid delivery. Various examples of classes and species of suitable cationic surfactants are provided below.

Cationic surfactants that can be used in the compositions of the present invention include, but are not limited to primary amines (e.g., hexylamine, heptylamine, octylamine, decylamine, undecylamine, dodecylamine, pentadecyl amine, hexadecyl amine, oleylamine, stearylamine, diaminopropane, diaminobutane, diaminopentane, diaminohexane, diaminoheptane, diaminooctane, diaminononane, diaminodecane, and diaminododecane); secondary amines (e.g., N,N-distearylamine, adrenolutin, adrenalone, adrenolglomerulotropin, albuterol, azacosterol, benzoctamine, benzydamine, carazolol, cetamolol, and spirogermanium); tertiary amines (e.g., N,N′,N′-polyoxyethylene(10)-N-tallow-1,3-diaminopropane, acecainide, adiphenine hydrochloride, adinozalam, ahistan, alloclamide, allocryptopyne, almitrine, amitriptyline, anileridine, aprindine, bencyclane, benoxinate, biphenamine, brompheniramine, bucumolol, bufetolol, bufotenine, bufuralol, bunaftine, bunitrolol, bupranolol, butacaine, butamirate, butethamate, butofilolol, butoxycaine, butriptyline, captodiamine, caramiphen hydrochloride, carbetapentane, carbinoxamine, carteolol, cassaidine, cassaine, cassamine, chlorpromazine, dimenoxadol, dimethazan, diphehydramine, orphenandrine, pyrilamine, pyrisuccidianol, succinylcholone iodide, tetracaine, and the like); quaternary ammonium salts, which include aromatic and non-aromatic ring-containing compounds (e.g., dodecyltrimethylammonium bromide, hexadecyltrimethylammonium bromide, alkyltrimethylammonium bromide, tetradecyltrimethylammonium bromide, benzalkonium chloride, benzethonium chloride, benzoquinonium chloride, benzoxonium chloride, bibenzonium bromide, cetalkonium chloride, cethexonium bromide, benzylonium bromide, benzyldimethyldodecylammonium chloride, benzyldimethylhexadecylammonium chloride, benzyltrimethylammonium methoxide, cetyldimethylethylammonium bromide, dimethyldioctadecyl ammonium bromide (DDAB), methylbenzethonium chloride, decamethonium chloride, methyl mixed trialkyl ammonium chloride, methyl trioctylammonium chloride, N-alkyl pyridinium salts, N-alkylpiperidinium salts, quinaldinium salts, amprolium, benzylpyrinium, bisdequalinium halides, and azonium and azolium salts (e.g., anisotropine methylbromide, butropium bromide, N-butylscopolammonium bromide, tetrazolium blue), quinolinium derivatives (e.g., atracurium besylate); piperidinium salts (e.g., bevonium methyl sulfate and thiazolium salts, such as beclotiamine); 1,2-diacyl-3-(trimethylammonio)propane (acyl group=dimyristoyl, dipalmitoyl, distearoyl, dioleoyl); 1,2-diacyl-3-(dimethylammonio)propane (acyl group=dimyristoyl, dipalmitoyl, distearoyl, dioleoyl); 1,2-dioleoyl-3-(4′-trimethylammonio) butanoyl-sn-glycerol; 1,2-dioleoyl-3-succinyl-sn-glycerol choline ester, cholesteryl (4′-trimethylammonio) butanoate), heterocyclic amines (e.g., azacuclonol, azaperone, azatadine, benzetimide, benziperylon, benzylmorphine, bepridil, biperidene, budipine, buphanamine, buphanitine, butaperazine, butorphanol, buzepide, calycanthine, carpipramine); imidazoles (e.g., azanidazole, azathiopropine, bifonazole, bizantrene, butacanazole, and cafaminol); triasoles (e.g., bitertanol); tetrazoles (e.g., azosemide); phenothiazines (e.g., azures A, B, and C); aminoglycans (e.g., daunorubicin, doxorubicin, caminomycin, 4′-epiadriamycin, 4-demethoxy-daunomycin, 11-deoxydaunorubicin, 13-deoxydaunorubicin, adriamycin-14-benzoate, adriamycin-14-actanoate, and adriamycin-14-naphthaleneacetate); rhodamines (e.g., rhodamine 123); acridines (e.g., acranil, acriflavine, acrisorcin); dicationic bolaform electrolytes, dialkylglycetylphosphorylcholine, lysolecithin; cholesterol hemisuccinate choline ester; lipopolyamines (e.g., dioctadecylamidoglycylspermine [DOGS], dipalmitoyl phosphatidylethanolamidospermine [DPPES]), N′-octadecylsperminecarboxamide hydroxytrifluoroacetate, N′,N″-dioctadecylspermine-carboxamide hydroxytrifluoroacetate, N′-nonafluoro pentadecylosperminecarboxamide hydroxytrifluoroacetate, N′,N″-dioctyl (sperminecarbonyl)glycinamide hydroxytrifluoroacetate, N′-(heptadecafluorodecyl)-N′-(nonafluoropentadecyl)-sperminecarbonyl) glycinamede hydroxytrifluoroacetate, N′-(3,6,9-trioxa-7-(2′-oxaeicos-11′-enyl)heptaeicos-18-enyl) sperminecarbo-xamide hydroxytrifluoroacetate, N′-(1,2-dioleoyl-sn-glycero-3-phosphoethanoyl) spermine carboxamide hydroxytrifluoroacetate), 2,3-dioleyloxy-N-(2(spermine-carboxamido) ethyl)-N,N-dimethyl-1-propanaminiumtrifluoroacetate (DOSPA), N,N_{I},N_{II},N_{III}-tetramethyl-N,N_{I},N_{II},N_{III}-tetrapatmitylspermine (TM-TPS), N-(1-(2,3-dioleyloxy)propyl)-N,N,N-trimethylamonium chloride (DOTMA), 1,2-dioleoyl-3-dimethyl-hydroxyethyl ammonium bromide (DORI), 1,2-dioleyloxypropyl-3-dimethyl-hydroxyethyl ammonium bromide (DORIE), 1,2-dioleyloxypropyl-3-dimethyl-hydroxypropyl ammonium bromide (DORIE-HP), 1,2-dioleyloxypropyl-3-dimethyl-hydroxybutyl ammonium bromide (DORIE-HB), 1,2-dioleyloxypropyl-3-dimethyl-hydroxypentyl ammonium bromide (DORIE-HPe), 1,2-dimyristyloxypropyl-3-dimethyl-hydroxyethyl ammonium bromide (DMRIE), 1,2-dipalmitoyloxypropyl-3-dimethyl-hydroxyethyl ammonium bromide (DPRIE), 1,2-distearoyloxypropyl-3-dimethyl-hydroxyethyl ammonium bromide (DSRIE), N,N-dimethyl-N-(2-(2-methyl-4-(1,1,3,3-tetramethylbutyl)-phenoxy) ethoxy)ethyl)-benzenemethanaminium chloride (DEBDA), N-(1-(2,3-dioleyloxy)propyl)-N,N,N-trimethylammonium methylsulfate (DOTAB), lipopoly-L(or D)-lysine, poly(L (or D)-lysine conjugated to N-glutarylphosphatidylethanolamine lysine, didodecyl glutamate ester with pendant amino group (C₁₂GluPhCₙN⁺), ditetradecyl glutamate ester with pendant amino group (C₁₄GluCₙN⁺), 9-(N′,N″-dioctadecylglycinamido)acridine, ethyl 4-((N-(3-bis(octadecylcarbamoyl)-2-oxapropylcarbonyl) glycinamido)pyrrole-2-carboxamido)-4-pyrrole-2-carboxylate, N′,N′-dioctadecylornithylglycinamide hydroptrifluoroacetate, cationic derivatives of cholesterol (e.g., cholesteryl-3(-oxysuccinamidoethylenetri methylammonium salt, cholesteryl-3(-oxysuccinamidoethylenedimethylamine, cholesteryl-3(-carboxyam idoethylenetrimethylammonium salt, cholesteryl-3(-carboxyamidoethylenedi-methylamine, 3((N—(N′,N′-dimethylaminoetane-carbomoyl)cholesterol), pH-sensitive cationic lipids (e.g., 4-(2,3-bis-palmitoyloxy-propyl)-1-methyl-1H-imidazole, 4-(2,3-bis-oleoyloxy-propyl)-1-methyl-1H-imidazole, cholesterol-(3-imidazol-1-yl propyl)carbamate, 2,3-bis-palmitoyl-propyl-pyridin-4-yl-amine), and the like.

Especially useful in the context of gene delivery and other applications are compositions comprising mixtures of cationic surfactants and nonionic surfactants including, but not limited to, dioleoyl phosphatidylethanolamine (DOPE), dioleoyl phosphatidylcholine (DOPC). This includes, in particular, commercially available cationic lipid compositions including, but not limited to, LipofectAMINE™, Lipofectine®, DMRIE-C, CelIFICTIN™, LipofectACE™, Transfectam reagents, and other cationic lipid compositions used for transfection of cells.

The anionic surfactants that can be used in the compositions of the present invention include, but are not limited to, alkyl sulfates, alkyl sulfonates, fatty acid soaps, including salts of saturated and unsaturated fatty acids and derivatives (e.g., adrenic acid, arachidonic acid, 5,6-dehydroarachidonic acid, 20-hydroxyarachidonic acid, 20-trifluoro arachidonic acid, docosahexaenoic acid, docosapentaenoic acid, docosatrienoic acid, eicosadienoic acid, 7,7-dimethyl-5,8-eicosadienoic acid, 7,7-dimethyl-5,8-eicosadienoic acid, 8,11-eicosadiynoic acid, eicosapentaenoic acid, eicosatetraynoic acid, eicosatrienoic acid, eicosatriynoic acid, eladic acid, isolinoleic acid, linoelaidic acid, linoleic acid, linolenic acid, dihomo-γ-linolenic acid, γ-linolenic acid, 17-octadecynoic acid, oleic acid, phytanic acid, stearidonic acid, 2-octenoic acid, octanoic acid, nonanoic acid, decanoic acid, undecanoic acid, undecelenic acid, lauric acid, myristoleic acid, myristic acid, palmitic acid, palmitoleic acid, heptadecanoic acid, stearic acid, nonanedecanoic acid, heneicosanoic acid, docasanoic acid, tricosanoic acid, tetracosanoic acid, cis-15-tetracosenoic acid, hexacosanoic acid, heptacosanoic acid, octacosanoic acid, and triocantanoic acid); salts of hydroxy-, hydroperoxy-, polyhydroxy-, epoxy-fatty acids, salts of saturated and unsaturated, mono- and poly-carboxylic acids (e.g., valeric acid, trans-2,4-pentadienoic acid, hexanoic acid, trans-2-hexenoic acid, trans-3-hexenoic acid, 2,6-heptadienoic acid, 6-heptenoic acid, heptanoic acid, pimelic acid, suberic acid, sebacicic acid, azelaic acid, undecanedioic acid, decanedicarboxylic acid, undecanedicarboxylic acid, dodecanedicarboxylic acid, hexadecanedioic acid, docasenedioic acid, tetracosanedioic acid, agaricic acid, aleuritic acid, azafrin, bendazac, benfurodil hemisuccinate, benzylpenicillinic acid, p-(benzylsulfonamido)benzoic acid, biliverdine, bongkrekic acid, bumadizon, caffeic acid, calcium 2-ethylbutanoate, capobenic acid, carprofen, cefodizime, cefinenoxime, cefixime, cefazedone, cefatrizine, cefamandole, cefoperazone, ceforanide, cefotaxime, cefotetan, cefonicid, cefotiam, cefoxitin, cephamycins, cetiridine, cetraric acid, cetraxate, chaulmoorgic acid, chlorambucil, indomethacin, protoporphyrin IX, protizinic acid), prostanoic acid and its derivatives (e.g., prostaglandins), leukotrienes and lipoxines, alkyl phosphates, O-phosphates (e.g., benfotiamine), alkyl phosphonates, natural and synthetic lipids (e.g., dimethylallyl pyrophosphate ammonium salt, S-farnesylthioacetic acid, farnesyl pyrophosphate, 2-hydroxymyristic acid, 2-fluorpalmitic acid, inositoltrphosphates, geranyl pyrophosphate, geranygeranyl pyrophosphate, α-hydroxyfarnesyl phosphonic acid, isopentyl pyrophoshate, phosphatidylserines, cardiolipines, phosphatidic acid and derivatives, lysophosphatidic acids, sphingolipids, and the like), synthetic analogs of lipids (e.g., sodium-dialkyl sulfosuccinate [e.g., Aerosol OT®]); n-alkyl ethoxylated sulfates, n-alkyl monothiocarbonates, alkyl- and arylsulfates (asaprol, azosulfamide, p-(benzylsulfonamideo)benzoic acid, cefonicid, CHAPS), mono- and dialkyl dithiophosphates, N-alkanoyl-N-methylglucamine, perfluoroalcanoate, cholate and desoxycholate salts of bile acids, 4-chloroindoleacetic acid, cucurbic acid, jasmonic acid, 7-epi jasmonic acid, 12-oxo phytodienoic acid, traumatic acid, tuberonic acid, abscisic acid, acitertin, and the like.

Preferred cationic and anionic surfactants also include fluorocarbon and mixed fluorocarbon-hydrocarbon surfactants. Additional surfactants useful in the present invention include, but are not limited to, the salts of perfluoromonocarboxylic acids (e.g., pentafluoropropionic acid, heptafluorobutyric acid, nonanfluoropentanoic acid, tridecafluoroheptanoic acid, pentadecafluorooctanoic acid, heptadecafluorononanoic acid, nonadecafluorodecanoic acid, perfluorododecanoic acid, perfluoropolycarboxylic acids, and perfluorotetradecanoic acid) and the salts of perfluoro-polycarboxylic acids (e.g., hexafluoroglutaric acid, perfluoroadipic acid, perfluorosuberic acid, and perfluorosebacic acid), double-tail hybrid surfactants, (CₘF₂ₘ₊₁)(CₙH₂₊₁)CH—OSO₃Na, fluoroaliphatic phosphonates, fluoroaliphatic sulphates, and the like.

The biological agent compositions of this invention may additionally contain nonionic or zwitterionic surfactants including, but not limited to, phospholipids (e.g., phosphatidylethanolamines, phosphatidylglycerols, phosphatidylinositols, diacyl phosphatidylcholines, di-O-alkyl phosphatidylcholines, platelet-activating factors, PAF agonists and PAF antagonists, lysophosphatidylcholines, lysophosphatidylethanol-amines, lysophosphatidylglycerols, lysophosphatidylinositols, lyso-platelet-activating factors and analogs, and the like), saturated and unsaturated fatty acid derivatives (e.g., ethyl esters, propyl esters, cholesteryl esters, coenzyme A esters, nitrophenyl esters, naphtyl esters, monoglycerids, diglycerids, and triglycerids, fatty alcohols, fatty alcohol acetates, and the like), lipopolysaccharides, glyco- and shpingolipids (e.g., ceramides, cerebrosides, galactosyldiglycerids, gangliosides, lactocerebrosides, lysosulfatides, psychosines, sphingomyelins, sphingosines, sulfatides), chromophoric lipids (neutral lipids, phospholipids, cerebrosides, sphingomyelins), cholesterol and cholesterol derivatives, Amphotericin B, abamectin, acediasulfone, n-alkylphenyl polyoxyethylene ether, n-alkyl polyoxyethylene ethers (e.g., Triton™), sorbitan esters (e.g., Span™), polyglycol ether surfactants (Tergitol™), polyoxyethylenesorbitan (e.g., Tween™), polysorbates, polyoxyethylated glycol monoethers (e.g., Brij™, polyoxylethylene 9 lauryl ether, polyoxylethylene 10 ether, polyoxylethylene 10 tridecyl ether), lubrol, copolymers of ethylene oxide and propylene oxide (e.g., Pluronic™, Pluronic®, Teronic™, Pluradot™, alkyl aryl polyether alcohol (Tyloxapol™), perfluoroalkyl polyoxylated amides, N,N-bis[3-D-gluconamidopropyl]cholamide, decanoyl-N-methylglucamide, n-decyl α-D-glucopyranozide, n-decyl β-D-glucopyranozide, n-decyl β-D-maltopyranozide, n-dodecyl β-D-glucopyranozide, n-undecyl β-D-glucopyranozide, n-heptyl (-d-glucopyranozide, n-heptyl β-D-thioglucopyranozide, n-hexyl β-D-glucopyranozide, n-nonanoyl β-D-glucopyranozide 1-monooleyl-rac-glycerol, nonanoyl-N-methylglucamide, n-dodecyl α-D-maltoside, n-dodecyl β-D-maltoside, N,N-bis[3-gluconamidepropyl]deoxycholamide, diethylene glycol monopentyl ether, digitonin, heptanoyl-N-methylglucamide, heptanoyl-N-methylglucamide, octanoyl-N-methylglucamide, n-octyl β-D-glucopyranozide, n-octyl α-D-glucopyranozide, n-octyl β-D-thiogalactopyranozide, n-octyl .beta.-D-thioglucopyranozide, betaine (R₁R₂R₃N⁺R′CO₂⁻, where R₁R₂R₃R′ hydrocarbon chains), sulfobetaine (R₁R₂R₃N⁺R′SO₃⁻), phoshoplipids (e.g., dialkyl phosphatidylcholine), 3-[(3-cholamidopropyl)-dimethylammonio]-2-hydroxy-1-propanesulfonate, 3-[(3-cholamidopropyl)-dimethylammonio]-1-propanesulfonate, N-decyl-N,N-dimethyl-3-ammonio-1-propanesulfonate, N-dodecyl-N,N-dimethyl-3-ammonio-1-propanesulfonate, N-hexadecyl-N,N-dimethyl-3-ammonio-1-propanesulfonate, N-octadecyl-N,N-dimethyl-3-ammonio-1-propanesulfonate, N-octyl-N,N-dimethyl-3-ammonio-1-propanesulfonate, N-tetradecyl-N,N-dimethyl-3-ammonio-1-propanesulfonate, and dialkyl phosphatitidylethanolamine.

Importantly, it is essential that the biologically active agents delivered by the present teachings, examples of which are listed later in this specification, are charged in order to successfully form a complex with the block copolymer of opposite charge. The term “charged biological agent” is used herein to encompass, without limitation, any biological agent that can produce either cation or anion groups in aqueous solution. This includes, without limitation, strong bases (e.g., quaternary ammonium or pyridinium salts, and the like) that dissociate in aqueous solution to form cationic group, and weak bases (e.g., primary amines, secondary amines, and the like) that protonate in aqueous solution to produce a cationic group as a result of an acidic-basic reaction. The anionic biological agents include, without limitation, strong acids and their salts (e.g., agents containing sulfate groups, sulfonate groups, phosphate groups, phosphonate groups, and the like) that dissociate in aqueous solution to form an anionic group, and weak acids (e.g., carboxylic acids) that ionize in aqueous solution to produce an anionic group as a result of an acidic-basic reaction.

### Nanocarrier Stabilization

The nanocarriers of this disclosure may possess functional groups which may be cross-linked or stabilized by a variety of processes, methodologies, and procedures. Cross-linking the components of said carriers is particularly useful in applications requiring additional stability of the nanocarrier corona, altered acoustic responsiveness, and/or increased retention capabilities of the encapsulated materials (e.g., nucleic acids). Cross-linked dendritic polymers covalently interconnected include (1) completely cross-linked nanocarriers, having all corona components covalently interconnected into a giant single molecule, (2) cross-linked nanocarriers having interconnected components throughout the entire surface of said nanocarrier, and (3) partly cross-linked nanocarriers containing patches of interconnected components. Possible stabilization techniques include cross-linking by sulfur to form disulfide linkages, cross-linking using organic peroxides, cross-linking of unsaturated materials by means of high-energy radiation, photopolymerization, cross-linking with dimethylol carbamate, and the like.

One of the most preferred stabilization techniques for use with the present teachings is photopolymerization, which is a methodology that uses light to initiate and propagate a polymerization reaction to form a linear, or cross-linked polymeric structure. This technology has been widely explored in a variety of industries for several different applications including, for example, the coating industry, the paint and printing ink industries, in adhesives, and in composite materials. Recently, the use of photopolymerization has been proposed for the production of biomaterial-based polymer networks that could be differentially fabricated for a variety of applications including embodiments of this specification.

Photopolymers have broad utility in drug delivery because of a combination of properties held by photopolymerizable precursors, and/or photopolymerized polymer networks. Exemplary characteristics include: ease of production, possibility of carrying out photopolymerization in vivo or ex vivo, spatial and temporal control of the polymerization process, versatility of formulation and application, and the possibility of entrapping a wide range of substances. Further, especially important characteristics for use with the present teachings include the ability to store photopolymerization formulation ingredients in easily accessible conditions until use (e.g., in a clinical situation when said components need to be immediately mixed together before production of polymer networks).

A wide variety of sources are known to those skilled in the art describing techniques, procedures, and methods for photopolymerization and biomedical applications, such as, for example, drug delivery using the nanocarriers of this disclosure and other structures. Preferred publications include, but are not limited to: Microparticulate Systems for the Delivery of Proteins and Vaccines (Drugs and the Pharmaceutical Sciences: a Series of Textbooks and Monographs) (Cohen et al., 1996); Polyurethanes in Biomedical Applications (Lamba et al., 1998); Biomaterials for Delivery and Targeting of Proteins and Nucleic Acids (Mahato, 2005); Chemical and Physical Networks: Formation and Control of Properties (The Wiley Polymer Networks Group Review) (Nijenhuis et al., 1998); Polymeric Drugs & Drug Delivery Systems (Ottenbrite et al., 2001); Photoinitiation, Photopolymerization, and Photocuring: Fundamentals and Applications (Fouassier, 1995); Photostability of Drugs and Drug Formulations (Tønnesen, 2004); Polymers in Drug Delivery (Uchegbu et al., 2006); Drug Delivery: Principles and Applications (Wiley Series in Drug Discovery and Development) (Wang et al., 2005), the disclosures of each of which are hereby incorporated by reference herein in their entirety for all purposes.

In their simplest form, a photopolymerizable system is composed of (1) a light source, (2) a photoinitiator, and (3) a monomer. In addition, this formulation can be supplemented with other molecules (e.g., monomers, cross-linkers, excipients, and bioactive molecules, drugs) to fulfill specific drug delivery applications.

The light sources that heretofore have been utilized in producing biomedical polymer networks and devices, and may be used in embodiments of this disclosure include UV lamps; halogen lamps; plasma arc lamps; light-emitting diode (LED) lamps; titanium-sapphire lasers, also called fernto second pulsed lasers; and other laser lamps. These sources generate a beam of light that differs in terms of emission wavelength, intensity, and associated heat.

Photoinitiators are molecules responsible for initiating the polymerization reaction by producing reactive species upon light absorption. There are many different photoinitiator molecules known in the art, some of which are suitable for use in the present teachings. A partial list includes eosin Y, 1-cyclohexyl phenyl ketone; 2,2-dimethoxy-2-phenylacetophenone (DMPA), 2-hydroxy-1-[(4-hydroxyethoxy)phenyl]-2-methyl-1-propanone, Irgacure 651; camphorquinone/amine, where the amine is triethylamine, triethanolamine or ethyl 4-N—N-dimethylamino benzoate; and the like. A photoinitiator can induce a polymerization reaction directly or in the presence of other molecules. A system, composed of a photoinitiator and other molecule(s), may have a synergistic function; and said mixtures are normally referred to as a Photoinitiator System (PIS). When a photopolymerizable formulation is irradiated with an appropriate light source, a series of events take place. In the presence of a PIS, the photosensitizer will absorb said light energy, thus passing into an excited state. Successively, the photosensitizer has either to transfer said energy to the photoinitiator or to react with the photoinitiator itself. Both situations will cause the photoinitiator to produce reactive species (e.g., cationic, anionic, or free radicals). In the absence of a photosensitizer, the excited photoinitiator forms reactive species directly. Further, formulations may contain other molecules, called accelerators, which speed these initial steps. Once the reactive species are generated, polymerization is initiated by (1) photocleavage, (2) hydrogen abstraction, or (3) generation of cationic species. The first two mechanisms of initiation will generate a free radical photo-induced polymerization, which are the most commonly employed methodologies for use with biomaterials.

Commercially and non-commercially available molecules and macromolecules used as photopolymerizable monomers and macro-monomers (i.e., macromers) have one primary feature in common: Their backbone needs to have a photopolymerizable residue that normally is located at one or at both ends of the molecule. Photopolymerizable monomers already known in the art and suitable for use with the present teachings include (di)methacrylic or (di) acrylic derivatives of PEG and its derivatives; poly(ethylene oxide) poly(vinyl) alcohol (PVA) and its derivatives; PEG-polystyrene copolymers (PEG)-(PST); ethylene glycol-lactic acid copolymers (nEGmlA, where n and m are the number of repeat units of EG and LA, respectively); ethylene glycol-lactic acid-caprolactone copolymers (nEGmlA CL); PLA-b-PEG-b-PLA; PLA-g-PVA; poly(D,L-lactide-co-∈-caprolactone); (poly)-anhydrides; 27 anhydrides; urethanes; polysaccharides; dextran; collagen; hyaluronic acid; diethyl fumarate/poly(propylene fumarate); and the like.

The introduction of specific properties (e.g., cell and protein adhesiveness or non-adhesiveness, mechanical strength and acoustic sensitivity, degradation rate, absence or limited mass transport constraints) in polymerized networks can be achieved by selecting appropriate monomer(s) and/or macromer(s), and supplemental molecules during the design of the monomer or its formulation. As an example, approaches to modify the degradation rate and cell adhesiveness of the polymerized networks are reported because of their significance for use in the present specification.

Degradation rate may be controlled by (1) the number of degradable chemical bonds in each monomer; (2) the type of degradable chemical bonds (e.g., ester, anhydride, and amide); (3) the molecular weight of the monomer; and (4) the hydrophobic or hydrophilic nature of the monomer. While the number and nature of degradable chemical bonds are obvious key factors, the molecular weight determines whether polymer networks will be loosely (i.e., high MW) or tightly (i.e., low MW) cross-linked. In the second case, the degradation rate is slow because degradable linkages are hindered within the densely cross-linked network. In addition, hydrophobicity of highly cross-linked networks will further decrease their degradation. Obviously, degradation rate may be critical in applications involving the present teachings because it can influence, for example, the release and retention of entrapped therapeutics and other molecules.

### Nanocarrier Size

The size of the nanocarriers of embodiments of the present invention will depend on their intended use. Sizing also serves to modulate resultant biodistribution and clearance. The size of the nanocarrier can be adjusted, if desired, by the preferred method of filtering, although, other procedures known to those skilled in the art can also be used (e.g., shaking, microemulsification, vortexing, repeated freezing and thawing cycles, extrusion, and extrusion under pressure through pores of a defined size, sonication, and homogenization). See, for example, U.S. Pat. Nos. 4,728,578; 4,728,575; 4,737,323; 4,533,254; 4,162,282; 4,310,505; and 4,921,706, the disclosures of each of which are hereby incorporated by reference herein in their entirety for all purposes.

After intravenous injection, particles greater than 5 μm to 7 μm in diameter are often accumulated in the lung capillaries, while particles with a diameter of less than 5 μm are generally cleared from the circulation by the cells of the reticuloendothelial system. Particles in excess of 7 μm are larger than the blood capillary diameter—approximately 6 μm—and will be mechanically filtered. In the size range 70 nm to 200 nm, the surface curvature of particles may affect the extent and/or the type of protein or opsonin absorption, which plays a critical role in complement activation. The fact that particle size may change substantially upon introduction into a protein-containing medium (e.g., plasma) must also be taken into consideration.

Therefore, since vesicle size influences biodistribution, different-sized vesicles may be selected for various purposes. For example, for intravascular application, the preferred size range is a mean outside diameter between approximately 20 nm and approximately 1.5 μm, with the preferable mean outside diameter being approximately 750 nm. More preferably, for intravascular application, the size of the vesicles is approximately 200 nm or less in mean outside diameter, and most preferably less than approximately 100 nm in mean outside diameter. Preferably, the vesicles are no smaller than approximately 20 nm in mean outside diameter. To provide therapeutic delivery to organs (e.g., the liver) and to allow differentiation of a tumor from normal tissue, smaller vesicles, between approximately 30 nm and approximately 100 nm in mean outside diameter, are preferred. For immobilization of a tissue (e.g., the kidney or the lung), the vesicles are preferably less than approximately 200 nm in mean outside diameter. For intranasal, intrarectal, or topical administration, the vesicles are preferably less than approximately 100 nm in mean outside diameter. Large vesicles, between 1 μm and approximately 1.5 μm in size, will generally be confined to the intravascular space until they are cleared by phagocytic elements of the immune system (e.g., the macrophages and Kupffer cells) lining capillary sinusoids. For passage to the cells beyond the sinusoids, smaller vesicles, for example, less than approximately 1 μm in mean outside diameter, and less than approximately 300 nm in size, may be utilized. In preferred embodiments, the vesicles are typically administered individually, although this administration may be in some type of polymer matrix.

### Nanocarrier and Contrast Agent Targeting

As described herein, embodiments of the present invention include nanocarriers and/or certain contrast agents which may comprise various targeting components (e.g., ligands) to target the vesicle and its contents to, for example, specific cells either in vitro or in vivo. A ligand or targeting ligand is a molecule that specifically binds to another molecule, which may be referred to as a target. In another preferred embodiment, the nanocarriers of this specification can be targeted by magnetic compositions associated with said vesicle, and then guided by a magnetic field. Or, in yet another embodiment, both targeting ligands and magnetic compositions are utilized for active targeting. All of the targeting ligands and magnetic compositions described herein are considered to be within the definition of a “targeting moiety,” and are thus suitable for use with embodiments of this disclosure.

The targeting ligands incorporated in the nanocarriers of this specification are preferably substances which are capable of targeting receptors and/or tissues in vivo and/or in vitro. Preferred targeting ligands are selected from the group consisting of proteins, including antibodies, antibody fragments, hormones, hormone analogues, glycoproteins and lectins, peptides, polypeptides, and amino acids; sugars (e.g., saccharides, including monosaccharides and polysaccharides); carbohydrates, vitamins, steroids, steroid analogs, hormones, and cofactors; genetic material, including aptamers, nucleosides, nucleotides, nucleotide acid constructs, and polynucleotides; and peptides. Preferred targeting ligands for use with embodiments described herein include, for example, cell adhesion molecules (CAMs), among which are cytokines, integrins, cadherins, immunoglobulins and selectins; optimal genetic material for targeting includes aptamers.

A wide variety of sources is known in the art that describes techniques, procedures, and methods for active targeting of drug-containing vesicles using ligands and other structures (e.g., the nanocarriers of embodiments of the present invention). For enablement purposes, preferred publications include, but are not limited to

Cellular Drug Delivery: Principles and Practice (Lu et al., 2004), Drug Targeting Organ-Specific Strategies (Molema et al., 2001); Biomedical Aspects of Drug Targeting (Muzykantov et al., 2002); Protein-Protein Interactions: Molecular Cloning Manual (Golemis et al., 2005), Using Antibodies: Laboratory Manual (Harlow et al., 1999); Molecular Cloning: Laboratory Manual (Sambrook et al., 2001); Drug Targeting Technology: Physical, Chemical, and Biological Methods (Schreier, 2001), Liposomes: A Practical Approach (Torchilin et al., 2003), and patents identifying and describing specific targeting ligands of medical importance and methods of use thereof, include, but are not limited to U.S. Pat. Nos. 5,128,326; 5,580,960; 5,610,031; 5,625,040; 5,648,465; 5,766,922; 5,770,565; 5,792,743; 5,849,865; 5,866,165; 5,872,231; 6,121,231; 6,140,117; 6,159,467; 6,204,054; 6,352,972; and 6,482,410. The disclosures of each of the publications and patents in this paragraph [0462] are hereby incorporated by reference herein in their entirety for all purposes.

Briefly, many different targeting ligands can be selected to bind to specific domains of various adhesion molecules (e.g., the Immunoglobulin Superfamily [ICAM 1-3, PECAM-1, VCAM-1], the Selectins [EWLAM-1, LECAM-1, GMP-140] and the Integrins [LEA-1]). Targeting ligands in this regard may include lectins, a wide variety of carbohydrate or sugar moieties; antibodies; antibody fragments; Fab fragments (e.g., Fab′₂); and synthetic peptides, including, for example, Arginine-Glycine-Aspartic Acid (R-G-D), which may be targeted to wound healing. While many of these materials may be derived from natural sources, some may be synthesized by molecular biological recombinant techniques, and others may be synthetic in origin. Peptides may be prepared by a variety of techniques known in the art. Targeting ligands derived or modified from human leukocyte origin (e.g., CD11a/CD18 and leukocyte cell surface glycoprotein [LFA-1]) may also be used as these bind to the endothelial cell receptor ICAM-1. The cytokine inducible member of the immunoglobulin superfamily, VCAM-1, which is mononuclear leukocyte-selective, may also be used as a targeting ligand. VLA-4, derived from human monocytes, may be used to target VCAM-1. Antibodies and other targeting ligands may be employed to target endoglin, which is an endothelial cell proliferation marker. Endoglin is upregulated on endothelial cells in miscellaneous solid tumors. Further, the cadherin family of cell adhesion molecules may also be used as targeting ligands, including, for example, the E-, N-, and P-cadherins, cadherin-4, cadherin-5, cadherin-6, cadherin-7, cadherin-8, cadherin-9, cadherin-10, and cadherin-11, and most preferably, cadherin C-5. Further, antibodies directed to cadherins, may be used to recognize cadherins expressed locally by specific endothelial cells.

Targeting ligands may be selected for targeting antigens, including antigens associated with breast cancer, such as epidermal growth factor receptor (EGFR), fibroblast growth factor receptor, erbB2/BER-2, and tumor associated carbohydrate antigens, CTA 16.88, homologous to cytokeratins 8, 18, and 19, which is expressed by most epithelial-derived tumors including carcinomas of the colon, pancreas, breast, and ovary. Chemically conjugated bispecific anti-cell surface antigen, and anti-hapten Fab′-Fab antibodies may also be used as targeting ligands. The MG series monoclonal antibodies may be selected for targeting, for example, gastric cancer. Fully humanized antibodies or antibody fragments are preferred.

There are a variety of cell surface epitopes on epithelial cells for which targeting ligands may be selected. For example, the human papilloma virus (HPV) has been associated with benign and malignant epithelial proliferations in both skin and mucosa. Two HPV oncogenic proteins, E6 and E7, may be targeted as these may be expressed in certain epithelial-derived cancers (e.g., cervical carcinoma). Membrane receptors for peptide growth factors (PGF-R), which are involved in cancer cell proliferation, may also be selected as tumor antigens. Also, epidermal growth factor (EGF) and interleukin-2 may be targeted with suitable targeting ligands, including peptides, which bind these receptors. Certain melanoma-associated antigens (MAAs) (e.g., epidermal growth factor receptor [EGFR]), and adhesion molecules expressed by malignant melanoma cells, can also be targeted with specific ligands.

A wide variety of targeting ligands may be selected for targeting myocardial cells. Exemplary targeting ligands include, for example, anticardiomyosin antibody, which may comprise polyclonal antibody, Fab′₂ fragments, or be of human origin, animal origin (e.g., mouse) or of chimeric origin. Again, in all antibody and antibody fragment embodiments, fully humanized species are preferred. Additional targeting ligands include dipyridamole, digitalis, nifedipine, apolipoprotein; low-density lipoproteins (LDL) including vLDL and methyl LDL; ryanodine, endothelin, complement receptor type 1, IgG Fc, beta 1-adrenergic, dihydropyridine, adenosine, mineralocorticoid, nicotinic acetylcholine, and muscarinic acetylcholine; antibodies to the human alpha 1A-adrenergic receptor; bioactive agents, such as drugs, including the α-1-antagonist prazosin; antibodies to the anti-beta-receptor; drugs which bind to the anti-beta-receptor; anti-cardiac RyR antibodies; endothelin-1, which is an endothelial cell-derived vasoconstrictor peptide that exerts a potent positive inotropic effect on cardiac tissue (i.e., endothelin-1 binds to cardiac sarcolemmal vesicles); monoclonal antibodies which may be generated to the T-cell receptor-β receptor and thereby employed to generate targeting ligands; the complement inhibitor sCR1; drugs, peptides, or antibodies which are generated to the dihydropyridine receptor; and monoclonal antibodies directed toward the anti-interleukin-2 receptor which may be used as targeting ligands to direct the nanocarriers of this specification to areas of myocardial tissue which express this receptor and which may be upregulated in conditions (e.g., inflammation).

In another embodiment, the targeting ligands are directed to lymphocytes which may be T-cells or B-cells. Depending on the targeting ligand, the composition may be targeted to one or more classes or clones of T-cells. To select a class of targeted lymphocytes, a targeting ligand having specific affinity for that class is employed. For example, an anti CD-4 antibody can be used for selecting the class of T-cells harboring CD4 receptors, an anti CD-8 antibody can be used for selecting the class of T-cells harboring CD-8 receptors, an anti CD-34 antibody can be used for selecting the class of T-cells harboring CD-34 receptors, etc. A lower molecular weight ligand is preferably employed (e.g., Fab or a peptide fragment). For example, an OKT3 antibody or OKT3 antibody fragment may be used. When a receptor for a class of T-cells or clones of T-cells is selected, the composition will be delivered to that class of cells. Using HLA-derived peptides, for example, will allow selection of targeted clones of cells expressing reactivity to HLA proteins. Another major area for targeted delivery involves the interleukin-2 (IL-2) system. IL-2 is a T-cell growth factor produced following antigen- or mitogen-induced stimulation of lymphoid cells. Among the cell types which produce IL-2 are CD4⁺ and CD8⁺ T-cells, large granular lymphocytes, certain T-cell tumors, etc. Still other systems which can be used in embodiments of the present invention include IgM-mediated endocytosis in B-cells or a variant of the ligand-receptor interactions described above, wherein the T-cell receptor is CD2 and the ligand is lymphocyte function-associated antigen 3 (LFA-3).

Synthetic compounds, which combine a natural amino acid sequence with synthetic amino acids, can also be used as a targeting ligands as well as peptides, or derivatives thereof. In view of the present disclosure, as will be immediately apparent to those skilled in the art, a large number of additional targeting ligands may be used with the present teachings, in addition to those exemplified above. Other suitable targeting ligands include, for example, conjugated peptides (e.g., glycoconjugates and lectins, which are peptides attached to sugar moieties). The compositions may comprise a single targeting ligand, as well as two or more different targeting ligands.

Preferred embodiments of the present invention include the use of magnetic targeting of nanocarriers and/or contrast agents. This is accomplished by using magnetically susceptible compositions bound to and/or associated with said nanocarriers or contrast agents exteriorly, and/or by other means, and then guided by a magnetic field. A wide variety of sources are known in the art describing techniques, procedures, and methods for active targeting of drug-containing vesicles using magnetically susceptible materials-referred to hereafter as “magnetic compositions” or “magnetic targeting components” (e.g., the nanocarriers of this specification). For enablement and other purposes, preferred publications include, but are not limited to Principles of Nuclear Magnetism (International Series of Monographs on Physics) (Abragam, 1983); Ultrathin Magnetic Structures I: An Introduction to the Electronic, Magnetic and Structural Properties (Bland et al., 1994); Drug and Enzyme Targeting, Parts: Volume 112: Drug and Enzyme Targeting (Methods in Enzymology) (Colowick et al., 2006); and Magnetism: Molecules to Materials, (Miller et al., 2001). Important peer-reviewed research publications associated with magnetic targeting and drug delivery include Widder et al., 1979; Hsieh et al., 1981; Kost et al., 1987; He et al., 1993; Wu et al., 1993; Wu et al., 1994; Chen et al., 1997; Rudge et al., 2000; Jones et al., 2001; Lubbe et al., 2001; Moroz et al., 2001. Patents concerning magnetic targeting and drug delivery relevant to the teachings of this specification include: U.S. Pat. Nos. 6,200,547; 6,482,436; 6,488,615; and 6,663,555. Each of the publications and patents listed in this paragraph [0470] are hereby incorporated by reference herein in their entirety for all purposes.

Magnetic targeting components for use with embodiments of the present invention are typically comprised of 1% to 70% of a biocompatible polymer, and 30% to 99% of a magnetic component. With compositions having less than 1% polymer, the physical integrity of the particle is less than optimal. With compositions of greater than 70% polymer, the magnetic susceptibility of the particle is generally reduced beyond an optimal level for the nanocarriers described herein. The compositions may be of any shape, different shapes conferring differing advantageous properties, with an average size of approximately 0.1 μm to approximately 30 μm in diameter.

Said magnetic targeting components have the general properties of having Curie temperatures (Tc) greater than the normal human body temperature (37° C.), having high magnetic saturation (greater than approximately 20 Am²/kg), and being ferromagnetic or ferrimagnetic. Examples of suitable magnetic components include magnetic iron sulfides such as pyrrohotite (Fe₇S₈), and greigite (Fe₄S₄), magnetic ceramics such as Alnico 5, Alnico 5 DG, Sm₂CO₁₇, SmCo₅, and NdFeB; magnetic iron alloys, such as jacobsite (MnFe₂O₄), trevorite (NiFe₂O₄), awaruite (Ni₃Fe), and wairauite (CoFe); and magnetic metals such as metallic iron (Fe), cobalt (Co), and nickel (Ni). Each of the magnetic components can have added to its chemical formula specific impurities that may or may not alter the magnetic properties of the material. Doped ferromagnetic or ferrimagentic materials, within the above limits of Curie temperatures and magnetic saturation values, are considered to be suitable as magnetic compositions for use in active targeting of the nanocarriers described herein. Specifically excluded from suitable magnetic components and the magnetically susceptible compositions are the iron oxides magnetite (Fe₃O₄), hematite (α-Fe₂O₃), and maghemite (γ-Fe₂O₃).

The term “metallic iron” indicates that iron is primarily in its “zero valence” state (Fe⁰). Generally, the metallic iron is greater than approximately 85% Fe⁰, and preferably greater than approximately 90% Fe⁰. More preferably, the metallic iron is greater than approximately 95% “zero valence” iron. Metallic iron is a material with high magnetic saturation and density (i.e., 218 emu/gm and 7.8 gm/cm³) which are much higher than magnetite (i.e., 92 emu/gm and 5.0 gm/cm³). The density of metallic iron is 7.8 gm/cm³, while magnetite is approximately 5.0 gm/cm³. Thus, the magnetic saturation of metallic iron is approximately 4-fold higher than that of magnetite per unit volume (Craik, 1995). The use of said magnetic compositions with the nanocarriers of this specification results in magnetically responsive compositions with a high degree of magnetic saturation (i.e., >50 emu/gm). The higher magnetic saturation allows the nanocarrier with biologically active agents (e.g., therapeutic macromolecules) to be effectively targeted to the desired site by an external magnetic field, and eventually be extravasated through blood vessel walls, penetrating into the target tissues of the patient.

The biocompatible polymers for use with said magnetic targeting components may be bioinert and/or biodegradable. Some nonlimiting examples of biocompatible polymers are polylactides, polyglycolides, polycaprolactones, polydioxanones, polycarbonates, polyhydroxybutyrates, polyalkylene oxalates, polyanhydrides, polyamides, polyacrylic acid, poloxamers, polyesteramides, polyurethanes, polyacetals, polyorthocarbonates, polyphosphazenes, polyhydroxyvalerates, polyalkylene succinates, poly(malic acid), poly(amino acids), alginate, agarose, chitin, chitosan, gelatin, collagen, atelocollagen, dextran, proteins, polyorthoesters, copolymers, terpolymers, and combinations and/or mixtures thereof. Said biocompatible polymers can be prepared in the form of matrices, which are polymeric networks. One type of polymeric matrix is a hydrogel, which can be defined as a water-containing polymeric network. The polymers used to prepare hydrogels can be based on a variety of monomer types, such as those based on methacrylic and acrylic ester monomers, acrylamide (methacrylamide) monomers, and N-vinyl-2-pyrrolidone. Hydrogels can also be based on polymers such as starch, ethylene glycol, hyaluran, chitose, and/or cellulose. To form a hydrogel, monomers are typically cross-linked with cross-linking agents such as ethylene dimethacrylate, N,N-methylenediacrylamide, methylenebis(4-phenyl isocyanate), epichlarohydin glutaraldehyde, ethylene dimethacrylate, divinylbenzene, and allyl methacrylate. Hydrogels can also be based on polymers such as starch, ethylene glycol, hyaluran, chitose, and/or cellulose. In addition, hydrogels can be formed from a mixture of monomers and polymers.

Another type of polymeric network can be formed from more hydrophobic monomers and/or macromers. Matrices formed from these materials generally exclude water. Polymers used to prepare hydrophobic matrices can be based on a variety of monomer types such as alkyl acrylates and methacrylates, and polyester-forming monomers such as ∈-caprolactone, glycolide, lactic acid, glycolic acid, lactide, and the like. When formulated for use in an aqueous environment, these materials do not normally need to be cross-linked, but they can be cross-linked with standard agents such as divinyl benzene. Hydrophobic matrices may also be formed from reactions of macromers bearing the appropriate reactive groups such as the reaction of diisocyanate macromers with dihydroxy macromers and the reaction of diepoxy-containing macromers with dianhydride or diamine-containing macromers.

The biocompatible polymers for use with said magnetic compositions can be preferably prepared in the form of dendrimers and/or hyperbranched polymers. The size, shape, and properties of these dendrimers can be molecularly tailored to meet specialized end uses (e.g., a means for the delivery of high concentrations of carried material per unit of polymer, controlled delivery, targeted delivery, and/or multiple species delivery or use). The dendritic polymers can be prepared according to methods known in the art, including those detailed herein. The biocompatible polymers for use with said magnetic compositions may be, for example, biodegradable, bioresorbable, bioinert, and/or biostable. Bioresorbable hydrogel-forming polymers are generally naturally occurring polymers such as polysaccharides, examples of which include, but are not limited to hyaluronic acid, starch, dextran, heparin, and chitosan; and proteins (and other polyamino acids), examples of which include but are not limited to gelatin, collagen, fibronectin, laminin, albumin, and active peptide domains thereof. Matrices formed from these materials degrade under physiological conditions, generally via enzyme-mediated hydrolysis.

Bioresorbable matrix-forming polymers are generally synthetic polymers prepared via condensation polymerization of one or more monomers. Matrix-forming polymers of this type include polylactide (PLA), polyglycolide (PGA), polylactide coglycolide (PLGA), polycaprolactone (PCL), and copolymers of these materials, polyanhydrides, and polyortho esters. Biostable or bioinert hydrogel matrix-forming polymers are generally synthetic or naturally occurring polymers which are soluble in water, and matrices of which are hydrogels or water-containing gels. Examples of this type of polymer include polyvinylpyrrolidone (PVP), polyethylene glycol (PEG), polyethylene oxide (PEO), polyacrylamide (PAA), polyvinyl alcohol (PVA), and the like. Biostable or bioinert matrix-forming polymers are generally synthetic polymers formed from hydrophobic monomers (e.g., methyl methacrylate, butyl methacrylate, dimethyl siloxanes, and the like). These polymer materials generally do not possess significant water solubility but can be formulated as neat liquids which form strong matrices upon activation. Said polymers may also contain hydrophilic and hydrophobic monomers.

Targeting moieties may also be incorporated into the nanocarriers of this specification in a variety of other ways. Additional preferred methods include being associated covalently or non-covalently with one or more of the polymers described herein. Photopolymerizable elements are most preferred, and photopolymerization may be used in cross-linking the targeting moieties with themselves and/or linking said moieties to the nanocarriers. In other preferred embodiments, the targeting moiety is covalently bound to the surface of the nanocarrier by a spacer including, for example, hydrophilic polymers, preferably polyethylene glycol. Preferred molecular weights of the polymers are from 1,000 daltons to 10,000 daltons, with 500 daltons being most preferred. Preferably, the polymer is bifunctional with the targeting moiety bound to a terminus of the polymer. Generally, in the case of a targeting ligand, it should range from approximately 0.1 mole % to approximately 20 mole % of the exterior components of the vesicle. The exact ratio will depend on the particular targeting ligand and the application.

Exemplary covalent bonds by which the targeting moieties are associated with the nanocarriers of embodiments of the present invention include, for example amide (—CONH—); thioamide (—CSNH—); ether (ROR′), where R and R′ may be the same or different and are other than hydrogen; ester (—COO—); thioester (—COS—); —O—; —S—; —Sₙ, where n is greater than 1, preferably approximately 2 to approximately 8, and more preferably approximately 2; carbamates; —NH—; —NR—, where R is alky, for example, alkyl of from 1 carbon to approximately 4 carbons; urethane; and substituted imidate; and combinations of two or more of these. Covalent bonds between targeting ligands and polymers may be achieved through the use of molecules that may act as spacers to increase the conformational and topographical flexibility of the ligand. Examples of such spacers include, for example, succinic acid, 1,6-hexanedioic acid, 1,8-octanedioic acid, and the like, as well as modified amino acids (e.g., 6-aminohexanoic acid, 4-aminobutanoic acid, and the like). In addition, in the case of targeting ligands which comprise peptide moieties, side-chain-to-side-chain cross-linking may be complemented with side-chain-to-end cross-linking and/or end-to-end cross-linking. Also, small spacer molecules (e.g., dimethylsuberimidate) may be used to accomplish similar objectives. The use of agents, including those used in Schiff's base-type reactions (e.g., gluteraldehyde) may also be employed.

The covalent linking of targeting moieties to embodiments of the invention may also be accomplished using synthetic organic techniques, which in view of the present disclosure, will be readily apparent to one of ordinary skill in the art. For example, the targeting moieties may be linked to the materials, including the polymers, via the use of well-known coupling or activation agents. As known to those skilled in the art, activating agents are generally electrophilic, which can be employed to elicit the formation of a covalent bond. Exemplary activating agents which may be used include, for example, carbonyldiimidazole (CDI), dicyclohexylcarbodiimide (DCC), diisopropylcarbodiimide (DIC), methyl sulfonyl chloride, Castro's Reagent, and diphenyl phosphoryl chloride. The covalent bonds may involve cross-linking and/or polymerization. Cross-linking preferably refers to the attachment of two chains of polymer molecules by bridges, composed of either an element, a group, or a compound, which join certain carbon atoms of the chains by covalent chemical bonds. For example, cross-linking may occur by photopolymerization and for polypeptides which are joined by the disulfide bonds of the cysteine residue. Cross-linking may be achieved, for example, by (1) adding a chemical substance (e.g., cross-linking agent) and exposing the mixture to heat, or (2) subjecting a polymer to high-energy radiation. A variety of cross-linking agents, or “tethers,” of different lengths and/or functionalities are described (Hermanson, 1996), the disclosures of which are hereby incorporated herein by reference in their entirety for all purposes.

### Contrast Agents

Most of the contrast agents for use with preferred embodiments of this specification have a high degree of echogenicity, the ability of an object to reflect ultrasonic waves. Thus, preferred contrast agents comprise small, stabilized gas-filled microbubbles that can pass through the smallest capillaries of the patient. For imaging purposes, the larger the microbubbles, the better the acoustic responsiveness; however, if the bubbles are too large, they will be retained in the capillaries of the patient and they are unable to cross the pulmonary circulation. Properties of the ideal contrast agent for use with embodiments of this disclosure (1) are nontoxic, and easily eliminated by the patient; (2) are administered intravenously; (3) pass easily through the microcirculation; (4) are physically stable; and (5) are acoustically responsive with stable harmonics and the capability of rapid disruption. The ultrasonic characteristics of these contrast agents depend not only on the size of the bubble, but also on the composition of the shell and the gas contained therein. The outer shell of preferred microbubbles is composed of many different substances including albumin, polymers, palmitic acid, or phospholipids. The composition of the shell determines its elasticity, its behavior in an ultrasonic field, how rapidly the bubble is taken up by the immune system, and the physiological methods for metabolism and elimination from the patient. A more hydrophilic material tends to be taken up more easily, which reduces the microbubble residence time in the circulation. In general, the stiffer the shell, the more easily it will crack or break when exposed to ultrasonic energy. Conversely, the more elastic the shell, the greater its ability to be compressed or resonated, a characteristic of considerable importance for the preferred contrast agents used in the practice of the present teachings.

The gas core is a critical component of the microbubble because it is the primary determinate of its echogenicity. When gas bubbles are caught in an ultrasonic frequency field, they compress, oscillate, and reflect a characteristic echo, which generates the strong and unique sonogram in contrast-enhanced ultrasound. Gas cores can be composed of air, nitrogen, or, for example, heavy gases like perfluorocarbon. Heavy gases are less water-soluble so they are less likely to leak out from the microbubble to impair echogenicity. Thus, microbubbles with heavy gas cores are likely to remain in the circulation longer.

Ultrasound contrast reagents, for use with the present invention, can be prepared as described in U.S. Pat. No. 6,146,657, the disclosures of which are hereby incorporated herein by reference in their entirety for all purposes. Briefly, a sealed container is used comprising an aqueous lipid suspension phase and a substantially separate gaseous phase. Prior to use, the container and its contents may be agitated, causing the lipid and gas phases to mix, resulting in the formation of gas-filled liposomes which entrap the gas. The resulting gas-filled liposomes provide an excellent contrast enhancement agent for diagnostic imaging, particularly using ultrasound or magnetic resonance imaging, and for assisting in acoustically mediated intracellular therapeutic delivery in vivo, as detailed herein. A wide variety of lipids may be employed in the aqueous lipid suspension phase of the preferred contrast agents for use with the present teachings. The lipids may be saturated or unsaturated, and may be in linear or branched form, as desired. Such lipids may comprise, for example, fatty acids molecules that contain a wide range of carbon atoms, preferably between approximately 12 carbon atoms and 22 carbon atoms. Hydrocarbon groups consisting of isoprenoid units, prenyl groups, and/or sterol moieties (e.g., cholesterol, cholesterol sulfate, and analogs thereof) may also be employed. The lipids may also bear polymer chains, such as the amphipathic polymers polyethylene glycol (PEG), polyvinylpyrrolidone (PVP), derivatives thereof for in vivo targeting; charged amino acids such as polylysine or polyarginine, for binding of a negatively charged compound; carbohydrates, for in vivo targeting, such as described in U.S. Pat. No. 4,310,505; glycolipids, for in vivo targeting; or antibodies and other peptides and proteins, for in vivo targeting, etc., as desired. Such targeting or binding compounds may be simply added to the aqueous lipid suspension phase or may be specifically chemically attached to the lipids using methods described herein, or employing other methodologies known in the art. The lipids may also be anionic or cationic.

Classes of and specific lipids for use as shell materials with preferred contrast agents for use with the present teachings, but are not limited to, include phosphatidylcholines, such as dioleoylphosphatidylcholine, dimyristoylphosphatidylcholine, dipalmitoylphosphatidylcholine (DPPC), and distearoylphosphatidylcholine; phosphatidylethanolamines, such as dipalmitoylphosphatidylethanolamine (DPPE), dioleoylphosphatidylethanolamine, and N-succinyl-dioleoylphosphatidylethanolamine; phosphatidylserines, phosphatidylglycerols, and sphingolipids; glycolipids, such as ganglioside GM1; glucolipids, sulfatides, and glycosphingolipids; phosphatidic acids, such as dipatmatoylphosphatidic acid (DPPA); palmitic fatty acids, stearic fatty acids, arachidonic fatty acids, lauric fatty acids, myristic fatty acids, lauroleic fatty acids, physeteric fatty acids, myristoleic fatty acids, palmitoleic fatty acids, petroselinic fatty acids, oleic fatty acids, isolauric fatty acids, isomyristic fatty acids, isopalmitic fatty acids, and isostearic fatty acids; cholesterol and cholesterol derivatives, such as cholesterol hemisuccinate, cholesterol sulfate, and cholesteryl-(4′-trimethylammonio)-butanoate; polyoxyethylene fatty acid esters, polyoxyethylene fatty acid alcohols, polyoxyethylene fatty acid alcohol ethers, polyoxyethylated sorbitan fatty acid esters, glycerol polyethylene glycol oxystearate, glycerol polyethylene glycol ricinoleate, ethoxylated soybean sterols, ethoxylated castor oil, polyoxyethylene-polyoxypropylene fatty acid polymers, polyoxyethylene fatty acid stearates, 12-(((7′-diethylaminocoumarin-3-yl)-carbonyl)-methylamino)-octadecanoic acid, N-(12-(((7′-diethylamino-coumarin-3-yl)-carbonyl)-methyl-amino)octadecanoyl)-2-amino-palmitic acid, 1,2-dioleoyl-sn-glycerol, 1,2-dipalmitoyl-sn-3-succinylglycerol, 1,3-dipalmitoyl-2-succinyl-glycerol, 1-hexadecyl-2-palmitoyl-glycerophosphoethanolamine, and palmitoylhomocysteine, lauryltrimethylammonium bromide (lauryl-=dodecyl-); cetyltrimethylammonium bromide (cetryl-=hexadecyl-), myristyltrimethylammonium bromide (myristyl-=tetradecyl-); alkyldimethylbenzylammonium chlorides, such as wherein alkyl is a C₁₂, C₁₄, or C₁₆alkyl; and benzyldimethyldodecylammonium bromide, benzyldimethyldodecylammonium chloride, benzyldimethylhexadecylammonium bromide, benzyldimethylhexadecylammonium chloride, benzyldimethyltetradecylammonium bromide, benzyldimethyltetradecylammonium chloride, cetyldimethylethylammonium bromide, cetyldimethylethylammonium chloride, cetylpyridinium bromide, cetylpyridinium chloride, N-(1,2,3-dioleoyloxy)-propyl)-N,N,N-trimethylammonium chloride (DOTMA), 1,2-dioleoyloxy-3-(trimethylammonio)propane (DOTAP), and 1,2-dioleoyl-e-(4′-trimethylammonio)-butanoyl-sn-glycerol (DOTB).

In addition, the aqueous lipid phase may further comprise a polymer, preferably an amphipathic polymer, and preferably one that is directly bound (i.e., chemically attached) to the lipid. Preferably, the amphipathic polymer is polyethylene glycol or a derivative thereof. The most preferred combination is the lipid dipalmitoylphosphatidylethanolamine (DPPE) bound to polyethylene glycol (PEG), especially PEG of an average molecular weight of approximately 5000 (DPPE-PEG5000). The PEG or other polymer may be bound to the DPPE or other lipid through a covalent linkage, such as through an amide, carbamate, or amine linkage. Alternatively, ester, ether, thioester, thioamide, or disulfide (i.e., thioester) linkages may be used with the PEG or other polymer to bind the polymer to, for example, cholesterol or other phospholipids. A particularly preferred combination of lipids is DPPC, DPPE-PEG5000, and DPPA, especially in a ratio of approximately 82:8:10% (mole %), DPPC: DPPE-PEG5000:DPPA.

Examples of classes of and specific suitable gases for use as the core of preferred contrast agents, suitable for use with the present teachings, are those gases that are substantially insoluble in an aqueous shell suspension. Suitable gases that are substantially insoluble or soluble include, but are not limited to hexafluoroacetone, isopropylacetylene, allene, tetrafluoroallene, boron trifluoride, 1,2-butadiene, 1,3-butadiene, 1,2,3-trichlorobutadiene, 2-fluoro-1,3-butadiene, 2-methyl-1,3 butadiene, hexafluoro-1,3-butadiene, butadiyne, 1-fluorobutane, 2-methylbutane, decafluorobutane (perfluorobutane), decafluoroisobutane (perfluoroisobutane), 1-butene, 2-butene, 2-methyl-1-butene, 3-methyl-1-butene, perfluoro-1-butene, perfluoro-1-butene, perfluoro-2-butene, 4-phenyl-3-butene-2-one, 2-methyl-1-butene-3-yne, butylnitrate, 1-butyne, 2-butyne, 2-chloro-1,1,1,4,4,4-hexafluoro-butyne, 3-methyl-1-butyne, perfluoro-2-butyne, 2-bromo-butyraldehyde, carbonyl sulfide, crotononitrile, cyclobutane, methylcyclobutane, octafluorocyclobutane (perfluorocyclobutane), perfluoroisobutane, 3-chlorocyclopentene, cyclopropane, 1,2-dimethylcyclopropane, 1,1-dimethylcyclopropane, ethyl cyclopropane, methylcyclopropane, diacetylene, 3-ethyl-3-methyldiaziridine, 1,1,1-trifluorodiazoethane, dimethylamine, hexafluorodimethylamine, dimethylethylamine, bis-(dimethyl phosphine) amine, 2,3-dimethyl-2-norbornane, perfluorodimethylamine, dimethyloxonium chloride, 1,3-dioxolane-2-one, 1,1,1,1,2-tetrafluoroethane, 1,1,1-trifluoroethane, 1,1,2,2-tetrafluoroethane, 1,1,2-trichloro-1,2,2-trifluoroethane, 1,1-dichloroethane, 1,1-dichloro-1,2,2,2-tetrafluoroethane, 1,2-difluoroethane, 1-chloro-1,1,2,2,2-pentafluoroethane, 2-chloro-1,1-difluoroethane, 1-chloro-1,1,2,2-tetrafluoroethane, 2-chloro-1,1-difluoroethane, chloroethane, chloropentafluoroethane, dichlorotrifluoroethane, fluoroethane, nitropentafluoroethane, nitrosopentafluoro-ethane, perfluoroethane, perfluoroethylamine, ethyl vinyl ether, 1,1-dichloroethylene, 1,1-dichloro-1,2-difluoro-ethylene, 1,2-difluoro-ethylene, methane, methane-sulfonyl-chloride-trifluoro, methane-sulfonyl-fluoride-trifluoro, methane-(penta-fluorothio)trifluoro, methane-bromo-difluoro-nitroso, methane-bromo-fluoro, methane-bromo-chloro-fluoro, methane-bromo-trifluoro, methane-chloro-difluoro-nitro, methane-chloro-dinitro, methane-chloro-fluoro, methane-chloro-trifluoro, methane-chloro-difluoro, methane-dibromo-difluoro, ethane-dichloro-difluoro, methane-dichloro-fluoro, methane-difluoro, methane-difluoro-iodo, methane-disilano, methane-fluoro, methane-iodonethane-iodo-trifluoro, methane-nitro-trifluoro, methane-nitroso-trifluoro, methane-tetrafluoro, methane-trichloro-fluoro, methane-trifluoro, methanesulfenylchloride-trifluoro, 2-methyl butane, methyl ether, methyl isopropyl ether, methyl lactate, methyl nitrite, methyl sulfide, methyl vinyl ether, neopentane, nitrogen (N₂), nitrous oxide, 1,2,3-nonadecane tricarboxylic acid-2-hydroxytrimethylester, 1-nonene-3-yne, oxygen (O₂), oxygen 17 (¹⁷O₂), 1,4-pentadiene, n-pentane, dodecafluoropentane (perfluoropentane), tetradecafluorohexane (perfluorohexane), perfluoroisopentane, perfluoroneopentane, 2-pentanone-4-amino-4-methyl, 1-pentene, 2-pentene {cis #0}, 2-pentene {trans}, 1-pentene-3-bromo, 1-pentene-perfluoro, phthalic acid-tetrachloro, piperidine-2,3,6-trimethyl, propane, propane-1,1,1,2,2,3-hexafluoro, propane-1,2-epoxy, propane-2,2 difluoro, propane-2-amino, propane-2-chloro, propane-heptafluoro-1-nitro, propane-heptafluoro-1-nitroso, perfluoropropane, propene, propyl-1,1,1,2,3,3-hexafluoro-2,3 dichloro, propylene-1-chloro, propylene-chloro-{trans}, propylene-2-chloro, propylene-3-fluoro, propylene-perfluoro, propyne, propyne-3,3,3-trifluoro, styrene-3-fluoro, sulfur hexafluoride, sulfur (di)-decafluoro(S₂F₁₀), toluene-2,4-diamino, trifluoroacetonitrile, trifluoromethyl peroxide, trifluoromethyl sulfide, tungsten hexafluoride, vinyl acetylene, vinyl ether, neon, helium, krypton, xenon (especially rubidium enriched hyperpolarized xenon gas), carbon dioxide, helium, and air. Fluorinated gases (i.e., a gas containing one or more fluorine molecules, such as sulfur hexafluoride); fluorocarbon gases (i.e., a fluorinated gas which is a fluorinated carbon or gas); and perfluorocarbon gases (i.e., a fluorocarbon gas which is fully fluorinated, such as perfluoropropane and perfluorobutane) are preferred.

A targeted contrast agent, for use with embodiments of the present invention, is a contrast agent that can bind selectively or specifically to a desired target. The same aforementioned preferred shell materials and preferred gases may be used in preferred targeted contrast agents, with the addition of a targeting moiety, as described in detail herein, or other structure, either alone or in combination. For example, an antibody fragment or an aptamer may be bound to the surface of said contrast agent by the methods described herein and/or in the art. If an antibody or similar targeting mechanism is used, selective or specific binding to a target can be determined based on standard antigen/epitope/antibody complementary binding relationships. Further, other controls may be used. For example, the specific or selective targeting of the microbubbles can be determined by exposing targeted microbubbles to a control tissue, which includes all of the components of the test tissue except for the desired target ligand, epitope, or other structure.

Specific or selectively targeted contrast agents can be produced by methods known in the art. For example, targeted contrast agents can be prepared as perfluorocarbon or other gas-filled microbubbles with a monoclonal antibody on the shell as a ligand for binding to a target ligand in a patient as described in Villanueva et al. (1998), the disclosures of which are hereby incorporated herein by reference in their entirety for all purposes. For example, perfluorobutane can be dispersed by sonication in an aqueous medium containing phosphatidylcholine, a surfactant, and a phospholipid derivative containing a carboxyl group. The perfluorobutane is encapsulated during sonication by a lipid shell. The carboxylic groups are exposed to an aqueous environment and used for covalent attachment of antibodies to the microbubbles by the following steps. First, unbound lipid dispersed in the aqueous phase is separated from the gas-filled microbubbles by flotation. Second, carboxylic groups on the microbubble shell are activated with 1-ethyl-3-(3-dimethylaminopropyl) carbodimide, and antibody is then covalently attached via its primary amino groups with the formation of amide bonds.

Targeted microbubbles can also be prepared with a biotinylated shell, using the methods described in Weller et al. (2002), the disclosures of which are hereby incorporated herein by reference in their entirety for all purposes. For example, lipid-based perfluorocarbon-filled microbubbles can be prepared with monoclonal antibody on the shell using avidin-biotin bridging chemistry, employing, for example, the following protocol. Perfluorobutane is dispersed by sonication in aqueous saline containing phosphatidyl choline, polyethylene glycol (PEG) stearate, and a biotinylated derivative of phosphatidylethanolamine, as described in the art. The sonication results in the formation of perfluorobutane microbubbles coated with a lipid monolayer shell and carrying the biotin label. Antibody conjugation to the shell is achieved via avidin-biotin bridging chemistry. Samples of biotinylated microbubbles are washed in phosphate-buffered saline (PBS) by centrifugation to remove the lipid not incorporated in the microbubble shell. Next, the microbubbles are incubated in a solution (i.e., 0.1-10 μg/ml) of streptavidin in PBS. Excess streptavidin is removed by washing with PBS. The microbubbles are then incubated in a solution of biotinylated monoclonal antibody in PBS and washed. The resultant microbubble has antibody conjugated to the lipid shell via biotin-streptavidin-biotin linkage. In another example, biotinylated microbubbles can be prepared by sonication of an aqueous dispersion of decafluorobutane gas, distearoylphodphatidylcholine, polyethyleneglycol-(PEG)-state, and distearoyl-phosphatidylethanolamine-PEG-biotin. Microbubbles can then be combined with streptavidin, washed, and combined with biotinylated echistatin.

Targeted microbubbles can also be prepared with an avidinated shell, as is known in the art. In a preferred embodiment, a polymer microbubble can be prepared with an avidinated or streptavidinated shell. In another preferred embodiment, avidinated microbubbles can be used by the methods disclosed herein. When using avidinated microbubbles, a biotinylated antibody or fragment thereof or another biotinylated targeting molecule or fragments thereof can be administered to the patient. For example, a biotinylated targeting ligand such as an antibody, protein, or other bioconjugate can be used. Thus, a biotinylated antibody, targeting ligand or molecule, or fragment thereof can bind to a desired target within the patient. Once bound to the desired target, the contrast agent with an avidinated shell can bind to the biotinylated antibody, targeting molecule, or fragment thereof. An avidinated contrast agent can also be bound to a biotinylated antibody, targeting ligand or molecule, or fragment thereof, prior to administration. When using a targeted contrast agent with a biotinylated shell or an avidinated shell, a targeting ligand or molecule can be administered to the patient. For example, a biotinylated targeting ligand, such as an antibody, protein, or other bioconjugate, can be administered to the patient and allowed to accumulate at a target site. A fragment of the targeting ligand or molecule can also be used. When a targeted contrast agent with a biotinylated shell is used, an avidin linker molecule, which attaches to the biotinylated targeting ligand, can be administered to the patient. Then a targeted contrast agent with a biotinylated shell is administered. The targeted contrast agent binds to the avidin linker molecule, which is bound to the biotinylated targeting ligand, which is itself bound to the desired target. In this way, a three-step method can be used to target contrast agents to a desired target.

Targeted contrast agents or nontargeted contrast agents can also comprise a variety of markers, detectable moieties, or labels. Thus, a microbubble contrast agent, equipped with a targeting ligand or antibody incorporated into the shell of the microbubble, can also include another detectable moiety or label. As used herein, the term “detectable moiety” is intended to mean any suitable label, including, but not limited to enzymes, fluorophores, biotin, chromophores, radioisotopes, colored particles, electrochemical, chemical-modifying, or chemiluminescent moieties. Common fluorescent moieties include fluorescein, cyanine dyes, coumarins, phycoerythrin, phycobiliproteins, dansyl chloride, Texas Red, and lanthanide complexes. Of course, the derivatives of these compounds, which are known to those skilled in the art, are also included as common fluorescent moieties. The detection of the detectable moiety can be direct, provided that the detectable moiety is itself detectable, such as, for example, in the case of fluorophores. Alternatively, the detection of the detectable moiety can be indirect. In the latter case, a second moiety reactable with the detectable moiety, itself being directly detectable, can be employed. The detectable moiety may be inherent to the molecular probe. For example, the constant region of an antibody can serve as an indirect detectable moiety to which a second antibody having a direct detectable moiety can specifically bind. Targeted contrast agents can also be modified by allowing larger bubbles to separate in solution relative to smaller bubbles. For example, targeted contrast agents can be modified by allowing larger bubbles to float higher in solution relative to smaller bubbles. A population of microbubbles, of an appropriate size to achieve a desired volume percentage, can subsequently be selected. Other means are available in the art for separating micron-sized and nanosized particles, and could be adapted to select a microbubble population of the desired volume of submicron bubbles such as, for example, by centrifugation. Sizing of the microbubbles can occur before or after the microbubbles are adapted for targeting.

The targeted contrast agents may be used with the nanocarriers of this disclosure by targeting said contrast agents to a variety of cells, cell types, antigens, cellular membrane proteins, organs, markers, tumor markers, angiogenesis markers, blood vessels, thrombus, fibrin, and infective agents, as described herein. For example, targeted microbubbles can be produced that localize to specific targets expressed in the patient. Desired targets are generally based on, but not limited to, the molecular signature of various pathologies, organs, and/or cells. For example, adhesion molecules, such as integrin αᵥβ₃, intercellular adhesion molecule-1 (I-CAM-1), fibrinogen receptor GPIIb/IIIa, and VEGF receptors, are expressed in regions of angiogenesis, inflammation, or thrombus. These molecular signatures can be used to localize contrast agents through the use of targeting molecules, including but not limited to, complementary receptor ligands, targeting ligands, proteins, and fragments thereof. Target cell types include, but are not limited to, endothelial cells, neoplastic cells, and blood cells. The methods described herein optionally use contrast agents targeted to VEGFR2, I-CAM-1, αᵥβ₃ integrin, αᵥ integrin, fibrinogen receptor GPIIb/IIIa, P-selectin, and mucosal vascular adressin cell adhesion molecule-1. Moreover, using methods described herein and known to those skilled in the art, complementary receptor ligands, such as monoclonal antibodies, can be readily produced to target other markers in the patient. For example, antibodies can be produced to bind to tumor marker proteins, organ or cell type specific markers, or infective agent markers. Thus, targeted contrast agents may be targeted, using antibodies, proteins, fragments thereof aptamers, or other ligands, as described herein, to sites of neoplasia, angiogenesis, thrombus, inflammation, infection, as well as to diseased or normal organs or tissues, including but not limited to blood, heart, brain, blood vessel, kidney, muscle, lung, and liver. Optionally, the targeted markers are proteins and may be extracellular or transmembrane proteins. The targeted markers, including tumor markers, can be the extracellular domain of a protein. The antibodies or fragments thereof designed to target these marker proteins can bind to any portion of the protein. Optionally, the antibodies can bind to the extracellular portion of a protein, for example, a cellular transmembrane protein. Antibodies, proteins, and fragments thereof can be made that specifically or selectively target a desired target molecule using methods described herein and/or known in the art.

Examples of other contrast agents for use with this specification, include, for example, stable free radicals, such as, stable nitroxides, as well as compounds comprising transition, lanthanide, and actinide elements, which may, if desired, be in the form of a salt or may be covalently or noncovalently bound to complexing agents, including lipophilic derivatives thereof or to proteinaceous macromolecules. Preferable transition, lanthanide, and actinide elements include, for example, Gd(III), Mn(II), Cu(II), Cr(III), Fe(II), Fe(III), Co(II), Er(II), Ni(II), Eu(III), and Dy(III). More preferably, the elements may be Gd(III), Mn(II), Cu(II), Fe(II), Fe(III), Eu(III), and Dy(III), and most preferably, Mn(II) and Gd(III). The foregoing elements may be in the form of a salt, including inorganic salts, such as a manganese salt, for example, manganese chloride, manganese carbonate, manganese acetate, and organic salts, such as manganese gluconate and manganese hydroxylapatite. Other exemplary salts include salts of iron, such as iron sulfides, and ferric salts, such as ferric chloride.

The above elements may also be bound, for example, through covalent or noncovalent association, to complexing agents, including lipophilic derivatives thereof, or to proteinaceous macromolecules. Preferred complexing agents include, for example, diethylenetriaminepentaacetic acid (DTPA); ethylenediaminetetraacetic acid (EDTA); 1,4,7,10-tetraazacyclododecane-N,N′,N″,N′″-tetraacetic acid (DOTA); 1,4,7,10-tetraazacyclododecane-N,N′,N″-triacetic acid (DOTA); 3,6,9-triaza-12-oxa-3,6,9-tricarboxymethylene-10-carboxy-13-phenyltridecan oic acid (B-19036); hydroxybenzylethylenediamine diacetic acid (HBED); N,N′-bis(pyridoxyl-5-phosphate)ethylene diamine; N,N′-diacetate (DPDP); 1,4,7-triazacyclononane-N,N′,N″-triacetic acid (NOTA); 1,4,8,11-tetraazacyclotetradecane-N,N′,N″,N′″-tetraacetic acid (TETA); kryptands (macrocyclic complexes); and desferrioxamine. More preferably, the complexing agents are EDTA, DTPA, DOTA, DO3A, and kryptands, most preferably DTPA. Preferable lipophilic complexes include alkylated derivatives of the complexing agents EDTA and DOTA, for example, N,N′-bis-(carboxydecylamidomethyl-N-2,3-dihydroxypropyl)ethylenediamine-N,N′-diacetate (EDTA-DDP); N,N′-bis-(carboxyoctadecylamidomethyl-N-2,3-dihydroxypropyl)ethylenediamin e-N,N′-diacetate (EDTA-ODP); and N,N′-Bis(carboxylaurylamidomethyl-N-2,3-dihydroxypropyl)ethylenediamine-N, N′-diacetate (EDTA-LDP), including those described in U.S. Pat. No. 5,312,617, the disclosures of which are hereby incorporated herein by reference in their entirety for all purposes. Preferable proteinaceous macromolecules include, for example, albumin, collagen, polyarginine, polylysine, and polyhistidine; and γ-globulin and β-globulin, with albumin; with polyarginine, polylysine, and polyhistidine being more preferred. Suitable complexes therefore include Mn(II)-DTPA, Mn(II)-EDTA, Mn(II)-DOTA, Mn(II)-DO3A, Mn(II)-kryptands, Gd(III)-DTPA, Gd(III)-DOTA, Gd(III)-DO3A, Gd(III)-kryptands, Cr(III)-EDTA, Cu(II)-EDTA, or iron-desferrioxamine; more preferably, Mn(II)-DTPA or Gd(III)-DTPA.

Nitroxides are paramagnetic contrast agents which increase both T1 and T2 relaxation rates on MRI by virtue of the presence of an unpaired electron in the nitroxide molecule. As known to one of ordinary skill in the art, the paramagnetic effectiveness of a given compound as an MRI contrast agent may be related, at least in part, to the number of unpaired electrons in the paramagnetic nucleus or molecule, and specifically, to the square of the number of unpaired electrons. For example, gadolinium has seven unpaired electrons whereas a nitroxide molecule has one unpaired electron. Thus, gadolinium is generally a much stronger MRI contrast agent than a nitroxide. However, effective correlation time, another important parameter for assessing the effectiveness of contrast agents, confers potential increased relaxivity to the nitroxides. When the tumbling rate is slowed, for example, by attaching the paramagnetic contrast agent to a large molecule, it will tumble more slowly, and thereby, more effectively transfer energy to hasten relaxation of the water protons. In gadolinium, however, the electron spin relaxation time is rapid and will limit the extent to which slow rotational correlation times can increase relativity. For nitroxides, however, the electron spin correlation times are more favorable and tremendous increases in relativity may be attained by slowing the rotational correlation time of these molecules. Although not intending to be bound by any particular theory of operation, since the nitroxides may be designed to coat the perimeters of the vesicles, for example, by making alkyl derivatives thereof and the resulting correlation times can be optimized. Moreover, the resulting contrast medium of the present disclosure may be viewed as a magnetic sphere, a geometric configuration which maximizes relativity.

Superparamagnetic contrast agents suitable for use with the nanocarriers described herein include: metal oxides and sulfides which experience a magnetic domain; ferro- or ferrimagnetic compounds, such as pure iron; magnetic iron oxide, such as magnetite, γ-Fe₂O₃, Fe₃O₄, manganese ferrite, cobalt ferrite, and nickel ferrite. Along with the gaseous precursors described herein, paramagnetic gases can be employed in the present compositions, such as oxygen 17 gas (¹⁷O₂), hyperpolarized xenon, neon, or helium. MR whole body imaging may then be employed to rapidly screen the body, for example, for thrombosis; and ultrasound may be applied, if desired, to aid in thrombolysis.

The contrast agents (e.g., the paramagnetic and superparamagnetic contrast agents described above) may be employed as a component within the compositions of embodiments of the present invention. With respect to vesicles, the contrast agents may be entrapped within the internal void thereof administered as a solution with the vesicles, incorporated with any additional stabilizing materials, or coated onto the surface or membrane of the vesicle. Mixtures of any one or more of the paramagnetic agents and/or superparamagnetic agents in the present compositions may be used. The paramagnetic and superparamagnetic agents may also be coadministered separately, if desired. In addition, the paramagnetic or superparamagnetic agents may be delivered as alkylated or other derivatives incorporated into the compositions, if desired, especially the polymeric walls of the nanocarriers of the present invention. In particular, the nitroxides 2,2,5,5-tetramethyl-1-pyrrolidinyloxy, free radical, and 2,2,6,6-tetramethyl-1-piperidinyloxy, free radical, can form adducts with, for example, polymers and copolypeptides of the embodiments of the invention.

The iron oxides may simply be incorporated into the contrast agents for use with the present teachings using methods and procedures described previously in this specification. A few large particles may have a much greater effect than a larger number of much smaller particles, primarily due to a larger correlation time. If one were to make the iron oxide particles very large, however, increased toxicity may result, and the lungs may be embolized or the complement cascade system activated. Further, the total size of the particle is not as important as the diameter of the particle at its edge or outer surface. The domain of magnetization or susceptibility effect falls off exponentially from the surface of the particle. Generally, in the case of dipolar (i.e., through space) relaxation mechanisms, this exponential fall off exhibits an r⁶ dependence for a paramagnetic dipole-dipole interaction. Interpreted literally, a water molecule that is 4 Å away from a paramagnetic surface will be influenced 64 times less than a water molecule that is 2 Å away from the same paramagnetic surface. The ideal situation in terms of maximizing the contrast effect would be to make the iron oxide particles hollow, flexible, and as large as possible. By coating the inner or outer surfaces of the nanocarriers of the present invention with the contrast agents, even though the individual contrast agents, for example, iron oxide nanoparticles or paramagnetic ions, are relatively small structures, the effectiveness of the contrast agents may be even further enhanced. In so doing, the contrast agents may function as an effectively much larger sphere wherein the effective domain of magnetization is determined by the diameter of the vesicle and is maximal at the surface of the vesicle. These agents afford the advantage of flexibility, namely, compliance. While rigid vesicles might lodge in the lungs or other organs and cause toxic reactions, these flexible vesicles slide through the capillaries much more easily.

In contrast to the flexible compositions described above, it may be desirable, in certain circumstances, to formulate compositions from substantially impermeable polymeric materials, including, for example, polymethyl methacrylate. This would generally result in the formation of compositions which may be substantially impermeable and relatively inelastic and brittle. In embodiments involving diagnostic imaging, for example, ultrasound, contrast media which comprise such brittle compositions would generally not provide the desirable reflectivity that the flexible compositions may provide. However, by increasing the power output on ultrasound, the brittle compositions, such as microspheres, may be made to rupture, thereby causing acoustic emissions, leading to nanocarrier disassociation, therapeutic release, and detection of said acoustic emissions by an ultrasound transducer.

### Therapeutics

The therapeutic to be delivered by embodiments of this specification may be embedded within the wall of a nanocarrier, encapsulated in the vesicle and/or attached to the surface of the nanocarrier. The phrase “attached to” or variations thereof means that the therapeutic is linked in some manner to the inside and/or the outside wall of the nanocarrier, such as through a covalent or ionic bond or other means of chemical or electrochemical linkage or interaction. The phrase “encapsulated in” or a variation thereof means that the therapeutic is located in the internal nanocarrier void. The delivery vesicles of the present teachings may also be designed so that there is a symmetric or an asymmetric distribution of the drug, both inside and outside of the stabilizing material and/or nanocarrier. Ultrasonically sensitive materials are especially preferred for use as therapeutics with the present specification.

Any of a variety of therapeutic agents, including those described herein, may be encapsulated in, attached to, and/or embedded in said nanocarriers. If desired, more than one therapeutic may be applied using the vesicles. For example, a single vesicle may contain more than one therapeutic, or nanocarriers containing different bioactive agents may be coadministered. In an optimal embodiment, compositions of this disclosure comprise a therapeutic and a targeting moiety. By way of example, a monoclonal antibody capable of binding to a melanoma antigen and an oligonucleotide encoding at least a portion of EL-2 may be administered at the same time. The phrase “at least a portion of” means that, for example, the entire gene need not be represented by the oligonucleotide, so long as the portion of the gene represented provides an effective block to gene expression.

Some of the preferred therapeutic macromolecules for delivery to the patient by embodiments of the present teachings, either attached to or encapsulated within, include genetic material such as nucleic acids, RNA and DNA of either natural or synthetic origin, recombinant RNA and DNA, antisense RNA, microRNAs (miRNAs), shorthairpin RNAs (shRNAs), RNA interference (RNAi), and small interfering RNA (siRNA), including other small RNA-based therapeutics. Other types of genetic material that may be delivered by the nanocarriers described herein include, for example, genes carried on expression vectors such as plasmids, phagemids, cosmids, yeast artificial chromosomes (YACs), defective or “helper” viruses, viral subcomponents, viral proteins or peptides, either alone or in combination with other agents including the therapeutics described herein; and antigene nucleic acids, both single-and-double stranded RNA and DNA, and analogs thereof, such as phosphorothioate and phosphorodithioate oligodeoxynucleotides. Additional genetic material that may be delivered to the patient by the present teachings, include, partially and fully single-stranded and double-stranded nucleotide molecules and sequences; chimeric nucleotides; hybrids, duplexes, heteroduplexes, and any ribonucleotide, deoxyribonucleotide, or chimeric counterpart thereof; and/or corresponding complementary sequence, promoter, or primer-annealing sequence needed to amplify, transcribe, or replicate all or part of a biological molecule or sequence. Additionally, the genetic material may be combined with, for example, proteins, polymers, and/or other components including a variety of therapeutics. Other examples of genetic material that may be applied using the nanocarriers of this disclosure include, for example, DNA encoding at least a portion of LFA-3, DNA encoding at least a portion of an HLA gene, DNA encoding at least a portion of dystrophin, DNA encoding at least a portion of CFTR, DNA encoding at least a portion of IL-2, DNA encoding at least a portion of TNF, and an antisense oligonucleotide capable of binding the DNA encoding at least a portion of ras.

In addition, preferred therapeutics include peptides, polypeptides, and proteins such as adrenocorticotropic hormone, angiostatin, Angiotensin Converting Enzyme [ACE] inhibitors (e.g., captopril, enalapril, and lisinopril), bradykinins, calcitonins, cholecystokinins, and collagenases; enzymes such as alkaline phosphatase and cyclooxygenases colony stimulating factors, corticotropin release factor, dopamine, elastins, epidermal growth factors, erythropoietin, transforming growth factors, fibroblast growth factors, glucagon, glutathione, granulocyte colony stimulating factors, granulocyte-macrophage colony stimulating factors, human chorionic gonadotropin, IgA, IgG, IgM, inhibitors of bradykinins, insulin, integrins, interferons (e.g., interferon α, interferon β, and interferon γ); ligands for Effector Cell Protease Receptors, thrombin, manganese super oxide dismutase, metalloprotein kinase ligands, oncostatin M, interleukins (e.g., interleukin 1, interleukin 2, interleukin 3, interleukin 4, interleukin 5, interleukin 6, interleukin 7, interleukin 8, interleukin 9, interleukin 10, interleukin 11, and interleukin 12), opiate peptides (e.g., enkephalines and endorphins); and oxytocin, pepsins, platelet-derived growth factors, lymphotoxin, promoters of bradykinins, Protein Kinase C, streptokinase, substance P (i.e., a pain moderation peptide), tissue plasminogen activator, tumor necrosis factors, nerve growth factors, urokinase, vascular endothelial cell growth factors, and vasopressin.

Other preferred therapeutics, such as for the treatment of ophthalmologic diseases and prostate cancer, for use with the nanocarriers described herein, include 15-deoxy spergualin, 17-α-acyl steroids, 3-(Bicyclyl methylene)oxindole, 3α-, 5α-tetrahydrocortisol, 5α-reductase inhibitor, adaprolol enantiomers, aldose reductase inhibitors (e.g., sorbinil and tolrestat), aminoguanidine, antiestrogenics (e.g., 24-(1,2-diphenyl-1-butenyl)phenoxy)-N,N-dimethylethanamine) apraclonidine hydrochloride, aurintricarboxylic acid, azaandrosterone, bendazac, benzoylcarbinol salts, betaxolol, bifemelane hydrochloride, bioerodible poly(ortho ester), cetrorelix acetate, cidofovir, vitamin E, dipifevrin, dipyridamole+aspirin, dorzolamide, epalrestat, etofibrate, etoposide, filgastrim, foscarnet, fumagillin, ganciclovir, granulocyte macrophage colony stimulating factor (GM-CSF), haloperidol, imidazo pyridine, latanoprost, lecosim, levobunolol, N-4 sulphanol benzyl-imidazole, N-acyl-5-hydroxytryptamine, nipradilol, nitric oxide synthase inhibitors, pilocarpine, ponalrestat, prostanoic acid, S-(1,3 hydroxyl-2-phosphonylmethoxypropyl)cytosine, somatuline, sorvudine, ticlopidine, timolol, Trolox™, vaminolol, vascular endothelial growth factor, and α-interferon.

Additional therapeutics suitable for delivery to the patient either attached or encapsulated within embodiments of the invention include, anti-allergic agents such as amelexanox; Anti-anginals such as diltiazem, erythrityl tetranitrate, isosorbide dinitrate, nifedipine, nitroglycerin (glyceryl trinitrate), pentaerythritol tetranitrate, and verapamil; antibiotics such as amoxicillin, ampicillin, bacampicillin, carbenicillin, cefaclor, cefadroxil, cephalexin, cephradine, chloramphenicol, clindamycin, cyclacillin, dapsone, dicloxacillin, erythromycin, hetacillin, lincomycin, methicillin, nafcillin, neomycin, oxacillin, penicillin G, penicillin V, picloxacillin, rifampin, tetracycline, ticarcillin, and vancomycin hydrochloride; anti-coagulants such as phenprocoumon, and heparin; anti-fungal agents such as polyene antibiotics like flipin, natamycine, and rimocidin; imidazoles such as clotrimazole, ketoconazole, and micronazole; triazoles such as fluconazole, itraconazole, and ravuconazole; allylamines such as amorolfine, butenafine, naftifine, and terbinafine; echinocandins such as caspofungin, micafungin, and ulafungin, and others such as amphotericin B, flucytosine, griseofulvin, miconazole, nystatin, and ricin; anti-inflammatories such as aspirin difimisal, ibuprofen, indomethacin, meclofenamate, mefenamic acid, naproxen, oxyphenbutazone, phenylbutazone, piroxicam, salicylates, sulindac, and tolmetin; anti-neoplastic agents such as adriamycin, aminoglutethimide, amsacrine (m-AMSA), ansamitocin, arabinosyl adenine, arabinosyl, asparaginase (L-asparaginase), Erwina asparaginase, bisimidazoacridones, bleomycin sulfate, bleomycin, bleomycin, busulfan, carzelesin, chlorambucil, cytosine arabinoside, dactinomycin (actinomycin D), daunorubicin hydrochloride, doxorubicin hydrochloride, estramustine phosphate sodium, etoposide (VP-16), flutamide, interferon α-2a, interferon α-2b, leuprolide acetate mercaptopolylysine, leuprolide acetate, megestrol acetate, melphalan (e.g., L-sarolysin [L-PAM, also known as Alkeran] and phenylalanine mustard [PAM]), mercaptopurine, methotrexate, methotrexate, mitomycin, mitomycin, mitotane, platinum compounds (e.g., spiroplatin, cisplatin, and carboplatin), plicamycin (mithramycin), procarbazine hydrochloride, tamoxifen citrate, taxol, teniposide (VM-26), testolactone, trilostane, vinblastine sulfate (VLB), vincristine sulfate, and vincristine; anti-protozoans such as chloroquine, hydroxychloroquine, metronidazole, quinine, and meglumine antimonate; anti-rheumatics such as penicillamine; and anti-virals such as abacavir, acyclovir, amantadine, didanosine, emtricitabine, enfuvirtide, entecavir, ganciclovir, gardasil, lamivudine, nevirapine, nelfinavir, oseltamivir, ribavirin, rimantadine, ritonavir, stavudine, valaciclovir, vidarabine, zalcitabine, and zidovudine.

Other therapeutics suitable for delivery to the patient, either attached or encapsulated within the nanocarriers described herein, include biological response modifiers such as muramyldipeptide, muramyltripeptide, prostaglandins, microbial cell wall components, lymphokines (e.g., bacterial endotoxin such as lipopoly saccharide, macrophage activation factor, etc.), and bacterial polypeptides such as bacitracin, colistin, and polymixin B; blood products such as parenteral iron, hemin, hematoporphyrins, and their derivatives; cardiac glycosides such as deslanoside, digitoxin, digoxin, digitalin, and digitalis; and circulatory drugs such as propranolol, DNA encoding certain proteins may be used in the treatment of many different types of diseases. For example, adenosine deaminase may be provided to treat ADA deficiency; tumor necrosis factor and/or interleukin-2 may be provided to treat advanced cancers; HDL receptors may be provided to treat liver disease; thymidine kinase may be provided to treat ovarian cancer, brain tumors, or HIV infection; HLA-B7 may be provided to treat malignant melanoma; interleukin-2 may be provided to treat neuroblastoma, malignant melanoma, or kidney cancer; interleukin-4 may be provided to treat cancer; HIV env may be provided to treat HIV infection; antisense ras/p53 may be provided to treat lung cancer; and Factor VIII may be provided to treat Hemophilia B, dyes are included within the definition of a “therapeutic.” Dyes may be useful for identifying the location of a vesicle within the patient's body or particular region of the patient's body. Following administration of the vesicle compositions, and locating, with energy, such compositions within a region of the patient's body to be treated, the dye may be released from the composition and visualized by energy. Dyes useful in the present teachings include fluorescent dyes and colorimetric dyes, such as 3HCl, 5-carboxyfluorescein diacetate, 4-chloro-1-naphthol, 7-amino-actinomycin D, 9-azidoacridine, acridine orange, allophycocyanin, amino methylcoumarin, benzoxanthene-yellow, bisbenzidide H 33258 fluorochrome, BODIPY FL, BODIPY TMR, BODIPY-TR, bromocresol blue, bromophenol blue, carbosy-SNARF, Cascade blue, chromomycin-A3, dansyl+R—NH₂, DAPI, DTAF, DTNB, ethidium bromide, fluorescein, fluorescein-5-maleimide diacetate, FM1-43, fura-2, Indo-1, lucifer yellow, methylene blue, mithramycin A, NBD, oregon green, propidium iodide, rhodamine 123, rhodamine red-X, R-Phycoerythrin, SBFI, SIST, sudan black, tetramethyl purpurate, tetramethylbenzidine, tetramethylrhodamine, texas red, thiazolyl blue, TRITC, YOYO-1, and the like. Fluorescein may be fluorescein isothiocyanate. The fluorescein isothiocyanate, includes, inter alia, fluorescein isothiocyanate albumin, fluorescein isothiocyanate antibody conjugates, fluorescein isothiocyanate α-bungarotoxin, fluorescein isothiocyanate-casein, fluorescein isothiocyanate-dextrans, fluorescein isothiocyanate-insulin, fluorescein isothiocyanate-lectins, fluorescein isothiocyanate-peroxidase, and fluorescein isothiocyanate-protein A.

Additional therapeutics suitable for delivery to the patient, either attached or encapsulated within the nanocarriers of this disclosure, include general anesthetics such as droperidol, etomidate, fentanyl citrate with droperidol, ketamine hydrochloride, methohexital sodium, and thiopental sodium, and radioactive particles or ions such as strontium, iodide rhenium, technetium, cobalt, and yttrium. In certain preferred embodiments, the bioactive agent is a monoclonal antibody or a monoclonal antibody fragment such as a monoclonal antibody capable of binding to melanoma antigen; hormones such as growth hormone, melanocyte stimulating hormone, estradiol, beclomethasone dipropionate, betamethasone, betamethasone acetate and betamethasone sodium phosphate, vetamethasone disodium phosphate, vetamethasone sodium phosphate, cortisone acetate, dexamethasone, dexamethasone acetate, dexamethasone sodium phosphate, flunsolide, hydrocortisone, hydrocortisone acetate, hydrocortisone cypionate, hydrocortisone sodium phosphate, hydrocortisone sodium succinate, methylprednisolone, methylprednisolone acetate, methylprednisolone sodium succinate, paramethasone acetate, prednisolone, prednisolone acetate, prednisolone sodium phosphate, prednisolone tebutate, prednisone, triamcinolone, triamcinolone acetonide, triamcinolone diacetate, triamcinolone hexacetonide, fludrocortisone acetate, progesterone, testosterone, and adrenocorticotropic hormone; local anesthetics such as bupivacaine hydrochloride, chloroprocaine hydrochloride, etidocaine hydrochloride, lidocaine hydrochloride, mepivacaine hydrochloride, procaine hydrochloride, and tetracaine hydrochloride; metabolic potentiators such as glutathione; antituberculars such as para-aminosalicylic acid, isoniazid, capreomycin sulfate cycloserine, ethambutol hydrochloride ethionanide, pyrazinamide, rifampin, and streptomycin sulfate; narcotics such as paregoric, and opiates such as codeine, heroin, methadone, morphine, and opium; neuromuscular blockers such as atracurium besylate, gallamine triethiodide, hexafluorenium bromide, metocurine iodide, pancuronium bromide, succinylcholine chloride (suxamethonium chloride), tubocurarine chloride, and vecuronium bromide; sedatives (i.e., hypnotics) such as amobarbital, amobarbital sodium, aprobarbital, butabarbital sodium, chloral hydrate, ethchlorvynol, ethinamate, flurazepam hydrochloride, glutethimide, methotrimeprazine hydrochloride, methyprylon, midazolam hydrochloride, paraldehyde, pentobarbital, pentobarbital sodium, phenobarbital sodium, secobarbital sodium, talbutal, temazepam, and triazolam; subunits of bacteria (e.g., Mycobacteria, Corynebacteria, etc.), the synthetic dipeptide N-acetyl-muramyl-L-alanyl-D-isoglutamine, and the like; and vitamins such as cyanocobalamin neinoic acid, retinoids and derivatives such as retinol palmitate, α-tocopherol, naphthoquinone, cholecalciferol, folic acid, and tetrahydrofolate.

The aforementioned therapeutics and their precursors and modifications are only representative of the plethora of compounds suitable for delivery to the patient by embodiments of this disclosure. A large number of molecular variables can be altered with nearly all of these illustrative embodiments; thus, a wide variety of drugs, genes, and other compounds and structures have the capability of being delivered alone or in combination with other materials by embodiments of the present invention. Optimally, said therapeutics will be specifically designed and engineered for acoustically mediated drug and gene delivery.

### Best Mode of Practice

The following is a description of a single preferred method for practicing embodiments of the present invention, and should in no way be considered limiting. As this embodiment is described with reference to the aforementioned drawings and definitions, various modifications or adaptations of the methods, materials, and specific techniques described herein may become apparent to those skilled in the art. All such modifications, adaptations, or variations that rely on the teachings of the present invention, and through which these teachings have advanced the art, are considered to be within the spirit and scope of the present invention.

A preferred method for practicing the present invention in a clinical or laboratory environment using free, unencapsulated therapeutic (FIG. 10A), involves the following:
1. Administering to the patient a quantity of one or more therapeutic(s) [act 901].
2. Administering to the patient a quantity of one or more targeted and/or non-targeted contrast agents (act 902; FIG. 10A).
3. Insonating the target region of the patient (act 903; FIG. 9A), which is a complex process composed of many subprocesses (FIG. 10B). Indeed, the first ultrasonic exposure will normally use lower intensity energy for imaging (acts 904 and 905; FIG. 10B). After an image of the target region is obtained, therapeutic ultrasonic pulses ensue (act 906; FIG. 10B), which typically have specific pulse sequences which will likely vary depending on the drug delivery application (FIG. 3). Act 906, FIG. 10B is actually composed of several subprocesses, as described in detail herein, which include microbubble initiation, membrane permeation, enhanced drug delivery, and feedback and monitoring (acts 907 to 910; FIG. 10B). Importantly, the energy level of sonication must be not so great as to cause significant sonolysis and cytotoxicity at the target site.
4. Simultaneously receiving ultrasonic and other emissions from said contrast and/or other agents and materials, quantitating the tissue damage and continuing to generate an image of said region from the received ultrasonic emissions and other data (act 914; FIG. 10A). Quantitating the levels of acoustic cavitation at the target region can include measuring one or more of the following: microbubble backscatter, microbubble backscatter speckle reduction, changes in microbubble backscatter speckle statistics, shear wave propagation changes, and electrical impedance tomography; possibly in combination with one or more properties of said acoustic energy, at the time of or subsequent to the initial application of said acoustic energy (act 914; FIG. 10A).
5. Utilizing the measurements and data gathered in acts 907-910 (FIG. 10B) and 912-914 (FIG. 10A) to adjust the properties of the administered acoustic energy, and/or add additional contrast agents, therapeutics, or other compounds to said patient (acts 912 and 913; FIG. 10A).
6. Ultrasound-mediated intracellular and extracellular drug delivery (act 911; FIG. 10A) can occur during one or more of acts 903, 907 to 910, 912, and 913 (FIGS. 10A-10B); or following said acts.

As one skilled in the art will immediately recognize once armed with the present disclosure, widely varying amounts of therapeutics and/or drug-containing vesicles, as well as contrast agents, may be employed in the practice of this preferred embodiment of the present invention. As used herein, the terms “a quantity of one or more therapeutic(s)” and “quantity of one or more targeted and/or non-targeted contrast agents” are intended to encompass all such amounts.

Therapeutics or therapeutic-containing vesicles may be administered to the patient in a variety of forms adapted to the chosen route of administration, namely, parenterally, orally, or intraperitoneally. Parenteral administration, which is the most preferred method, includes administration by the following routes: intravenous, intramuscular, interstitially, intra-arterial, subcutaneous, intraocular, and intrasynovial; transepithelial including transdermal; pulmonary via inhalation, ophthalmic, sublingual, and buccal; topically including ophthalmic; and dermal, ocular, rectal, and nasal inhalation via insufflation. Intravenous administration is preferred among the routes of parenteral administration.

The useful dosage to be administered and the mode of administration will vary depending on the age, weight, and type of patient to be treated, and the particular therapeutic application intended. Typically, dosage is initiated at lower levels and increased until the desired therapeutic effect is achieved.

The patient may be any type of animal, but is preferably a vertebrate, more preferably a mammal, and most preferably human. By “region of a patient,” “target,” or “target site,” it is meant the whole patient, or a particular area or portion of the patient.

The method of the present invention can also be carried out in vitro. For example, in cell culture applications as described herein, where therapeutic or other compounds and contrast agents may be added to the cells in cultures and then incubated. Therapeutic ultrasonic waves can then be applied to the culture media containing the cells and compounds.

This is a description of a single preferred method for practicing the present invention. A plethora of variables can be altered with this clinical or laboratory treatment protocol, therefore, a wide variety of techniques, materials, contrast agents, and other properties are available for practicing the invention, as well as administration and activation procedures of said components.

While practicing the present invention has been described with reference to specific embodiments, it will be understood by those skilled in the art that various, sometime significant changes may be made and equivalents may be substituted for elements, thereof without departing from the true spirit and scope of the invention. In addition, modifications may be made without departing from the essential teachings of the invention.

As one skilled in the art will immediately recognize once armed with the present disclosure, different nanocarrier preparation methods may be used, as well as widely varying amounts of nanocarriers and contrast agents may be employed in the practice of this preferred embodiment of this specification. As used herein, the phrases “a quantity of said targeted and/or non-targeted nanocarriers” and “quantity of one or more targeted and/or non-targeted contrast agents” are intended to encompass all such amounts.

Nanocarriers may be administered to the patient in a variety of forms adapted to the chosen route of administration, namely, parenterally, orally, or intraperitoneally. Parenteral administration, which is the most preferred method, includes administration by the following routes: intravenous, intramuscular, interstitially, intraarterial, subcutaneous, intraocular, and intrasynovial; transepithelial including transdermal; pulmonary via inhalation, ophthalmic, sublingual, and buccal; topically including ophthalmic, dermal, ocular, rectal, and nasal inhalation via insufflation. Intravenous administration is preferred among the routes of parenteral administration.

The useful dosage to be administered and the mode of administration will vary depending on the age, weight, and type of patient to be treated, and the particular therapeutic application intended. Typically, dosage is initiated at lower levels and increased until the desired therapeutic effect is achieved. The patient may be any type of animal, but is preferably a vertebrate, more preferably a mammal, and most preferably human. By “region of a patient,” “target,” or “target site,” it is meant the whole patient, or a particular area or portion of the patient.

The method of the present teachings can also be carried out in vitro (i.e., in cell culture applications, where the nanocarriers and contrast agents may be added to the cells in cultures and then incubated). Therapeutic ultrasonic waves can then be applied to the culture media containing the cells and nanocarriers.

The aforementioned is a description of a single preferred method for practicing the methods of the present disclosure. A plethora of variables can be altered with this clinical or laboratory treatment protocol; therefore, a wide variety of techniques, materials, and other properties are available for the preparation of targeted and non-targeted nanocarriers and contrast agents for acoustically mediated drug delivery, as well as administration and activation procedures of said components.

### EXAMPLES

A more complete understanding of embodiments of this specification will be obtained from the following Examples, all of which are prospective (i.e., prophetic). These examples are intended to be exemplary only and non-limiting to embodiments of the present invention. The chemicals, materials, reagents, glassware, equipment, and instrumentation components necessary for the synthesis, purification, characterization, and evaluation of embodiments of this disclosure are readily known and are available to those skilled in the art.

### Example 1

### Prospective Example: Synthesis of polyethylene glycol-block-oligo [desaminotyrosyl-tyrosine octyl ester suberate]-block-polyethylene glycol [PEG-b-oligo(DTO-SA)-b-PEG]

This prospective example illustrates the synthesis of polyethylene glycol-block-oligo(desaminotyrosyl-tyrosine octyl ester suberate)-block-polyethylene glycol (PEG-b-oligo[DTO-SA]-b-PEG). In a 100 ml round-bottomed flask, 2.21 gm (0.005 mole) of DTO, 0.96 gm (0.0055 mole) of suberic acid, 0.59 gm (0.002 mole) of 4-dimethylaminopyridinium-p-toluene sulfate, and 25 ml of methylene chloride (DCM) are combined at 293° K. While stirring continuously at room temperature, 1.8 gm (0.014 mole) of diisopropylcarbodiimide (DIPC) is added to the suspension. At regular intervals, aliquots are withdrawn from the mixture for gel permeation chromatography (GPC). When the number average molecular weight (Mₙ) and the weight average molecular weight (M_{w}) of the reaction mixture reaches approximately 7,000 and 15,000 respectively (relative to polystyrene standards), 1.1 gm of poly(ethylene glycol) monomethyl ether and 0.4 gm of DIPC are added. Following 2 hours of additional stirring, the reaction mixture is filtered through a sintered glass funnel, and the filtrate evaporated to 10 ml, and then precipitated with 2-propanol. This precipitate is dried, dissolved in 10 ml of DCM, and reprecipitated with 50 ml of methanol, and dried under a vacuum at room temperature.

### Example 2

### Prospective Example: Synthesis of polyethylene glycol-block-oligo [desaminotyrosyl-tyrosine octyl carbonate]-block-polyethylene glycol (PEG-b-oligo [DTO-carbonate]-b-PEG

This prospective example illustrates the synthesis of polyethylene glycol-block-oligo(desaminotyrosyl-tyrosine octyl carbonate)-block-polyethylene glycol (PEG-b-oligo [DTO-carbonate]-b-PEG). A 0.5 liter 3-necked flask is equipped with a mechanical stirrer and pump and purged with N₂ for 15 minutes. PEG is added to the flask followed by triphosgene (TP; solid) and DCM (HPLC grade). The mixture is stirred to obtain a clear solution (10-15 minutes). At this point, the reaction mixture contains “activated PEG”—PEG-chloroformate—and an excess of unreacted TP. DTO and DCM are placed in a bottle with a screw cap, and the TP solution is added to the reaction mixture using an fluid metering (FMI) pump (over 1 hour). After the addition is complete, 10 ml of DCM is added to the bottle and the reaction mixture over 10 minutes using the pump. After addition is complete, a 150 ml aliquot is withdrawn, evaporated to dryness with air and diluted with 1 ml of THF, filtered, and the filtrate analyzed by GPC to determine the molecular weight distribution of the synthesized compounds. GPC chromatogram should reveal a mixture of products and reacted monomers.

The entire reaction mixture is concentrated by evaporation to a thick syrup (˜10 ml total), which is then precipitated with 60 ml of 2-propanol (drop-wise addition) and allowed to settle. The product obtained should be a yellow thick oil, and the solvents are then decanted off. The precipitate is dried (under N₂) for 20 minutes, redissolved in 10 ml of DCM and precipitated with 60 ml of 2-propanol. This process is repeated 2 more times with (1) 50 ml of methanol:IPA=1:1, and (2) 50 ml methanol. The product (a thick gum) is dried under a stream of nitrogen followed by vacuum drying.

The polycarbonate middle blocks of the present disclosure can be prepared by the conventional methods for polymerizing diphenols into the same, as described by U.S. Pat. No. 5,099,060 (Kohn et al., 1992), the disclosures of which are incorporated herein by reference in their entirety for all purposes. These methods involve the reaction of amino acid-derived diphenol compounds, including those described in U.S. Pat. No. 4,980,449 (Kohn et al., 1990), the disclosures of which are incorporated herein by reference in their entirety for all purposes, with phosgene or phosgene precursors (e.g., diphosgene or triphosgene) in the presence of a catalyst. Suitable processes, associated catalysts, and solvents are known in the art and taught in Chemistry and Physics of Polycarbonates (Shnell, 1964), the disclosures of which are incorporated herein by reference in their entirety for all purposes.

Polymerizing oligomers having pendant free carboxylic acid groups from diphenols, with pendant free carboxylic acid groups, without cross-reaction of the free carboxylic acid groups with the co-monomer, is now possible. Accordingly, homopolymers or copolymers of benzyl ester diphenyl monomers such as DTBn may be converted to corresponding free carboxylic acid homopolymers and copolymers through the selective removal of the benzyl groups by the palladium catalyzed hydrogenolysis method disclosed by U.S. Pat. No. 6,120,491, the disclosures of which are incorporated by reference herein in their entirety for all purposes. The catalytic hydrogenolysis is necessary because the lability of the oligomer backbone prevents the employment of harsher hydrolysis techniques.

### Example 3

### Prospective Example: Self-Assembly of Nanocarriers from Tyrosine Triblock Copolymers

This prospective example illustrates the self-assembly of nanocarriers from tyrosine triblock copolymers. Briefly, vesicle self-assembly is performed by dissolving, for example, 10 mg of PEG-oligo(DTO suberate)-PEG in 0.2 gm THF, and adding said mixture dropwise to 4.79 gm of water (18 MΩcm⁻¹) under mild agitation. To achieve uniform particle size, the resulting turbid dispersion is sequentially filtered through 0.45, 0.22, and 0.1 μm size syringe filters. All subsequent characterizations are performed using this final filtered preparation. Trace organic solvent contamination is removed by either gentle nitrogen blow-drying or size exclusion chromatography.

The prior art has established that self-assembly of amphiphilic molecules depends on several correlated properties of the underlying material, i.e., its chemical structure, architecture, and/or molecular weight. However, assuming that the driving force of the self-assembly is mainly governed by hydrophobic interactions, the design of a self-assembling block copolymer inherently depends on its molecular weight, and hydrophobic to hydrophilic balance. The self-assembly of the triblock copolymers in dilute aqueous solution is induced by simple dropwise addition and may be facilitated by sonication, high shear mixing, nanoprecipitation or emulsification methods. Active hydrophobic products are complexed by premixing the triblocks and hydrophobic products in a suitable solvent prior to nanocarrier formation or by forming the nanospheres in solutions or suspensions of the product to be completed.

Acceptable pharmaceutical carriers for therapeutic used are well known in the pharmaceutical field, and are described, for example, in Remington: The Science and Practice of Pharmacy, (Gennaro et al., 1995), the disclosures of which are incorporated herein in their entirety for all purposes. Such materials are non-toxic to recipients at the dosages and concentrations employed, and include diluents, solubilizers, lubricants, suspending agents, encapsulating materials, solvents, thickeners, dispersants, buffers such as phosphate, citrate, acetate and other organic acid salts, anti-oxidants such as ascorbic acid, preservatives, low molecular weight (less than approximately 10 residues) peptides such as polyarginine, proteins such as serum albumin, gelatin or immunoglobulins, hydrophilic polymers such as poly(vinylpyrrolindinone), amino acids such as glycine, glutamic acid, aspartic acid or arginine, monosaccharides, disaccharides, and other carbohydrates including cellulose or its derivatives, glucose, mannose or dextrines, chelating agents such as EDTA, sugar alcohols such as mannitol or sorbitol, counter-ions such as sodium and/or non-ionic surfactants such as Tween™, Pluronics®, or PEG.

### Example 4

### Prospective Example: synthesis of peptosomes based on poly(L-lysine-HBr)₆₀-block-poly(L-leucine)₂₀

The aforementioned peptosome publications in paragraphs [0158], [0159], and [0160], now apart of this specification, describe in detail methods and compositions to generate copolypeptides, peptosomes, and related materials, including, for example, hybrid structures with polymers and copolymers, each with varied properties. These materials can be successfully employed in acoustically mediated drug delivery using the methods described herein, either in their form know in the art, or modified specifically (e.g., through stabilization and hybrid structures) for acoustically mediated intracellular drug delivery in vivo. For enablement and other purposes, the representative synthesis and characterization of peptosomes based on poly(L-lysine-HBr)₆₀-block-poly(L-leucine)₂₀ is presented.

All α-amino acid-N-carboxyanhydride monomers are prepared using previously described methods (Fuller et al., 1976; Deming, 1997). Polymerization of NCA monomers are performed using Co(PMe₃)₄ as initiator. Purified yields of the copolypeptides should be in the range of 95-98%. Copolypeptides are purified, and then characterized using tandem gel permeation chromatography/light scattering (GPC/LS) and infrared measurements.

In a nitrogen dry box, poly(N_{∈}-benzyloxycarbonyl-L-lysine)₆₀-block-poly(L-leucine)₂₀ (Z-Lys NCA) (200 mg, 0.20 mmole) is dissolved in tetrahydrofuran (THF, 4 ml) and placed in a 20 ml scintillation vial with a stir bar. A Co(PMe₃)₄ initiator solution (100 μl of an 82 mM solution in THF) is then added to the vial via syringe. The vial is sealed and allowed to stir in the dry box for 4 hours at 25° C. After 4 hours, an aliquot (50 μl) is removed and diluted to a concentration of 5 mg/ml in dimethylformamide (DMF) contain 0.1 M LiBr for GPC/LS analysis (Mₙ=16,050; M_{w}/Mₙ=1.05). The remainder of the aliquot is analyzed by infrared spectroscopy (FTIR) to confirm all the Z-Lys NCA has been consumed. In a dry box, L-leucine-N-carboxyanhydride (Leu NCA) (35 mg; 0.23 mmole) is dissolved in THF (0.7 ml) and the added to the reaction vial. The polymerization is allowed to continue with stirring at 25° C. in the dry box for another 3 hours. Afterwards, an aliquot (50 μl) is removed and diluted to a concentration of 5 mg/ml in DMF containing 0.1 M LiBr for GPC/LS analysis (Mₙ=18,430; M_{w}/Mₙ=1.13). The remainder of the aliquot is analyzed by FTIR to confirm that all the Leu NCA has been consumed. Outside of the dry box, the copolypeptide is then precipitated by adding the THF solution to methanol (50 ml), and then isolated by centrifugation. The polymer pellet is then soaked in methanol (50 ml) for 2 hours before a second centrifugation which yields the protected copolymer. After drying under a vacuum for several hours, a white powder is formed as product.

A 100 mL round-bottom flask is charged with (Z)K₆₀L₂₀ (200 mg) and trifluoroacetic acid (TFA, 8 ml). The flask is placed in an ice bath and allowed to stir for 15 minutes, allowing the polymer to dissolve and contents of the flask to cool to 0° C. At this point, HBr (0.6 ml of 33% solution in HOAc, 5 equivalents) is added dropwise and the solution is then allowed to stir in an ice bath for 1 hour. Afterwards, diethyl ether (20 ml) is added in order to precipitate the product. The mixture is centrifuged to isolate the solid precipitate, and the product is subsequently washed with diethyl ether (20 ml) several times to yield a white solid. After drying the sample in air, it is resuspended in water 10 ml), LiBr (100 mg) is added, and the solution is placed in a dialysis bag (pore size: 2000 Daltons). The sample is dialyzed against EDTA (3 mM in deionized [DI] water) for one day in order to remove residual cobalt initiator, and then for 2 additional days against DI water (changed every 8 hours). After dialysis, the sample is lyophilized yielding a white fluffy powder as product.

### Peptosome Formation

Solid copolypeptide powder is dispersed in THF to give a 1% (w/v) suspension, which is then placed in a bath sonicator for 30-45 minutes until the copolypeptide is evenly dispersed and no large particulates can be observed. A stir bar is added followed by dropwise addition of an equal volume of deionized (DI) water under constant stirring. The stir bar is then removed and the mixture is placed in a bath sonicator for 30 minutes, after which the mixture is dialysed against 5 changes of DI water for 24 hours.

### Extrusion of Peptosomes

Aqueous vesicle suspension of KY samples, 1% (w/v) are extruded using a commercially-available extruder (e.g., Avanti Mini-Extruder). Extrusions are performed using membranes with different pore sizes 1.0 μm, 0.4 μm, and 0.2 μm (e.g., Whatman Nucleopore Track-Etch polycarbonate). The membranes are soaked in Millipore water for 10 minutes prior to extrusion. After two passes through the extruder, the resulting suspension is allowed to stand for 16 hours and then analyzed using differential interference contrast microscopy (DIC).

### Characterization of Peptosomes

Peptosomes are characterized (minimally) by differential interference contrast microscopy, dynamic light scattering, transmission microscopy, circular dichroism, and critical micelle concentration. Brief summaries of these procedures are as follows.

Differential Interference Contrast Microscopy (DIC). Suspensions of copolypeptides (1% (w/v) as described above) are visualized on glass slides with spacers between the slide and coverslip, which allows for the sample droplet to adhere to both surfaces simultaneously, limiting the disturbance to self-assembled structures. Samples are imaged on a DIC/fluorescence inverted optical microscopy (e.g., Zeiss Axiovert 200).

Dynamic Light Scattering (DLS). Assemblies of all KₘYₙ compositions are prepared as 1% (w/v) suspensions and passed through different polycarbonate filters (0.05 μm, 0.1 μm, 0.2 μm, 0.4 μm, and 1.0 μm) prior to analysis. Light scattering data is measured on a digital correlator with a vertically polarized 35 mW HeNe laser at a 90° angle with extended baseline positioning. Histograms detailing diameter distribution are prepared using a non-negatively constrained lease squares approximation.

Transmission Electron Microscopy (TEM). Copolypeptide suspensions (0.1% (w/v)) are extruded separately. One drop of each respective sample is placed on a 200 mesh Formvar coated copper grid and allowed to remain so for 90 seconds. Filter paper is then used to remove residual sample and liquid. One drop of 0.1% (w/v) uranyl acetate (negative stain) is placed on the grid, allowed to stand for 20 seconds, and subsequently removed by washing with drops of Millipore water and wicking away excess liquid with filter paper. The resulting samples are imaged using TEM at 80 KEv and ambient temperature.

Circular Dichroism (CD). Copolypeptide solutions (0.5 mg/ml in a 1% (w/v) TFA/DI water mixture) are prepared and subsequently passed through 0.45 μm PTFE filters and then lyophilized to dryness. Dried samples are then processed by a previously published procedure (Holowka, Pochan et al., 2005), and the resulting suspensions analyzed directly in a 1 mm path length quartz cell on a RSM spectrometer.

Critical Micelle Concentration (CMC) Determination by Pyrene Fluorescence. Aqueous copolypeptide samples 1% (w/v) are diluted to give a series of concentrations within the range of 0.01 M to 1×10⁻¹² M, and mixed with a solution of pyrene (400 μl; 15.4 μM) in acetone according to a previously published procedure (Napoli, Valentini et al., 2004). The mixtures are then stirred for 24 hours in air to allow for acetone evaporation. Fluorescence measurements are performed using 1 cm polystyrene civets on a fluorescence spectrophotometer. The emission spectra are recorded from 350 to 500 nm with an excitation wavelength of 340 nm. Normalized intensity for the 371 nm and 393 nm peaks are measured, and the normalized ratio (I₃₉₃/I₃₇₁) is plotted against copolypeptide concentration over a range of 0.01 M to 1×10⁻¹² M. CMCs are determined as the intercept between linear fits of the baseline pyrene fluorescence and the tangent line of the inflection point on the plot of (I₃₉₃/I₃₇₁) verses log concentration (M).

### Example 5

### Prospective Example: Synthesis of Polymersomes from Amphiphilic Diblock Copolymers

This prospective example demonstrates the synthesis of polymersomes for use in acoustically mediated drug delivery from amphiphilic diblock copolymers. Polymeric membranes assembled from a high molecular weight, synthetic analog (i.e., a super-amphiphile) are produced with a linear diblock copolymer, EO₄₀-EE₃₇. This neutral, synthetic polymer has a mean number-average molecular weight of approximately 3900 gm/mole mean and a contour length ˜23 nm, which is approximately 10 times that of a typical phospholipid acyl chain. The polydispersity measure, M_{w}/Mₙ, is 1.10, where M_{w} and Mₙ are the weight-average and number-average molecular weights, respectively. The PEO volume fraction is f_{EO}=0.39 (TABLE 1).

A thin film (approximately 10 nm to 300 nm) is prepared by employing electroformation methods previously known in the art (Angelova et al., 1992). Giant vesicles attached to the film-coated electrode are typically visible after 15 to 60 minutes. These dissociate from the electrodes by lowering the frequency to 3 to 5 Hz for at least 15 minutes, and by removing the solution from the chamber into a syringe. The polymersomes are typically stable for at least one month if kept in a vial at room temperature. The vesicles also remain stable when resuspended in physiological saline at temperatures ranging from 10° C. to 50° C.

Thermal undulations of the quasi-spherical polymersome membranes provide an immediate indication of membrane softness. Further, when the vesicles are made in the presence of either a 10-kD fluorescent dextran, sucrose, or a protein (e.g., globin) the probe is typically found to be readily encapsulated and retained by the vesicle for at least several days. The polymersomes prove highly deformable, and sufficiently resilient that they can be aspirated into micrometer-diameter pipettes. The micromanipulations are done with micropipette systems, as described above, and analogous to those described by Longo et al. (1997) and by Discher et al. (1994).

The elastic behavior of a polymersome membrane in micropipette aspiration (at ˜23° C.) appears comparable in quality to a fluid-phase lipid membrane. Analogous to a lipid bilayer, at low but increasing aspiration pressures, the thermally undulating polymersome membrane is progressively smoothed, increasing the projected area logarithmically with tension, t. From the slope of this increase (i.e., in tension units of mN/m) versus the fractional change, α, in vesicle area, the bending modulus, K_{b}, is calculated (see, e.g., Evans et al., 1990) with the following equation:

K_{b}≅k_{B}T ln(t)/(8απ)+constant Equation 3

When calculated, it is typically found to be 1.4±0.3×10⁻¹⁹ Joules (J). In equation 3, k_{B} is Boltzmann's constant and T is an absolute temperature. Above a crossover tension, tₓ, an area expansion modulus, Kₐ, is estimated with

Kₐ=t/α Equation 4

applied to the slope of the aspiration curve.

Aspiration in this regime primarily corresponds to a true, as opposed to a projected, reduction in molecular surface density, and for the polymersome membranes, Kₐ=120±20 mN/m. Fitted moduli are checked for each vesicle by verifying that the crossover tension, tₓ=(Kₐ/K_{b})(k_{BT}/8π), (Evans et al., 1990) suitably falls between appropriate high-tension (i.e., membrane stretching) and low-tension (i.e., membrane smoothing) regimes.

Measurements of both moduli, Kₐ and K_{b}, are typically found to yield essentially unimodal distributions with small enough standard deviations (i.e., usually 20% of mean) to be considered characteristic of unilamellar polymer PEO-PEE vesicles. The moduli are also well within the range reported for various pure and mixed lipid membranes. SOPC (1-stearoyl-2-oleoyl phosphatidylcholine) in parallel manipulations is found, for example, to be approximately Kₐ=180 mN/m and K_{b}=0.8×10⁻¹⁹ J. Lastly, at aspiration rates where projection lengthening is limited to <1 μm/s, the microdeformation is largely reversible, consistent again with an elastic response.

The measured Kₐ is most simply approximated by four times the surface tension, γ, of a pure hydrocarbon-water interface (=20 to 50 mJ/m²), and thus reflects the summed cost of two monolayers in a bilayer (see, e.g., Israelachvili, 1995). The softness of Kₐ, compared with gel or crystalline states of lipid systems is further consistent with liquid-like chain disorder as described by Evans et al. (1987). Indeed, because the average interfacial area per chain, <A_{c}>, in the lamellar state, has been estimated to be <A_{c}>/2.5 nm² per molecule (see, e.g., Warriner et al., 1996), the root-mean-squared area fluctuations at any particular height within the bilayer can also be estimated to be, on average, <δA_{c}²>^{1/2}=(<A_{c}>k_{BT}/Kₐ)²/0.3 nm² per molecule, which is a significant fraction of <A_{c}> and certainly not small on a monomer scale.

Moreover, presuming in the extreme, a bilayer of unconnected monolayers d/2 thick, with d estimated from cryo-TEM (data not shown), the PEE contour length is usually more than twice the monolayer core thickness, and therefore, configurationally mobile along its length. In addition, molecular theories of chain packing in bilayers have suggested that although at a fixed area per molecule there is a tendency for K_{b} to increase with chain length (i.e., membrane thickness), other factors such as large <A_{c}> can act to reduce K_{b} (see, e.g., Szleifer et al., 1988). Thus, despite the large chain size of EO₄₀-EE₃₇, a value of K_{b} similar to that of lipid bilayers, is acceptable.

Related to the length scales above, the root ratio of moduli, (K_{b}/Kₐ)^{1/2}, is generally recognized as providing a proportionate measure of membrane thickness. In addition, for the presently described polymersome membranes, (K_{b}/Kₐ)^{1/2} is approximately 1.1 nm, on average. By comparison, fluid bilayer vesicles of phospholipids or phospholipids plus cholesterol have reported a ratio of (K_{b}/Kₐ)²=0.53 to 0.69 nm (Evans et al., 1990; Helfrich et al., 1984). Typically, the fluid bilayer vesicles of phosholipids plus cholesterol have a higher Kₐ than those of phospholipid alone.

A parsimonious continuum model for relating such a length scale to structure is based on the idea that the unconnected monolayers of the bilayer have, effectively, two stress-neutral surfaces located near each hydrophilic-hydrophobic core interface. If one assumes that a membrane tension resultant may be located both above and below each interface, then

(K_{b}/Kₐ)=δ_{H}δ_{C} Equation 5

where δ_{H} and δ_{c} are, respectively, distances from the neutral surfaces into the hydrophilic and hydrophobic cores.

For lipid bilayers with d/2=1.5 nm and hydrophilic head groups equal to 1 nm thick, estimates of δ_{C}=0.75 nm and δ_{H}=0.5 nm yield a root-product, (δ_{H}δ_{C})^{1/2}=0.61. The numerical result for PEO-PEE membranes (i.e., 1.1 nm) suggests that the stress resultants are centered further from the interface, but not necessarily in strict proportion to the increased thickness or the polymer length.

Elastic behavior terminates in membrane rupture at a critical tension, τ_{c}, and areal strain, α_{c}. With lipids, invariably α_{c}=0.05. This is consistent, it appears, with a molecular theory of membranes under stress. For the polymersomes, cohesive failure should occur at α_{c}=0.19±0.02.

Another metric is the toughness or cohesive energy density that, for such a fluid membrane, is taken as the integral of the tension with respect to area strain, up to the point of failure

E_{c}=½Kₐα_{c}² Equation 6

For a range of natural phospholipids mixed with cholesterol, the toughness has been systematically measured, with E_{c} ranging from 0.05 to 0.5 mJ/m². By comparison, the EO₄₀-EE₃₇ membranes are 5 to 50 times as tough, with E_{c}≅2.2 mJ/m². On a per molecule basis, as opposed to a per area basis, such critical energies are close to the thermal energy, k_{BT}, whereas such an energy density for lipid bilayers is a small fraction of k_{BT}.

Despite the comparative toughness of the polymersome membrane, a core “cavitation pressure,” P_{c}, may be readily estimated as p_{c}=t_{c/d} (5) yielding a value of p_{c}=−25 atm. This value falls in the middle of the range noted for lipid bilayers, p_{c}=−10 atm to −50 atm. Bulk liquids such as water and light organics, are commonly reported to have measured tensile strengths of such a magnitude as may be generically estimated from a ratio of nominal interfacial tensions to molecular dimensions (i.e., ˜γ/d). In membrane systems, this analogy again suggests an important role for density fluctuations, which are manifested in a small Kₐ, and which must become transversely correlated upon coalescing into a lytic defect.

Because the previous estimate for <δ_{c}²>^{1/2} is clearly not small as compared with the cross-section of H₂O, a finite permeability of the polymersome membranes to water is expected. To verify this expectation, polymersome permeability is obtained by monitoring the exponential decay in EO₄₀-EE₃₇ vesicle swelling as a response to a step change in external medium osmolarity. Vesicles are prepared in a 100 mOsm sucrose solution to establish an initial, internal osmolarity, after which they are suspended in an open-edge chamber formed between cover slips and containing 100 mOsm glucose. A single vesicle is aspirated with a suction pressure sufficient to smooth membrane fluctuations, after which the pressure is lowered to a small holding pressure.

With a second transfer pipette, the vesicle is moved to a second chamber with 120 mOsm glucose. Water flowed out of the vesicle due to the osmotic gradient between the inner and outer surfaces, which led to an increased projection length that is monitored over time. The exponential decrease in vesicle volume is calculated from video images and then fit to determine the permeability coefficient (P_{f}). The permeability coefficient, P_{f}, should be approximately 2.5±2 μm/s.

In marked contrast, membranes composed purely of phospholipids with acyl chains of approximately 18 carbon atoms typically have permeabilities in the fluid state of at least an order of magnitude greater (i.e., 25 μm/s to 150 μm/s). Polymersomes are thus significantly less permeable to water, which suggests beneficial applications for the vesicles, especially in acoustically mediated intracellular drug delivery in vivo.

### Example 6

### Prophetic Example: Preparation of Block Copolymers, Surfactant Mixtures, and Characterization of an Example Supramolecular Assembly

This prophetic example illustrates the preparation of block copolymers, surfactant mixtures, and characterization of an example supramolecular assembly. The amphiphilic block copolymers may be synthesized by any method known to one of ordinary skill in the art. Such methods are taught, for example, by (Hillmyer et al., 1996a) and (Hillmyer et al., 1996b) both of which are incorporated in their entirety herein by reference, although the practitioner need not be so limited. Nevertheless, use of the Bates method results in very low polydispersity indices for the synthesized polymer (not exceeding 1.2), and make the methods particularly suited for use in the present teachings, at least from the standpoint of homogeneity.

Poly(ethylene oxide)₂₁₀-b-poly(tert-butyl methacrylate)₉₇ block copolymer is synthesized and characterized as described by a procedure know in the art. tert-Butyl groups are removed by acid hydrolysis. The block ionomer is converted to the sodium salt form (PEO₂₁₀-b-PMA₉₇) by precipitation from a tetrahydrofuran/methanol mixture with an isopropanol solution of sodium hydroxide. The precipitate is thoroughly washed with excess isopropanol, dissolved in water, and lyophilized. The concentration of the carboxylate groups in the stock solution is determined by potentiometric titration.

### Preparation of Surfactant Mixtures and Supramolecular Complexes

Stoichiometric amounts ([COO⁻]=[NR₄⁺] total) of PEO-b-PMA and of a cationic surfactant mixture is dissolved in 5 ml of methanol to a final concentration of 2 mM of each. Water (0.5 ml) is slowly added to the solution under constant stirring. The solvents are allowed to evaporate at 60° C. When the residual volume reaches 0.8 ml, an additional 0.8 ml of water is added, and evaporation at 60° C. to a 0.5 ml final volume and repeated. This cycle of water addition and evaporation is repeated twice to ensure the elimination of methanol from the mixture. The final volume of the solution is adjusted to 2 ml with water. Suitable surfactants for use in this example include hexadecyltrimethylammonium bromide (HTAB), didodecyldimethylammonium bromide (DDDAB), dioctadecyldimethylammonium bromide (DODAB), trioctylmethylammonium bromide (TMAB), and N-hexadecylpiridinium bromide (HPyB). While fluorescent probes useful for the characterization of said complexes include: 6-hexadecanoyl-2-((2-(trimethylammonium)-ethyl-)-methylamino)-naphatalene chloride (Patman), and 4-(4-(dihexadecylamino)-styryl)-N-methylpyridinium iodide (DiA).

### Characterization of Supramolecular Complexes

Effective hydrodynamic diameters (D_{eff}) of the supramolecular complexes is determined by dynamic light scattering (DLS). Measurements are made in the 0.1 mM-0.5 mM surfactant concentration range. Fluorescence measurements are carried out using a spectrofluorophotometer, typically at 0.5 mM total surfactant concentration. The concentration of the FRET donor, Patman, is 1 M, and the concentration of the FRET acceptor, DiA, is varied between 1.25 and 5 μM. [FRET is used in microscopy to measure how close two fluorophores are together. Resonance energy transfer is a mechanism by which energy is transferred directly from one molecule to another. This only occurs over a very small distance, usually less than 10 nm, which is on the order of the size of a typical protein.] Fluorescence intensities are corrected for the inner filter effect:

F(corr)=F×10^{(A}^{ₑₓ}^{+A}^{ₑₘ}^{)/2} Equation 7

where F is the fluorescence intensity measured in the solution with optical densities of Aₑₓ at the excitation wavelength and Aₑₘ at the emission wavelength. As the concentration of DiA in a mixture increased, the fluorescence intensity of Patman (λₑₘ=475 nm), illuminated at its excitation maximum λₑₓ=375 nm, progressively decreases. Simultaneously, the fluorescence of DiA (λₑₘ=540 nm) increases, indicating that the energy is nonradiatively transferred from the donor to the acceptor. The FRET efficiency is calculated from the change in the relative fluorescence intensity of the donor:

$$FRET = 1 - \frac{{F}_{D / A}}{{F}_{D}^{o}} \qquad Equation 8$$

where F is the fluorescence intensity of the donor (Patman) in the absence of the acceptor (DiA) and F_{D/A} is its fluorescence in the presence of the acceptor.

A negative staining technique is used for the transmission electron microscopy (TEM) studies. A drop of the sample solution (0.25 mM total surfactant concentration) is allowed to settle on a Formvar-coated copper grid for 1 minute. Excess sample is wicked away with filter paper, and a drop of 1% uranyl acetate solution is placed into contact with the sample for 20 seconds. The samples are air dried and studied using a transmission electron microscope.

With most embodiments, the compositions of the present invention normally form smaller sized complexes that are thermodynamically stable and do not aggregate after storing in solutions for an extended period of time (weeks or months), depending on the type of polymer. The ability to produce particles of such limited size is important because small particles can easily penetrate into tissues through even small capillaries. The preferred size of the acoustically responsive supramolecular complexes described herein is less than 500 nm, more preferred less than 200 nm, still more preferred less than 100 nm. These systems can be lyphilized and stored as a lyphilized powder and then re-dissolved to form solutions with the particles of the same size.

A plethora of molecular variables can be altered with these illustrative supramolecular complexes and derivative embodiments, therefore, a wide variety of material properties are available for the preparation of the acoustically responsive supramolecular complexes, specifically engineered for in vivo drug delivery.

### Example 7

### Prospective Example: Synthesis of an Amphipathic Polypeptide Dendron

This prospective example demonstrates the molecular modeling and synthesis of an amphipathic, polypeptide dendron for use in acoustically mediated drug delivery, pictured in FIG. 11A. Molecular modeling of the dendron is described first, followed a description of the synthesis and purification of the dendron.

Molecular Modeling. The initial topology file for the dendrimer is defined using, for example, QUANTA/CHARMm and Accord Cheminformatics software (Accelrys, San Diego, Calif.). In one procedure, half of the molecule is defined as a fragment and joined to form the complete dendrimer. The absolute configuration of the lysine residues is “S” but with the possibility of the tetradecanoic acid adopting the S or R configurations in the synthesis. In addition, 2048 conformers are possible. For later molecular dynamics trials, an S configuration is applied throughout, and also to apply an alpha helical conformation to the central section of the polymer that forms a stretch of seven lysine residues. The amino groups of lysine are given a formal charge of +1, to become NH₃⁺. Minimization in CHARMm using charge templates provides the starting conformation for further dynamics procedures.

For greater control of dynamics parameters, the structure is transferred to, for example, the Sybyl software program (Tripos, St. Louis, Mo.). To show that the molecule was not adopting preferred stable conformations, a series of dynamics heating (1000° K. over 1 ps) and annealing (200° K. over 2.5 ps) procedures are conducted. In addition, dynamic characteristics of the molecule under different conditions of simulated solvation are carried out. Using Sybyl software, the Gasteiger-Huckel method of charge assignment appropriate to a system of single and double bonds is used. After energy minimization, dynamics simulations are set up to run over, for example, 40 ps, sufficient to allow large internal movements of the molecule to take place. Bond vibrations involving hydrogen atoms are constrained with the “Shake algorithm” to allow a dynamics integration time step of 1 fs. To allow for the gross effect of solvent water, a distant dependent dielectric constant of 2 is applied. For comparison, a constant dielectric of 1 is used to simulate in vacuo dynamics. The same initial minimized conformation is used as the starting point in each case. Simulations may be carried out using, for example, a Silicon Graphics Indigo2 workstation (Silicon Graphics Computer Systems).

Synthesis and Purification. The dendron in FIG. 11A is prepared by stepwise solid-phase peptide synthesis on an MBHA resin with a loading capacity of 0.67 mmole/gm, using a previously published tert-Butoxycarbonyl (Boc) methodology (Sakthivel et al., 1998). Briefly, α-(tert-butoxycarbonylamino) tetradecanoic acid is coupled to the resin with HBTU (four equivalents), HOBt (four equivalents), and EIEA (eight equivalents) in DMF followed by N-termini deprotection with 100% TFA (2×1 minute). The completion of coupling is monitored by the Kaiser test, a test commonly used to detect the presence or absence of free amine after deprotection or coupling, with typical free primary amino groups giving a dark blue color. Three successive couplings with the liposamino acid are performed, each followed by N-deprotection and washing twice with DMF (twice resin volume) before and after deprotection. This is then followed by three successive couplings/deprotections with Boc-Lys(boc)-OH (4, 8, 16 equivalents, respectively) under the same conditions, then the final coupling/deprotection with the liposamino acid (32 equivalents). The resin is washed with 95% glacial acetic acid and lyphilized, dried over P₂O₅ for 3 days, and stored under silica gel. The molecular weight of the product is confirmed by mass spectrometry.

### Example 8

### Prospective Example: Formation of Dendrisomes

This prospective example demonstrates the formation and characterization of dendrisomes formed from the amphipathic polypeptide dendron synthesized in Example 1.

### Prospective Experimental Methods

The polypeptide dendrons synthesized in Example 6 are used to form supramolecular aggregates (dendrisomes, by combining said dendrons with different ratios of cholesterol (CHOL). These dendron/CHOL molar ratios are (1) 1:0 (20 mg dendron:0.0 mg CHOL), (2) 1:1 (18.03 mg dendron: 1.97 mg CHOL), (3) 1:5 (12.93 mg dendron:7.07 mg CHOL), (4) 1:7 (11.33 mg dendron: 8.67 mg CHOL), and (5) 1:9 (10.09 mg dendron:9.91 mg CHOL). Dendrisomes are prepared by reverse-phase evaporation method as described in (Torchillin et al. 2003), the disclosures of which are incorporated herein by reference in their entirety for all purposes. Briefly, after dissolving 20 mg of dendron/CHOL, following the molar rations listed above, in 40 ml chloroform:ether. A mixture is produced (1:1) by injecting 5 ml of deionized water into the organic suspension. The mixture is then bath sonicated for 2 minutes, followed by removal of the organic solvent by rotoevaporation under reduced pressure. Afterwards, the resulting dendrisome suspension is bath-sonicated at 65° C. for 2 hours.

### Characterization of Dendrisomes

Transmission Electron Microscopy (TEM). A drop of the dendrisome suspension (4 mg/ml) is placed onto a grid with a support film of Formvar/carbon, previously glow discharged in an Emitech glow discharger unit. Excess material is blotted off with a 50 hardened filter paper and negatively stained with 1% uranyl acetate prior to viewing with, for example, a Philips CM 120 (Einhoven, The Netherlands) Bio Twin transmission electron microscope using a lab 6 emitter and 120 kV. Images are captured on, for example, Kodak SO-163 negative film and printed on Ilford multigrade paper, so the appearance of dendrisomes can be evaluated and their diameter accurately measured.

Measurement of Dendrisome Diameter and Zeta Potential. The hydrodynamic Z-average diameter and the zeta potential of all dendrisome preparations in deionized water are measured by photon correlation spectroscopy (PCS) using, for example, a Zetasizer 3000 (Malvern Instruments, Malvern, UK, He—Ne laser), with a 90° angle of measurement. The average of three measurements are typically used, and the results expressed as Z-average (nm) f SD and zeta potential (mV) f SD.

### Prospective Experimental Results

Dendron Self-Assembly and Dendrisome Formation. The lipid-modified cationic dendron self-assembles into dendrisomes (vesicular structures), with a typical Z-average hydrodynamic diameter of 300 f 8 nm. The zeta-potential of the dendrisomes is positive, +54.5 mV and TEM micrographs show membranes of 6.6-10-nm in thickness (data not shown). Bilayer formation is most likely, with the hydrophilic polylysine head directed towards the aqueous phase and the hydrophobic alkyl chains associating with the hydrophobic regions of neighboring dendrons, as shown in FIG. 11B. The diameter of the dendrisomes, d, is determined from the molecular area found during the monolayer studies. The area per molecule is calculated by extrapolation of surface pressure to zero pressure (400 A°²) (400 A°²=πr², d=2r=2.26 nm). The length of an alkyl chain, L, based on, for example, CoreyPauling-Koltun (CPK) molecular modelling, should be approximately 2.2 nm. The polylysine head may make the membrane bulkier and thicker compared to phospholipid bilayer membranes, whose thickness is generally around 5 nm. TEM also typically shows a population of smaller size (<100 nm) structures compared to the 300 nm Z-average hydrodynamic diameter measured by PCS (data not shown). In PCS, the hydrodynamic diameter obtained is an intensity mean size. The intensity of light scattered is proportional to d⁶ (d is the particle diameter, from the Rayleigh approximation), so the contribution of the light scattered from small particles to calculation of mean size is minimal compared to that of large particles.

Cholesterol is found to have an effect on the morphology and size, but not on the charge of the dendrisomes. TABLE 1 shows dendrisomes in the absence of cholesterol to be smaller and more uniform than dendrisomes with dendron/CHOL molar ratios 1:7. On the other hand, the effect of cholesterol incorporation is less significant on the zeta potential, which varies from 54.9±4 mV for cholesterol-free dendrisomes to 52.3±3 mV for dendrisomes with the highest cholesterol content.

TABLE 1
Prospective experimental data showing the Z-average Size and
zeta-potential with different cholesterol ratios.

| Dendron/CHOL | Z-Average Size | Polydispersity | Zeta-potential |
|---|---|---|---|
| Molar Ratios | (nm) ± SD^{a} | Index | ( ) ± SD^{a} |
| 1:0 | 309 ± 7 | 0.235 | 54.9 ± 4 |
| 1:1 | 371 ± 4 | 0.310 | 50.8 ± 3 |
| 1:5 | 402 ± 7 | 0.521 | 51.6 ± 4 |
| 1:7 | 561 ± 14 | 0.710 | 52.3 ± 3 |

^{a}Mean ± SD; n = 3.

### Example 9

### Prospective Example: Encapsulation Efficiency of Radiolabeled Oligonucleotide)

This prospective example demonstrates the encapsulation of the radio-labeled oligonucleotide, Vitravene® within dendrisomes formed from the amphipathic polypeptide dendron synthesized in Example 21, using a modified technique know in the art (Al-Jamal et al., 2005). Vitravene® is an FDA approved oligonucleotide used to treat cytomeglavirus invention retinitis in AIDS patients. Oligonucleotides are an emerging new class of therapeutics consisting of short nucleic acid chains that work by interfering with the processing of genetic information. Typically, they are unmodified or chemically modified single-stranded DNA or RNA molecules. They are relatively short (19-25 nucleotides) and hybridise to a unique sequence in the total pool of DNA or RNA targets present in cells. New technological advances in molecular biology have led to the identification of genes associated with major human diseases and to the determination of their genetic basis. And now, oligonucleotide technologies are providing a highly specific strategy for targeting a wide range of diseases at genetic level. In this example, the encapsulation and retention efficiency of an oligonucleotide, a 21-nucleotide phasphorothioate based product with the sequence 5′-G-C-G-T-T-T-G-C-T-C-T-T-C-T-T-C-T-T-G-C-G-3′, in dendrisomes is evaluated and compared to the encapsulation and retention efficiency of the same oligonucleotide in a conventional liposome formulation prepared by the same technique.

[³²P]-Oligonucleotide is reconstituted in doubly deionized water to make a final concentration of 0.9 μCi/50 μL. A mixture of cold (2 mg) and radiolabeled (5.7 μg or 0.9 μCi) oligonucleotide is dissolved in 5 ml water (drug to lipid percentage was 10%) and injected into the lipid solutions (dendron/CHOL molar ratios; 1:0, 1:5, 1:9) prepared as mentioned above. The suspension is ultracentrifuged in a Sorvall CombiPlus ultracentrifuge (Sorvall, Dupont, USA) at 42,000 rpm for 1 hour at 4° C., and washed to remove any unentrapped/non-interacting oligonucleotide. The pellets are suspended in 1 ml water for encapsulation efficiency and release studies. Radioactivity is measured in 10 μg of pellets suspension and supernatant. The weight of entrapped oligonucleotide is calculated accordingly (0.9 μCi is equivalent to 2 mg oligonucleotide). The percentage entrapment is calculated as the number of mg of oligonucleotide entrapped in 100 mg of total encapsulation material (total mass of dendron and cholesterol). Entrapment studies are carried out in triplicate, with the results expressed as percentage ±SD.

In vitro release of oligonucleotide from dendrisomes (dendron/CHOL molar ratios; 1:0, 1:5, 1:9) and the comparator DSPC:CHOL (1:1) liposome formulation is measured using a dialysis technique. One milliliter of oligo-containing dendrisome or liposome suspension is pipetted into the dialysis tubing, where the tubing has a molecular weight cutoff of 3500 Da, and then the tubing sealed. The dialysis tubing is placed in 250 ml of deionized water in a 300-ml conical flask with constant stirring at 25° C. At intervals over 48 hours, 1-ml samples are taken and replaced with water of the same temperature. Each 1 ml sample is then added to 4 ml Optiphase “Safe” scintillation cocktail for quantification (LS 6500 multipurpose scintillation counter, Beckman, USA). Release studies are carried out in triplicate, with the results expressed as percentage ±SD.

### Prospective Experimental Results

The influence of the negatively charged oligonucleotide on the morphology of drug-loaded dendrisomes, should be minimal. Neutral liposomes (hydrodynamic diameter 730±13.5 nm, polydispersity index 0.35, zeta potential—2 mV) are used as a comparator to avoid the complications of electrostatic interaction. Dendrisomes of different compositions are found to have different encapsulation efficiencies compared to a typical liposome prepared by the same technique. The encapsulation efficiency is directly proportional to the percentage of the dendron in the total encapsulation material (dendron+cholesterol). FIG. 12 illustrates that cholesterol-free dendrisomes have the maximum entrapment efficiency (6.15% w/w oligonucleotide in total lipid), although they are smaller in size. Lower entrapment efficiencies are achieved by decreasing the percentage of dendron, despite size increases. Percentage entrapments of 4.7 and 4.0 in the case of dendrisomes with 1:5 and 1:9 dendron/CHOL molar ratios are achieved, respectively, compared to 1.4% in neutral REV liposomes.

### Example 10

### Prospective Example: Exposure of Mammalian Cells and Calcein-Containing Nanocarriers (Dendrisomes) and Mammalian Cells to Controlled Ultrasonic Energy, and Evaluation of Cell Viability and Intracellular Calcein Delivery

This prospective example demonstrates the encapsulation of calcein within dendrisomes formed from the amphipathic polypeptide dendron synthesized in Example 21, followed by disruption of nanocarriers, calcein release, permeation of cellular membranes, and intracellular calcein delivery mediated by controlled ultrasonic energy. Calcein (623 Da, radius=0.6 nm), also known as fluorexon, fluorescein complex, is a fluorescent dye with an excitation and emission wavelengths of 495/515 nm, respectively. The acetomethoxy derivative of calcein (calcein AM) is used in biology, and in this experimental protocol, as it can be transported through the cellular membrane into live cells, which makes it useful for testing of cell viability and for short-term labeling of cells.

### Perspective Experimental Methods

Ultrasound. Ultrasonic energy is produced using an immersible, focused, piezoceramic transducer. In this experimental system, two different matching resistance networks are necessary, allowing production of sound at 1.0 MHz and 3.0 MHz, similar to a method known in the art (Guzmán et al., 2001a, 2001b, 2002, and 2003; Schlicher et al., 2006). A sinusoidal waveform is produced by, for example, programmable waveform generators (Stanford Research Instruments, Sunnyvale, Calif.) used in conjunction to control pulse length, frequency, and peak-to-peak voltage. The sinusoidal waveform is amplified by an RF broadband power amplifier (Electronic Navigation Industries, Rochester, N.Y.) before passing through a matching network and controlling the response of the transducer. The transducer is housed in a polycarbonate tank (FIG. 13 [701]—34.5×32×40 cm; containing approximately 34 liters of deionized, distilled, and partially degassed water at room temperature (22° C. to 23° C.). A thick acoustic absorber is mounted opposite the transducer to minimize standing-wave formation (not illustrated). A three-axis micropositioning system (10 μm resolution; Velmex, Bloomfield, N.Y.) is mounted on top of the tank to position samples and a hydrophone at desired locations in the tank. A PVDF membrane hydrophone (NTR Systems, Seattle, Wash.) is used to measure spatial-peak-temporal-peak negative pressure to map and calibrate the acoustic field produced by the transducer versus the peak-to-peak voltage signal provided by a function generator.

Cell Culture. Cell culture and preparation is performed by a previously published procedure (Guzman et al., 2001). Briefly, Henrietta Lacks (HeLa) cells are cultured as monolayers in a humidified atmosphere of 95% air and 5% CO₂ at 37° C. in RMPI-1640 medium, supplemented with 100 μg/ml penicillin-streptomycin and 10% (v/v) heat inactivated fetal bovine serum. Human aortic smooth muscle cells (AoSMC) are initiated from a cryopreserved stock and harvested at passage seven before each experiment. The cells are cultured as monolayers in a humidified atmosphere of 95% air and 5% CO₂ at 37° C. in MCDB-131 medium, supplemented with 100 ixg/ml penicillin-streptomycin and 10% (v/v) heat-inactivated fetal bovine serum. Both cell types are harvested by trypsin/EDTA digestion, washed, and re-suspended in pure RPMI for HeLa cells, and pure MCDB-131 for AoSMC cells.

Preparation of Nanocarriers (Dendrisomes) and Samples. Before Ultrasound Exposure, dendrisomes are prepared containing calcein (15 μM initial solution) by using a procedure of Example 22, Samples are prepared at a cell concentration of 10⁶ cells/ml, dendrisomes containing calcein, and the ultrasound contrast agent Optison® at concentrations of 0.30% v/v (˜1.6×10⁶ bubble/ml), 1.8% v/v (˜1.1×10⁷ bubble/ml), and 15.0% v/v (˜9.3×10⁷ bubble/ml). Optison® is an ultrasound contrast agent, a suspension of perfluorcarbon gas bubbles stabilized with denatured human albumin that is used to serve primarily as nuclei to promote acoustic cavitation activity in sonicated samples. The product name “Optison®” may hereafter be referred to as “contrast agent” or “ultrasound contrast agent.”

Before every experiment, the desired placement of the dendrisome and cell samples in the acoustic field is found using the PVDF membrane hydrophone FIG. 13 (704). This location is approximately 1 cm and 0.5 cm out of the ultrasound's focus toward the transducer (701) (for 1.1 and 3.1 MHz, respectively). The acoustic pressure is calibrated versus the peak-to-peak voltage of the signal created by the function generator, using the PVDF membrane hydrophone (704) at the desired location. These out-of-focus locations have a broader acoustic beam than at the focus, approximately 10.4- and 2.4-mm wide at half-amplitude (−6 dB) for 1.1 and 3.1 MHz, respectively. This broader acoustic energy wave is more favorable for the experimental system shown in FIG. 13; allowing a more uniform acoustic exposure across the sample chamber and reducing “dead zones,” where cells or contrast agents are not uniformly exposed to said acoustic energy (Schlicher et al., 2006). In addition to the broad exposure zone, vigorous mixing, probably caused by microstreaming and acoustic cavitation during ultrasound exposure, further enables a more uniform exposure of all nanocarriers and mammalian cells in the sample chamber.

Samples are placed within chambers (705) constructed from a cylindrical bulb of polyethylene with an approximate dimension of 1.4 cm in height and 0.6 cm in diameter. Sample solutions are slowly aliquoted into the sample chamber (705) with a syringe; making sure to fill the chamber completely; however, without the production of air bubbles. A metal rod (706) is immediately inserted into the open end of the sample chamber and then attached to the three-axis positioning system (703); placing the sample in the desired location as determined by the hydrophone (704). After sample placement, a computer program is initiated to record hydrophone (704) output, and the exposure is initiated by triggering the function generator with the desired setting.

Exposures are performed at a burst length of 1 ms, 1% duty cycle (i.e., 10 pulses per second); pressures of initiation sequences of 1.7 MPa or 2 MPa followed by sustaining pulses of 0.6 MPa, 0.8 MPa, or 1.0 MPa; total exposure times of 2, 10, 20, 100, 200, 1000 and 2000 ms; and frequencies of 1.1 and 3.1 MHz. Total exposure time is simply the amount of time a sample is exposed to ultrasound, calculated by multiplying the number of ultrasound bursts times the burst length. After ultrasound exposure, samples are immediately transferred into 1.5-ml microcentrifuge tubes and allowed to “rest” for 5 minutes at room temperature. Samples in microcentrifuge tubes are then placed on ice and allowed to incubate until all samples have been exposed (1 to 2 hours).

After all nanocarrier samples and cell suspensions have been exposed, sonicated nanocarriers and cells are washed with phosphate buffered saline (PBS) and centrifuged (900×g for 3 minutes) three times to remove non-ruptured nanocarriers, nanocarrier components, and extracellular calcein in the sample supernatant. The subsequent cell pellets are re-suspended in a final volume of PBS containing propidium iodide (2 mg/ml), a viability marker that stains nonviable cells with red fluorescence.

### Prospective Experimental Results

Measurement of cavitation activity. As described herein, cavitation activity can be measured and characterized by analyzing acoustic emissions from cell samples exposed to ultrasound. This technique is especially attractive for use with the present invention because it can be used noninvasively, and it provides a direct measure of inertial cavitation activity. However, a clear correlation between ultrasound-induced tissue effects and this methodology for measuring said cavitation must be established.

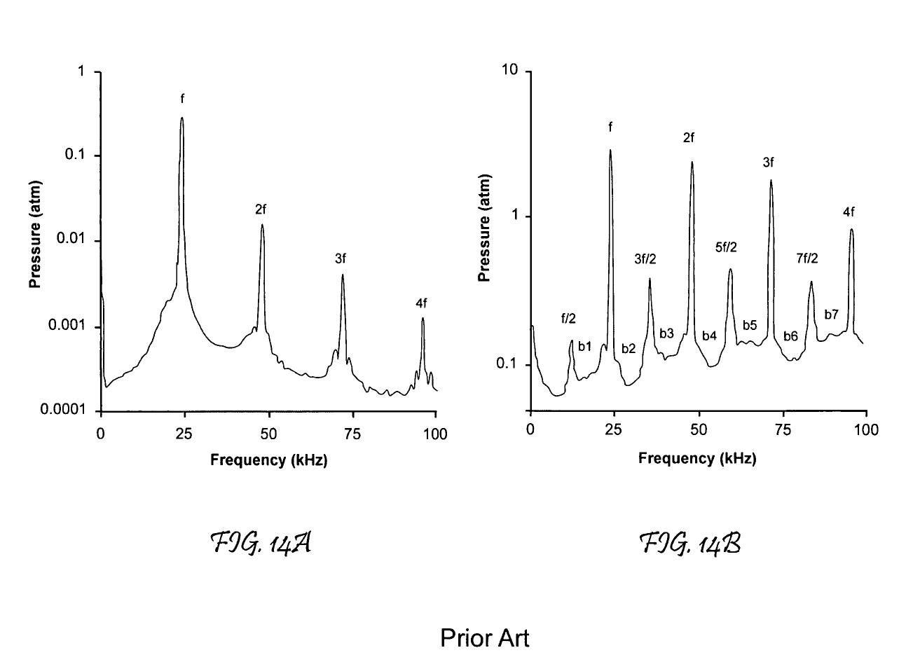

In the experiment system designed to evaluate these relationships (FIG. 13), the sound spectrum emitted by the sonicated samples is recorded with a hydrophone and later analyzed by Fast Fourier Transform (FFT) analysis to extract frequency spectra; FFT analysis is a powerful tool for analyzing and measuring signals from plug-in data acquisition (DAQ) devices. FIG. 14A-14B shows representative acoustic spectra measured during ultrasound exposures at low pressure (non cavitation; FIG. 14A) and during high pressure (extensive cavitation; FIG. 14B). As illustrated, when cavitation occurs, typically acoustic energy is shifted to a spectrum of other frequencies (FIG. 14B).

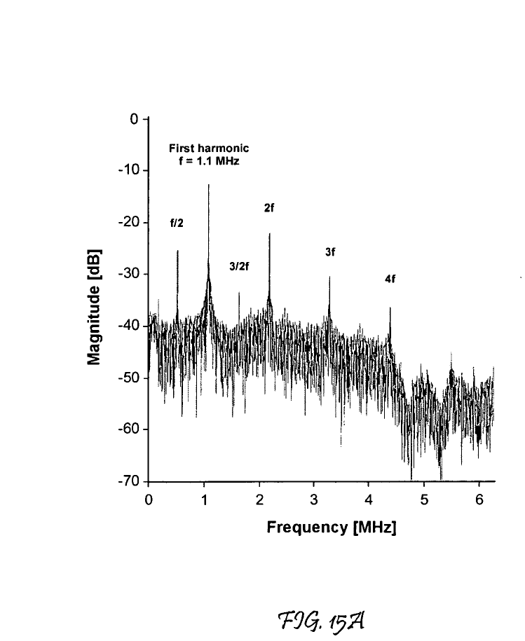

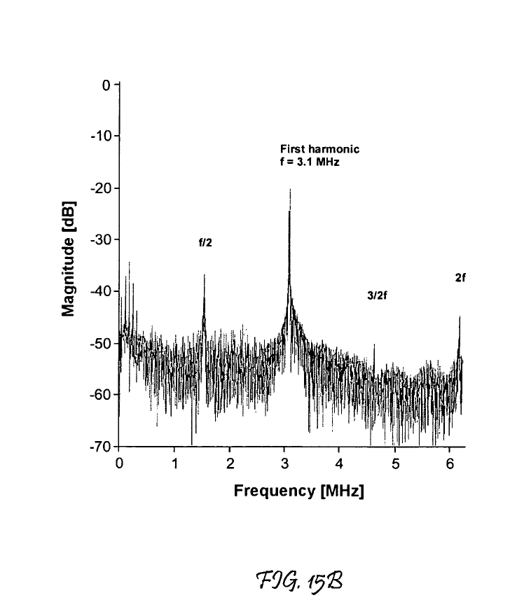

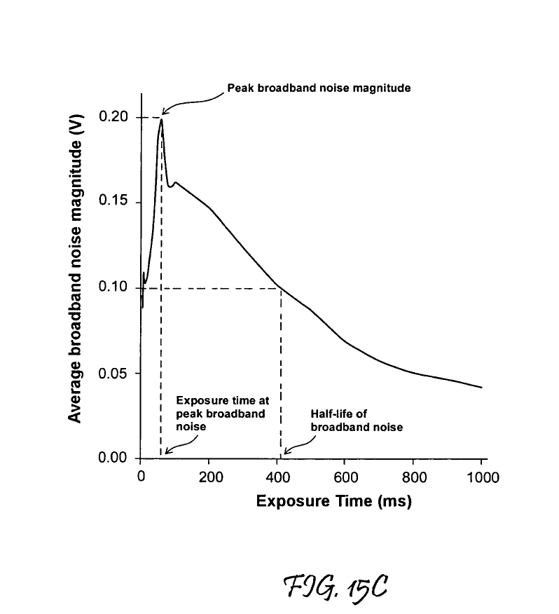

FIG. 15A-15B illustrate prospective frequency spectra when nanocarriers and cell suspensions are sonicated using the experimental system described herein (FIG. 13). Features of these acoustic spectra consist of a strong signal at the driving frequency (FIG. 15A −f=1.1 and FIG. 15B −f=3.1 MHz) as well as characteristic markers of cavitation, such as subharmonics (i.e., f/2), ultraharmonics (i.e., 3/2f, 2f, and high levels of broadband noise (FIG. 15C)

Harmonic, subharmonic, and ultraharmonic signals are commonly produced at the onset of cavitation (FIG. 14B). While not wishing to be bound my any particular theory, increases in background noise likely arise from stable cavitation bubbles oscillating linearly and nonlinearly, as well as erratic oscillations and bubble collapse (FIG. 15C) with the pressure transients of ultrasound (Neppiras, 1980; and Leighton, 1994). The present invention, described in detail in the next section of this specification, utilizes broadband noise measurements as a measure of inertial cavitation based on the expectation that observed increases in cellular permeability and viability result from primarily inertial bubble collapse (Miller et al., 1996).

When average broadband noise measurements are evaluated as a function of the ultrasound exposure time, frequency, pressure, and contrast agent concentration; the magnitude of said background noise should show a strong correlation with cavitation activity of the sonicated samples. Said cavitation activity is a transient response, increasing to a maximum within approximately 20 ms of ultrasound exposure time, and then decreasing to background values over tens to hundreds of milliseconds (prospective data not illustrated). The total cavitation activity in the sonicated samples, as measured by the time integral of broadband noise, should generally increase with increasing pressure, decreasing frequency, and increasing contrast agent concentration (prospective data not illustrated).

In addition, broadband noise is generated by both the initial rupture of stabilized gas bubbles added as nucleation sites, and the destruction or dissolution of secondary bubbles generated and recycled over time. Again, while not wishing to be bound by any particular theory, strong broadband noise during the first milliseconds is believed to represent the emission and scattering of sound by the initially large concentration of contrast agent bubbles. In most cases, broadband noise should increase during the first several bursts, which is believed to be caused by the initial collapse of contrast agent bubbles, and the resulting increase in secondary bubble collapses. This characteristic increase has been previously observed by monitoring cavitation emissions (Chen et al., 2002). The subsequent decrease in broadband noise is likely due to the loss of bubbles that are destroyed by inertial collapse or loss of stability, which reduces both sound scatter and emissions. Eventually, broadband noise decreases until it remains relatively constant near background levels; the point where inertial cavitation activity should no longer exist or be significant enough to induce changes in cell membrane permeability.

Dependence of Cell Permeability and Viability on Ultrasound and Experimental Parameters. The influence of a variety of ultrasound and experimental parameters on cell viability, cell membrane permeability, nanocarrier disruption and calcein release, as well as intracellular calcein delivery can also be determined with the experimental system illustrated in FIG. 13. Only some of the parameters that may be evaluated in this simple system include (1) the influence of ultrasonic pressure and frequency, (2) ultrasonic exposure time (3) ultrasound contrast agent concentration, and (4) characteristics of the biological system (i.e., cell type) where extracellular and intracellular drug delivery is sought.

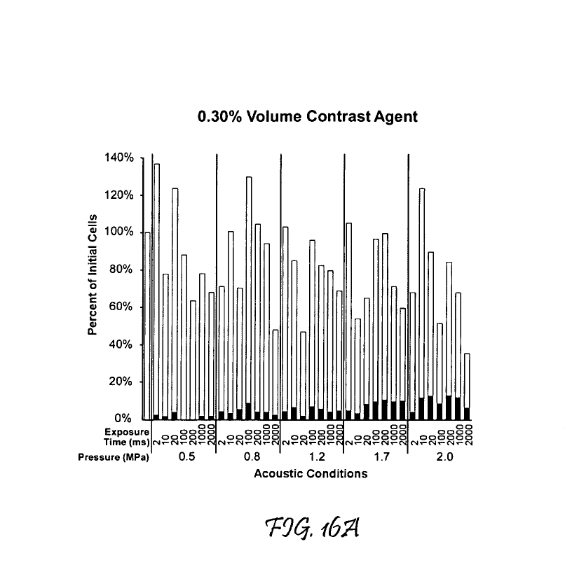

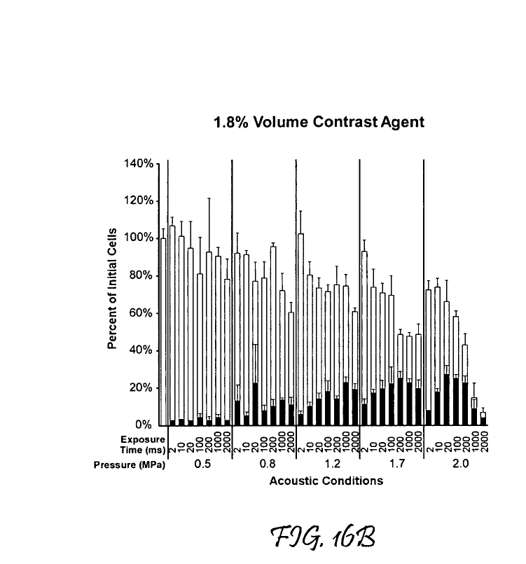

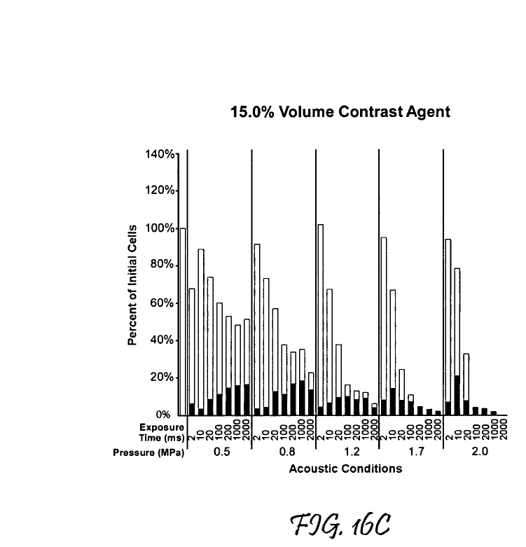

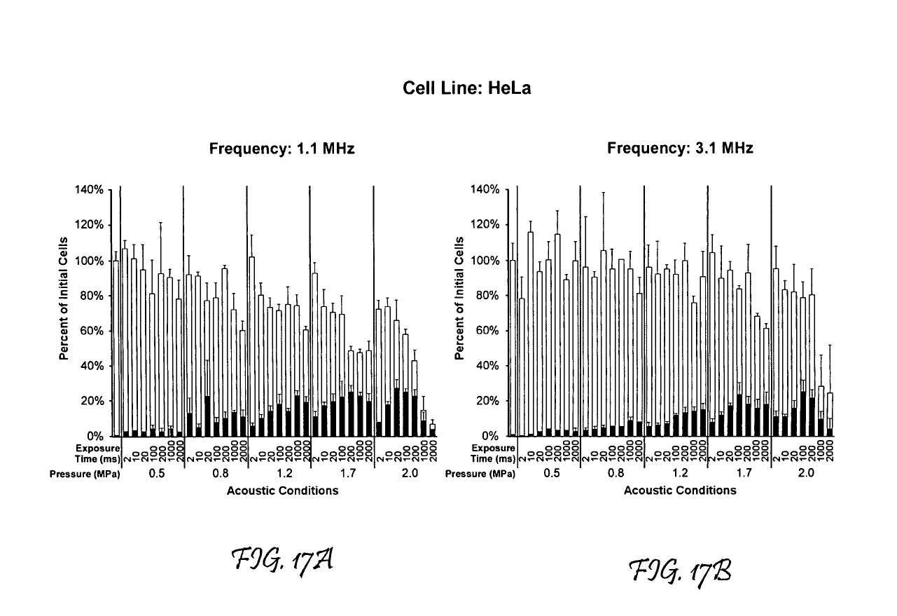

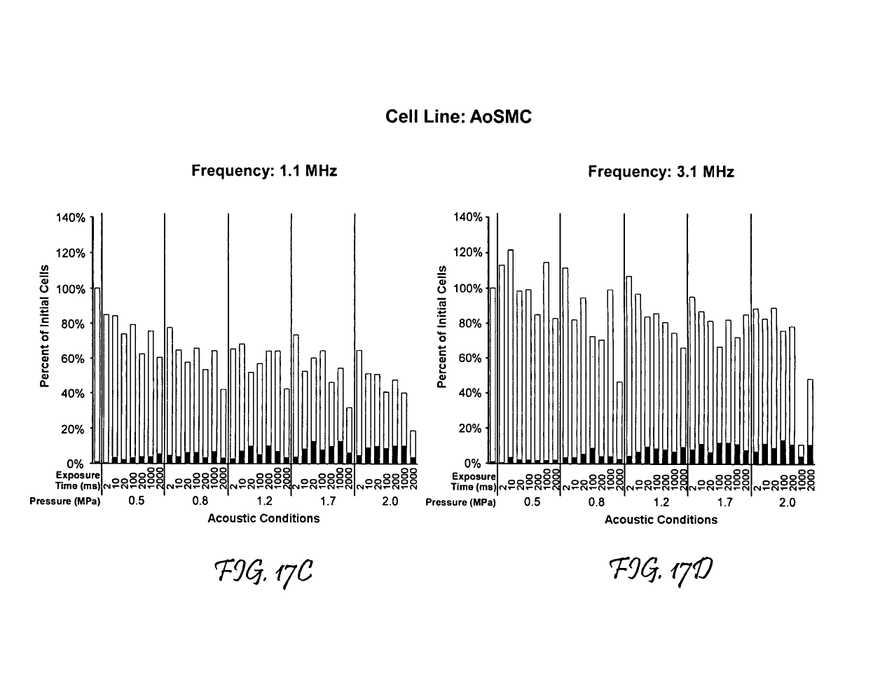

FIG. 16A-16C illustrates the effect of changing the concentration of contrast agent, i.e., nucleation sites for cavitation, over a range of pressures and exposure times. Further, the effects of frequency and cell type are shown in FIG. 17A-17D, wherein two different cell types, Henrietta Lacks (HeLa, FIG. 17A-17B) and aortic smooth muscle cells (AoSMC; FIG. 16C-16D), are sonicated at two different frequencies, 1.1 and 3.1 MHz, over the same range of pressures and exposure times. In FIG. 16A-16C, the total height of each bar represents the percent of cells remaining viable after sonication, which is subdivided into a black bar, representing the percent of viable cells with uptake. All samples are normalized to the control sample (i.e., “sham” sonication) taken to represent 100% viability and 0% uptake.

In FIG. 16 and FIG. 17, independently increasing pressure or exposure time increases the fraction of cells affected by insonation, increasing both the fraction of cells with uptake, and the fraction of cells killed. The interaction of pressure and exposure time also has a statistically synergistic effect. Increasing contrast agent concentration (FIG. 16A-16C) and decreasing frequency (FIG. 17A-17B) also increases the effects on biological materials, by increasing uptake and cell death. In general, greater uptake and lower levels of death are seen in HeLa cells compared with AoSMC cells (FIG. 17C-17D).

As with the present invention, a variety of conditions and parameters can be altered and evaluated that cumulatively result in mild to profound effects on nanocarrier rupture, calcein release, and on the alteration of insonated biological materials. These include (1) mild conditions that cause low levels of uptake calcein uptake and almost no cell death, (2) moderate conditions that cause uptake into as many as one-third of cells and some cell death, to (3) strong conditions that kill almost all insonated cells. While not wishing to be bound by any particular theory,

The studies conducted in this relatively simple experimental system (FIG. 13) do have limitations. For example, the cell sample is exposed to a nonuniform acoustic field in said system. Acoustic scattering in the direction of the ultrasound beam by high concentrations of contrast agent can cause significant attenuation, which can approach 100% during the initial bursts of ultrasound (data not shown). Given these nonuniformities, only a fraction of the sample volume is exposed to a pressure above the threshold for to, for example, inertial cavitation. Therefore, the size of any “cavitation zone” is expected to depend primarily on ultrasound pressure, frequency, and contrast agent concentration.

A far better testing environment would be to apply similar methods and techniques, but have cells or tissue samples [FIG. 13, (706)] suspended inside one of several different commercially available ultrasound phantoms. An especially valuable apparatus is the CIRS series of ultrasound phantoms manufactured by CIRS, Tissue Simulation and Phantom Technology (Norfolk, Va.). Unlike human subjects or random scannable materials, this device offers a reliable medium which contains specific, known test objects for repeatable quantitative assessment of ultrasound performance over time. The phantom is constructed from the patented solid elastic material, Zerdine (see U.S. Pat. No. 5,196,343, the disclosures of which are hereby incorporated herein by reference in their entirety for all purposes). Zerdine, unlike other phantom materials on the market, is not affected by changes in temperature, and can be subjected to boiling or freezing conditions without sustaining significant damage, and should be suitable for use with therapeutic applications of HIFU. At normal or room temperatures, the Zerdine material found in the Model 040 accurately simulates the ultrasound characteristics found in human tissue. Thus, this type of phantom should be ideal for use with a similar, but modified type of experimental system with characteristics similar to the experimental system illustrated in FIG. 13.

While a variety of materials, methods, and systems for use in acoustically mediated drug delivery have been described with reference to specific embodiments, it will be understood by those skilled in the art that materials, methods, and systems may be used by the present invention and that various, sometime significant changes may be made and equivalents may be substituted for elements thereof without departing from the true spirit and scope of the invention. In addition, modifications may be made without departing from the essential teachings of the invention.

I claim:

1. A method suitable for the controlled intracellular and extracellular delivery of one or more therapeutic compounds to a region of a patient, the method comprising the acts (steps) of
(a) administering to the patient one or more therapeutics;
(b) administering to the patient one or more contrast agents; wherein said contrast agents may be the same as or different from one another; where steps (a) and (b) are performed
(i) in any order, or
(ii) simultaneously;
(c) alteration of the permeability or structural integrity of said region of said patient comprising
(i) administering to the patient acoustic energy at one or more frequencies, inducing acoustic cavitation at said region of said patient;
(ii) measuring the level of acoustic cavitation at said region of said patient by measuring acoustic emissions either (1) alone; (2) possibly in combination with one or more additional properties directly or indirectly related to the level of acoustic cavitation at said region of said patient; and (3) possibly in combination with one or more properties of said acoustic energy; at the time of or subsequent to the initial application of said acoustic energy;
(d) utilizing the measurement(s) obtained in act (step) (c) to modify continued or subsequent application of acoustic energy to said region of said patient, and possibly administering to said patient one or more additional contrast agents, therapeutics, and other compounds; wherein said contrast agents, therapeutics, or other compounds may be the same as or different from one another; where said acoustic energy is applied at a level below the threshold level for lethal sonolysis or cytotoxicity;
(e) allowing said therapeutic compounds to traverse said disrupted cellular membranes and/or other internal structures of said patient, in said region; and
(f) possibly repeating acts (steps) (a) through (e), in whole or in part, either independently or in any combination, one or more times.

2. The method as defined in claim 1, wherein said one or more contrast agents are targeted contrast agents.

3. The method as defined in claim 1, wherein said acoustic emissions include measuring the broadband signal of the spectrum.

4. The method as defined in claim 1, wherein said one or more properties of said acoustic energy measured in act (step) (c) of claim 1 is selected from the group consisting of microbubble backscatter, microbubble backscatter speckle reduction, changes in microbubble backscatter speckle statistics, shear wave propagation changes, electrical impedance tomography, and combination thereof.

5. The method as defined in claim 1, wherein said one or more properties of said acoustic energy measured in act (step) (c) of claim 1 is selected from the group consisting of pressure at one or more frequencies, energy input at one or more frequencies, pulse sequence repetition frequency, pulse sequence content, pulse sequence length, pulse sequence period, duty cycle, and the length of time said acoustic energy is administered.

6. The method as defined in claim 1, wherein said therapeutic compound is contained within or attached to or embedded within a vesicle.

7. The vesicle according to claim 6, wherein said vesicle is a nanocarrier.

8. The nanocarrier according to claim 7, wherein said nanocarrier is comprised substantially of biodegradable triblock copolymers or mixtures thereof.

9. The nanocarrier according to claim 7, wherein said nanocarrier is comprised substantially of branched-chain polymers or mixtures thereof.

10. The nanocarrier according to claim 7, wherein said nanocarrier is comprised substantially of dendritic polymers or mixtures thereof.

11. The nanocarrier according to claim 7, wherein said nanocarrier is comprised substantially of polymersomes or mixtures thereof.

12. The nanocarrier according to claim 7, wherein said nanocarrier is comprised substantially of peptosomes or mixtures thereof.

13. The nanocarrier according to claim 7, wherein said nanocarrier is comprised substantially of supramolecular assemblies or mixtures thereof.

14. The nanocarrier according to claim 7, wherein said nanocarrier is comprised substantially of materials selected from the group consisting of biodegradable triblock polymers, dendritic polymers, polymersomes, peptosomes, supramolecular assemblies, mixtures thereof, and combinations thereof.

15. The method as defined in claim 1, wherein said therapeutic compound is contained within or attached to or embedded within a polymer matrix, such as a hydrogel.

16. The method as defined in claim 6, wherein said vesicle is contained within or attached to or embedded within a polymer matrix, such as a hydrogel.

17. A system for administering acoustic energy to a region of the patient for use in cavitation-mediated ultrasonic drug delivery comprising
(a) one or more transducers with each having one or more array of elements;
(b) a transmitter connected with the transducers, the transmitter operable to both generate an imaging transmission of acoustic energy from one or more arrays, and to generate a therapeutic transmission of pulsed and continuous acoustic energy from one or more arrays; wherein said therapeutic transmission is below the threshold level of lethal sonolysis or cytotoxicity;
(c) a broadband spectrum analyzer;
(d) possibly one or more geometric (3-axis) positioning systems;
(e) a computer controlled data collection and analyzing system for evaluating information and measurements obtained in act (step) (c), (d), (e), and (f) of claim 1; and
(f) a display or monitor operable to display an image representative of the imaging transmission and one or more characteristics of the data collected and analyzed in (e).

18. The method as defined in claim 1, wherein said acoustic energy is administered to said patient by the system of claim 17.

19. The method as defined in claim 1, wherein said acoustic energy is applied at a frequency between 1 kHz and 10 MHz.

20. The method as defined in claim 18, wherein said acoustic energy is applied at a frequency between 1 kHz and 10 MHz.

21. The method as defined in claim 1, further comprising administering an agent to said patient to enhance diffusion or transport of said therapeutic compounds through said disrupted cellular membranes and/or other internal structures of said patient, in said region.

22. The method as defined in claim 18, further comprising administering an agent to said patient to enhance diffusion or transport of said therapeutic compounds through said disrupted cellular membranes and/or other internal structures of said patient, in said region.

23. The method as defined in claim 1, wherein said acoustic emissions are measured at one or more frequencies other than the frequency or frequencies at which the acoustic energy is applied.

24. The method as defined in claim 18, wherein said acoustic emissions are measured at one or more frequencies other than the frequency or frequencies at which the acoustic energy is applied.

25. The method as defined in claim 1, wherein said acoustic emissions are measured at a frequency, or frequencies corresponding to integer multiples of one-half or one-fourth of the frequency applied.

26. The method as defined in claim 18, wherein said acoustic emissions are measured at a frequency, or frequencies corresponding to integer multiples of one-half or one-fourth of the frequency applied.

27. The method as defined in claim 1, wherein said acoustic emissions are measured at one or more frequencies which do not correspond to peaks in the broadband acoustic spectrum.

28. The method as defined in claim 18, wherein said acoustic emissions are measured at one or more frequencies which do not correspond to peaks in the broadband acoustic spectrum.

29. The method as defined in claim 1, wherein the information and said measurements obtained in claim 1 are analyzed using a mathematical algorithm, such as Fourier Transform or the Fast Fourier Transform.

30. The method as defined in claim 18, wherein the information and said measurements obtained in claim 1 are analyzed using a mathematical algorithm, such as Fourier Transform or the Fast Fourier Transform.

31. The method as defined in claim 1, wherein the application of the acoustic energy is modified in act (step) (d) by changing an acoustic parameter or acoustic energy pulse characteristic selected from the group consisting of pressure, energy, frequency, pulse sequence repetition frequency, pulse sequence content, pulse sequence length, pulse sequence period, total exposure time, duty cycle, and combinations thereof.

32. The method as defined in claim 18, wherein the application of the acoustic energy is modified in act (step) (d) by changing an acoustic parameter or acoustic energy pulse characteristic selected from the group consisting of pressure, energy, frequency, pulse sequence repetition frequency, pulse sequence content, pulse sequence length, pulse sequence period, total exposure time, duty cycle, and combinations thereof.

33. The method as defined in claim 1, wherein the application of the acoustic energy is modified in act (step) (d) by changing an acoustic parameter selected from the group consisting of temperature, fluid gas content, administration rate of molecules to be transported, sample collection rate, device position, and combinations thereof.

34. The method as defined in claim 18, wherein the application of the acoustic energy is modified in act (step) (d) by changing an acoustic parameter selected from the group consisting of temperature, fluid gas content, administration rate of molecules to be transported, sample collection rate, device position, and combinations thereof.

35. The method as defined in claim 1, wherein the application of said acoustic energy is modified by interrupting the application.

36. The method as defined in claim 18, wherein the application of said acoustic energy is modified by interrupting the application.

37. The method as defined in claim 1, wherein the transmitter of said ultrasound system is operable to generate a therapeutic transmission with a single frequency.

38. The method as defined in claim 18, wherein the transmitter of said ultrasound system is operable to generate a therapeutic transmission with a single frequency.

39. The method as defined in claim 1, wherein the transmitter of said system is operable to generate a therapeutic transmission with dual frequencies.

40. The method as defined in claim 18, wherein the transmitter of said system is operable to generate a therapeutic transmission with dual frequencies.

41. The method as defined in claim 1, wherein the transmitter of said system is operable to generate a therapeutic transmission with multiple frequencies.

42. The method as defined in claim 18, wherein the transmitter of said system is operable to generate a therapeutic transmission with multiple frequencies.

43. The method as defined in claim 1, wherein said acoustic energy is composed of cavitation initiating and sustaining sequences.

44. The method as defined in claim 18, wherein said acoustic energy is composed of cavitation initiating and sustaining sequences.

45. The method as defined in claim 1, wherein said therapeutic ultrasound is applied externally to said patient.

46. The method as defined in claim 18, wherein said therapeutic ultrasound is applied externally to said patient.

47. The method as defined in claim 1, wherein said therapeutic ultrasound is applied endoscopically to said patient.

48. The method as defined in claim 18, wherein said therapeutic ultrasound is applied endoscopically to said patient.

49. The method as defined in claim 1, wherein at least one of said therapeutics is administered intravenously.

50. The method as defined in claim 18, wherein at least one of said therapeutics is administered intravenously.

51. The method as defined in claim 1, wherein at least one of said contrast agents is administered intravenously.

52. The method as defined in claim 18, wherein at least one of said contrast agents is administered intravenously.

53. The method as defined in claim 6, wherein said vesicle is administered intravenously.

54. The vesicle according to claim 6, wherein at least one targeting moiety is associated with said vesicle.

55. The targeting moiety according to claim 55, wherein said targeting moiety is comprised of at least one component useful in magnetically targeting said vesicle.
